SEQUENCE LISTING (1 sequence)

SEQ ID NO: 1 - DNA, 218948 bp, Bacillus thuringiensis
ggagcagggagagaaagggacgaaaagaaaactatttcgcccatatacgttcgtcgattgcccgatgatgcgtgcgaacgcaatcatataaagcatcaagcgtgcgaatgcgtttcgaatgaaagcaatgcgacgcaaaatgaagatgccatatatacagggacgacgaaccataactggtagagcgtaccagttgtacaagaacgacgctgggtggtgaacggctcgctctgctcgccgtcgtaggtcactgcgtttccccacaacacccttcctgcgaatacgccggaggcggggtatcacacaagcagaacctctattgactgcgaacacggtctgaatgtacgaccgtactacgctccacatcttaaccatgcatctcttatttcataaccggttggctgacgttcattgttgctaacgaacaactcacgaaattcgaccaaccacgatgcatctactgttccgctgtatcttgtcactagagtattttccttacgctccgcaggactcacattctcctgcttcggtttattatgcgtctgtactcgcttacgctcctcttgttttactcgtacggatgctggcatcttacgtactcggactaacgcttttactatgcgtgcaatgtcaccatgcactccattactgaatggcttaaatgaatcaatcgctgactgtatcatcattggtttcataggtttcccatccacctttatatatggtcgtgcatcagatggattccatccaatcttaactgttactcccttttgtttgaagacaacgcccagctcgttgctacgcacatactcaaatactttcgtgtacgctggtgctgtcttttcttccggctcgctgtcgctcgccgaatcatatcctagttcctctgctacatagaagtcattcagacactctatgcaaatgtgtccatcatcctctgtgtaacataggtgacgagactcctcattgcagagttcacattgctcgctgacgctcgcaagttttacagtcatcactttataactacgaccaagtttctttgttttccaatccatccataatacatatctctcttcattcactactgttaaattcatcattttaaattcctccaatttatatgttttttgtttaaagccggggactcattagtggtagcctgttcagggcatctcagacttcaccaatcgtgtccatgaagttgctaggtcaactgacactgactgaacaactccgtagagattgttttcacatgtgcgtgcgttatgtcttctagtcatgacgtactctccctcgaccaagagttagagtcggcgtcctagcttcttgcacttccctcgaaagggtggctatccactaggggcgtcctccattcttgttagtacggagagcagcaatgacatacatgatgtcacgcaatgaggactcgtgttgtcttaacccgaagcaatctgtgtcctcactgcttcgggatgttagctggttgctaagtttcctagtaccatgtttgtccgctaccctatcgcgctatcactgattacggagtgaccatccgggatactgaataaatgctacatatatctcacgacatactgcattgcctgattcctcaacagatctcttgtttataccgacgttgattcagacgtcgaggcatacttgtgaaacggtggatgcttaccgtgcctgataggtcgctactcgtctactgttatgagtaacaacgatgttaggtctgatgattctacgagggcaaccaaccctcctctctggtgtagttatagttgacaccataagagacacgatatgctcatgtctcctagctaccaactatattactgacgacctaacaacatagcttctaactgtgtatttaacgcttcttcatctcctgtctcatggaatagtgccatcgcatcacacaatggtgttgatactaacttctctgctgtcattcttaatacgccgttgtcatcgaagtctacatcagctacttgttcatgattgttgtacggccataacactaagtcttttgatacgaagccactgttcatcatgttctcttttgtatatacggtgtatcctctcttacgtaatgtatcagcaacgatttccacactgcctttatactcaacacgacctgtatataactcttcaatgaataaggaatgatcttcaccaatgcggattaagaatctaccaccccatctgttttcaccttcatcgaaatagccaacgattgtcgcaccggatacattacccaacacaccgatgttgttgtcattcaccttacctagttcccacttcatacatgacgtccactccttatctgttgatacacgtagtagatcatccttcataccactgaacttcacatgacctgtcttcttcgtattacgtgtttctacaatgtgttttactttgcctaatggaatacgaatgtcatcacccaatatttccatcacttgcggaaactctttttcaattctgttgatatacttcacgacgctgatctttctgccatcatctagttgtaccttgttctccatgaaatcatcctcatctaatcgttgtaatcgtaatagtctcatttggaatattactgtgtgtttgtgtaatggattctcataatcgacacgcaatgcacttgatacatctcgcaatgctgaatggaacatctcatgtgagtgattgattaactcaatgtcgaatgaatctacaagcaatggatgtgctgctacttgtttgtgattcgctatcttcatcatgtgattgattaatactcttggattgttatctaatcgtgctaagtaatacatatcaaccatcatcacattaccgatgtcgtacttacctgtgaatgtcatctctctatctacgtatgcatacttcttagttacatagttttgtagaacaccttgttcatatagatactcacaatagtctctgcttaacttgtattcatgtccgagtttgaatccatacttcttcgcacataatgatgctctgatataacctgttggttcgaacttgagtggacgaatgatacgttgtttgaatccaatctctgctggttctgtgataacaaccttgccgtcgtagtaacatgcagtgatgttaatgtcgaagtcctcttgtacatactgacgaatatcatctgtgttcaccatcatgatctctacacgatacttacctatcgtaccttttaatactgatacaattttactgatctcaatgacacgtactgattcatcatcatcttctactgtgatacttggacgttctctgattacctcgctgaacataaagcccttgcttaatacgtgatataatacctctgacttacatgcgtcggatgtagatacgaacacatcaatgtcattaggttctttacccatcaatgcatctaatacacatccacctgcgataatacctttgaaaccttttaccactttgtatacttctttaatacctgtacttgctgtcatgtttattctctccttttaatttaaataaggagccgagatatacccgactccgatgattgttattatttatttaatacttctgctactttcagtttccatacacctcttgttggcttaccttgtttacctaagtctctcatgtatgcttgatacccactgttgaacttactacctaccttcttgaacttgttcgctgcatatacttctgcaccaatctctttatgtgcttcgatgataagattagagattctttcatccattgctgattcatatactttaacttccttcacttcctcttcttcttcctgtggagtgataacaggctcttcttgttgtggcacgatacttacttgtccttgcatcataccttccacgataccacgcatgataccttgtactgcatctgctaattcctcttgtacaacttctcttactgttgctctgatctcctctttaaacactggcattacatcctctctaactgttttgatgattgtattctgtacgttgtttgcccacatttgtgttagtgtcccaatactatcacgagcgataacagcgaatatatcctctgttgatactggcttcttcttaatagttgatacgatttcttttgctacttccatttcagttttctgttttaacttacctgcacgaatgatttcttcatcacgcttgtttgcaccataacgtttattccatctgtctaaccctgattgtgtattcttcatagtcattagattgtatcctcctgtgtagatgctgtttcagccatcatcattatttgttcaaacataatctcgaatcttacttcacttcctccattaccttgtgcattcagtcgaataccatgatgtgcatacatttgtttaataatctttgcttcatttgtgctttttaatcttacgtttgttttgattcttgttacagtcaatctgtcgataccaagagataacatgtacgatacgcaatcttttaaataagccttagcttcttgttgttggtctaggattagcttttgtgctacgttgtgtgataataccccttcaatcattgagattgtttcatttgacaacttcattattccttatcccctttactaattagttcacttgttgcacgtaatgttcctgatgctaaatccactcctgcatgacctacttctccgcctagcttgataactgttctaccggcacgttctgttacattcaatacttctcttgttaagccgagtgcagattctaatacactgtttaataattgtgttcctctgtctacacctgcatgaccgtacttatctactacagcagctcctttcaatagcccttgtgcggcacttaatgttaaacctttgtacttcgttctgattaattcctgtacatcacctcccttcaatgcatgtaatttctcttccactgaacggtactctaatactagttcgttgtacttttgaggcttagtgatattcttgttcatctcttgttgaatatgacctagttgtttatctagcccatcaatgaccttttgcttatccccttctgttaataccaattcctgtactgcttcttctactacctgtatgctatcctctgctactgcctgtgctgttgcatctgttacttgttgcatcttctctgctgccatagccaatacttcttgttctttcttagctagtgcttgtttcgttaactctctatgctgtgctgttagttcttcctgcattgctttaacctcttcctctgtgtataccttaccttctgttgttgctgctacttgttctttagccatgttcattacctccatttgttgttgtattgcagtgaattgattgatgatctctttcagtatctctgtacgcatacccatgttgacgttagcacctaagttgttagcctgttgtactagtgataatcgttgactatgcattactgttactcgttgatatagcttgatgatgttctctttctttgttctatcaccttgatactctactaattgtttttctgctactaccttgatgtttaagcccatatccttacatagcgcccctgtaatacggttaatatcctttactgtcataccttcgattgtttttaagtctgtcataataattagctccttttataattagttgtttttgtttttattgattaactcacctagtgttggtgctagtttaataccttgtttctttttgactatgctagttgataccttaccgatacctgctttctccactgtgttaatgaatgtaactactgctctcttcttcatacctaactcctcctatgcaaataagtttttaatcttacctaatagactactgaatacaccctgcttaccttctcctggtggtaattcattgttgtcctttagtctcttgatgtagatgatacgtttctctaactcaggtactagatgatggatacctagtctgataccctctgctctcatgatgttaagcattagtaatacactctcctttccacactgttgtactgcatactcatcacacttaaactcttgctctgtcacctcgtgctgttctgttagatgtccagtaggatacatgatatgacctagttcatgagatactagtgcttgtcttacatactggttactgttgaatgatttatagtcatatacaataatcatgtaatctaagtagtatcctgttagttcagcgatttgttcattgtcatacactgtcatcattacagttaagatacgagtgtcgatgtggtgcttctcgatgtcctcaatggatagttgatgatgacttgctaagtcttgtggttgttcgatgatagcaacatgttctgtcttgccgttgatgtttacagtgaatacttctttcaccgattgtcttacttccattacttctgcttgcgttagtttgtctgtcatttgtcattcctccattagtttaggtgtagtcaaaaggggtggggcatgaaacaaccctatacccccattgctatatataagacgtggtactccccaaaaatatctgtaattttttcgcctatagggaatttccatatcgtaccagccgactcctcttctttagcatgttgtcgtcgtcggctgtagaatgttacaaatatgtgccgaactgttgtcagaagactgtatcctgtgctacgctaggtgcagggagataatcgaggaggactgtaacaatggatggagatactatcggtggagaagaatcgtttgtagaggttaaggtagcgatggatcacgaagagaagaagtcgttcatgtctcgtccgggactggatgcgaatggagtaagtaggtttgtattaccaaacggtggagatatgaatgtcatcgtccacggcagggtcgcacacgaggtagacgagattctattcgaagagaaattacctagtatgatgaagacgtcgaatgaagacggtacattagaagcgccgataaaaggttgtatgactacaatcttagatggctctcacgatcagatacgtgatgatattcgtcaaggcattatgcgtaccgaagaaacgattctcggtaaaccggactctatacttgtcgctgacttttgggtagaagaaactcaaagaggttatcgtgcatcattccgtgatgtgaagtttacgaaagtaaagaatgatttcaatcgtatggatatcgtattgacgaatcgtttcttctttgaagcgcaggacgttgtgtttacgccgtacaaacgtgatgaaactggatgtgtctccgtagcgttaagcaaggagacagaagaaagattaaagaggatggtgctaaatggctaacattcaattctttcgtaagatgttagatgaagacttaggtcatcctgagaagatcatcttgcctgatggcaaagaggttttatggaacgaggagagtcagacgatagacttagactcattacagccgtacatcggagagcaattggattactacgaagatggcgtgtataaatggtctgcgaagatcgaagtgattaagccgactaaagatggcgtgaacgcagttcacgttgatattaatagtattaaaaagaacgattaagtatatatggggtgatgacgatgactgagcaagacatcaatcgcatccgttatctctcgcaggaactcggattatttgacgatgaaattgcagagcagatggatgttcaccgagttacggtgaatcgtgtacgtatcaaacacaatataccccgcccaaatttaagtaatagagtagataaaaaacaagtttgtaaacgttgcggtcacgttgatctgataccacgacatagacgtagaaggaacttatgtgtactatgtaaatcggattcgttacaggagttgcaacagaagaaaagggactatatgagagactacaataaaattaaaaaacattccgcaggaatgttgcaaaatggtagttaaactaggtatagtagaagatgaggagcggtctatcctcattacataatgagagaagaataaagggcgactctagtctcgaacaaaccattgtggaagtgtcgaaggaactagtagtcgtcttttatgttatggagggataagttatgagccaagacatgaagtacaagccatctccaaagaaatgccctcctccattacctgttgcaccgaaagtacgcatcgtagaagatggaatgagcgcaactgctatggtaatcctttcgttatcaatacctttattgattatgttggttgccggcattcttgaggttataggaggataattatggaaatcgagaaagaagaaatcattgacgacgaagaagtatgggatgatgaagaagaacagaatgttcctgtcctcgtcacaaaggtagacggtgttcagctccctatgggctggggtcaatccgcaacgaatatggacgaagtacgcaagcatagacagtcgtttaacttgaagcatggtatgtttgcaaacgtaccgatggtgtgtaaaggtgcagactgtccgatgcacgctatgtgtactgtccctctaaacaatcgtcctgtcggtggtcgttgtccgattgagatagcggcgatcattgatagatacgataagtattatgaggaacttggacttggccccgaagattactttgaccaatcacaggtaaaagaccttgtagatgtcgaagtaaaactcctacgtgcaaacggtcatctcgcaacgagtgctagtttcatagaacaagttgtcactgctgttgacgagaacggtaatgctcactaccgtccggaactaaacaaagcaacagagtacgaagagaaactgttatcacgtaagagtaagatactatctgacctaaacgctacacgtaaagcgaacaagtcagatactagcgtcaatgacccatcatcattcgcagcggacttaatgcgtcgtgcgcagaaagctcgacaagctacagttattgatgtcgatgatgagaatgtcgatgacgactcacaatatggtggtgaagaataatgggagcaaaaacaggtttcttaaaagggttagttagtaaccaagcaatcgagaatgtaccagtattcaataagttacttccgcaagggaacttaggtattcgagatggtggcgctcaaatcaaaggcgcatacgctggcgctcgcgccgacaaagctcgtatcaaaggatataagcaacgtgctgcggaacaagacaagaacttagcagaagcagttgcactcaatgaaaaaggtgtaacgagtgacgacatgttagcgatgcaacgaactgctttcgatgcgaagaaagaacgttacttacaacgtgtaaaagataacgaaaatcgtttaacaaacctagcagaagaccgtggcggtatggtatctagtggtatcgctggtatggcaaaaggtgtcggcggttacttcgctgcttctgatatgcgtggtatgagtggtcgtgcgatgacggcggcagttcgtacaggtacagccggtgctgcttatatgggagcaaatgcaggattacgttacttgtccggcggaggcttaacatacaacaatcagggagagcgtgacatcgcaggtatcccattcatttaagaggtgattccatatgataatgtgggcaataaacaaaactatggaagccgctatgaagaccgcttggaaaccagtacgtagtttctacgatggtgcaagtggaacgaagaagtttttgaacgaagcaggggaacaggtaaaactgaaccctgctcaaaaacttgcacagcggttgaagaatgatgtaaaacaaatcgggtcagggaaacgtcgtgacctaaaagggaatatcattgaggaagacttaaatgcattcgacagacatgtaaccaaccgcattggcggagcattacacttagtcggtgatgctacggtaggtacactagctactggtctaggctggggcgctcgtacaattggaggcggagctttagcgatgggagcaatggcatttagacctacgatgaaaggtgtcggtcatgtaaccaaacagtcgattgatttagttggtgatacagcgttaggtgctgctgatcttgttcatcaaatgaataagtctacgacaggacgtaacttactattcggcgcagggtttgctggtgtcgcaggaacaagtgcattcaattcgaatatgctcgctcctgagaactatcgtggaggagaaagtgcaatcaaactcgcaatgaagaacatgtacgacggagagaagttagatgcccttccgggtacgataggggatacgcaagtcgtacaacgtaatccactagacaatatgggtgcagatggagacttagtgtttgctatgcacaatatgaggtgatgtaagtgggagcaattaaaagtttcgtatcaggtatgagaggtgcgggaaaaggtagaggagcttctaacatcggtgttatggcaatcggaggagccggtcttgatggtgtaaccacatacgcaatggaacgtgcagagaatcctgacgaatccaaactatgggctggaacaaaagcagtcggtacggcggcggcatggttattcgctgaaccgttaatgtggggtattacacttggagaaggtgcggccgctcttggaggcatggcacttgatgaagcagaacgaaatcgtaaacaacgtaacgaaattaatatgcacgtacgagaaggacgaagtggtgaaatgggaggaacactcggtgggaacttccaagattcacaacaagcggcgacgatgcgtcagagacagatggatttattacgtcagcatcgtatgtctaccgaagctgtactcggtagcgaggcacgacaactacataggtaggtgaggagtaatgtgggggccaacagcagttgaactaggagaaaaagagtacaatagtttcagcatgagttctatcacatccaatgatactgtacagaatttaggtatttatgcaggtcttatggcgggtgcaggtgcagtcggattagcatacggcgttgaagcgtcgagtcctgtgtatgcagaacaacgacgacaaaatcaacaaagaatgttagaagcacgaaatacggcggcacttcctgcgcctgtagccgaagaaccaaaagatagtttaatagcaaaggcagtagcgtggaatgacaatcgtagggagcaacgtgaaattaaccaagcaaagaaacgtatggagaaaaacatgagtggtattaacacggatgcgctccttaacgtagatgagtcaagatacgctgatgacctagctagattaaatcaacaggcagatgaggcattaagacaagaaaaacactactactctaaaaagtctgttcaacaactacaagcgaagaatcgtaatgcactcaagaatctaagagcgattacgagagctttatgaaataggtgtcggctatgaaaatactacaaaacgtttcagatgaagtattatcaccttccgagcgtcagctatgggagacagagtatgatgatctaattgtacgaacgaagaagtgggcgagaagtacaggtgagaaattcaccgaagaagaacttcactatttagcgattctagataaaccgaagttttgggctgccgaaacattgaaatggttctgtcgtgactatcaagaaccaatgctacaagagatggcagacagtaaacgtacggtactacgtcttggtcgtcgtcttggtaaaaccgagacgatgtgtatcatgattctatggcacgcctttacgcaaccgaataaagggccgaataaccagtacgatattcttatcattgcgccgtatgaggaacaggtcgatctgatatttaaacgtctgtcccagcttattgatatgtcgggcgacgtgaatccttctcgtgacattgataaacatatcgaacttccaaacggaactgttattcatggtatcactgccggttctaaatcaggtagtggtgcggcgaatacccgtggacaacgtgccgacttaatcgtactcgatgagatggactacatgggtgagtctgagattaccaacatcatgaacattcgtaacgaagcgccggagcgtatcaagatgattgttgcttctacgccatccggtcgtcgtgattcttattacaaatggtgtgttggtgcgacaaagacatatgcacaagatgatgaactaacaagacaaaacggtggtcgtgttacatacaatgtgaagatgaagccgtggaagacacttgctgatggttcatacgagttaaaccaacttggtgacaagatgcgtgaaggtaacggttggacgacgatacatgcaccatctaccgttaaccctgagttactgaaagtcaacccggatacaggtttaacatacatcgaagagttacgtctagacttaacagagatgcgtttcatccaagaggttatggcggagtttggcgaaagtatctccggtgtattcttgaagaaacatatcgacattgccatctctcatggtgaacgattaaagctagactatgcgaagaagaaaactgaccgtgtgaagaaaggccctcgtattctcggcgtggactgggataaacgtaaagccgatacaaacatggtcggcgtagagtgggatccacaagaacagttatttattccgttcttccgtaaagcaatacctcgtggagaatttacatactccaatgcggtagatgccatcaaggaagcacactacgactttgattttgactacatgatgattgacgctggtcacggtgaatatcagattgaacaattacacatatgggctgacaagtatcagaaaggtttagcgaagcgaatcattcgtctgaacttatctgagaaagtaaaagtaatggatcctatcacgaagaagatggagagcaatcatattaagccattcatggtcaataatgccgttctcatgtttgagagggagatggttgcacttgctccgaaggatagagataccattgtacaatttgaatcatatgaggtaaaatcatggggtagcgatggacgacctacgtatacagatgagaacgagcatattttagactgtatcgttttcgctttgtatggatttacaaagtattatgatgatatactaaaggtaacgaaatcggcagcagttcgaacaattaaccaaccgcttgaccgtatgagaagtgacatcaaagatagggacatacagaaagaaacaaaggatgagaaacagacacgacgtgtagccgcagcattaggtattaagccgaatcgacaacgcagtacagagagtagaagaggatatacatctgtacgtcggtctatgggaatgccagcacggaagtccttctaagggcttcctatttacggggaagaaaacttccccatctaatggctaacatggtggctgtcctccaccatgattgactgcttttctctgataggctaggtggacacctcctcgatccatctagcccttttctatttaggagggtgaattatgtcaggaatgaaacaatgggaacgggagataattgaataccgtccgtatctcggacttcatcgtgatatattaagctcggttatgagtaggaaagatgaagacggacgtatcccgcctcccacacaagttgtaaagaaggcagcctcgcttcttggaattgatgtaattgaaaacattattgcatcactcaatcgtatcactcgtttactagaacaactggaaacagcactagccaatgacgaaacattcttggcgtacaagcaagcattagcagagaaggacattgtgaaggcacgagagatttatacagaccatctgtctaatcttcacgggaatcccgacttcgatgcgtatccgattctattagatgcggcaacagagattgagaattatctcaccttcttaaacggagaactgtacggtggtaaagccgacttcggcaacatatcaagtgtacgctcacaagaagaagatgaggtactaacgctggtgcagtttgaatccgaagacttgcaaccgatattgcttgagaatctaacagacgatcaacgacagttaatcgagaagattatatggcagaacaacggcggtgcgcagacagttgaagagtatatcgagatgctgacgaaagagaagtatgaaagcggaagtcctcgcattaactacaacgaagtggcgatccgtacgcaaatcattgcaacagcaaatgagaagtcagagttcttcaagaagattgcggacaatacagatgcgtatgtaggaaaacgaatggaagaacatctcggcggtgatatgaaaggttctctatcgtacctcgtgaacatagaaacatctctagaggatattaaaggttcactggcgattggattcaatcatagtgccgacaagtcaaaagaagcatacgtcaatcaggttcgtttaacgaatcaatcggtgcgtgagttcttagacatgaagatgagtcagactcgtaagaggaagcaggacttatccaataaggttaagacattctataagccggatgattatcctgataagggtgcgcagtatttaatgagtgccattgctgacggcgtagaatccgttaacggtgcatggacgatgatactcactgagaatatcggtgcaacgcatatggcggctgaccaaaacgatgtcatatttgacaacctacataagaagcgtcgtaatcaagaactctatagatacgtcgatgtcgtagataaagagtttgattacgagaacaaagagaaagagatgcagaacttcttgaactctcgtaacttacaatagacccacacaatatgtgtgtttctatataggggctaacccgctccatgttttactcgaaggagtgttgtatatgtcttacaacaaacttagcggcaaacaaattaaaccgggcgcacttgaccaaacgcatttatctccatctacgaaaatctctgagtctatgttaaacgtagactgggcggcgaaggcaaatgaagcattagcatctaagcttatcattgacttcgtacaagtagctggcgtaaaagcaaacggaaagagtgtcgatgtcacagcatctatctctgctccggctgctgatgcagataacaaaaaaggtgcagttgttcaagccggtaaaaacaaagttatccttcgtgatagcgtaacaggtgaccctgttatctctgctgaccatacagaggtatacggtaaacttggttacagtggctctgtattcacggtagcattcttctacaaagatgcatccaaagcagagaaagagtacacattcgcttctgagaaaacaatcgacttccagttcccacagcgattcgacttagcgacgattgtagaaacattcgcttctaacgagaagttcgttgatggtgcatctgatgtatcagctcgccttgatcttgagcaaatcgcaaaagatgctttcggcacaagctatacattagaccacgacggaacagcaaaccgtggaaagtctcttgtagaatctcttacagaaaagacacaaggtacattaaacacgcaggtaactgcatctgatttaattgacgaagttgtcactgcaagaggcggagaagatacgcttgaagacagattggtgaaactggctggtagcaatcaagaagtcattgatgctcgtaagtctacgacattaggagacaaggcgacacttgatgagcgactagaagcaggagagctgattgttgatactatcaagaaggacattacggcggcagttggtaatagtacattagcagcagtcatcagtgaccttcaaggtgcaatggaacaagcaggacattctcacttcgcagaagataagcaagtattagcgggcgacccattgatcgacacaaaacaatatattttacctgagggacgtttctacgctccgggtaataaatcattgtctgtttacatcaacggtcagttgcaaatggaaggtgtacattacactgaggtgaaggactcacaagaccctacgaaggggattggtgtcgatttcacgccggaagtaatcgtgcaagatatggtcatccagttacgctggtatagataaaatgattgctccgctaggattctagcggggtttttcatgttttcctccttgttttctatcgtaaagtcacggtatagtggaatcagagctatgtcaattctgacagtgtatgctgtcgaaaggaggagagagaaggatggaacctacattaacaagtgaaaaagctgttaagattaagtgttgtttctgcgaactaaaagatacttgtatccgccgtgcgcagaaggagaagtacgaagaatccggctggacgactcgctgtactgttacaccaaaccgtccgggagcaaagcgtaagagccgtaaaaagcgaaagaaagccttaaaatcgtaatgtcattattgttattcatagttacataggataagataacaatagaggaatgattgtcgaaccatatacatagctccatcactacgtaagagggaggtgggaatatgaactttatggctgacatgagtaatcttgtcgtgatgttacttcttgtggcatttctgacagagacatttgtggagtgcattaagaacttcgtgattcaatcgaagatgagcgcaaacgttactaacggtgttgctgcaatctttggtgtcatgatgtcatttgtactcaaggtgagtctattcacagatgagaatactgctgcttattacattggtatcgtcatctgcggattggtagcatctcgtggtgcgaactacgtacataactttgtaggtaacttaccgaagaaagacaaatagtttatagcctccggctcctcggccggggttatattcattgattcataggaggttatcaaatggcgagaaaagtgtggggtcgtggtttttggtcacgtatatctctgatgtggcgtgatgaagacgacgataaagaaccactagtcctcgaagaatccatgcagttaggtgaagctcccggagcgatgccaaaaggtggaggcggcgggggtagtgcaaagcgtgaccctaagatgtccctcgtcaaaagaatcggtctagctatcatggatggtggcggtggaggtcgagactttgaagaacccgaatttgatttcaatgaaattacaagtgcttataacacagaaggatatgtacgtcaggctgtcgataaatacatcgagatgatgtttaaagcagactgggatttcgtcagtaagaatccaaatgcggttgaatatatccgtatgcgttttacactgatggcagaagcaacgcagattcctacgaatcaactcttcatcgagattgcggaagaccttgtaaagtattgcaacgtcgttatcgcaaaatcacgagcgaaggatgccttgccgtttcagggtatgaacgtaatgggtgtaggtgacagtatgccagttgccggttacttcccgctgaacttagcttccatgaaagtgaagcgtgataagttcggtatgattaagggttggcaacaagagcaagagggtcaagacaaaccgcagaaattcaaacctgaggatattgtacatatttactacaaacgagaaaaaggtcgtgcttttggtacaccgtggttactacctgcactagatgatattcgtgctttacgtcaagtagaggagaatgtcttacgtctagtctaccgaaacttacatccattatggcatgtaaaggtcggactcgaacaagagggcttcggcgcagaagagggcgaagtagatttagttcgtggtgaggtagagaacatggatgttgaaggtgggatggtaacgacggagcgtgtaaacatttcgtctattgcatctaatcaaatcatcgacgctaaagaatacctgaaacatttcgaacaacgtgcctttacggtactaggcgtgtccgaactgatgatgggtcgtggtggtactgcttcccgttcaacaggcgataacttatctagtgatttcaaagatagaatcaaagccctgcaaaaagttatggcgacattcatcaatgagttcatggtaaaagaaatcttgatggaaggcgggttcgatcccgtactaaaccctgacgacaaagtagagtttcgattcaatgaaatcgacatggattccaagattaaacttgagaatcaggcagtgttcttgtatgaacataatgctatcagtgaagatgagatgcgagaacttatcggacgagatccagttgacgatggcgaaggtagagcgaagatgcacttacaaatggttactatcgcacaggctactgcactggcggctcttgctccaacaccagcaggtggttcatctgcatcggcgtcaggagacaagaagaagaaagcaaccgacaacaaaaccaagccgacaaaccaacacggtacaaagacatctcctaagaaacaaaccaatggtcgtcatgtacgatacatgcaggagatgctcttagaatatacaacgctgaatgaagcaatcaaggcgctgattgaacgctactaccaatacggttctaaggaacacttaaaatctatcaacggttctcttatgtatacagagggacgcttgttagaacttacgactcaatactggggcgaagaggtaaccgaaaaggttcgtattccattccatcgtatgacagaaaatcttcgtgaagaagtcatgtcaacgattgataaagtggagggtgtagcagaagcatctgatattgcacaagcagtgtttgatgtattcacagaccgtttgggacacatctcaaacgaagcatttgcaatttcagaatcacttgcggaggtgaatggcgatggataaaaataatttaacactagaagaacagtttaaacatggtatgatagaaccggggcaaccaattaagttcaatcagacaattgaattacaattccaagatggacagattgctgacctaaaagaatcatatcaacaaatcaatcttacagaatcgtctacagggaagaagaacgaaaagaaacatttgattgtaacagtagagggtatccatactggtatgacgaagaacatgacgttctatcccggcagtgcattagaggaaagcgtaccgacttggacggctcctcattacaaacctgtactgaaaaatcacaacgtttctactgagcctttaggaagaattatcggcgcagagtatgttgagtctacccttacggacaaatatacggtaagattgaaattaagagtaactgatccggatgcaattgagaaattgatggatggtcgttaccttacattatctgttggtggctctgcaaaacgagtgaactgtagtatctgtggtaaagacctgattcaagaaggttggtgcggtcatgctcgtggtcgtacgtacgaaggaaaacaagcacactggacaataggtaactatactggtgatgagatttcattcgtaaatatgccagcagatgtatactcacaagttatcgctgtagagatggtaacaggcgaaggagggaagaacatggatgggaagaatacggaaggcactcaaacgggcgctgaacaaacacctgaaaaccaaactcccgcatcgcaaaccgaaggaagtattatcgacaacttactaggtggcgaaggagaaggtgcaactcaaactcaagaaggtgacaatcctgagagtggcgaagctggaacacaagttcaagaaggcgcaggtgctactgatcctgaaccggaaactggtggacaagcagaagaaacacttgaagagaaagttgctcgattagagggcgaacttacagaagcaaacgaacaagtaactgcaatcacaaaagaacgtgataacgcacttgcggaaaatgctactcttactgcacaactaagtgagaaggatgcacacatcaaatcagcagagtcagagcgtgatagtttcaaagagcaagctattactcttgcgaaatactctcgtaaacacttagctgaacgtgtggctgatcttcgcatcttcaaaggcaaagagcaagcggaaaaccgcgaatcacttgttgccgagtggtcaacatctagtacgaaagtgttagaatctcaaatcactgacttactagcggctcctcgtcaaatcgctaccgttccaaatccgggtcttgctgttacagataacaacagcatcttggttgatgataatggtaacgaagtacatacagaagagtctacaccggctgctaacaaacctaagattaaaacattaggtgacttaacagaaggtatgacgaaattcatgactcgcaaaatttaattactaggaggcaataccgatggctatttttgaagcagatgcacgtcgtaagccgggtacacgttctaatacgaaacttgtagtatctggcggttcttcaccagcaggacaatgggtcactgaccctaacttaccaatgttattccactatgattatggtgggccgggacaaaaagaagtagtagtatcaaaaggtttactagtaggggttgctcctcaacgttacctagacgatgcactaggatataaaaagaatgctttaacaattgcgactgacaagattcgtccaattggtatggctccttataactttacaaaacacactcgtgacttcttagatggtaaccaaccttctatcatcactcgtgaatatgtagagttaccattgatccgtaacgcagatgacgctgccgaagttaaatggggcgctgcttacaatacagcaggtgcagaattacaaccaaatgatttagttacttggagccgtgatactcacaacttcggtaaagtaatcaagtgggatgaaacaaaacactctgttgctgacatcatcggacaagttggggaagtcgaagacgaccaagaaccgttcggttggttaaagtgggcaatgtgggatgaggctgctcgtcgtcaagacgaagggccgggtaacaaatcaggatacaacgcaccgggcgatgagggttacccttacgatcctgagtatgttcgtcttggtaaaaacggtgagaatggttacttagatcaatacactacattaaacgatgcacaaggtattccgggattacttgatggtcgtaacaaagcgttaacaccacaaacccgtgacttcactgttggaactattccagcagacggtacgattgcacaattcaacttaggattgaaaaacgtaatcgaaggtacggttaaagtgttcgttgataacattgaagtacctcgtgaccgttacacagttgactacggtcgtggcgcattaaacttcatcggtaacgtagctgatagcggcaagccaatgaaagtagacttccgtgcagagttcttcggtactcctgctggatgggatcacaaaggtgctgtagcagcaattcgtatcttactgaaaatgtagttcggagggagttcgctccctctcaattctgacaatgacaatagggtaacctattaaggaggatatacataatggagaactggttaaagcaacttaaagagtcaggctttacagaaacacaagtacaagagcaaaagagtttacgtacacgcatggagcgtggcgaaacattagcggaagctgatgcaaacaagttagctcttaacgaggaagagacacaaatcttagaatcattcgctaaaatgatggaaggcgaaacgccaacgaacgaagtaaacttacgtgagttcatggctactccatcagcacaaattttaattcctcgtgtaatcgtaggaactatgcgtgaagctgctgaaccattatacattggtacaaaaatgcttcaaaagattcgacttaaatcaggtcaatcaatgatcttccctagtatcggaatcatgcgtgcttacgatgtagccgaaggtcaagagattccagaagacagcatcgactggcaaactcacgagtctcctgagattcgtgtaggaaaatcaggtattcgtcttcgcttcactgatgaaatgatctctgactctcaatgggacttaatgagcatgatgattaaacaagcaggtcgtgcaatgggtcgtcacaaagagcaaaaagcgtatcaccaattccgttctcacggacacactgtattcgataactacagcacaaacaaacttgctcatacaacgggtcttgataagaacggtgtacaaaacgatacattctctgcggaagacttcttagacttaatcatcgcagtaatggcaaacgagtacactccatctgacttaatgatgcacccgttagcatggacagtattcgctaagaacgaattaatgggtagcctacaagctaatccttacggcaactacccagcgaaaggcgctccaagttcaatggcgttaggccctgattcaattcaaggtcgtctaccattcaacttcaacgtgaacctttcaccattcattccattagacaagaagtctcgtcgcttcgatgtatatgctgttgaccgtaacaacgtcggcgtattactggtacgtgatgaccttaaaactgaccaatgggatgagaaagctcgtggcttacagaacatcaagatgatcgaacgctacggcatcggtatcctaaacgaaggtaaagcaatcgctgttgcgaagaatatctcaatggataaatcttacgcagagccaatgttaattaaaaacgtaggtaactaatacacctaattgctagggtgctaggcttctagcaccctagttttatatctaaatgaggagagtgctgaacaatggcagaagtacaaaacgtagaacaaaaggacattctatttgcattaggtcgtgggtataatggtttctattgccctgagtctggattccatcttatcggtgtagttcgacctcaaggtttctggccgaaaggtttcccattaacagatgatgcaaaacgtgcgcttgctggcggtacgattgttgatgtaaacaaagtattaacggaagaagatatgaagtttcgtatccacggatcgactccaacttcatccgctgatcgcaagaagattcaacaaatccaagccgaagaagtaatcgctgaacaaggtgaagaacctgttgctgtagataaagagaagaatgttttatctgagccagacatcgacactgctacaaagaaagagttagatgcattcatgaaagagaacgaaattactcttgaaggtgtaaactctcgtacaaaattagaagacttacaagaagctcttaaagtacacttcggttacaaagaagctgctaagtaatctataaggggtgaacctacatggcaggaagacctaccatcgtatcgctttcacctacgaaatatcaaaagtctgtgccgataagtacatctatccgtgcggactttagtattgacctcgactctcgctatatcgacgggaacgtctttatgacagacggcaaaggtcaacgtgttgatattcgaacttcataccgtgcgaaggcgctgactgtcatgccgctccaacctcttagtatgggtacgacgtatcaagtaacgttcgtaggtgattcgaacttagatgatgatgtgaagcaaggacttcgaagtatcatcggagactgcatgggtgggaatgaggtattaacctttactaccgaagtcgatgagtctcttacagttcccgaagtattgctaccggtacaaggtgcagtattaaaagcaaagcctacgttcaaatggaatccagtaaatacagcgaaggggtatcatattgaaatatccaaaaccaatacgtttgcctcgctgatctatccgaaagaacaacaagtcattacaacgacagaacttgacgtcgatgttgtaatggaagatggattatactactggcgcatccgttctgttcgtaatgatggagcgacgggagaatggagtaaggcttaccagtttaacatagatacactcgaagaaggaaaggtatcagaagaagacgatgactctgcgactgatgatttccttgagtatgatgagaacgactttgaacttgagattgttgaacgattcccaaaagaactagaagcgttagttcctacgaacattaaatgcatgttcttccgaatcatcggcaagttcgatttaaagttaatcactcctgacagcattacattaacaggtcatcacatttcaggtgacttccaagaagaatcacatggtactgtcaaaggcgacattacagtggttcaatcaaaagatggtacgtcgtatattatttttacgccggaacctatccctgataaagaggcaggtgaggtataatgggtgttgtacagaactataacgtcggtggatggatagatgagattaagaagattaacttcccgcctcatttattcccacgtaaaatgcgcccctatgtgaaaggcttcaagattgaagtccctgctttgcccggtatccattccgttcagtatatgtgcgaagaagacatggagttgattaacatctctctagcgtgttccggttacaaggacggcgactactgggaagcatacacaaccgaacccggcaagaaagagtattgcgcagtggaaacaatgccaacacgagaacttgctgaatctattaacatgggtaacgtgctttacatcgttcaccgattaccaaaaggtagtatggtacgatttgattttcataacacatcgggaaccagtaaaacggtttggccgtctttacgattcctaagataatcaaaaggagtgaaatacatgtcatcattaaaacgtgcaaagtggattgaaggtattacccagtctaaacgtattttacgtgatatggctgaaatcttaacaactgcaattcgtgatgaagaatcagttgtacccgaagagaactggagccttgtgtatccacgaccattctctgatgagaagttaactcgtactcgcatgatgcaagatacaacaaacaaacgtaagtatgtccctgttgcatctcgtactgaccgtggtacattagttggcacaactgctatgaaactaacagaaaaagaacctgtagaatctagcattgttgtgaagagtgaagatggttctgttacatatacggtaacgaaggactatacatacaataaaacagagaagagtattgtgcgtacggctgcttctactatccctgatggtagtaaagtacaagtcatctatgatgcgaaattgtttgctatcttagatacagtaccagttatcgtagagaaatcattaaagattgcggaaccaaaccgtgagcttgctcgctctacatacaatattgattatgctggaaagcgtatcatgttcttaggcgatgctcctgctgatgcagaatacttcgcattaacattcactgaggttactggtacatatgaccgtatcgaacgtgaactattacgtcttgaaaaagacttactagatccggacggacgtacattccgattaccagcaggatacggcgaaatcgcacaaactcctgatactcgtgaagtagccggaacaatctctggtggcgcacttggttctaacggtagcggaaatatccaatacacattagacggtaagaatcacacagtgaaattcacaactggttctaaaacgttactagaaacacaaacactttacatcaactgttcagagtatacggacgcttctaagtctgcgactcgtaagattcgtgtacagcttgcaattgatacaagtgatagcacaaacaaaacgtataaacttgttggtacattcaatgagttaaacgatggtgaagctcacaatgttgagattgtcactcaaacaactgcacctgctttattcaagcatctaagttctttacatgctggatatgagttagttgcatctgatgaaatcgttgtagattacaaaggttgcgttattacaacaatccctgaaatcacagatgacttagtggctacattcactgcacgtcgtgaagatgatgtaaaagcaggtcttgagcgcatcaaggatcgtgttgtattaaaaacaactacgaagccggaacaggaatataaaccagttatcggtgaccagtatggtgcaaaagatacagcgaaggaattaacaatgtacgttgagattgtaaaacctgaacgtcttgtcaatcctgagactggtctagaacgctacaagaactggaaaggtgttatggttcagactggtgcaaacaatcactacattcaaactcgtatgtttgatgcatgggatgatgcacaacaaaaaccaaaagatgctgtatacgataaagacggcaagctaacagagaaaggcgcattcgtttcagactggacgaagttctcttggttcaaagactggaaagaatacatggtcgatgagttagatgatgacgctggtatcagtgacgtatctgatggtatcgtattccaagaagttgtaacgcaaggtatgacagaagagttcccgattcagttctggatttctacaaacaataaccgtgctgctgttgtgttaatgggtgaccctactcttgaccaagataacttcttaacatcattcggttacttaggacgtattcatccgttctacgactcacaatgtgttgtgaagaaaacagaagacggacaaatcgttcttgaccaaaaaggcgacccagttgtagaagagaaacgtacgtacttcgaaaacgacgttgcgggtaacttcgcaatgacattcggttcatctacaatgccagccgctatcggtacaccacctcgtggattgccattagtagaaggtatcgagctgaacgttgatcgctctacttctcctgcaaaaccaatcgttggtgagttatacgataagacagtattctcttacgttatctcttacttaacagaggttggggaatctaaaccaactgcacttgacggcgcacgcttagttgttcctgctggtacagtgacaaaaggtgctgtaaatccggaacaaggtatcagcgttaaacttcgcttccgcttaccggatgaagcaactggttaccgtatctatcgttaccacaatgcaggtgtaacttcatttggttctgatgcgaataaatacgaaaaccacaaacttgtaacgtctgttgaaaaactagaccgctcaagaataattgaatacattgatgaaggtactgcgttaccaatggcggaagcaaaagtaaatgcttctgatggaacgaagacattcacattacttactgattcactaaacgtgttctacaaaaatgtacaggctgcttctaatactgctcgttcattcgaggcagtacgtcgtgacctttacactggtgcaatcatggatgtgaagttctcgaacaagtttggtaaagatacaggtacaggcgtaaatgatattatgatgtatcaaacacgctctggtctgaaataccaacgtcaccaagctgcattcattacgacagaggagtttatgcgtaaggaaaaatcaggacagtctcgctggactggtaagttccacttatctccgatctacatcgaacacagctacgataaacaacgtggttggttagatggtgtaatggcagtagacgatagcggtattgagcatttggatgaattaatcgtagacaaagatacgccagaagaagaagtgtacaagttcttccgtatcaatgcaccgtttagcttattcaataactcaccaaactacggatacggcattgccatcatcaaatcatctttaaagtggaagtaagctaaaaggagagtgcaggaatgaaaacttcatatgacctttcgttcttgaactcctcaaagaacccacggcaggataattggataggacaaccatttgtgttgtcctatcttctttctgagccgggtacaaacttcatgttccgttaccaaccttactctctaagtcacagtcgtattacgcaagaccgcatctatgcaggtcgtcattctaaagaaggagatgtaaatggtacgtatgtagatggagttgtaaaccgggatcaacgaggaatcaaactaacagaacgaccaatggtggaagtaaaccgggtattagaagcaatcacgcaacatggattccatgccacacttgaagatgcaagtgccgtcattgaatcgctactgatgattgataagaataacaagaatggtggcatcgacataacgcctgttgtttcagagcagcatatcatcgacaaatacattaatcctgttctcttacgttcttatatcagtggttctcgtgcggagcaagtaaaagataccatcctacatatctaccgaatcgcttcgatgaatacagaccgcattggtcacgtatcgcttgtagaaattatgaaacgcatagatgagacagatgaagtaactcgcatggggcaatacatacttggttacatggatcagctacatgatggtacgaagtctgttcataaattaggtcttcgtgataatgacaatgtagcacgcattacgggactgctaaacaatgtagagaaacgggaaaagtatttacaggtgacgaaacttcaattcgctagtagatacaaagaacgtaccggacataagcagttcttctacgatacaagtgctaggctcattacgcatgatggtattgtgataaaaggtgttatggaaggtattacgtcagagcaatatgcgtgggtgtctccgttacctatcatggtacagaaacgtattacactagaaggtttggaaacaacgcatacacttgtggacgctctcgtcccgaatgaggctcgtataactgaagacgacacattatatgcctacggcgtgtcacaaatgtcagcgaggctaataagccccttaaacgtagcgtcacacggtagaggaaaagaagtacgcatggaagagactatttatgcgacgatgcagaaaacagccggtgcaagaatagatatgcaaacctctctactagatggcaagaatacgaaggagtctgacattatagagcatgacgaaaagaaagcagaccatcacggcagagatgcaggtaaaggtgattataacaaggtcgcactattcgatgagaaagtagcgaaggattctgcgaacaatcagccctatagttttattgacgagattgtaaagaaagatgcatggatgcaagatgtattcttttatgtagacgagaagctagataaagacgcacgtatcatcgaacaagacatcgagttcttcgataagcaaaagagtgcagatgcatggattatatctgaccgtacgtggctcgatttattaccgcaaaactctcgcattgattgggacgcatctgttaagttagctaagatgaataaagatgcttggattgtagatgaggttcgctggtctacaatgatagatgagaaggtagacaatggtgcgtggattgaacatcgcttccagttcttcgagaaggtaaataaatcagcactagaggagaaggaacttatcgttcgtgttgataaattgcataaagaaggatggaagaaagttgctgttcacctgatggatcgtcaagaaaaagtatctctattatcaaagcactggacgattgcatctaagatgttacttgaggcctacgagattgaggagaatattcgtcagcagatttcaacgctcatggataaagaagcaaaagcaggacgactcattaatgatgtagaaaaagtcgtgaaggaaatgaaacctgcactcatcattgaagaactgcgttcagatgaagtacgactaaatgccatgataaacaaactgattgattacggtgatagaccggataactttagttcggttgtgcagaaggtattattgtcaccggaacaacagcgacgaagcattgcgctaacggagatgaactttgctgatagagatattaaccttgcactcgttgaacaaatgattatcgctaacgatgaagcagaagacaatggtcgtgacgcaatgattgtagaaggtcacttcctgattcaagaccaatcggactgggatgagatttggaacaaacgagtaacaggtgtcgatattcttgacccacctgattcagactacgattatagtaagctcgcttcacaagtctacaatttagagactggtgttccgtataaaccaatgtcgccaacgaatgtaccggacgtacgtgtacgcacgccactgcatcatccgatgccggagcatttcgatataggggtagacccgacacgagaactggttgtagataataacatcttcaaagatgtcgtgcttgcgattgaatcactcaagttccgtaacaaattgcggtatgcaggtatgccagcagagaaagcaatgcgcgaactattctcgaaactattcctttggatccaagaagcaaacccggatagtgcagagtataagcgtatgttccgttttgcgagatggtatgcagagtcagccgtattaaaactaagtgaacacattcttcatcgtacgtatgattcatgggtatcggaaatcgagacaggtaaaggattaggtgtacagtatacgaataaaggctggttctattttgctccggcttatgcgatgcagacacaatccacatacgctgtcatgagttttgaaaaagaaaactacattgatggacaaattattctcatgggatactttgataatccattagcacaaggtacaatggaactaaaggtcgatggcgagacagtagattcatggagttcacgaggtacttttaaaagagtcatagacgtagagcaaggctctcacaaatatgacattgtttttaaaggagacagtggagtcgtatctatatctcgtatcgaagtcaccgggtgcgaatttgtttcggcatatacgtcatccgatgatagtaatacgaatgggttaaaagccattcagactttaattgatatgctgttatcttacttcgacctgcatcatggcgacgataagattaagggtacaatggaagtcaaacaaagacgagtttggaatgtccaaacataaggaggaaatacatatggtagtgaaatatcaagtagagcgtcgtcctatcggatcgaacggtaaaatctttcgaggcacaagcaaggaagaccgtattgctgtaatcgaaccaacggctcgtaaaacaatgacgtcaggtacatctatgggtgacgtagcattctacgacattcaagatattaataagccaacgtacagctctgattactctcgtgtgcaagcacagaatggtgagccgtggttagttcactctactcacttctctgttgattctgctacggaatctgctgctccacttgcgagtgcaattggttctgagaacgtacgtatcgtacaagtattaagccataaaacattcttcaaattaaactaacgggatgaggtgagaatcttggcactagttaaccgcatttctaatggacttgtttttcaagatacatttgatagtgatgtgttagatgcgaagtggcaagtttctccgtctgatgatactcgttatagccttacagagagtcggggtacacttcgtttaaaacacggcgaccccgatctttttgttctcatgcttgctccacgatatgatttcgtgtttgaagcagaaacaaattatgcaccgacccgtgcttcggatcagggaggcattgttgctttccgtgataaggacactcgtttagaactacttgaatactttgaccctgcaactggtacacggtttgggtacgataaaatccgcatgattcgtaaggcggatttatttgaaggatacggtagtaatgacggcggtaaaacgtgggagcttatcggtgtaagttatttgtctgcgccaaagattggattcgtactgcacggtatcaaggaagcacaatctgtgaacttagatattccggaagtacgtatgtatcgttcaacgacggttcaagtcgggaacttaattcccggtcaatcggtaaagctatgtgatgaaaagggaacagttatcaagacggatacttgcaaagacaatagcgaccatgtggatttagattgtaccaatgtagtattcccgttaaaaggtaagattcagatatacgatacgacaggtcatctcttggaggacggcgaagtcgtagaagatatttggggcggagatgtattttggtatggtgtaaaactagacttagaagtagatggcgtactgatgcgacaagaccgtgagtaccaattaggtaacatggaacaaggtattatcgagcgtgcgatgtacgtaatcaataacaatgacattccaatcacaaacgtccgtgttagcatcgctgcacttggcgggtatcatggttgggaatggacggatgtagcgcaagatgtgtttggacagccgggtacatatcaggataaagtcttcctcggtacaatccgaccaggagagaaagttccgttatggtgtaagattacaaggcaacccgcacaacagcttgcaagtttacaagattacaaattccgtatcatgtttgaaagtgggtgatgagagatggcattgaagttcgtacaaacaggtaataatgccggcggtagtggaagtggtacatatgaccatagcctattaatcaaccgtggtatgcctgaccaacatacaattgaatctattacaggactgcgtgatgttctgaataagaaatacgaaaagccatatggtggtatccctcgaacagatttaggattcgaagtggtaactccgcatgacttctctgtatttaagcagacggcactgcgtgatgtagaagataatgtcgctcttatcacaaaagaaattgtcgatgcacgaggagataagaaaacacttcgtgaatacattgatacgaaaatctcacaagagaactggactggtgggggaggtactggcggatcagagtcatctgtcgggtatccaatctatgaagagttcactgtacaaaataaacaacgccgtttccaaacggataaaacgtatcgcatgggtactcgtcaacttgaggtgtatgtagaaggcttgaagatgattgaaaaacgtgactacgtagaagtggatgaacatacgattgagtttctgtatgatttagaagaaggcttcgttgtactgatgatggtgcgtgctgttgtaaatagcggactccatgaagagtacacggcaactgctggtcaaacaaacttctcgttagtatctccgtatgcgattaaccaaaacatgttgcaggtatatcgcaatggtcagcttcaacataaggggcgtgactacaaagaggtcaacgataagctcattgtattccagtttccgcttgaagaaggaatgctggttaccttccatcaatccggttcaagtgaccccatacaaggtagtatctatcaaggtatgttaaatagtatgcgcatgaataatgcgtacctagctatctcgatgcaggatgcaatgaagtcggatgttgtgaaacacttcgatatgtatgcagatagtcttgttacgctgtctcatatcgacatgaatgcttcggataatttcctattcgagaatggatctatttccgtaaaagaaatgaatcgtacactcttaaatagaaaagacttcgagactggtagtgcatcatatgtagacttcattacctatccggatgagattcgcttactgaatgtaccgggcggagaagcacgagactttgccgtgcatgaacaaatctcggataaagtaatcggtgaccttgttccgtttacaaacaaccagcaccagcgcatgttcttcgttgcagaagaaacagcagccggacaatgtatactgcaagcaaacattaacggcatgatatatgaactagcgacaacagatggtcacttcttctcgctgaatgtagcaggtgatagtgacgggaatgcacacctcgtcttccacgaacaaggaagtgcaaagggtgtctctattgtgtactacctcaagttcaatacgactacattccaagttgagtatcagcgtacgctaagtgatacgaagctagatgcaatgcgacctgatatagatgtagatgctgacggtagagcgcatgttgtgtttagttcaaagcgtgccaatccgaatacgttcaatgttgagtaccgtacgattaaaagcggaacggtatcagcaagccgtaacattacatcggatgagttagtcgcaggactgaatccacgtatctccgttggatacggtcagcgtcctaacattgtatatgaacaaaacaaaacgatttacttcgttcacttaaatggtgacgtcatcgagaagcaaatcaaattaacagatagtgattgtgtacagcctgatattgatactgacccgaactgtacttctcatattgtatggaagtcaaaacgactcggcacaaactacggcgtagattacacaacgatctcgaaggattatatcgtaagtggtacgaagagtgttgcaactggttccttctcttgtgactatccagccgttggtgcagactacaataacattgcacatgtcgtgttccaagctaatagcgttagagccgaccatgaaaacatttgttatgcaagggtgtacccaaatggggacgttcttccgttcgttgatattgcatcccgtgtgggcgtgcagtaccgagaaccgaagctggctatctacggtgagtcattcatctgcggattcttaggcgacgaccacggatatgaaacatggaagttacttgcgaactattcagatacaggtagttacgattatgtagtagatggtggatcaaaagaagccgagtggcgcaaagtaacagttgcctcccttgctccggctggtacatcggttattgctgaatacaggttgagcgatgataaactatcatggtcagcatggaaacctgtagaagaattaacagggcagggacaatacttacatattcgtgttacgctaaaaacaacggacaatacgatgacaccttctgttgaagcgattgatattacatatcttcctgcattcgtagaagtacaatccgtgaagaactatgtagagagaaatgtggattcggtaatcgtcgttggtacaccgggagttacatatcaagtatctcgtgatggcgggaaaacatttgtaaatgcagacctgcacgtaccaacaagtgttgtgcaaacgccggatggacatgacattgtactccgtgcgaaaatcacgaacggttctaagctagaagcatggggcgttctatggtaggtgaatcagatgaaccatatactagaagcactgcgagaaggtatgaagaacaaattaattatcggcaagggacagacggatgtcgtccctgctggtagtgaacaagtttggttcgtaccactagaaacaaacaaactacttgtaaagtctattgaagtcgtagccaacaaacaggctacattctatgtcgagttctttgaaaatagtaagctcgaagactctcgttacaatagcggggaagtagcagggcgatgctatgacaatctcgacctaccgtttgtgaatggagacgatacaaagcaaatgtatgttgtcattcacaatacaagtacattcgacacttcgtaccgcatcgaggtacggggaactgaaatgaaataggaggatatacaatggctactttttctccgcaagaagtgtaccttgtcgaagatggacaggtagaaatcatattagattcaccatacgttgttggtagtaacacggtgcaggtatatctaaatgggatgctatcgttactcggtaaggactatcaagaagcaagcgaaacaacactcaagttccagtaccaactgagcagtgaagatgttgttatcgttcaacgtaaagtcgatgtagaaggtgcgagtgtttctgttatcgggaaagccggtagttcattgtatcagaagtacggtgacaaacaaacactactgccgaaccaaaagtatacactgacgttccgctatggtgaccaagtacatgagacaggattctctactcgcattgatccgctgtactctacatttgagacaatcaactttgacttaggtgagatcgcagatgacattccgatggagcgtatcttattcctgattttccaaaacagtatcctgtcgcaaaacattgcgtccgaagagaaccttgagttactaaagacacaagcgaagacaccatatgtatttaagcagttcgttcgttaccgtacggagcttgatattatgacggcagtctacttcgcactatccggacgacaaggtatcgagaataagatactaggtgaattaacaatcatgcgtcgtaaagaatttggtatggctgaccttcgtaccatcttagctgacttaaagaataagctacgagaatgggaacgaaaacttcgtggttcggataaagtatcaccactacgtagtgcggttcgaggcggtacagcaaatccatatccactgaatacacctcgtcgtagttacgacccagtacagaaaggagcgagttcataatggcaattaatttacgacaagagattacgaagctcattgaagatacaggtcattatgttctgcttcaacgtacgagccgtaagatacgatgcagttgttggaatgagaaacatcaagaggcagacccttcgtgtccatattgtttaggactcggatgggtaagccgtattgagcgacataagattcgccgacaaacagcagtcaatgtaatctcactgccaaacaacattcagcaaacaccaatcggtcagctatcgacagatacacgattgttcttcttcaagcatgatacgatgccgaagaaaggtgacatcatcatggaggttggctggaaaggacaacgaccaacacatctcattacgacgcacgagatttcgcactccgatgatatgcgtgaacaaaatggtcgtattgagttctttcaagtgacgacgaaggaaaagagtgtcgatacaaagatacgaggattctccgtacgtaagattgggccggtacgaaactacgaacctatttttaagaaggtgacattatgatgcgttatgactttctactagatagagatgttccgaaaggtattggtaaccggttgttcctacttggcgtaagtgaagatggcccctacatggagcctcttcttatacgtacaaaagaacaagcacgcaaggtcttcggcgacgagaataagggtacgcttgtaaaagcatttgatgaagcgtatgatagaaaagaagatatttcaatttatttaatgcgaatcaccggaaattttgctacattagaagtagaaggttttccgactgatccattagatcgtcagcatctctttatactccgaagtgtttacgctggtgaaaagtataacaagctctccattaagctaaacaacgacgataaaagagagatacattccttcactctgaaaacactcgatcatgagtttgtttatgaaattccatacgacatgacagtcggtgcattcgtcaaaggcattaatgaagattgcagaatgggtcgtcataaaatcatggcttccacagactatccgcaagcagacattggtttccttgcaggtatgttgtcagaagacgaagaagttcgctccttcgatgatggtgacgacgggctacatccaacaagaaacgaactgtatcttgcttgtgatttagcgtatcagattttgcaaggacgacaggtagacattgttgtacctgttggcatgtatgtcgatgatgtacatccggcttacttgtatggtaaggcaatttacggtagctcttattattcatcttcgtctgactatcttcaattgatagatacgcagaatggtaataaggtagtaagtttccacgaacaacttatcgagttttgcagagagcagatgagactcggatatatgtcgaagggcgtgatagggattcgtccttttactcatgttcctaagaatatagaagaggacaacactgcatacatatctcgactggcacaatcctctgcgtttcgtgaccgtcatggatttactgactacaagaatggtctttggtatgataagggatatttcgtatccgttgtcggacatgaacttatatatcatccgggtaaaccggatgagtacactgcaaatggcgctgttcgttacgctgcaattctagcaggaaacttcgatactactacgaacgttgaaattggtgatgacgttgaactacgttacgaactatccaattacacactacaagaactcagtcgcattggtgtggtaactttccgcaacagcattcgtcgtggtaacgtggttactagcggggtgactgccggtagtcctgaaagtgaattgcatagtgtggcaaatgtgagaatggttcaaataacactagcttatgtgaatcaggcagtcgaaatgatatatgaaggcgagttcgatgcaggtatgcgccgtaactatttggaggagttgttaaggaagttccttgatgacttacaaaaggaaggcgtgatcttgcagtacgactacaaaattcagttcagaaacgatgacgtcaaaggcgaaatccttttattccttgaaggtaaacatacaatagagggcattcaaacttctgcggaagtgacccaacaggaggtataacatggctaacgaagtacgaagaggaatgcagacggcagagggttcattggactacgaccagtttacggatgagattaatcgtcttctccgtttagcttggggagatgattggggtactttcacagaggacgagcctacaggtacagacggaaacgatgttcctgttccggtgattacgttcgacttagaaagtaggaagagaacgaagtatggcgtcaagtcattggagcctgttcacttcgatacgattcctgaccctgagaatccagcgcactctatgaaactatatcgtcagtggtttgatgttgaaatggtcttccgagtatttcacaacacaaaccgtgctgctcgcctcctcatggaagagtttgaagacttcctatttacatataagagtcatcttaaacacataggaatctcggagcttattttccaagaggagaagaaaccaacggttgaatcgagatggagtcaaaagcttgcagctagaacactcctatatctcgttcgcatagaacgaatcactacggttcgttcaaacaatttcgttgaaattgagactacggttaaggatgcgaaaatcacaaaccctcctatggatggtgtacgtgagacaattgttggttccaaaatggatccgatgattgcgcagtaccgtagtcaaatgaagatcacagaataagggaggatttcaacatggctggattcgatcaatatcaagctattccgggtcataacgcccgcttcaaagacgggaacttaaacttaaagtccgacccgaatccaagagaaactgaatcagttgtactcttaggtacggcgactgacggccctgtaatgcaacctgttcgtgtaacacctgagactgcttacaacatcttcggtaaagttgcacacgagaacggtgtatacaatggtgctacattgcttcctaaattcgaagaactttgggcagcaggtaaccgtgacattcgcttaatgcgtacgactggtgtgaacgctgtttctagtttactaggtacttcttacagcaagaatagtaaagaagttgcagaggacaagctaggtggcgctcaagctcgtggtaacgtagcagcaacatttactttacctaacggtggtatcgttgaagcaacgttcctattaaaagcacgaggcgtaatcattcctccaaacaactacacgcttgatgtaggtactgaggaagatatgaaagctggtacacaaccaacattcgcacaagtgctattaaatgaaaacgtagcagacatggaatctgagattacagtaagctatgagttcacttataaagatgcacaaggcgaaacaaaaacatctgaggtgttagacaataacactgataaagatggtaaaccgatgattgcgaaaggcgctgacgtaacaatcaaacttgagcatgttgctttagctggcttgaagctatacgctgatggcattgaagtagtagatgcaaaagcattcactgttgctggtgaccaattaacaatccactcaaacagcaagatgaagttaggtgcgtcactagaagcacagtacgcttacaatcttgtggaagtgatccaaccggttatcgagctagaatctatcttcggtggtggcgtgtacaacgacatcatgcgcaaagtagaatctaaagacggtgcggttacggttacaatcacaaaacctgaaagcaaacgtggcatgattagtgaagacccacttgtattcaaatcaggagattacacgaacttcaagatgttagttgacgcaatcaacaaccatccgtttaacaacgtagttcgtgcaagaacaaagcctgagtttgaagcaacgttcactagcacgcttcaagctgctgctgatgcaaaattctctggcggtaaagacgagctttctcttgataaagaggaaatgtacaaacgtcttggcggagagaaaaacgaagaaggcttcgtaacgaaacaaggtgcttaccagtacctagagaactacgaagtggactacgtaattcctttaggtgttcacgctgatacaaagttaatcggtaaatacgatgacttcgcttaccaattagcattagcgtgtgctgtaatgtctcattataacagtgtcacaattggaatcattccaacgacaacaccatctgacatctcacttgctggggttgaagaacacgttaagaagttagagaactatgcgaacgagttctacatgcgtgaccgtttcggtaacatcatctttgatgctgaccgtaacaagattgacttaggtcaattcattgaagttgtggctggccctgacttcatcgttcgtaacactcgtttaggacagatggcttcaacgcctgacgcttcatacatcggtatggtgtcacaattgaaaacacaatctgctccaacgaacaaaccattaccatctgttacagcattacgttatacgtactctgctaaccaattgaaccgcttaacgaaagctcgtttcgcaacgttcaagtacaagcaagatggttctatcggcgtagtagatgcaccgacatctgcacatgcaggtagtgactacactcgcttatctactgctcgtatcgtaaaagaagcagtaaacgctgtgcgtgaagtggctgatccattcatcggggaaccgaatgatactggtaaccgtaacgcattaacggctgctgttgacaaacgattaagcaagatgatcgagaacaaagctttactgggctttgacttccgacttgttgtgactcctcaacaagagttacttggcgaaggttcaatcgaacttagcttagaggctccaaacgagttacgtcgcttaacaacaatcgttagcttgagcgcataatacatgggggttaacgcccccgtgttatacaaatacctcatttaagggaggctacatagatggctggtaaggattacgcaaaaacactaacaagtttctccggttctgacttagtagtaaacttcgggccgaaaacaattggtgaattacagcaaatctcatggggtatcaaacgtgagaaagcgccagtattcacattaggttctcctgatgcacgttcattctctcgtggtaaacgtggtatcggtgggtcattagtaatggctcaattcaaccgtgacgctcttatggaagagttagtagacgatgcaaacgtatggaagcaaattgctccggttgcaatgtttacagcggcgggtaacttatctcaacgtagtaatgaaatgttcgagaacgcattacatgctgctggttggggtgactacgctgactatactcgttctaatcctactccggatattccgggtgttactgtaggtgcagcgcagtacggttacacaggcggtacacaaattacagcaggaacaacaatcggtaacgatggtgcgactactgtacgtgttccaccgggcttcgctcaaatccgcgcgaagaacattatctatgctgatatgattccagcattcgacgtaacgttaacgtttgcgaacgaatacggtcaagcagcattccaaaaaatctatgacattgatattctagatgagcagtctggtgtatctgtagactctatcatcatggaacgttctatgagctatgttgctcgtcgtatctctccattaatgacaggtgtttatgaccgtgctacaggcggtagtgttacaggacgaggcgtttacgcctaattaacaagtagcccggctgagatgccgggttatttttataggaggaatactatgtatgttcgaggtggagcaaaagctcctgaggtaagtcgctttgaaagtgctgatagattcggagaaacatattccggtgcagacatgaacgtatatatggcgttccccggctacaagccattatgggtaggaaccgtatcgactgttagctacacggtatatcgtgagaagaaacaagtacgtacattagggaagatcaatgcgaagggaattgtaaaaggccctcgtacaattagtggtcgattaatctttactgttatatcggaacacatcgtagaaagcattcgccaagagattccgtaccttcgtagctataaagtattactaatggatgaactacctccatttgatttattggttagcttcggtaatgaatacggcgcaggtgcaggtctagttgtcgaaggtgtaacaacagttgatgaacagaaaacaatgtctatcgaagatttatttacagagaacatctttacgtatctagcacgaggactacagccgatgcgagacatctttgcaagtaatgcgaagaaaccatacaatccgcttgattggtatacatcagacttccgtcacccgcagagtgaagtcatggcgaagttcaaagtaaatgacttgatggtagcaaaagaaagcttactacttgctgacccgactccgttcttaggtgggccggacgaatgggattcagaagccgcaccgacgatggatattacaaacgtcaatattaataacggtggtggcagtaacgggaacacgagctggggcggtaaaggttcgaaagtatttgtacaggtatacgatggatcgaaatcagcgcaacctccgattgaaggagctaccgttgtatgtgcaggtcagtctttaacgacagataagaaaggtgaggtatcgttcttcacgcagagtgctgaactcgaagtatctgcaacaaaggatggattcaaacaaggaggcggaaacccaacgacgattaagacagataaaacggatgtatcgaaatatgttcgtctgccattaatccctcgtggcggtgtcgcaaatccgggcggaggagcttgcggtggtagtgataaaacaatcattggtgaagttcgtccgattggtgtatggtttaaagagggagcaacaacgcactacattgttcctcgtgattcgaagggtaagaagtacctgtatccgaaaccatctgtacaagtattaaaccgatgcggtgaacctgttaaagatgcgaaagtaatgtggcgctatgagattgcaaacttctcatctgttaagaagaatgattgctggggagatacatggctaccattctcttgtatcggtcagatcaaacagtcgtcaggttttattacaaaaggtgtgtcggtagataaagatggtacatcaacaatgccggagtttaactttggtaaattcccaacaggtgcaggagtcaagattatcggtgagtccggtgactatggtatccatgcaacatactactttgaattatcagagggagattaaggggtgaagtaaatgccggatacaacgccgaatcaggtcaagctagaagacctgcaatccacgaagctacttcgtgaatataagaaatctgtatacgaaccgcaaggtatcgactttaataaacgacaagtagactatcagttattcccggaagaatacttcacagggacagacgtattcgtttacttcaatgacttatggctagatgaaatcgtaagtcttagttttacactatctgaaaacgtaaagggtatctttggatatgcgtcgaatacatgggactatgtaggtcgtggtaaacgacaagtagttggtcagttccgaatcgcattcaaagaagctggctatatgttcgccgtacttgaccatattggtaagcagggcaaccataagaagacagcgataagctggattgagaataaggatgcgaaagtagagaatggtatcgacggcgtaccggctggctatgctgatgtactggaacgcattgaggatgcacttggtcgtatctatggtgatccgggggcgaaagcacaggataagtcttaccaagagaagtatcgtcgtgacttcgaatggaacttaccaatgcagttaggctctccgaatacaaacatgggtgaaccacgtacaagtacaaatcaaacgggtcaagtatcacaattacaacagcgactcattgacctcggctttggattccctgaggcgaagttcaactggggtaaacattatgcaggtgatagctggactggttcgaaaggacaatgggtatcgccgggtacaacacaagcatccattgattatatcaacaaacgtacagggcaacacctcggtacatccagtggtgattggtatgtatgtcttcgttacttccctgagggtggtatgattcataaatctatcggtgcaaaagatgcgatgagtcagttcgaagtacaacttcaaaaacgtttagaccgttatccgggtgaactgtccggcttaaacatgggtggtatgtacggacgatacgatggacgttacggttcggggccattaaaaggtgtgcagttattccaaaagttagcgggcatcaatgatggatcgcaagggtattacatcacatctcaaacgaagcgtgcgctagaagctggcttaaaagtaacaggtgaatatgatgtagcgacacgagtagctgtatggctttatcaagcgaagcaaattgcaagcggtaatccctataatattcgtgaagctaatggtatagtagataaagagactctactcgcaatgggtgaagattcagaccgtacagtagttgttcccggtgagaaccgatacaaacctacagagatggctgaacatttatatgcaacctatgagcgtgaagtatggggacgaccatttgtggagcgtgcagagaacgtacgaagagaggacagcttcttctaccgtagccgacgtaatgagaatggtgaccgtcaccttgagtccttatcgcaaaacgggtttgatatttatatcaactatggcccgttgccgcagtacatacaaaacaaactcaataagttacctgatggcggaagttttaacacaaccgtcaaagcgattcgtaacgtacagcttactgatgttcagcaagtgatagatgcgaatacagggcaaccgattgaggagatttatagtttcgtagcaaaagatttagactagtcaattgtgagagtgacaaagaggaggatgttacatgttaccaccaaaagaacaaatgcaagcgatgaaagcagatcaatctagattcgaagcgccgaacccaaatgcaaagatgccgggcttcgggcctcctgatattggcggtgaaccaaaaggatacaatccgcaggaagtacaagatgcatgggcaaacatggatgatgaagaaggattggaacaaccaattcctgatgatgcagatatgggacaagaaacaggtgaagtattcttcacagagcctcctgcatgggaaggtgttggcgaatatcctgaaccagtaagtctaaaacaaaagggcaactccttcttacaagaagaattgatttgtccgggcgggccaccagttcatcaagtaaactcatggaagaaacagtttagtgaagatggtcatggtgtattcttatctaccctatacgatgaagtattcatttggcgtacattatctcgtacagagtaccgtgaaattatggcactgcctaatactgaccctcttcaacgtgaagaagttatctgcgagatttgcgtactattcccgtacgattacaacttcactgagatggcgaaccgtaaagctggtatccctgctcaaatcgcagaagatattatgaaagaatcaggattcgaaaaagctgctcctccaatcagactgtaggtgatatatatgaatccacaacagattgaagcaatgattcttagcttccggaatgagtacaaggaagtatatctcactacagttggtaacgaggattatatatggagaacattaacacgaaaagaacatcgtgagattgcggagtttgcaccggatgaatacagtgcattcgaacgtatctgtcagacatgcgtgctatatccggtaatggattggacaagaactcttgcctacttaccggaagaacttgcaccgcagatcattgaggagtcagggtttggtagcttccgtaaagagaagcccctccttgacatcttccgaaaagaattagaatcatttgaggcacaggcggaaatcatcgtcaatcgtgcctttccttatatcacattcgagatgatggagaactggacaaaagagaagctactcaagtatgtagcgaaagcagaatggcaactcatcaacatcgaaggatataaacatatggcgcttgtaaccgatgaggagattcgtcaaatgaaggaagaagaaggagaagaggttgaggaaacagaagaggaagaggagttcgacattatggtcattgccaacgagatgcgtaaagcagggcaagacccaatgttcgcattacgctccctgtaccaaaaaccaaagactccttatgtagaacgacctatgattggtggattccatcaagtagatacaatgattgcagggacaacggcttggagaggacgtggcatgaactatggcagatacgacgcaatacaggaacaagtacagagggtatctcgaagatgacgctgacacatatctaagtcccgaaatggatcctacagcaaaagtaataatgggaggaatagccactgtcggtcttgttgccggtggctatttagctttcaagaagggtgcgctaaaggatgtcatgcactctacgattgcgaaggcaggtagtttccgtagagggaaaacaattgcaatgaatgatgcggttcggaaatggtcacaagatgaccgcatggatgaactaggcaacgccgtgatgaacttaatgaaaggcaagccgaaggaaagtaagaagagtgcaaaagagtttttagatgtcatgagagactttaagcgtcacaaagatggtgcattgcaagaacatgctgaccgtgtggctcgtatgaaggagtctcaattacgtgatagagaaatcgaaatcaagacagagatggcttctcataaagcgatgcaagaacgcttccgtaaagataataatgatgagaaagctgtacagcgtgtatcgaagttatttgaccaaacaatattcgatagacgtaaccttgataagaaactagagaaagctcgcgaactgcaaacaggattccgtcatgcgacaatcaacgacctgattcgtttaggtgagatcaacgaggcagaggattgggtgcaagaagggattactatctcgactcgtgagtttatgaagaaagcacaaggcgaagaggcgattaagaaagctggcgaggatggacgcagacacttcttaaacaaagtagctgacaacagcatactaattgataagaaggataagattgctgacctgcgtgacttccgtgatacattcgaaggtatggtacatagtttaacaacagactttacgattccacttgtaaagattaacccgcttcgtatgttttacatggatcacttcttcacagataaatcaaagccattgttccacgttgctacgtctgatacaaagaatccaattgtaacgggtcacaacggtgcgcaaggtaaaccgattgtctttgctgacggaaaactatttgatgtgatgcacaaagatgaagccggtaatatggtgacgaaacaaatcgacggtgactacttcttagcagacgcacagaaggggcctgtcgcacgattacttcgtaacatgtccggtatatctatttcagagttcagtaagcctgaggcgaacgctccgttcatcaaaaaggcaaagtatcatctaggttcattctttgatgtaggtttccaagatgagccgggaggacaattagatttacttgacccgacgagttggggtacatccttaatcaatacggtaacaggacgcttccgtcaaagtgaatatgtaaaacgtatggactatttatcaaatgccttcggtaaagataaagacttcatctacatgcgtagacacatggacttaaaggatgcgcctagctataaggattacctgaatcagttccgtgcaggtcgagacaatatggatgaagtaacaatgtctacgatgtttggatatggattcttcgaacgtctaaacgctacgctgaaccaagtgaacttaggtctatcgaacaaagcgctcggttctgcttacgatgtattcgaaggactgttattaaaacgtgtcgctccaatatgggctggtatggaactatgggattacatgaactatgaatctgagaatctattagggttccaattcgaagaccgattcgcacagatgtatgcaaacagttctgtagagattgcgaaggttcgtgataacctcggcattacagattgggcgaaaggtgttgcgccgttattagtaggcggtagcgaacttgcggagattccaatactcggtaacctgctagactggaatgatacagcagaagaaacgcaggactactgggagaatggtgaagtcgctgttcgtaaaggtcgttggtggcaattaggtaatacaccatacatcggtggtaaaacagaatactaccaaccgaactgggtacgaagaacattagctgatgtgaagttctctgaatcacagtatggatcccgtgaagaatattatgagaatagctggatgccatcactacgtcatccattcgcaccaatcaaacacttctttactgaccagtatcactgggaggagaagcactatcaagacagaccgtacatgattaccggtggtattcctgagattgaaaacttcccgttaattgggcctatcttaaatagtacaattggtcagctattaaaaccgcaacgtcagatgcatatggaagcgtggaacggagatgtacaaggtatgccgttaccagtccctcctgaaacacttgagtatgcacaagagcgtactgtgccgatgacatttgataatgacgggaatgcatccttcggtttcgcttcaaatgaagataacgagacagatgaagatggcaatacggaaattgatccgggcgcaagcgtacctatcttaacgcagggtgctgcttataatatgttgcctcctgaaacgaaggaacaaaaccaagccttacaagagaagctattctcttacattacggctggcggtcaagtgcaattacttcgtggtgatccaggggcaacaatatgggatgcaacttctgttatggatacgaaggctccaatgagtaccggtctattcaaacaggaacgtctgccgatgaataaggataaagtagagacaggtgcgccactagagacagatgtacaactacatcaaatgttagggaacctgcactacaacatgacagagatgggcggattctacggattcattacgacaagtatctttggtcaagtcggtaacagcaatcctatcctgcaatcatcatctgacctgtcatcttatactcgtgccttttgggataatgacatcggtggtttcggaggagatgccaatgagatattccgtcgtttcttaccatcggaaacatttgaacataaacaaaatgaaatcaaccctgtagcaaatacgatgccggactggttaccgggtagcgattacttcgttaacttccaaacaggtgacccctacatcaaagtgaagaagggtgaatcacgtttaccgggcgaagggtatgaacgtttatacaacattgattctgagaagatgatgaagctagatattggtgcatccttcattggatatgacgaacaacagattcgtgaccatatgttaaagcgtgatgcctataaagcagaagccttcaataagattctaaaagctggtacgaagatacatgagcaagtagagaaagacttactggcaaaaggcgtagcagatgcgtctgaggaatatgtaaaggatgaagcaacaggcattggcggattcttcgacttgtatgcaaacaatacaaagctactcgactgggcgttacaacagaacgtagcagagtttaaattctatcaaacgccacgagaggaaagcggaagagaaccgtcagaagcactcggcggattctatgaagaggtcgatgtattaaatgacatggataatacccagcgtcaaagtttccttgaacaattcgtagatgtgtatggacaggaaagtgcgacagtcgatattaagacccgtggtgcaaaagcattcgctaaagaagggatgcactttgagaacgtacaacagcttaacttctatgcaaagcaaaagggtacacgcatgaactacttcattgagttaaaccgtgacaaccccgatgcagggattaagatatttgcgttccaacaaagtgatgacctgctgaactatacgacggaaaaagtacaacgagtgcgagaaggtatccgtcaagacatggaatccggtaagctacatcgtggtgacctctatgatccaatcgacagattcagaatactagctgacgttgcgccgtactcacaagagtatcgtgatatgaaaggtcagctatcgaatatgaacttagatgaagatgatatgaaggaagcgcaggagattaagaagcaagtatcagaacgaaagcacaagtatcgtttctatgactaccgcttcaagactgcgaatgtcgatgaaaagatggttacagtagaccatgtaatcgacagtaatacattcgtatctatggagtttcctgataatccaatccgtctggctggggttcgtgtaagtaccgcaaaggataacccgattgcagacgaggcggctcgtgtcattggtcaaaccatcaaggaaggttcgaagattcgtatacagtatgatgctgacgagcagaaccaaatcaaggatgatacgtataagacgctccaagcggttgtatacgaccacaaaggacgtaacctaaacaaataccttatcgacaatgaactcgcaaaagagaaagagaatgactactctgcggctgcggttcatgcacgatttacaccgggtgaaattaagttcggttcgatgtgggaaagctttgctcatatggatactatcctgcatacaaagttactacaagttcgttctccgcttgagtcttatgagcgccgtgaagtatacggaaaagactggcaagaatggactgacccaattgaagacttcctgattcctgcgattcagagttctgctattcataatcccgttgtcgcaatcggcggtggagcaattgctggtatgatgtttggttctctaaaaccatctgatattggcggagagaaagttattggtcgttacgggaagatcgtcggcggattcatcggtgcatctgttatgggtatcgctgtactgaaccgtatgcttacagaagccataacaggagaagcatggattccagaacgtagacagaaagagcgtgacacagaagaatacttcgatgtgttagagtacattaagtacaacgcactgtatgaccagtattcaagaaaggcattaaaggaagaaggcatcaacgttgaatcgtatttaagcaacgagaagttcgaaggagataagcgtaagcaagataagactgccttacaggaaatcaagaaagaactgtatcgttctcgtgcgaatcaagttgggccactcattgcgaaactacgtgagcagtatggcatcgaagcagagacaaaagaagaagcgatgaaagccatcaataaagaaatcacgagtcttacaacgcacagggagattaaagaactgtctcccattgcagcaaaagcaatcatgtataaacaggctgcgaaacaaacgatgtatggatacgaagcaggtgacccaattgcgaatatcttatcggcattgccgaagaaagaccgtgagtacctgactccatttatcaaagcgcctgaggaagaaagacagagaatcctagacatcgtgccgaattacatgaaacgtgtactacaatcagcatggggattagatgtagatgaaaagacaccactgaaagattactttaagaaacatgcactgccgggtgcgaactgggaaggttggagagagaacgtatctcttgatgacataaaggtcaagtttgttgaccgagtgggactcgacccgtcagaatttaatatatgggaaaatgacaaacaacgtgctgataggctgaacgtaccaacgcctgatgtgttcaatggacgagaaagtgcagaagagtattctcgtaagttaaaagaaattttaacaggctctaatattcatggactaaatattgatataatagagtcagacaaaccgggaacacatataaatatgaacattgcgcatgatcgacgtgacgatgttcagaaaattatcaaccgtgatggtcattacatgttataaggagttcggttgcggactccttttttatacagaaaggggttgcgatgatgaataactataacgaaaactatgcacgagaaatattcttcaaccgcatgatgtactttatcaatgagataaagactggcatggtgaaagatggtgttcagaatcctgcggctgaacaacaactattaacacaggcgcacaatgtgatacaagcgcagacgatggcggatacaaactacaagttccaactgaataacatctatgcattaacacgaaactctgacccggttacattagggaatacaatggcaaacaatatcaaggacttaggttacttacctgattcgatgaaacgtgttgccgaacgacaagcggtatcctttacggaggaactaaactcatttatgtcagggttacaatcgagaggtttaacgaatacgtcaacatccgttgtaaaagcaaatagtatgttaacgaataccgaaacatttatccgtcatgatctgtatcaaagcgtggaggacatcggtatcattggtcgtcgtattcatgaccaaaacttctatcacgataatgtattcgcacaagttaacgaggcaatcttccagtatacacagggagctgatgcagttgaccagcaaggacgtaaagttggattctccgacttagaaagaagctatcgtgattatctcatgacacaatctattacagcgcaagagatggatagatggggcgtaatcgggtataccgacgacaagattaataaggtacttgccaacattgaccatgaccctgcttccttccgtatcttcgctcatgatttacaacaaggtgtgaatcaatacactcgtttaatcaaaggcgacagacaacagctaaacggcgtgcgacaaatcttaaaggatgcagaagcgcaaaagaaacctttagttgaagttattgagaagtcgtatgcgaatacgaagatcgggcaagaggtattaaagaaagtacgagcaagtgggaataagacagatgattacgttcgtaacatgatgcgtaacttcatgatgatgaaaggcaatatggaagtaccggtggttcttgcatctagtgtcactgaccaaacgcaacgtatggtagatgctatcggcggagcaatgaaagagatgaacaatccgaagaatcctgactatgccttgtatcaaaagtaccgtggcatcaaaggtgagcatacgaagtttatgaacgaactgtacacgattcgtaaagaggcgaacgtagaaggtagcttatacataaacaatcaagagagtttatctcaccgtgcaaacagtatgaatatgaatgaagatacgaacgttgcttacaagaaggaaactctccctgatgtaaacggtgaagaccgtccacgattaaatggagagaaacgttcaacactaccatcgcaaagcaatgagtatgtatcgagaacatatgatacgaaaatgggttctgttattcatattgatgaagcaaacaatacgtactatgaatccaatcgtcttgctatgatcttggatgcaaatgacgaagcggaacgtaaacataaacgagcaatcgaggaacaattggcagaaggtcgtgaacgtccacaacaaccaatgaatagattccgtaatatcttcgcccagtattttgtagagacaggaagcgaagaagcaaaacgattagctggcaaagcagaagcagacctatcggtggtattcaataaggataagtatgacccgtctatgcgtaacgttatatcggaagctaccttcaatgatattaaggaacgtattgctaacaaagaagtatcatacgaaaactttagcatcactcgttatcaagcgaaagaccgtaaccaacgtagcgttgataagtgggaatcggcactagagaataacgagtccattcgtatgaagggtagcgaggttggtcaccgcatcgttccgaataaagatggtcgtgccgtaagttcagatggctacgttacattagaacgtgaacatgaactggtagagaatgcacgtatcaagatgaaggttgaacaggagaatgtacaagacctactcgaccaaatggatgaacgacgtagacgagcgaaggttacagaagaagcgtctcgtaaagttgtagaagaaatccaagacgacgcattcattaacattcgatacaagcaagaggctgtaagcgaagacctgaaaaaccgtagtgagttcttgtacgaacgactatcgaatacagataaatacttacaagataatgacctaacgaagtacggcattaaagaagcgaacgttcgagcaattgatattgcgaatgagttcaatgtatatcgtaagaatttaacggaattggatccggaaggtatcgagcatcgtaagtcattaccgaagttcttattacgtatggcagaatctcatgcgccgtggatgatggatgatacatctgaactggtgagtcattctgaggttgcacttcgtgaagtattaaaccgtatggcgaatggtcaacagactatcgacttaacggacgaaggtttactaaaagagttcgatgtatatgcagaacgtgatgcaatggggcaactcacacagcgtgctgaaaagcaacgtaaccagttcattgaaaacctagcaggtcgtaatatctcgcaacgactagaagataatttattcttaaaagtgcttggcgagatggtacaacagaacttccaagacaagggtacaaatggcggagttgaaagtttaatccgtgcggctgctgatatggcagagcaaaatgaccgccgtgggattgcgcatgatattcattccatgattaagtctccaacaatggagttcttatttaaagaacacggatactatgcacaggcaagatcattaacacctgaccaaattggacgtatcattcgtgaacgagaaatgacagaagcatcaatggtatcgttcgaacgactaatacagaatgatgtggactttagcacggctgatgagatatggtcacagcgtggtatcaacccaatggagggtcaagaagttcgtgtaggcggagatgcttacatcaagcaagcctatgacgaagtatatgatactcgaatagcgaatgcttacggtaatcctgaacaaagcgttgtaacaaaactaggtagcttaacagatacagactacaaaggtggtcgtgtcacagatgaagctgagattatggaacgctcgaaggcacagctattacgagtaaaagaagaagagttaacgatggagtacaaggcaaacaaaatgaataccttcatcgaagacctgaaaacaaaaggtgtatccttcaatcatattgatgatgttaacacgatgcttgatttagcgaaggaacaaggtatggaacatatcgaggctacggttcgtgaccaacctgcttacatctacaaaggtgagaagggcgatgttatcgctcacttacttcgtgatgatcgtcgtattcagttagtaaactcctctgctggattacaagacagcgtgcatgtggacgataagtatccaacaaagttatacggcggagcgcagattcaatatgactacggtgatggtggtgtaccagttcgtcaccaatacacttcttacgatacgattcaaggtagtggtaagtttgctcgtagttccaatccatctgttacggtagaaggcttccagcgattcctacgaggtacagataagatgacgtaccttgatatagaaacaacaggtttatcgggttctactttaccggaagaagttattcagcctcttgaggttcatatgcagaaggttcagtgggatagaagaaacggacgtttattacagaacgaggatggagagttcgtgattcgtcacgctggtcgtgagactgttcgtgagcaacagcttatcatgggtctgaaacctgagacaaaagcattcatggaagatgttattgcgaacgaggacttccgattcaccgttggtgatagaagctatgacctgattgattacgaacatcacaagggtcgtattaatgacttaatcaacaagtccgaaaatgcggtagacattcgcagacaaatcgtagagaagcaagataagttatggttcctacgtaacattgcgaagtatgcattccctgatacagaagatcatggagagattatgcgataccaacagtatgcaggtaacatggaacttccgaagagtggttctgagttcctgactgtattgcgagaagatgtacaaaaggctgctgataacttagcgagcgcaggtacatccggtggtgtgaagttctcaccagtgcaggttggtacgacagaagaaggtatgattaaacacgtatctcgattcgtcggaaaaggaatgcttgcaggtcagaacgtagcagatgccgattggagtaagttcgttaacatctcaaataagttagtgcaggaggcgacgttaccatttaagacagcgcagaaagcgttgatggatggtatcacagatgtagccaacacacatgtactggatacacttcgtgacattcaattagccgcgaacttatctgatgtaaatggattcgacaaagcaacgaaagcctatatcgaaccagtcatcaaacagatgatgaaagaagatacaggttacaagtcactgagcgacgtatttgacgatatacgtaaagctaatagcgaaggtatgggacatgaacgattcactgctatgctagattctctgcaagctggcaaagatgtagacggattcgatagcaagttaacgatgaacctacatcgtagaatcgaagacttcgctcacattgatgatttatatcaaagtgcaagtgaaggaaactctatgccgaagtgggcgaagaagggtaaacttaaccacttcaatgatgagtccacaaaagaaatgctaacaggtattgataagctagttaaagatgtgaatgtaactggtgacgaattgcgacgtgtacagaagatacaagcagacttaccacgacctaagattgtagagcaaatgtacttatacaatatggtcaatccaaatgcggtaggacgtagtgcagaagcacagttcgaagcaatgaatataaaagtagatgatgcaacaggtcttcacttggctcgtgccgatgttcgaaacaacttaaagttaattgaacgatacggacaagagttaggttatgaccctgacttcatgttctttgatacaacgccactacaaaaaggtgatgtcgttgacctacatacatcattcgatgaccgtattgcacgaggcgtatactcggtagacagtattgataaagcaaagcatacaatgacattcggacgtacactagaagatggtacagaagagatgcatacgatatacgggaaatcaagtgctgacctttctcgtaaagtaaacaaccacttcgctttcattggtacaggtggtgacgcaggtgcgacagagttagctattgaccgattcacacaagataatgcaagacgacaagtgaatcgtgcgatgcgacatgctgatgtatttgataagtacaacaatgagattgatgaactggttgagaataacaaactgacgcacgaccctcttcgtaagatgcaagctatttatgaagaagcacaggataaagtaaatcctgaatatggcccagcgatgcatccaaaagatttatcactagcagagcaagtcgccctgcataaagataacgtcaacttctttgaaggtacggcggtgcaagaagcattagatgaaacagcatcgaatacaaagcgtcttgcctttgataagacggaagactggatgttatcacctgagggtagacagcgttatactttcttagatgaggttcgctcaatggaagcagccggagcattggatccgaagatgagtaatacacttgtgaaacaatggaacgatgcgattaaagaagagggattaagacgaggagcaaaacactctgtcttcaaccgtgcgaatcttggtacactcgataaccgattcggatatatgaatggtcgtcagtttgtattagatacatctgatccatctcgtatctcgaacggattatggagaatggcagaagggttaaaaggattaacaaacgaaccggatgaagtaaaggcaaaagcgaaggctcttaattcccttgtacttccgttcttacaggaacaaggattcattaaagagtttgaccaagcgtcacctccgggaatggtcagcgttgtaaatcagcttatgaagaataaacataaccttccggcttatgaagactatgacccaatcgtaagtgctgaaatgagtaagagtgatatgggataccaaaagttcttacaagataagcatgatgaaatccttgaaccacaccgtcaatcgttaacgactctgcaacgtagtaagcaacaagtgtatcaaagtcagttatcggagttacgtgaacaaggactctatgatccacggctgacagttgagccacgcattagtggattcgatttagatacaatgcgctttggtcgtaacggcagtgtatcaactatgcaagcagatgaactaggacgattgctacaagtgattgacccaagaacaagtgctgatgcagttgacgcacgtaataccattgctggtgaattatttaaccgtgcgattggtgatggacgtttcaatatccaagacgttatcgacgctggtgagagcgacagacttgcagcaatgcgtgccttaaactgggtgacacctgataaccaagtgaacacgaacgttgccactcacttagtcggtgagccgggcattcatgcaagtgaacctcttggtatgcaaagtgttcagatgttagataagattgctgaacaagatgtcggaaagtatactagagataacggatatagttcttaccaagataagattcgtaactggaaaggatacgaagatacggatggtaaatgggtaggtgcgtatgatacaagcggtaaccgtcttgaatatccgggtcgattccctgaggctcccgacaatgaatcacgaagagtatccaccttcgatccgaaggaagcatctcgtgcttcgaatgaacagattaaagctgatggacaaggacgacgcacgcaaacaccaacgccgggtgagggcggtcagaatgctctacgttactaccgtacagagaaccttgacccgaataccaattaccaacacagcaatgcgattagtgaagccctgcattctgcaaaagagaaagctactaatctagcatctgaaatgtggcacggcacaccgggtaaagcaatgaagtggatggccggtatcggtgctgctgcttttgcggtgaaccagtttatgaatgccgcttctcctgttcagttgcaacgtaaacctatgggtcacggcgttgaaggtgcaacaggtcaagcgaacgacggattaaatcaacagcagaaccaacctgctactggcggtaaaacatacgtcaatagcggagagagtcaaccacctgctggttatgaagtgaatgtaaaaggtacggctaaaggagatgtcgattacaatgcgatgcaggagaaggttaatcagaccatcggtaacttcaacgctaacatctcggatgaccgttctacattcaaccgttcatggctagagaaacagttcggtgactacattgatagaggatacgtggggcaggaataaactgcccctatctataaaagaggtgaggaatatggcattagcaacatcggctaattatcatggccccgaaagtaatagttaccaaaccggtttccgggcaaagctcgccggtacagataaatataatgcactaattgaaaaagtagcggcagagacaggatgtaacccactactgatgaaagttctgatggctatggaatcgggcggacagatgcttggaacaaacagtctcggatacttcggtctgatgcagacgaactacaaacgattcagtaaagaacttggtgcagcctatcgaactgacccgtacctacaaatcaaatcgggtgcgatcgagttaatcgagaagaaagattacatcatgggtgtaatcaaacgtggtaacggtaaaggcccgaaagaaaaatggacagaagaaatcttccaaacagcatgggcttacaatggatatacaatcaacgcaagttctaagatgatggatggatatagatatgcacaaaccttccaagctgtatacgaaggtatgggcggtagcatggctgataaagtttcgtctggtcagatgggcggaggcactggaactggcggtggtggcggtactggtggagaagtagccaagccaaagtatgacggcggtgcgccggactggaaaggtggaggcgaacgaggtatcggcggaacagggccgggaacaagtacgaagatggatgactatgagaagaatgatccgaacagcgcaatgttcatcggtatgtcacctgaggtagaagaggatgattatccgaagcatcgttatgcagggaatccattcgttcttcgtattggtgatagtcagttctttattccgccaacgtccattaaaacagataaaacgtcgagtatcgcagagcaacatatactgcgtgcgaagacacctatcatgacgaaatccggttacactttacatacgctttcgattcagatgtacttcttcggtgaagaacaaatcaatggatacccaatagaggggccgggcgggaagacgtattatatggatggtcttcgtcctttactagcgcagttctataagaatccattcttaccgattcgaaatgagttaatcaataaccagcacaacatttataacgttgccttgcaggacatttcttacacagttgaccctgacttctcggacgcaattgtcgtaaatctaacactgattgaatgttgcgtggaaccatacatcaactaccctgagtttgtatacgatagaatctttgtctatccgttattccgttggtggtatcaacaacagatgctcggcaaagagacagagcgttattcgggtacatggctaaagcctgttaacaaacatctaaacgggaaagcattctttaaagtattagaccgtgacctcattgaacaaatgaaggacgatgtaatgaagaagaatgacgggaagacaacgaaggatggtcaaacgattaatccggagtacgcacttgctcgcatgaagatcggcgacatacgaaaactgatggtcgaatgggagactggggatgcggtacttacaaacgtcacggtatccgttggtaaaaacttaacaccaatgtatatggacgattatgagaaaccattgttccaagacttaggcggaatggtacgtggctttacgctagagtattattgtacaaaccgtggagagttagaatccttccaagcacttgcgtcacacttagaagagttaagccgtgactatcgtttccgattcgtatcgggttaccttgcgattgataatgaactgattaacttagccgggatccaaaacgctatgataacaaatatccaaacaagtaccgttgaaggtatgtcggataactatatcgttcgtatcacatgtcgtgagtttaatgcgaaccaaaagaatgaggagcgcttaaacggtatcaactacacaatgaaagaatccctaaacaaatacggattcgtagatgcgataccaacgaagacaaaccgtaagagcgagattgcatacgaagcagaggttatgaaaatcctaaacgacttagagctatatcctgaccttgagttacctacattcgaagtagcgaacaaagcggtagcagaaatcaatgcgtaccgtgagaagagaggtcaatcgaagttaccatacgataagctcaaacaacctgacaatgcgacgtggtgtgaccctgacttctatatggcatatccatctacgaagcaagcctattctcatatgcagttaggagatatgggtgacaagattatcaaccaacttcgtaacggatcctatgatgaaattaaaaacatgagtaacgacgaaggcttttggaaaacagcagatgacattcagcagttattcacaaaaggtgttgtcgataattcatgggatgcagatgcagacggaaaacgtaagttactcaagagttcatctgtcacatttgagaatgccacaaaagacttcttagtggatgcaggacaaagctggggcgatgacaatagtattccggaagacaaacaacttgttgagatgatgacacatgacatgcttaaatacagtcaccgtggtcgtatgactcgtgccttcccttcttacatgctactattcgtagatgaaggacaatgggtagacggcaagcgattatggaataactattatacctatcatgcgattcagcagatggatattatatcggataaagagaatccagttgaccttgcgtttgttactttatcaaacgtatacggtacattcgacttccaagcgaagatgagtgacccacgtaagtatgcaaaagaaccaattggattaccgggtcgtatcggtgcattaatcgacgactttagtttcaccgttaccgctaaagtaatggcagaacgctcgcagttaatggagcaagtgaaactgcgtgaaggcgcacgagtacatatgcgaatgggttatagtgcaacagcaggtaatatgccgattgtatttaacggtaagattgcttctgttaatgaaggggaagttattcagatgctatgtcaaggcgacggcgcagaactgattaaccaatactacagcacagatccaaaaggtgatacgccaaatgaaccgcacaatacattccaagagatgctaacgaaacgtacaagtaactactggttcactgtatcagaggactgggaattcggtgacaactatctaagttactacggcattgaacactttggattcgtagagagtgaagcaggtggtttctggaaaggtctaggcaagaacttcgaaatcctaaagagcttcctaacggactctgtaccgttctctgcttacgacattatgaagaacatctataagggttcgaactcaccagtaacaggacaggagacaggatggagtccatttgacggcgagaagaatattaacatcgctgcttacaataagacgccgtgggatattggacagatactatctatgttcgttccggaatttatctgcgcccctcatcatcacggattccgtagtaccttgttcttcggtatgccacactggcctgtgaaatatgaatacattttaaaagacggcaaagaaggaaacagctacacggattatcaagagaaggtaaaaccattccagcagttccatatgattacgtcgggtaatgatattatcgccaacaaaatacaggcaagcagtgagaagttaaaacacattgcagtcggtatgtataagatgggtagcgggcaagatgctgctgagtcctatacagtatgggcggatagaacgatcattaaagaacatcaaaagatgatgcttgtagaaactggcgtatggcaagatttacttggccccgacttcttatatacagcgctcggtaagtacggattaaaaccggtactcgaagcgccgggttccttactatctacaatcggtgacttattagaatcagcaccgctaggagatacaatggatgatgtcggtaagaagttccaagaatggggcgaaggatggattgaatggttagatgcagatagtattttcactcctggtcaagtgcaagcacgtaccgttgcgattggtgcattgcaacgtaagttcatggaaatgtatcagggcgaacttgttatcctcggtgatccatctattaaaccgtgggacatcttctacttagacgatacacacatgctgatgaacggtacagcgcaagcagggaaagtatcacactcacttagtttgcagacaggatttactacagttatcaagccggacttaatcacaagtcgtaccgatggtaagggaatgcgtactggtgttatgaacggactcatccttatgggttctgcacttgctgttattacatcacgtaaaatccttatgtcacgcttcgttaactcggtagcgagactcggattaaaaggtgcaatgaaaacaggtagtggtgcaggtaaggttgtatcaaagttcacaccaaaagctatcaagcaagcaagggtagcgaagtggggtgcgaaacaactgaaggctggcggtacgttcctaaaagaaggaaagtatttaaccaaaggtctgaacttgttaaaaggtaatatcgtaaccctcgtactattcggtgctgtatcagagtgggttggcacttggttcgagaagaatacgaagtacaacaatatgatttatatctatccattatggaagatgggtgagccattcgccgcaggtatcactggcggagcgcacatcattccgggctacatggataaacgattctccgacccttactcaactggcaaattcgttcaaccgaaaatgcagttcatagatgagaaaggtagggagaaaaagaagaaaggtggatcatcgggtagcggtggttccggtggcggaggaagcagtgcttctcgtaatgctatcgttgagggtggtaaatcatatgaagggaaactgaaatacgtattcggtggtacaaacatcgaaggcggtacaggtgactgttcaggatttacaagccacgtattcaagaagttcggtaacctaaacattggtcgtacgacaggtgaacaggtaaagaacggtacgcaagttgagaacggaaaagagcaaccgggtgacttaatcttcttcaagaatacatataacagtacacacatctacggtgtatctcacgttggtatcgttattggtgataagaagatggttcacttaggagatagcggttgtcaaatctccgactatacaacgaaatattggaaagagcatttccttatgttccgttcgtatattactgacatgggttctatcgaagtcatttcaggcggagcgttccgtcatccacttgaacgtcgtacttctattacatcaggcttcggaatgcgtaaaggcggtatgcataaaggaacagacttagctccactcggatatgcaggtaaagggcaagcacttgaaaccaaagtctatgccgttgcagatggtaagattatagatatgggactaagtgattcaatgggtaactatgtaatccttgctacagcaaataaagcaggtgcgccgcacgttattacgtacatgcactttagacaacatgctccgggcgtttcaaaaggtatgcaagtgaaagccggtacgttcattggatacatgggtacgacaggtgaatcaactggtgtccatcttcacattgaagtcaatccgggaacgtcacgcaatagggaaggaaggattgaccctgttccgtggttccaacaacaaggggtgcagttataatgaatccagtacaacaaaagatatttgaaagtgtcgtatccccagccttatctcaaatggctgggatgtgcattggtacggtaacggatttcgtacccggtcataacttcgggagtgtcgtgtattcgactcccgatagtgctgaccaaattgaaaaacaaggtgtaccatatatgaacgtcattggcgtaaagacaatgtgtccaatgccgggtgatgatgttctgattggttttatcaataatagctacaagcaacctgtcatacttggtatcatggataaagagtatgcgtttcaaacaagaccgttatatcaatctcattttcgtaacggtagtaacatacctgattactattctgaaagagaaggagagagttggtaatgtgggttgatccatatgcagttaatagacatccgagtagattcggagagcatcatgataaacaattaggtgtcgatatacaagttgatccaatggaagaagtcatgagacgagtggaacataaattccagtatcgtcctgaggagcaggggatattccatcctctgcttcatcacggtatgaagctacgaaataatggtgcagttgaaatctttacgaacgatgacaccggtattcgtatcgacagaaactcacaaagcattaacaacttcgccaaccattataagaaccatgttcacaatatgacgtcatggctgacgggttccgaagcatcttatattgaaggtgaacgcaaggtaaagacaggcgggaaacatatcatcatatccggtgacacgattgaagtaactggtgcgaaagatatgaacctgaaaattagcggagatgtgaatgcaaacatatccggtcatgctacaatagaagttgaaaaagacttaacgcttcatagtaaacacctagaggtaaaagcagatacctatcaaacgccgtggggtaagaagtaggaggtacgtatggctaggaaagatacgattgatttagaacttacgccggacggtgaccttgtacttgaccgtaaaacgaacgacattgctgttgtacataaacaacattacattgcgcagtcggctcgtattcggattcaagtaacagaccccgaatggcaggattaccaagtagatcaaatcggtgcgaacttagaagatttaatcgggctaccgaacacccaagagacagcgattgaaggaatcgaaagaatcatatacacactcacaaaagatggcttgctagacacagaggagatttatatcaaacctgtaccagtaagtaagtatgtgattgcgttctatatattcattaagattccggacgtacctgaatcaattgggtttgaagtcctgttcaacctagcaactgggttggcaataaggagtgtataaccatgattctaatgcaaaaagatttagaacaaatcatgcaagagacattagacgagttaaaaggaatgagtctcgatgcaaatcccggttcggtgacacgcttaatcttgaacatcatgaaccaacaaatcaatgcgttctatgaaacgttcacactgaatcatgcacaagcctttgtatcgaaagcgaagggttcgttccttgaccaaatcggtatgttactgaactgtcagcgattaccggaagaagcacatgatgacgatggctatcgcttccgtatcacaaagcagattcaagttgtagcttctgcaaatgaaatggcagtacgactggctgcactatctgtagatggtgtgcaggatgtgaagatgaaacgattcacacatggcagcggttctttttctgtctatgtcgtttctgacagtcctattactcctgacttcattctgcaagccgtacaacaaaagattgatgaggtagaatcattcggcgtacgaggtgttgtattccgacctgaattattaccagtcgatatgagtgttcgtttaatcttcaagaagactgtctctgatcttgagcgtaaacttgctgtcgcaaaagcacaagaaacattaaagacttacgtgaacagccgtaacgttggtgaggcattatctgtcgctcttgttgaaagtgaattaacaaacattcatgaaggcattacggacatcatcatctataacttcaaggtgaagaatcgtccggcattaccagtcaatcaaacatgcgcatggaatgaacgtttcatcgaaagcgataaaccaaatgccttaatggtaagttaggaggtgaaagcagtatgaaagatacacaactggatactcggcagatacaagcatcacgagatggattcaagagcttgcacgagcaacttgagttcggacgattcaactcggtgaaaagtggtgctgctcctacgatgtttcaggagttacaagcaacagatggtcagactgttttcacacttcctaacggaacgtttacagtaggcgaccatagtttgcaggtctatgcaaacggaatgctaatgagagagggtaaaacaaatgactacatcgaaatcgacaaccgaacaatcgagttcttattcccgcttactagtcgggacatcattgtgttccgtgtggcaggtggcacaagcggcccatcacttagtgagcgacatcaagcagtggctaatcaaaccgattttaaattggcaggtagctattcaacaggtaatgattcactcatcgtcttcgtcagcggggcataccaaacgctaggtgaagattacatcgaggtcgatgcgaagactgttcgcttcttacaacctttagaagaaggtgaccttgtaacattccgtgtcgaagggttgcccgcaatcgaaagtaaatataagaatgttcatatccatcgtcagtatgattccaacaagcaactcatcaaagaggaaatcactggcgatgaacgtctaagtaaagaatatatttacgacgaagaaggtaagccgaagatgatgattacaaaagatagcggttataccatcaccaagacatatgagtgggatggattcaactgcatcaacattagcgaaaccgtgaaggagggcgtgtaatggcacaagtcttagatgagtttgtaatcaactacaccctccaacgtatgcaaggacaacacgaagcactagataaacatactaaagaatacgaggagaaaacagatgattctatattacgggtatctaatcgtgtgggggatcttgagcaaaacggcgtggattattcgtccgatattaaagacttatacaaagctatccagcttatgcaaaaagatttacgggaaactaaaacggaactgaataagacgaagctcattaacttgaagcaggagattatcatccgtacactgaacaacctgaaatattttgaagcacatgatatgtttaccgatacattcgctgatgggtcgggcatagattgggagaatagtttacgagcagaactcttaaacggacaacaagccgtaggggtaacgcagaagtcgattatctctgcacaacaaacgtcgcagaaatcaactctacttatctcgaagaatggttctagtgacgaagcacttgcacaatcattcgtcgttgataaaactcgtaagctagataaactcggcttatggctagagcctttcagcgtagaaacattccgtccgattcatatcggtattacggatgtactcggtggttcatggcttcatagtgtgacacttgaagttgaccaagtagcgtcagactggatggagattgacttccctgaactattacttgaaggcggtaaggattactacattgtcattcgtacagatgatatttacggatacaaagtcggtattgatccggttgataaatatattgctggttcttcttacagctattacaagaatgtatggacggataacaatcatgacttatcattcaaagtatggtgcttccttgctgatgatgagaatgatgcgattatcctaacgtatccaaaaacattgccaacacttcctgagatgattgtattcgagaaggaagatacattgtttgatggtacagctaactattacatatcacgagatggcggagcgcactggaagatacttcatccgggaattgaaacagacctgaatgatttacctgccgggaaaaccgttattctaaaagcaattgttacaagcacggcacgtattgaagcgtggggctatgtaatcaaaaggagtgaggcataatggtaacacgcttaaatcagacacgagttaagggtggcgatccgcagtacgagaatctacaagaagaactgcgtgcaagaaccttcggtggggattataaaatccctatcggcggtatacctcgtagtgacttatctgatgatgtcagaactgcactacaaaagggcgagtccgcctacatgaaaccagagggagggattccaaatggcgaccttgatgtaaacgtccgtaactcgttaaccaaagcgagtacgtcctatcaaaagccgagtttaggaatccctcttgctgatttaacagaagacttacagaagcgtttaagcgacttctcgaacttctatgtgtatccgaacggtggtattccattagtagactttgatacacaagtaaaagatttattgtcaaaagcaaagactgcgtatcagaaacctgctgacggcattagcacgaaggatttacacaaagatgtactcgatacattaggaaaagcgacgacggcatatcaaaagcctgagggcggtattccattaacagacatcgctgttccacttgtgcaagctggtgagctagaaccgatggaagaacatatgaaggatgcgtcaaaacatatcactgaccatacgaagctgcttaacgttggtaagaaaacgcatgacgagattgatacgtcgatagacaatatgcagaacgacatgattctcttacaaaaagagatgtccgatgcccgtgatacattcgtatcgcttgatggtcgtttcaatgcttcgattggtcgtaatacatcttatgaagtaaagacttataatgactggcgcatgggtggattcacgggactacaaccaacaaaagaagatctcgttgtatttaactatccagtcgaaacagtcatgacgagtattcatgatatatcgggtcaagaaacattgactgctgataacaaagttggtgaccttgcatttctatcctactccggattcagaccgggaagcgaatggatagcgggatactcgacaaatgtcggtgtaaaagtaaaagcatttgtatatgcaccaatcactggtgagtatcaattctccatgcagtttacagggcgttgccgtctacaagttgcaggtaaactcttatttgatacaagagatacatcaactccaattgatgcttacactggtaaaggctctgtattcctagagggtggtaagttatatccaatcgtcggtgagggttggtataagagtacgaatacgagagtgttaacattgcaatggagtacacctgcaatggctggctttacaaacattccattagagaatatgaatatgtcgggctacacaaacaaagatggtgtctatgaatctcatgttattgatttaaaagatgcgcaggtgaatatgtggtatctgcaaacggtgatgcgagattacttgcaggaagatgatgttacatctgaaatttgtacgagtgatgatgggaatgtattctctgaatgggttgcaacagaaagcaacggagagatacgagtaccgccgaagcagtatatcaaagttcgtcacacgcttcataaacgtgatggacaatatacaccggctctaattagttactctatccgtatgatctcggctcgcaacaatgaaatcatcaaggaactatctgatgctcgtgctacatttgattccttgcaaaagcgattagacaacatggcggaatccattcagacaattgctgaactgtatgacaacatggatcaatcgtttattcatccggaacaatttgcgagtgtaagactacttgcgattgaactgaacttactacaaacctatgtccgtgaagcaagagcgaaggcagactttgtgccaatcgtaaatggcttcgttgacccgtttaagaatgacaaaggtattgatagaagtaagagtgatgcattcaatattacagcgaatggattagttcaagtcgaaaccaaaatggaaatgaagacatcaaatgattgggcgcaatgggacttagatagactggatttcgcaaaaggtgaactaacactgggtgcaaactatgatgcatatccggtttatgaatcaaagctaagtaatagaaagtatgacacatcctatgatgttgattacggagatggatataaactagcgcaagccttttatccaaaggatgaatcgcagtttattaaatccttcaccttctacggaagcatgaacgggtatagtacgaatgttagtatgacaatctatgaagatgtgaatcgtggctattctccgggcggtaactctatcttttataaaagttatgtaggaggaacagaaacatttatactgaatcataaaatcaaaccgggtgcaaaatattgggttgtctttgatttacaaagtgcaagttccggtacacgattctactgtagtaatcttgcaaatgctgaccctcgattaatcagcgataacccggagaaagcaatgttaaaagcatggttccgaccattctccggaagagaagtctttgaacctgtcaatgcgtgcttaaactttgagattgtagagacaatcggttacgagccggaaggtaatggcgaaaagattatcgaccacggcagacctcttacttatgtaaagtcagaagtaacagcagccgatgtcaatgacggagcagtcaatgtaacctaccaaagttcagataataaaatcgactggtcacctgagacaacgaagattgaagagttaccgtcaagccgttatctcaagattaaggtgaacatgaaaaagagtacgaagaagtacggtacaccacgactgcaaaacattacgctctacacactcaaaaagaatgcagagattgtgtctgtaccacttgagatagagcgactgccgactcatacaatactgtcggcatctacaagtcgtgactcggctgtaacatatgccgtatctcgtgatgatggtaaaacgtggcttccatgtttacctgctcactatgcagagctatcgaagttacctccgggtaaaacgattcgcttaaaagcaatcatggatgcgaagtatcctgatacagctttaaaagagtggggcgtaattggacttcactacagagatgtaacgggtcaaaacattacagcactgtatgaagaatacacagcgcaggatggacaaacaatattccatcttgagaatccatacccggtgggtaaccatgcattgcaagtattcgtcaacggtatccgtcaatccgaaggtaaagattacaatgagattgaccagcatacaattgagttcacagaaggactgtataataacccggctggtattgaccgtgtaacattcgttgtagcgacaggtgcttatgatgttcatgattctacactggtaagtcgcatcgagaacatggagcgattacatcaagaagaacttgtggattatgagaagatacatgagtacaatgaagcaggtcagctcattaaaacaaagtacgaaggtacgcttgagtattatacaattgagtacacgtactatgataatgggaaagaacacttccgtattgtcacaaaaggaaatacaattaagacaacggaatacatctatgacaaattcaacaacattgtcatggagagagtgaaaattagtgaggtgacaagtgcatgacgcaacgtgcgaatctacaccaattacaacgtacaagcatcggacacccgaccttctatgaacaatacacacagttagaaaagaactcggctcatcaacgtgagccggttcgttatgctggtgacttacctatgacgggtaaccgtgatgggagtcagtgtcttgtactggatgaaggtacggtgtactactggaatgatggattacaaaagtggcaatctgtattagggaaagcatacgaggtatctagtaatatgcgacgcttcaagcgtaagtatactgccgaagaaggacagacagtcttccacttagactttatttatgaagtcggtgcagatgctttagatgtttatgtacaggggatgttgcaagatgttaaccaagattatgaagagactgatgacagaacgatcacgttccgtaacccgcttccaaaagatattatcgtcactgtcgcaacaccgatggtaattgaaggtacatacggtgtacgtgaaatcgagaagcgcttaaaagaactggaacacaataactatcaaatgattatgtcccagtattatagcggtaagcctgtagaagttcgcggcatactattcgacggcttccttgatacaaaatacattgactttatgatgacatcattaaacatgaagtatgataacatcttaaagacaatgcggctcgacggagaatctattgtgatgttatatgaaacattcgataacaatacgctcatagatccaacgtcagaagtattactaaagaactcggagattaccttgccgattgaagcggtctacaaggaagtattcaaagatgacttctcaactcgtcgttatatcaatgaagaaatgacaaatgcctaccacgaccttgagaaacaattgattacaacagtcgatacatttgcaggggcgaaccattactataaaggtgactttatgggtacgacagggaaggctgaccaagtaggatttaacagcactcaatctgtaaacagagtttactatcaatctactgcatacgatttcacaaaccaaagtactagaagtacatccccttacaatgtgttcagtgttattccgacgcagtattatatatggggtgctagtgatgaacatatctatggtaaaacgaagtttccttatgatactaattactatttacaaggagcagagagctaccgaaatgcaatcgttcatccctttgcaggctcgccagctacgtacgaatatcgttcattacctcttgcatctgattttgatggagggaaatataagtacatgatggtatatgtaaaaagcgctgatgcgatttatgcactgtaccaatacgccgaagggtcagataaatattatcaaaaattgattactagtaagatgagaaattcgtatgccatcaatgacatggttaatgcgggaataccagcgaatcgaccaccgaatatatttggttccgatacaacaaatttcacacagtcaactgattacatctatcaaattgctgcaacagaaaaagaagttttacttcgtacaaaagacaaggtacatttccttgacgttgcaaccaataagattgtaaagacattagactggtctacatctgatgtaagacctgctattttcaataggggttctcaacagtttgcggctgacgctagatacttatatttcccgtcttcttataccatcaatagtgtaactggatattacattgaagtgtttgatattaacacaggtgcgtttgtgaatcgcatcttagtatcacaagcgaatcaaacgtatcttacgacagctatgcattttgatttccatacctcccgcttgatactaggtcagaatcaatctggtaaatatggtttaatgggaaaagaagtaaatagctatacggtgagtaccagctatttatcattcgttatattcgatacgccaaacgtaaacgaacgtatcgttcaatcgaaaccaatcacaacgaatctacctgttattaactatcgtctgcgagctacacaaacattgaatgatggattgataacatactatattcgtattggcggaggcgcatggattaagattgataaggacaaagagaacacttatgtgaatccgaatggtgcgaaggaaacaacgttagaactgcgtgcggtattacgtaccacaagtcaatctacaacgtcgccgatcttaacggactggacattagaagtgatgccttttaaaacaactggtatctacaaatctatcacaaagcaaatgtcactcaaggatggtactggcggacggttaatcacaacagaatctattcctgttacatcttccctgcaatggtcactacaagtgcagaaggatatgccggagattacattctcatctagtaaggagttcacgatacctgataatgtatttgaaggtgatgtaacactctttgcgagactcggtacaaacaatgtactgatgtcaccagtcgttcaagatgcacagatccaactatataaagtaaatgctggtatacttgagtcaaccatcttcgaacaaacggatgacgtaaagaatgttacgatgtgggtaacgtccggttcatctaatgactactacaaagcacatctatctcgtgacggaggtaagacatgggagcttggtaagcgtgtgaatgccgtaaaaggaaacgacggcaacgtagaaacagagtggaagtttgatatgaagacagatacatccgaaaagcgtaaaataaaaattaaattagagatgaacggcgtaacagagattcaccagtacggtgctgttattagtccatcgaaggagtgatcgcatgaagcaacttcaccctgaacagttgccgacagaaatcattgagttactacaaacactcaatggtaaaatcgacagcttacaaactgaactaaaggagctgaaagaccttatggcaatttcagaagcgagtttcaatttactacagaacaaggtagaaacattagagggtcagatcgttgagataaacgaatctgtattcgaactagagaagaaattcacaaaagacttcacagacttccatgattcaacaacagaaacaatgactgctttactgagtcgcattgttgcaatggaaaaaacacatcttgaattagcgaagaaagaacttgtaattggattaagtggtgcagaattaatggcgctacaaattaaactacctacaacagatgagttcggttgtgcgattacatggaaatctgataaaccacaatacattacagatgcaggtgctatcacaagacctactcacgaggagggtacgcaatctgttacaatgactgcaacacttgcttacggtggttcaacagatacgaaatcatttgtactagaagtaattgcattacctgctccggaacctgagcctgaaccggaaacagaaacaccacaaagttaaggcatctcaattctgagaatgctataataaagacgaacgtgaaccatgcgttcgtcttctttgtaaaggaggaaaatcatgcgtacccagttaaacgattggcaaatacgagcaagggagaatgaaaacgaaatgaagtacgttcacattccgtatcaaacagttgcgcatagtccggcggagattgtaacgtatccagttccattccctgccgacatcaagggtatcaacatcaagaccattcgcattgtatcagaagagtccctgaacttcgttgtgagcttgatggatggaaacaccaatgatgtgatttacgaaagtctagatgagttaggaatgcaatacgaccaagtagacattccgtacaaaccggcggaacagaaactatttgttcgcatacataacaaagggagcgtcgctacacgctttacaatcgacgttcgaggattagaggtgaagtaggatggcaatgtacaaaggaaaggtcaagaagtcagacctagttggattgctcgttgataaattaacaactgcgccagtaggttccggtgaagcatactggaaagcagtgcagtccggaaaggtcgatacagaaggctatgttcttttctcaaaagggaaaagcgggaaggataaaatctttattcgtattaaagacgatatgacgaatggtcgtatcttcatgagtatgatggaagattaccagcctaataatgtagtaggtttagcgggtacggcaattaatgaatcgtatcaacaacacgtttcttattacaactcaacatacgcatcaacgatgcctgttatctatctcttaaactttgaccgagataaaatcatgcttgtgttacgaggcgatccgggatactctactagttttccggggtatgtacttgcgtggattggaatgccggagcgattaagtaatgagcctgattcaactgcggtatcatttgcggtcagtcgatatgggtatcaattaactgctgctggttcatctgctggtggtgcttatgctacgctaaaatcactacgtaatcgtagaagattaacacagcaaaactacagtatgacttacgtagctaatccaagaacaaaaggtgcaggaggaatcatctctacatctcctatttacttacgagatggacaggaaggcggacgcagtaagcttgctggtgttcttcctatcttccaaagtacaacattacctgactttaagaatggtgatgagattacgattgattccaaacgatatgtcatttatgaggtaggacaaatcagtggcgcaagttccgatgtaaactgcttcccttctccatacatggcaatcgaatacttaacgtaaggagggattctcatggcgatctataaaggaaccattaaggcagaccagttctataataaaatctgcgaactgctcattgccgaaggataccaagatgtaaccatcaatcaatcgaatgaaggtcgagtgttttatacacaaggcgtgagtgggaaagataccttctatatccaactgtataaagcatacttaggttcgattaacattggtgtgtacgaaggatataataacgacggcacaggtaaaggcgcattccaaaacggaaaccaatatgtatacatcaactggatgaacgatatatcaacaactgagtctacttattctgttaagtacgttatcaatgtaaacaaagatagactacttatctacgtagaaggtctatatgtcaactcaaacaaaatggtttcacttacttatgcaggtcttccaaagcgttatgatccaaaagacctaagtgggtatgctgcatttgtaacaggaacatctgttgtaaagactgccgtatctcatcaaacaagaatgcttcgtgaccgttctcttgcaaagaatgttgagtatctagcaacagcgatggacttaggcgcacaacccggatggacaaataaattgttccgtagtcctatctatctatatcatcgtacgaatggtgcgtttgaaggattacgaggagaactagaaggtatcttctacattccaaaaaacgatacagtcaatggtgacatcgtaacggaagacggaaaacaatatatgcttgttagaccaacatataatagtacaacgactggcgtgctatacgggcaatggttctacgctattgaaatataaaggaggaattatagatggcatatcatactggtacagtacaagcgaaagacttgcttgctacaatcatcaataaagtaacaatggttcagccgggcgaaactgatgcttggtggaagaaagaatcatctgtagaagcagatggtgtatacacatcaacaggttcaacaggaaaagaacgtatcgtaatcatgcttcgtcccggtacagatggacaaacaattatcgtcggtacagcaaaagattatacaccgggtgcagcaaatgttgcaggtgcgtttatctcatctagtctattctcgatgcaatattattcagcaacgcaagaccctgctgttcttgtaagctatgatttaagtgtaacgaaagaccgcattatcctacatgtacaaggtgataaactaatcaatggatggtataaccctgttgcctttcttggtatgccagttcgctacgacatcaacgataagaattgtattgttcgtgcaacaaacgagaacgttccatctgctgaaagcgttggacttatggttgtagaaaatagtgtaggaaatgtatggtcgaaatacacatggaaatacgtagactcaccaagcaatccatcatggggtaatacttacttccttgagacattccactttagttttccgggtgaaggtcttcgtggagaactagaaggcatctacggattaccatcagataatgttagtgacggtgatattatagatgtagatggtacgcagtttaaaatcatcaagcgtacgaatacagctcaatataacggattcccacgagatacacttgcaatgaaaatgaagtagggagggatagatcatggctgatgtaaaacatggacgtatccttcgacctattacaaaaggtgcaagacagataacagccgttatgacctctgtagaatctcgttctgtcgcgaacttgataccgacagatatatcagatgctacacgtaaatataaagttgtgaatccattgaaagccggtatcagaccatcacaaggactggtaatggagtcaccggaagcacgcatactagcagaacaagtaccggtgatttacacaggtagaggagcaatgcctctgacaatgaatgaccaaccaacaacagaacgaaaaccgattcgtcgctggcctcgtgactggcacggtgaaaacatgtagcagaggtctatcctctgctctttttataggagggatattatgcaatcacgttttgcagaaataacagaagtcatgcagaaagaattaccaagatggtttaagatgcgtaaggataagaatagtatgggtgcgcagttccttaacgtattcggcttacaattcgatgacattaagttcatgcttcaatatgcattagacaatcagttcatcggtacggctgatgtacatcaaatcgacatcatctataaagccaacataccagctacgttaacggaagaagatacgttcatcttaattggcgggaatacaaagttacaaacctgcaacagcttaaaagagttcttcgagggaatcgacacacgtttcttagaacgtaaagaaatctattacccaaatccatattacatcgactgggtgcagcacatcgtttatttcaaacgtgcgtatgatccaacggaatcttatccggaaggaagagtcacactgcgattactagatgatgaatttaagacaattgcagaacacgaactaccacaacacattcatcatgtgcataacttctttgatgagtttgggttgctactagatacagcacgattatatggcgagaagaaccgtgaatacaaagaacgattacttgatgtattccgtcaccctgctaactctacaaagactggtttagagtttcacttggctcgtgagttaggattatggaaagaagtaaagtggtatgacggcggtgcagaactggtattaaaccaaagtaatatcttaccgcatagcattgaggtcgatggaatcaagtggggagaatctatgattaaatacgaccatagcggacgagtggtactgagcgcagatgaagagttccgaaacatctcacgaacagttcgatttatctccggactcaagatgcacaccttccacgataagcgtgactacgcctttcagcgagagttgtatgacattgatggtacgccgacgaccatgctacaatactatgtagatgtcataacaaataagattccgattctatggaatcactttgtatggaacgaatcattttggaatgtagctgacacagaaatgagtgggtacggagcaatccctacatttaacgatgctcgcttcttaaattggcagaaatataaggggtaggaggaaacaagcatggctactcgtaattttaatatgacgctacaacttgatgcgaaatacatcggtgagcgttggtgggcgggtgaagagattcaccagttatggaatgtagaaaacatgaatacactgcaacgttctgaatggggattagatagaaacggttggatacaatatcgtagccaactggattcctattcgattcgttatagtgcatgggataccatcttaaacgattactacttcgaaacaaagatgcaacctgctggtcaagggaactatgagattgggttagcattccgtatgaaagaccgttactcattctactacttaacatataacggtggatatgcagactggggcggtaagaacattcgcctgatgaaagtaatcggtacgcaacatgtaaagctggctgagtatgaatgtcctgtattcgatacaaagaccgtttacaaagttcgtgttgatttaaaaggtaacaacattaaggttacatgggatgaccaagttatctttaacttcaacgacccaaacccaattccaaacggtgcattcgggccacttgtaatgggacaagagtttgcgaagtgggaaggcttccaagcgaagaacgtaacgaagttcatcttgcaagaacgctacaatgatttaggagtagagtccaactacaattcacctgaaactggtaagctagtagactcgaaaactgttgaagacttaatgcgtaagcaacttgatacatacttaaacggacgagtatatgaacgtatcatctatgatgcattccgtcttgtatcgtctgaaccaagagtcaaactagtctttgaccgtgtaccaaataaaaacgttacatctgacccgacatctcgaatctatgcttatcaagatgcgccgtcagtaccgcctgtcgctcctattaacttactgggtaaagcattaagtacatctagtattcagcttgactggacgcatgtcgatgacagcgaagatggattctatatactagatgagaacggtgagacaaaaggaattgtcggggtagacacattccaatttatcgaggaaggcttggaggagaataccagttacaaacgtaaagtggttgccttcaacgtagcgggacgaagtaaagaatcaaatacagttatcgtgaagacattgcagtccgtaccaattgcgccatctgacttcgaggggaaaccaacaggtgatggtgaaattacatggacatggaaagaccattcattcaacgaagcatcatttgaaattattgagtgggatcagtacggaaacattcaagtcattggtacagttggtgagaacgtaacagtatttgttgagaagggattaattccgttacaagaatatactcgtgctgtaagggcaaagaaccctgcgggcgtgagtcctccttctaataaagcaacagtaaagacgaaaaaggacgtaccaccaaaaccaaacaaagcaccaatcaacttctacggcgtagggatttcagacgatacaatcgtttggtcatgggaggatgaaaatacagtaccgattgatggctacgagttattggatccggacgataatgttatcgcaacaattaaaggcaagattacgaacttctacgaaagcggactatatgcaaagatgaagtaccgcagaaagattcgtgctttcaatgaaggcggagtggggccagcatctttaattgcagaggcggaaacattagactacggtagtaactttaaagataagccagttgctccgtttaatctatacatcatggaagtcggtacagaccgatgtaagttacgttgggaatacaatgaccatccattaatgccggcagtcggattcaacttatacaatgagttagaccaagtagtagatactgtaccacttgaagtacgtgaacgtaatatcctgcacttaaatcctgactcggtttattacttctacgtaacggcgtacaacaagaatggcgacagtttaccgtcgaataaagtacgtgttaaaacactcaaggtatatgttgacgaaaatgagaataacgaagatgacaagactccttcgaaacctctcgatgatccgctaggagatttaacatatgaccaagagaaaccgggcatggaaaagattcgtgcattccaatccggtattggtgaccgcttagatttagccgtacgtaatgtaatgcatacaggcggagtcaatatggaagagttcgattgtgcgatgtacatcaaagggttgtatcaaaaagaagaaccgaagtttgttgacgtgcaatttaagttccgtgttacatgtaacggtattgatgttcgtaaccgcaccgcttactcaaaagtatccgaatggatgggcgcaacagttcgcggcggtgaagatggtattacattcaatacaccaacaaagatggaaacgccggactatgtaacagagaaaatatacacaatcgaagtgcaggacatgaaaggtaatacaattccgatggacggcacagagaaaggcgatgtaaaagtacactggactcttgacccgtatgaagcgaaagacaagattggatgggtacaagaaatctatatgcgtaacgtatttaacaactggaagaagttctcgcataaaggtgtatttcaaccagcgaatagtttagagatgaatgcatggcgttacaaagaagatacggatgagttaatctgtacgcagaatacagatacatatgttggtgcagtaagtcctgatgtatatgatgactattacatcaaagcgcagttccgttcaaacaactgggacgatgatgcaatggcattcgtcgttgcttatacgctagatgagtacagccgtgaacataccatttcagcagtacgtacacattcatctgctatcacatggtcgttagtttataactactcatcaactggtcagaagacattagcttctaacatcgaggttgataaggaacttgtaagtaatgacaatacaaaaggcggttgggctggcttctatccaaacggtacaatcattgaggttgttcgtgaaggtacgaagattacagcaagaacatcggctgcgggtaaaacagaattaggctacgagttaaagatagacctagacagcttacctgaacttgcgaaattcaagaagccttgtcatatcggcgtagcatctttatcccagcaggattcagcactcaagattcaagagttccgtgggaataagacagaagtgtacacgatatataacttacgtgcatggacgaactacaaagataatgtagggaatcaaacagaatggattggatctatttcgaaaacgaagcgcatgaaagtaaaagcatttgagcaattacgattcactggacgcattcgttcaccgaagtatgagattccgtggcagaagttaaatgagaaatacttcttcgacccaacgggatataaggttgttgtgaactgtagcaatccaaacgtggatgtaacgctagagtttaacataacagacttcttcccgaaagaatcaacgtacattgacattccgatgatagcgaagatactgaaccacacgcaaactccgtggaatccaagtatccatcatggctattactatctgaatcataaggaacatttcttattctcgaatccggaagtgttaccgaaagagacatccaatagtggcgtctatgtctacaacttcccgtacagcattcgtcttgtcggacagcgtgagtatgcaggaaaagacatggtgtacagtgatgatacaatggcacgattccaattagggataatgaaggacacgaagtatgatgtcgataagaaagtactaacgttagatacagaaccgactggtacattcatctctcgcatctttgacttccaatatgaccttgacatgttccgtgcagttgacgtcaagctcaagaaagcattagcaggttctagctatacacttgaggttggtatccctgatgagaacggtgatgtagcgacatgggtaaaacagaacggtaatttaccggtagagtttcgtgacaagttcagccgagttcgttacaagctaacgctaaacgaaggacaaagtaaagagccttatgctgctaccttcccgttagatgccttagcacttgagaagggtaagaagaaatacatcactgttgacggtaaaacaatttacatcgacgatccaacattccgtgacgttggtacattcttaacaagtccaatggaatacggaaagcaaatcgaagagatgggtattgtggaactggatatggaaatccctgacggcggacgagtagagttatactctgtcacggctgatgaagaaacggctgactttgaaaatccatcaagagatatgccgtggattccgctagaactggtatctcaaaaaggcaaacgattcacataccgtgtgaagtcggcaatgaagagatacgtggtacttgtggcgaagctattccgtggactgacttatacctctcaaaacactgtcacactaagtgcagatacatacgtaccgcagtgggctgataatgtaacatggggcgttaatggtatggagttagttgacgcaagtaaaccgggtacatatcaatcaaaggcaacgaacttaggcttcatcaagttctacgataaagttcagctagaaacaacgatgaacaatgcgaaccataagattgatatttatacggtaactggttggacggcagaagaggtagaagaagctgctaaaacagaatccaactggaagaaggttgtagatggacagatacaaagtgacattcgttactgcattatgtatcgtgttgttttaacatcgggtacaggcacaacatcaccacagctacagaagatgacaatcacgccactggttgatacatacgtttcaccaaagctcgataacatcgtagcaagtgcaacactattcaactgggtgagagatgtgccggagattacaagcgtgaatgtatcgggctacattgaaaagggtatcgttgcagaagaatacttcatgccaatgacaggagagttaattgccaacggtagtgaacaaacaatcacggcattacctacagaacgtctagcatgggaatgggtacgtagagaagggattccaaaccctgatgaactcgtactaaaagacttcttcgcagagattgacccagcttatccagtagacctatacacagacattgctggtacaggatacgtaacaggtaagacgacggcatctgttggtgagcttgtgaagaagaaagagaagttatacttcgacgagaagacacaagctattgaagtgaaaccaatcccgcaagcaggtacaccaatcttaatcacaaatgcacaaggcacaagacttcgccaagttcatttccgtgatagtacgactggtaaaccaacgctgacgaatatggaaaacttaaaagcagatgagacacgttacctattcttagaacatacaggcattgatccacgctctgctcatgtatgggtaatgattgacgggaagtggacagagattctaaacatcaaggttgtagagaatcgtattgtgctgcctcacttactcattccggggtcggatgtacgtgtaacatataaactgcttgagtcctttaccgtggattataactatagtccgaagacggatgtagcgcagatcaaactgcatactacatttgatgtgaatgaaaaggaaagtcgcttactggatattcagtacgaaacgaataaagagtcggcgtactatcaagcgacagaggtaaatttaaatccgttacatacaaagattagctccggcttcttatacttaacggacgagatttacccgcctcataagttagacatcaaagcaaatccgacgacgctatatcacggtaagaaggatagagtaaccgtacatggatatgtgaaagatgaacacggtaatccagtcgttggcgaacgagtagacttcgatgtgaagtacggctcactagagattatgaatcctttatcagatgtgaacggaatggttatggcaatctacactgcgccaaacaataactcattaacatctgacacaattgagatgcatgtggtcagtcgtgacacaacatatcatgtgacaaacaaagtgacgattaatcttgtcaaagaaacgttcgaggagaaattagccattaagctagaaaaacatatggcaactccgggagatgttgtacaattgaaagtgatagcgatgggtatcaactacgaacgcctgatgaataaagctattgaagtaagctgtgacattggctctgtcagtccagcgaaaggtacaaccaacgccgatggcgaattgttcgtcaaatatacagttccaaatacacaagagaagttcgcgaccatcaccgtgaaagcaaagcgtgaagatggactgcaacttatagaacaaaacatactaggtataagtggggtgtcataatgttcttaccaccaaaagcccacactagagaaattgtagacggtcagttcaaagttaggctaggtagccgtgtccctaaggatgcggttaacctagcttatactcatataccgcctgttaaagcggataacaacgtactgattacggacttgtcgaatgcgatattagaaaacaatcgcaacacaacagccgtggatgtcgttacttatcccgacagttctttagtgttacaactggaagatggacaaacggaattaccgacagaagatatttacattacaaacttcttcgagaaaggtgttccgttatattatgcgtacaaactcgcatacaaacactacgatcaaaaaggccctgatgagtttggtgtataccgacgagatggtatctccatcattgaccagctaggtaatccagtaacagaaccgtatcagattcatttagttgcagacccaaacaatgccaatgtatatgatgtaattatctatacgtcatttaaggatgagataggaaagacttataccgttatttataatgctatctcatttgaaaatagcgggcggatgataacgaaggcaggtcatcaagaaccactgaatgtactgcgctcctttaaacgagtaagtaacatcggtgagattctaaagatggtaaaagagggcagggttgaacctgcttactatcaagcaaacggaagcgtgccgggtacaagtaaaatctatgtaccatcaccgcatatcaaagatacaagagagtatcagcgattccattatcagattggacttgagattaaaacggcggaaggtacgaaggcctatacaactccgtggtatattgaccaggtaatcaatccaatgtatctcaacggagaagaagccgaagagtatgagaatggctataagcaattaacaaaactgactgccgaggaaatgatgcatgactttgtaccgaaggaagcatacgcagaccgtaaggctttgttccgatactttgtcagaattgacaaccctaaggtagaagagttcttccgtaccgatggttcatctcctatctatgctacaacgatggtggaagatgaacgtaacgtactcaaagttccagcgagtgcgaagaagattgatgtaccggtaaccatctctacgaatgtagaatgtgtggctcgaccaacgagagcaaatccactagacgcagcttatgttacattcgtcatggataactcggagtcgatgattacgaacgacccgaataaagcacttcgcttgcagatcatggacggcttaattaaaaactgtaccgacttctttgcccgcaacggtatgaacggtatgttctttaatgataaggtgtggcaattccaaagcaactggaagaagggtgataaggagattgtagaagcctactcgaaaacgatgaaggactttgacatcactagaccactcgttgctttagataagatgattgcagactacaagacggtatccaacagcaatggttcgaataagaactggaagattgctatcatgattaccgatggtcagttctttgaaaagaagacactcgaagataaattcattgaagcaaagaagaacaacattatcctaaacgttatctgcttcaataagtatgatgagatgaaggctctatgtgataagtacggaggcatttgcttaaatgcaaagtcaccacgtatcttaatggacattcagtatttcttcttcaatatacttggcatccattcatctacgctgattaaatctataccagttaagatgagtccactcgataacgacaaactgattatcgaggtgacaaaggatacgttccaagttcctgactggatacgaaagctagatgaccgttacggcatcgagttccgtacgtcaaagccaattgatacactgagcttctatatcattcaaaaggatacaggaacgccgttaggaagttatcaaggtgcaacgttaatcccgatgaaggatgtattcaaaaataagacatacaaggtgatgatgcattcgattgcgtgggaacatttctatgcgaatagttattccttgcagttcaatgatacacgccgtatgtatatcgagaagccccgtcaacagcttgaaacacaaagctggtacttgcgtattaagaacgggcgcttcgataaacagatacaggataagtcactaggtaacccaattcatacatacgctattcctgaatattatcgtcagaactttatcaaaggttcgatgccatatatgcaggtacgaaatgaaaagcctgagttactgaatacgaataagatacgagtatcctgcacaccacttgtcgttacattcaacggggaatatgcaacgaacatttccctgcggatcaatggcaaaccagttcgagtgattagctgggatgcatctgacggtatcatgacaatagatggtcacgtaaccgacaatgacaatatgctgattgattatcaattcgaagagagcttctatgagtaccgtggcttctacgatattgaaacagataagttttggtcactcgacctaaatccaagtagcggtcattacattacaatccgtgacccggctgatggagaagtaaaagacttacctagcttcaagctgattgataaaacaatctacatctatgtgaagccagcagccaagattacgaagatgataaacggagagttccgattactagaacccgttcgaccaaacacaatcttccatagctttgaattaatcgaaggtacggaagctatgttactcggcgaagtcagagtgcgacctagttcgaatcagcgtagcattcagattgtagatacaagggttcgaggcggaggacttaaaccggaaatcacagaagagatcatggggatgttcgaaaaagaatcacacttctattgggacattggatactgggatggtaaaccattccctgagaatgcagtcatcaatatcaagattgcgaagagtgtcctaaaagagtacggcggtcacatgacccgtgccgaagtagaagataaacttgagaagcatttaggatacggtgtattccctatcatagagtttatggatgaccctgattccttactacagattccggatggtcttgtggttgaagtaattgatatagacgaacaaacaacggatattgagaaaccaacattccgtttaggattggaggggtaacatggcacgtcgtgtacgattatttatagatacaattaatgcggataacgatgtggtatttgaagtataccgtgcagaagaaaccggtgtaagtgagaagtccacacacgttatgacagtagacgatgtgacagcaaagaaggagagatacaaggaggttacggaagttttacgccgtgaccccgatactccttatgcattctttgcacaacgtcactttgagatactaccgtttccaagtgtaaccgtcggttcgaaaaccattcgaatgagtgatgtcgatgtattccctgacgacaagatgattgagattcgtaatgaagatttcggtcccggtgatggcaaaacagttcgcatgtcctatgagtatttagcgataccagtattcgatgatggacgtatcgaagtagggaaagaatatatcgggccacctgctacgggacttagaaaaccactaagtttacaaatcatgcaggatccggcaacaaagaaaattaagattaccgtatcaccggatgcagctacatcacactacttctataagatatatgcgaaagacaaatacggaaacaagtcaccgtggtctgatgaacatcatatcgctttaacaccgtcccatgtatactatcgtattcagcgttcaaaggacggcaaggattgggaagaagtaacggtaacagacttgcaggaatggctagatgatttacatgcatctgacaatccaatgaatgtaaagaacatgcaagcacttccgattgcttctaaaatatcgaagctgacatttgataatccgtggtatgaaaatacggacaagcgtaactcctatcgttaccgtgtccgtgcagaagatagcgacggcatggcaacagactgggtatacatcgaaccaatcgagattaaggttgacctaagtaaacttatcattcgtcgtaagctagacaataaagcaccgtcctcattcgataagacagatgcgattaccgtgtttacgattgagaagaaagatgtagatgtaacgcagaagaccattacactagtcgatgaccagttaacggacacttctaaatatagctacacattctttgcgatagatgaactaggcatgatggctgacccagtatatgttgtatcggataatagaacatggaagaacgttatcctgtttgtgggcgatacggaaacagatacaatcaaggaaaaagatttccacttatcattcgaactggctgaccgcatcatagaaattggggaaggagatggcgactgatggtaaaacgagtaaggcttacatggaatagaaacttagacccggaaacggatcactatcgtatattccgaaaagaaacgcctgacatactagaacagaatcgtatggaatctttagtcatgcgtgtcgctcatccaaaagcgaagaaccccgttcgcatgaccggtgagagagtcaaacgattatctccacgaacgtaccagcttgcgcataagaatatcttactggctttagacaaagagaagttcccctttgaattgcaagtcgatggtaagatttctacacgctatacactggatacgattgacgggaagattatgttcgacgaagatgttgcgccggaatcaaaagtcattgcgactgaatatacattcgacggcgtagaagcatgggactatgctatcgaagaaaagaacaaaacgtattatgggccggacgcaaaagatactgcaccacctagcccacctacgaaccttgagcttgtaaaagatttagacaacaataagctaacgattaactggtcaccctctgtcacaaacggcaagacattttactatcgtatagatgctgcggctagttcgaagcgtgcttctatgctaagtgaaatgaaagcagcgaagattgcagaaggattaacagaccgaccatatatcgttgagcgttctgaggatggcgtacgctggctgaaagtagcgaaggtgaagacaacatcatacaatgaatacatggttgatagacaggctcctgacatggttcgtaacttcaagagttctgcatatctacatgcgaaccaaagtctagcgcaagtgacactaaactgggagcgtgtaagagatacggtattatctacgacagcattataccgagtgcgtgcgatgaataaaatgcaagctatctctgaaccaagtaatatcattggccctgttccgttccaagtgaagctaaaagaaattgttatccgtcgtaaagtatttgatggtacacttccaacattcgacggagcagatgccgtaaccgttgcgaagattggtgacctaagtgttacgcagtatgtagaagatgtaaccgacaatatcaaatatacatatggtatttggacagtcgatgcaggtgagaacgtatcaccaacagcctacacgacaattgaaatcggcgatgcaactccaccagcgttcccaatgaatctgacagttgagccattccaactaatagtaggctaatgtataatacaaagtaggggcgataacctcgccctatttttcttataaagaggtgaagctttatgccatcagatggacgttacgttcgttttgacccaaagcaagatgatgccaatcaagttatctctgccgatcacgttaacctgttgcaagatacatcggaacgtactcagcagggactttttaaagcacaagaccgtgacttccttgataaagcgttattcgttttagaacatcatagttcagtcaacggtatgtggctcgacatgtttgaggatacgaacaagatagatttacctcgtactagtaacttagtattctctcaagtggagcagggcattatcttccctgacaataaagaagtatcagaaggctatctatactcgaagccttatgtgaatgaaaaccaaaccaacatgaagaaggttatggtgattgcgaatggatgggttccgaacttatccagcattacaatcgaagtgagtaacaataacgtagattggttcgaagttcctttatctgaaagtgaactatttgagattccaacgaacggtcacaagctgtatctccgtgcaagattcttgcgtgcgaatacgcaagtgtcacctcgactagatgcgtgggctatctattactatgactaccgcaatgatgtaatcgagctagagaatggtgacgatattattatcgttgacccaacagacccgcctgagattcctgatgtcattaaaatcatgcatcatcagttaatgggtattggccctgacgatcatcatccgcaggaacatagtcatgatggttcagatggttccggtacaatctcgcataacgtattaaccgaagtcggccccgacgatcaccatccgaaatctcactatcatggtgaagatggtatcccgtacgttcgactagaatcagatgtggttggtacactgccggtagagaatatgtcctaccaagtatggacagggaagccgggcaagacaggattgtactttgacccgaaaatcggtgacaagttagtctatgtcaaaacacctgacgatgaaacgtatatgttctacgacctagtgaatgaccgcttatcacatacgattactatcgtacaaggcgtagcgaactgggaacaaatgatttacggagaatacaatacgagtacaggtgaaacaacaatcgtactgcaaggtacaaacaagaaacactttgatgcaacggactctgttatccgtcaagagatacagaagattacagcgccggaagcaccaaaaggtttacgagcaacagacacaggcacaggtggcacggtagacttaacatggtcaccaaacacagaactcgatgtagtcggatacaatgtattccgttcatacgacaaaggtatcacttggattcaaatgaacacgactggtgtcattgcttcttcatcgttcaccgtaacgaatgcaaaagacggtcaatccgttttatattctgttacggcattagatgagatgggctttgaatcaccacgctcacaagcagtggagtgtacatcgacaacaagagatacaattgcaccgagccaagttacaggtgttgcgattaagccaaactcaaaaggtattcttgttctatcatggacagagaatacagacatggacttaaagaactacaaggtgtaccgttcttatagcggagcagttggtacattcgaagtgttgaactcacctgttaagggtacaagttcttacacaccagcagatttaattccaagcaatacatatttcttctacgtaacagccgtagatacaagcggaaacgaaagcatcccgtctaacgtcgtttcagcaatcgcataggtggtgagagagtatggtgcaaattaaacagaatgctgatggtagtttctccaaagggtatagtaactttgagacaacgatcatcaagagccttcaaaaagaaattacgcaacgacaagaagctgacctttcccttcaaggaattacgaatggtttgaatgaacgcctgaaagaacttgaagcaggaagctttagtttagctgacctgcaacaacttatcacgcagaacaaacttaccatcgaagaccatgaaagacgtatggtgaagctagagaacatctctcttatcgagtcttatagtaagagcgagatgaagaagtacatggttccgtggcgagtagaagtcattccgcacgcctttgacattccaacaaacaaaatgtttgatacagcttcactggatgtaaagcgtagcgggattatacattatccgaacggtgacatcaatgaaccaaacgatatgcacgttccggaaaactctatgatgcgttttaccatttgggcatatgcatcatcaccacgtaaagctgtactgaacattaactacgctgaccgttgcgttgtcattgctgatgaagtagtagtcgctacgttcacagatagaggcggtaacttcggtcaagtaccaaaaggtgtgcaagttccaatgaagcaaggatggactcgtatccagcttcttattgcgaacgaaacacagtccggtgggttagtggtgaaatcagacctgtatgataaggcggattacttaacgagccttgattacttcgacggtatgattacgaacaaacatatcaagccgggttcgattacaggagagaacttatcaccgaacatggatttagttgtacatacgattcatgcaacggcgacagatgttccgggtgttatcattggtaaccctgaggaacaaggtatcctgcaaatcgctgatggtacaatctcaaaatccaaagacgagccgtttatctttagtcatggtatccgtgtaaacggagcgatccacgttacgcagttacttattgacgaagactttatccgtgccggcgatggtatgattgtcaaaacactaaaggatgaatacggatatacaaagatgtatgaaatcatcaatgaccttcgtatgcatactggtggtggtgtatcagtcgatggtaacgggcaagacggttacatcgtaacaaatacgatgaagctgaacgttgaccctgagggtggtcttgcaatcagtggtaatgcagtagatggttactggttatcaaatgatatgcagctacattctgacggcggtatttgggtagaaggaaatgcccgcaaaggataccgcattaagaataacatggggttacgttctaacgtagacttccttcgtattttcggaacagcgtgggaaggatatagcatttcactggatgaggatgcattctacaatcgtatcaaagaaatcatcaagaaagaatcgcacgtaaaggatatcatttctgacattgactttattaagattactgacaatggtgacggtgtacatcgacttgcaatccctgattacaataagatgattccgtggattcagaaaggacagtccggaatgctacatgcatctccaatgccgaactctatcgggccgggtatgagattcaatgcaggtaacccaatgttcgcacagtctcaagatacgaactactctgtacttggtttttggggcggtgaagttccagcaaacacggctgatgtcatctgcattagttcacaaggtggttggacaaattcatggtcagcagtagacttgaagttcgttatctcgattgaagaccccggcgtatctggtggcgtatggcgtaaagagttctatcgcaccattacgaatcagttagacggcggagattcacaatggaacaataacgaaatgattcatgtcattccgattccagttcgcgactattggagacactgggactgcacattctatatgcgtaagtctagcggaagcggttgggtaaatgctcgtagccgtgaagcatggacggatcactctactcatgctggtagcgtgcatacagcagcaatgcgtacgcaagaaagctatggaggtcgataatatggagaaggaatttatagacgtaggcgtgcttaccttaaagggtaagcgcctctgcggttttacaaccaagatggtagaaaagaaagaaaagcgttacttattagatacgcatgatggcatgtttactcatacacacgttgacatgaatattattaatctattaagtcagcgcatgaatatgtactgttactatgaagatggaaagatcaagaaccaaacgaaattgtatggctctatcgacaagaaagaagttgtcgcagaaggtgatgactacctgacttacacactggaaacagatgaacagacagatgtgttcattacgttcgttgttggcaacaaagcacaaacatctcatgctacctaccatccagtggacggcgtagtcacagttgagattgaccctaaagccattggggagattcgcatttgcaccatctcggatacagcatattcggaggatgttacagcaaaagtgatataggagtgatacgatgaaattcacagagaagaatggacgtatcgtaattgagccggaaactatcgacgcagaatctattgagttagaagaacttcgtcgtgacaaggatgctcaaaaagaaacagaacgtaaacttgctattgtattagataagttaggacttacagatgtgtaggagactgttataatgacagtagtgaggtgacgagatgaataatataaaacaatctacggcgagtatggatgacactcgccgtaatatgatatacaaactcttagaagaacggtacaaatacaaggaagtaccgaagtattacattgagcaggaagtagacttcttcttagaacagctcaaggacggtaaagcaattttaacaacacgaccacaatcagaactgacaaacgccgaagacattaatcttgaaatgttggagtcggagattgatatacatacaatctatgcgcagttagaacatgttgagagtcaaatgaatcaacaccaaacattaaacagttctatcattaatgatatacaggctcgtgtttataaagcagatgaacgcatggctgaaatctcatggcaattgcgtgaagatgcggtacacgttgcttactatgatacgttccttgatatgggtaacatggagaaggacggtaagttttatacagaccgtgatggtgtgcaggttgcaccgggttggtattctacactagacacgaaccataatgcaattaagctaccaatgatctacacggagaatcagcttatgagtttttccggtgtgaagttagcagaagtgaaaatcaataagcaacttggcggaggattcattcgtagacgtaacccggaacacacgcctgataaaatgattgacgacagtatgaatacctattggagtgaatcggtgtacatggatgaaccgttcagagtcaatatgggcattgacaattatgacatcaatttcggagcgatgtgtgaattagaaatcgtattcgattatgtatccaatgtaaatgagattaccttcacgccgttcactgagttcccgatgcaagtcgtggcaatactggcttacacaacagataatcctgatgaggagccgtatgaactaatcagtccaacgaacgtgaagaagggaaaagaaagtgcagatgtgatgagcttccagttccaaaacatcgtagccaaacgaatgaaaattatcctgaaccaacagcactatacaaaacgtgatgtactgattgatgtggatgataagacgttagtcgatgcgtggctagatgtacaagagaagccgaagattgcacctgacacaatctttaaacctgtttaccatgaccagctagagatgaacccgcagtgggaatacatgaagcgttacttgtcacacaaagatattgcagaggagattcagaaatatgaagatgctgatgcaaagaataagatgcaagtttcgaagtatgaataccagtacggattatacaatgtggcaattcgtaaaaacaattattatcataccggtattaacgtcacaaaacctttggctcatgcaaacgttagatacgtatcgctcgaagcaaaagaagaacacccaatcctagaagagattgagattcctgttacgggtgttgaatactatatcaccgatacagagaatccaacgaaggacgactggattcctatcttgccgaagaatatcagacgcataggatcagagcgacttatcgttgagttcaagaaagagaagtaccaagcgatgacgttgttcccgtctaaacatacgtatgcagtacgtaagaacggcggagacttattaccgattacagattacacggtaagtggtcgtaacgttgtgattaaagagtatgacccaacggcaatgtacacaattgattacgagccggatgcgtctgcttatgaagtagacttcctaaaacgatatacgaaaacagaactgattccatctgagaagaaaatcatttcgtacatcgaccctcgtaagaacattgagacattcaaagagttcgagaatggtaatgtcgtaacattaaagtatcatccattcatcgacaaggataaactgaacgttcagccgatagattggaatccaagtatgttgtctagtaagtacctaccggttgtcgtacgtgttacactgccgaatggtaaacttgttcagcaacgtacagacaagtgggataagacagacatctaccttgtgaaccgcacagactactttgatacaaacaagagtctgctggaaccattcacaggcaagaactatcagtaccgtgtagaagataacaagattaaatttaatacagtcctaccaaaaggaacaaaggttacagtagagtatccgtatttaactggccctattcgtttgaagacgattctaagacgtaaccttcctgatgcagaaggattaacaccgttcttgcatgagtataaaatcggactacaaacattacgataggaggcacagcatgaatactgatttattaattaagaaacgtatggaactgacgaaagatcgtatgctgcaaatcctaaaggacttacgtaaccagtatcagcgggggcaaattcagtcacccgctgaactacagcataaggtatatcgtgagttgcaaaagttctatgacacgattggaaagtccactcttgaaattatcgaagcatgggggcctccgtatagtgaagaccacaatgccatgatggaacagatacttaatgacttcgttatgttgtacggcgagattacgatgttaacggacgatgtaaaagctggctttgaccaaacagaggtagagcgcaaaagctttgagaagcaaatgcaggagattgaatcgaacattaataacctgatgcccgcagcgaaagacgtatccgacatcatggtattccgtgaggactttaccacaacggataagttcgacaaggatgcagtagatggaacggttgcgacgattatgacagacggtggtattttaacactcggtcatatgatggcagaacactttaatgagtatgcctacattcgactgaaaagcggtaacggattccccggtaacagtcgcatcattcgttctacagggcaagggttaaagtatgacggtgaaggcaacctgcatttaacactgtctcatgttctcgaccaaaatgctgatacatggtttgaatacgagaacttccaattaacaaggcaagccgatgacaaaacaaagcgtatgaactttacatacaaagaaggaatgaagtgggcgcaggatgagtacatcaaaatggatccgcttgaacttgtagtagaaatctcattcccgaaagagcgtaagctaaactggtttacgatagacccgtacattccgccggagcgtgggtcagttggagcaaagataagtaagatagtagtggatgatggtaagggtcacatacaggcaattacatatgaagaaatgatggagaagaagaaggggtatctatttgaaacgcagaatgtgtccaccattaccatgtatcttcgtcaagacacgcattatatgacgaacataggtcacgtattctatcttgactccaacaaagatatattgaaagcactagaggatttatcagaatgggctggcgctcgtgtatatggaaaggaagtaccgtcggtcacgaacctcggcgtaagattccatagtaatacaaatacaattgagtatccgggcttgaagtacggagaagagattccaaacgatgcagagcgcaaacgtgcgttatacacaattccgaaagtagatagtacaatgcttgtcggattagaatcggtaacagcgaagcgatggatgattggacttcgtgatgcatctatgtcttgctatcagttcgctgacgtaagccaatacatatccaagccgttctactcggatgcaccgattgaagagttacagcttgatgctaactacagtttccctacatcgtttgcggaaggggattacgtagagtttcatattagtatcgacgacggtcagaactggcatcgcattcatcgtaagaatgaacacatcaaagatacgaagattaaatatctcattaactctgatgtgccggaggaagcacgactacctgacgttggctatttagaaagccttcatgaagtccatgaagtacgagtgaagataaaactcacacgcccgaatacgaagtatgattacaacttctattcaccgatggtatttgattatgagttgtatgcgaaagtaacagagggggcgatctaatgatacgagatacacaacgagcattagttcaaaaggaagtattacaatccaagttgcgtgaaggcattagccctagcgctcttgtgattgaccaattggtacaggaagcattccgtgtaaagcctgagggcgtaccacgttttacttaccgtgaaatgccaaagggtcaagtatcttccgttgaagattacaatactctatttaaagaattgggattcgatttagaggtagcctttgaagagaatcgttatttaaacaatcgtctgatggcggttacgtcgtactacgaaacagaacgtagacgtattaataaagagttagcacgtattacagaaaagacgaagtgggtacagatgaagcaagaagctgctacgaatacagaagtagttggcgatacgttgcagaacttcctgcacatcgactttcacggggacgagaagcgtaacatcccgcagacaaatgcattcgttgatttagtcaacagcaatgtacagatgatgcgagtagcgaaacagacgatcaagcacgacttaacaaaggcgaagctaaacttctacgctgaaagtccggagttccctatcattcatttaacaccagtcgaaagtgccatgcaggataccatctatgattcatggagagctatcgtgataaataaactggctaccaatctcaattcattccttgatgcagaactgcctgaaccagtgccggcaacgaatgtttcgattgatacgcaagtcgggaatcccgtattcgttacactcttcttatcaaaggacgggaaagtattcttaccactggaacgtagaaaaattattaacacgtatcagtggaactttgaacgacaagaggtaaaggcaatacgattccaattcgagaagaaggaagaagatagagcgaacggcagtgactatgaatacttattcggagcgaagaacattacactcatggaagagaagtacatggataagtcctattttgtatcacaaccatttgacctacctgaccatgaagacattgaaaaggtatcactagaaattgatgacctaacaccagcaggtacaaacattcgctactatgtgggacgagattacgaggataacgtaattgaatgggaagagttgcgtaaggatagaccgattgtcagtgaccaaatcaagagtcgtcgtattgatattaataagtacacgccgggatacggtaagctgattaatgaaacgttcggtcagaacttccatgcgattgcaacactaccgcaccgaccactcaagaaaagcatgaagctgatgattggtcgtaacatgtggctacgtgaaacgatacctgcaccttttacacacgatgccaatgatgatacaaacaagactgtttatcaaacatcggtaaaggactgggtgaggatcggcacgccgaagaaagaatatgtacgtgtaaacaacagagtagactacttgcagaaggataccttccagcgttacacgacattcatctacatggatgacccgcagaactatcgtgcgattatcagtacaggagagaactcctcacacattgctttattaaacggagcagaactgaaacttgtgggagacaattacaacctatccttacaaaagggatggaacaaactacaggtgtatgcatatgcacaagacattggagaagagattgttttagacttctttacaccgcaaatgtcatcacgtatctatgctgataaaaaccctcttcaacaggtgtcaatgtatgatatgctaaataatacgaacagtcgtctgaatggacgattcgccgtagatgagaacaacaacgtcgtcgtgaactatcaccctcgtattgcagatgtcgcacacggtttaacaaaaccaatggggcaaacactagaggaagcacaaaacggcgtacagcttgcagagggcgtggaatacacgcttcaatataagtattctctacgtaaagaacgacaccataagctacgctttatgggtatcttaacgaaagagaaagaacgtattgaaacaactcctagcttcaaaggttacaagttgattgtggaataggaggatggaatgtgaagtatcctgagattgtaaaaaccaatgctcgttaccgtggcccacgagaaagtcgtaagcaaagtaattccaaacgtgacatagaagcatctctctcggtgcttagaaaagaactaagcgtgagaagaaaagttggagaggaactacgtaaagacatgtttacattgtataatgaaactataccccgtcagcgtaccgaagtaattaacggggtactctctgtgaaaggaggtgagttacaatgacacatgcgccgttttatagcggacaatcagaacgctttagaagtaaagcagaatccagtaggtacaaccagcacgttgacgaaacgctatacgacatcacggagctatgtaatatagcaaatgagctagaaagggaacaggaacggacaagagcattcttcgaagtcggttctcatttcgctcaagagcagatggatcagatgaagcgtgagctacaaactctacaggaagatatgactgcgctgcaacgtcccggtaaagaatacatcaaagtattattaccgtccgatgcaaggcacgacagtagtgttgatgaatatgagagagccttaatcgacttacaacatgacatcatcacgcttccgttctcatcttattccagttccaagctatatatccacgacgatatagactcggagtacatcgtaccgaatacacttcgttatgacattacaccgaaagcggatggaatcacaatcaaggagaacgacttcattgatgcgttaaccccggatgacactaagttttggcatcgtagttatacatacttctccggtttaaaggactcggtagatgcacagatcatcgtggaactacctgacgacattatttcaaaccgtgatgtaaacacgatatttattcatccgtttccattaaatacgatggacatcatgaatgtagagtatcagttggatgcaggttggaaatctgtacccggatttaaaccaatcgagaatgcagggaatgtaaagttctgtttcccaccaactgagatacgacaaatacgtgttagccttcgtcagcgtcactttgttacaaaagggaatcaacatacattccatatggggattcgtgagattggcgtttcacacagtgactatcagcttgaggtagcacgatttgaaatacctgtctcattcaataatgcttttctaacaaaagagattctcgatatacaaccggtataccaaaacgagcaagcattgtctgtataccaaaaggaaacaaaactcacatcctttaaagtatatgagataggagagaacggcaaagaacgatacttaaatgataccttccctgttcaagtgagagagaagaaaatactcatcaagtgtctcattgccttcgacccgaacactcgaaccgcacctgcgataagaggcatcgagttgacatacaaaggcgactcgtaatcttcccttgagggggttaacacaatgaaaccattacgaaaaatacgagattggtataagcgtgcctttcacaatgtaaagattatcgaatcagcaatcgtaggttgcttattatggtgggctttcattttgattgtgccgttaaatacatttgaaacaagtattgcatatgaagcgatggccggaatcgcaagtgaggaggtgtggtctggtgcattcttcattgttggaatactggcactctatggtatgctgacagagcgttattggatccggcaggttgcgttagttttatctagcgggctatggttctttgtagcgtccatgtttgccattagtacattggcaacgacaggaactgggatttatttcatagtagcctgtttaaatgcctacgtggtatataaagtaggtgaggaatatgggaactagcgtaatcgtttctatcatatctggacttggcgtaatcggtgctgctattctttcttacttcggtcagaagaaaggttccgttgcgacgtccgagcaaaaattccgtgaagacattcttgcacaaaacgagaagttacaacggagagtagatgaacttgaacagacaataaccaacatgcgaattgagaaccgtaaacttcaactcaaagtcatcgaccttgaggatgaaaggaaaggtggtgcgtctcgtgaagtggctgacgtttctgaatgacgaggatggattctcatctaaggacttcataatggtcgcattcatttgtacgtacttgattgagcagacggcagtatttatatactcgttatatggtactgtcgattctgacactctcaagattgtatcttcattagatccaatcgtcatgacggttattggtggtatattctctgttcatgcagtaaaagaatttaagaaagacagcgagtcaaagtaatacatactcctttcacagagtggcagtccttcggggctgtcatttatttttatgtcataaataaaaaatgttacaaaaaccgttgaaaaaggttacataaaaagatatactatagttagacatctattacctgaaaggggcaaatactttgaacataaacgagtgttttcaagtgtcgcattatgaggttgataagaaccttcgcaagctgattgatgaaccagtaaacgaggatttaatcgacaagaataaatatgctaataatagcgagtttgtacctgatagtatttacaagcgtgtgctaaataaagcaacgaactatcaatggtcattcgtaccgtacgacatctctacagtagaagggaaatacgtacagttcattggcttactgatcgtaccgggctacggtgtacatactggtattggaacacagaaactacaaaagacggataactccaatgcactatcggctgctaagacatacgccttcaagaatgcttgtaaggagatgggtatcgcaccgaacgttggcaacgatgactttgatgaagcattgtttgagaacttcgacgatgatgaaattgaagtagaggaaaagccaaagaagaagccggtcaagaaggaagaaaagaaaccggcaaagaagccagctaagaaagaagccaagcctttaaaggaacgtatcgaagagattcgacaggcttatgagttggacgatgatgacttcgtagcattcattcagatttgggatgagaatatcattgacctaaaagatatggatgataagaaatggaaagcattcttacaagatgtagaagagaatccagaggaatacgaagacttctaaaatgggtggccttaacgggtcaccccacatttatacctaattttactaggaggaaaactagatgacatctatttacgacatcgtgtatgattatatggatttgttttatagcggtgttccgttggataaggaacaaaagaagaccatccatcaagaagtaaagaaactccttcaatctggatggtgttcttctgagctttcttcaacattcaagatggtgaaaagaaaaagcccagacctgcaaacacttaaagcgtccacggtggtgagaggtaagcgtaaaaaggaagtgaatttactaaagcaaggtaggttctatttccacaatgctttacgattgacaagtcctccgccgaaaagggagatcgactacgatagcggagagattacttctatcagcacaccgtactttctcgaaatgcgtgcaagctacaccgtggatgagttggtcgaatactacggtaggcaaatagggattaaactccgtagccacgagaaggcgagatacataggcagcttcaagtggctccttaaaacatatgatgtagaaatgatattattcatgatagatgcaacagtcaacatgtgcattgcagaagatatgccaatgccatacagtccgcttgatatacaaaagtatatgaaggaagcggaacttgcttacaaccaaaagcaaacagaaacggtagtgtcagggggaaagaaaattgtcaggaagaaacgggttagacgaaatcgagttcggcgttaggcttccgtcggggaagttcatacgtaacatatttaaaacgattgctgacattcctgcaatgagaaagcgctataagaatcaaggcgtatacatatcagcttacagctacgagaaggcaacgaaagagaaaacagatgacatgcttttatatggcaatctctacatcgaccttgatgctcctgatatgaaagatgcaagtaaggaagacgaagcattcgaacagattagggaggacggcattaaagccattagcttcctatctgctattgtcggtatcgacgaggaaatgattaagatttattacagtggtcaaaaaggaatacatatcgtagtacctgcaaaagtattcggcattaagccgatgaaagaactgaaccatgtgtttaagtgtatggcgaaggaaatactcaagatgagtaaacataaaacaatcgacacgcaaatatacgataacgcccggttattgtcattgccgggaggtaagcatcctgcaacgggacgctataaaatacctctaacatatgacgagtttcgaacgttagactttcaaacgattaagaagatgtctcgtaaaaagagaaagctaacttataaacctgctagatacaatacgaaggctcatcgaatcttccaaacttacataacggattgggaacgtgagaaacaacaactatcaagcaagagcaagaagggatataaatctacactggacttctgtcctccgtgcattgaatctattctgaatcgtccatgccccgaagggttccgtaaccatactgcggcggcactggcaagttactttaagcaacgtggattcagtaaagataaaggatggaaacagattgtacagtggaacgatgagtacgccaagttacctgctagtgaactacaaacaacattcgagagtatgtacaacggggagtatacatatggatgtgcgacactagaaacactcggtgactgtcaaaagaacaattgtaaaatcggtaagagcaaggataagaaatagggggagcagtcatgccagcagttgcagaacgtaaacaaagaaagaaacgtccaatagatagaaagtatatcaagaaagcgccaagcgatttaagagactacgagttagaacaatgggaaacactaaatgactttgaagagaaagcatggagtgaacagaacaaaggtttatcaatgggttggaagtcaatggatcaagcgataggtggactccaaacggggttccacgttatcggcggtgattccaacattggtaaaacatcattcatttcacagatggcatggaacgtagcaacggttaatcctgatgcatacgttatcgacatttcgctcgatgatcctatgcttgataaagtatctcgtgttattgccgcaggtcagaaggtactgattaactcggtaaagacaccacgactatacaagaagtatccgaagatgctacaacgtcgtgcaattggactaaagcaactgcgtgacatggttgaccgctataagacttatgaccaaaatcatagctctaatatcgaggacattaaagagactgtcaaacgtcacatcgtagagttaaagcaagcaggagaaaaacgtaaggttgttgtcttcattgataacttccatgacttaacgacagaagctcgtgaagcacaaggcagtgacaaagcaaagtatacatacctagcatcattcgttgcagatatggcgactgagttagacattcctgtagtatgttcatgtgagttcaagaagattaatggtttccgccgtccatctattgatgatatgcgtgaagctgttaaaataaaatatgaagcgaagtcagttatgctttgctacaacgaagtatcactaaaaggtgaagctgcttccgtttactttgagaaggctaacgaccctgagaaacaacctgtatacgaagtgaagttcgcgaagaacaaatacagcaagttcaaaggtcgagtattcttcgaggggtatcctgaaatggcgtacttcgtagaagccgacgaagcttcatctcaacgctataacaatgttatctatagcaatgagtaagaggaggtgacaatatgaatgaccaattctttatcggcgtacttctcctaatgttcttttgtgttctattaggagtatgcgcaacgatggcagtgtataaaacatacctctatcgcaagaagagggaactatcactacagatcatcgaaatcaaattgcttaacaagcagaacgaacgcaatcagaacgttgtcaaacctgactatgatgaagatgactgcgaaggcttgttaccataaactaaggggaggaatacatgatgaaaacgaaccaaaacttttttgaggaacgtatcacggatgagcagtttgaaacattgaagacaaaaggtcttcgaggggaactagaagctgctaatccgattgatgctacgaaggttacatttgaaaaagcaatcttcgaaagcgagaagcatctccgtgctaaagaagaacagttggatgagcttcttaatagtttaacaagtatgcacagcgctattaagaaggaaaaggttaaacaggatacgctgattaaagattacaaaaagaactttccggaggcgaaataatatgagagcaaaggggcgcattaatcttacaggtgaattactaaaagggatgcttggcattccaaagaatatctcgttagataaaatctacattgatgatactcgtgacgttgtaagtgttgttgtatcatctgatgcacctgcttacgttggtgatggtgtacataatattcgtccagtgacgttcgatgtgaaagaaggacaagaagttcctacttggcagattgatacgctgaacggattacgtaaaggggaattgaaataatgtttggtacacaagttcaacaaactggttctaacgcaaaagaaatcaacaaggaactatcgaacaactgcgacatgtatgagccatgtccaatctgctttaagtgtcaaaacaaagcaactcatttatatcagcgatgcgaggaatgtccagtgcagttctgcgggcatagtcacaaacaacgttcattcattattcgtcgtgagaacttcgccatcaaagtaacagatgagactggtgagaagtttaaagaagcggcggagcaggttaatcaaacttgcacatgcaataagggagagtaagagacgatgaaagctaaacgagggtcatggaaggcagttccgaagattacaccattcgaaagaagaaatgcgaatgaagttggggaaatcatatgtaagggatctaatccgctaggtgtcactgtacattcggcggagggttcatatacatatacatacaaagatgtagcacactactatgagcgcagccataaaccgttcagccaaacggtatctttatttattatgaaggatggtaaaaatgcgaatgactacaagcggaaagctatatgttaatcaactacagaaacaaatggatttagatatggaggaactgctttcttatgaagctcgacagtacgatgtgggagatgaccgcaaagtacaaccagctttcatgttaggggattacttcgaagagaagaaacaagagtcgaagatgagagcattacgaagaggtgagcagtatgagtaaatacaaagcaggttcatctatacaacagatggcacagcgttctcgtaaggagcgtattgcaccaactgagtataagaactatgatgactatttaaaagcgcagaaacaagtgcatgaccacttacatagcaaacctgctatcagttgtgaactatgtggttacccactaacgtataacggtcataaaccaactgagcgtgaacagaagtggagtatccatgatgtgtgtcaagaaaagatgaatagaatgctagaccgtgagactggtattgcgagagagcgtaaagcactagcaaaccaaaacacgggtaaacgattctaatgcgtatacacattgaccaggtaatagattatgttagttgccctgcgttgtacaagtttcgtaacgtagataagttggaaccgccccagcctaaaaaggggcgaccaacgaagaactctatcgtagaactgtacgacgaggcactgcataaggctgttgcctatatttttaactcggtacaagacggtaggtatccatctcttactaacatgaaaaatagatggggcaagttatggatcgctccgagagcgaagcaagaagatattaagttcaagcaggtaagctggcgtgacacacacgagttaaagcgcaagcaaggctgggataagatagagaaactatggcttcattacaaagaagaccccggttctcctatcatggtaaattatccgtatactgtacaaattggtaagcatacattagaaggaacgattgaccttgttcgagttgccaagaaagagaatggtagagaacatattgagatggtcgagtttatcactgacgaacggaacgcaccattcctacacactcgcagggattggagagtcactgctgcttcctatgctttcaggaaaattatgaaagcgaatgaggagaaaatagtgtatcatggaattataagcgggaagttattagataccacaagggatgaacaggattataagcaacttgaacatcttctagatgcaattgaacatatgcaaactcatgagatatattatcccgtattcaacgagagatgtaactcatgttcatatcaaaagtattgtgagaaaggatggttcgatgttaaaactagtaaacagtgatggtgctgaaatcatgcgcctccatgacaataacacagaggaatacgcagataagaagattgaggaacaagcgaaaaaggcaatacaaaaacaggagcaaaaggacggagaataatgcgaatcatgggcattgatgcatcactatcgacaactggatatgcgatttacgatactgaaatcggtgacttcatcttaatagataagatacgtacttctatcaagaaggcaacaccaactagatccaagcgtatacaatatctttgtgcagagcttagtcatattatgttctggaactatgttgacgtcgttgtaaccgaggacatttacgttgcgcagaaatccgccgcacttccattagggatggttcgtggagcgattgagcaacttgtatatgatttagagttcgaaggcttacacatcatcgaaagtacgaagatgaagaaagctgttactggcaacggtaatgcgaacaaactactgacatacgaaacgctaaaagaaatctacaaagacagtccgcttgtaatggaagcactcggtaaagagttgattagtgataacagtgccaagaagaatgaagacatggctgatgccgtcggactcgttcacgcttataaagccgacccttcacaagctcacattgcctagttagtagtgaaaggtagtgttacgagatgaatcataatatcagctatgaagaactcacactattggtttcggcctaccaagatggaagtacagatgcagcagagcaaatcgttgcaaactatgacggatacttccttaaatttatgtctgtcatgcatcccgttcgttttcgtatgaaggatcgcacccagcgtaacttcttaaagttattctgcggtgacgaatccgtacgaaagaatgcacatctatacgctattaacgaagtgatttatcgtaagctaatggaaacgttatcgaacgttcgtgatatgtttagacactatcatgaagatgaactcatgagtgagatggtcatcatcttacttgaaatggcgagagaacataatggaaaagctccgtttcatatttacgttgctcactacttcccacgcaaactatacaaacgtctgcgtaaaatggtacaaggggatgtaaaaccaagagaagtgtattatgacgaggaggacattcgtgtagcgcattatgatgatgaatacacggacgacaaaccacgttattatatcgaaccaactacaccgacggactatgatgaaaactgggtcaacggctatggttgcggagaagtgtttgaagatttaacgatttacgaacgtcgccttattaaatggtattacgaagcgaaggcgttgaatcctgaatcaatggacaaggatatttacttagagcgtagagccgcttgtaagtgtacggagtccgacatcgcagatcgtctaggttgtaaccgtaaaacagtgaacctaaaacgtaacgatgcaaaacgtagagtcgagcaatatgcatccgacctacatttacttacgggtagccaacccaaagcacataaggagtaggatgtgattgtatgaagtatacaacttgcaccgagtgtggaaacaaagttggctacacaacgaatcgtccaaagcgatgcacagcttgtaaagaagaacgtgaaaagaaattcacgtataagaaacgcaagccacagcgttctaagaaagaaggtttaatgcagaaagtattagatgaacttcttcctgacgctgaatacatagataacggttactactcatggatgcgttctcctagaggagcgtgcttgcagttagaccgttattaccctgacctaaatctcggatttgagatgaacggcagacagcactatgaatttaatcgttacatgcataaagacgaagaggcatttgaatacttacaagaatgcgatagaagaaaacgcaaagaatgtaagagaaatggtgtcactttgataacgattaagtataataaaactatcacgaaagaatatcttgtgaagcgattccgccaagcaggtatcctatctgatattaaaaaacaaactacagtgatggaatagggtgtcttcgtgacactctttctgtctgataaggagggttcttatgttagctcacaacaataagactggtgtaacggcacagccgaaccaagaagcactcatcgcagaatggactgctgatggattcagttacttaattggattcacaggtacaggtacatattcgggtgagtttctattatatgtaggcggtgaaccacactacgtatatcaaacatcaccgggtaaccgtacagcttacattgcagatcgcggcgtacgattagatgcaggtgtgaaagtacaactgaaagtaaaacacgaagacgacgaagcacaaacattcaaaggaacaattcttggaggacgttaacatgaagtatgtaggtagactagatgtagaaatgggtggaatgttcagtggaaaatcagaagaacttattcgtaaatgtagacgagcagaaatcgctggtaacaaagttcaagtgtttaagccgaagcttgatgaccgctatagtgatacagaagttgttactcactatggtcgatccatcgaatgcgaagttgttactaactcgcaagacttataccgcagggttcaacctgacacaagcgtagttgccattgatgaggtacaattctttgatgatagaatctacgaaatcatcatgcatctcaaacgtgacaaaggcatagatgtcatcgtagcaggattagatatgtgggcttcgggtgaaccagtgatgattgtagcaaagctggctgctgtatcgaatgatgttcagaagttccacgctgtctgccaagatacaggagaggatgcctacatctcctattgcacggtagaaaaagatggtgacgtactcgttggtggcacagacaaatacattgccctttcagagaaagcgtacctgaaacgactaaacaagtaggtgatacctatgaaatggaacgaagcatacacgaaggtatttacaacaacggttgctggcgcaacaaagacagtctcaacaccgtaccgttttcacatgggtcaaggtgatgtacaagtattcgtcaatggtgtatacgcaccactcggaaaagaatacaaagaggtgtcaccgtattcgattgagttctttgaatcacttgaagcaggagacaacgtgacattccattatcagaaaatgtggtgatactatgatatatgtcggtcagagtttagttgctttagaccataagtcatgccgcctcaaatgggcggccgtgaccgataaagaaatagacggttactatgtatacatgagcgaagacaacgaaaagaactacaaaagaatggtcgataagccacttactgctctcgaatgggtgagtcctcctctacgttcagatcgtcgttacttcttctacattacagccgtagaccggtcagacaatgagtcacgaccaagtgcaaccgtactattttcaaacgaaagtgaagtgaataagagtttaattcctgtacctgctcccgtgaacaggctccttaacggacaggagaaggtaacagataatctacctgctcaagacttcatgctttcgtttcagttaggaggaaactcatgaactacaagtttattattggcggagatgccaaacattatgtagagtttttcttaaatcaaaacgactacacgattacagggcgttacgcaaaggctggcgtcattgagatggaacacaaagtagttgtcgatgatgtattatctgtagagcctaccatcaacccacgctttgaagtgtccaagaaaggacgcatatgggacttccatgtaagtaaacatgatgtgtatggtaaagagtaccgccgaagcgtagccgtgtttacagcagaagatgtaagtgctgataaggtagaactatacaacgatacacaaaccgtgatgacattaaaagattattccgttcatccactcgatgtgtataatagtcagctttccatcggtaactttgagttccgtactgtcgatggcattacaggtcagtcctcaacgcaggacgggaaaccaaagctaggcacagacagtttactcatgggattcggcggtgacattcagattgatctattcggcgaaggcgtacttgatgaaacaaagattccatttgtgatgatattaaaagtggattcattagcgaagaatgatagtagtgggcaacaccagttaggattccaactaggtgataatatgtttaaaattgatacgacaaaaggtatcatcttgtctcgtcctgatacggcagacaaaacattgttcacaaaagataagattccgactgacccgtttacactacgtctattcgtaagcaagacagaggcgaaactattcttgaatcacacatctgtatctattccggattataaccgcagtacacgagtggacttcttgaagttatatagcttctgtaacaatggttcgtattcgattatcgaccttgtgcagttccatgcactgacgaactttagacaagatacagttcctccaaaagccgttcgaggtttagtagctaccgcaggtaataagaaagtatccttacagtggttaccaaatacggagaacgacttcaaaggctatcgtgtttatgtcaacggcaagcaacataacatctcgccgatcgcactgaatatgtatgatgtgaatgcattagaagatggtaagccggtacgtatctctgtaaccgcagaagacttagctggtaatgaaagtgtaacgtcatctgttatcactatcgcaccgatcttagatgtggcaaaagaggtttccaacttctctgcacaggtgacagaaatcggtgtacacttcgattgggttccgccagtctatacagatatgcaaggtattaaagtataccgcttaaatccaattgatggtacacgtattgaattaaagaaacttccaactgatgcgaagacgtattctgatgaaccaaagccgaagccggggatgtacacgtatatcgttacaacctacgacaaagcaggtacagagacatacggtgtgcaaaagacaatggaagtcttataaaaaggagcaggggcgcactgggatatgcgcccctttttcaaacttgttagagagaaaaccaagtgacactcacaagcagcaagctaggagttaatgatagtataaacacaaagtaaattctttatgcttgtagattaataattttatctcggaattgccccacaggtagaaaattggatttgcactcgacttcttccgcatgtttactgaatacctccatccgtctatttacctttcgctcgttacctgaaaataaatatagtacagtaggaaatttccttcggtagaattgcgaccattcctcattgatccattgaccactaagctcccaatctcggtaacgttccagtttcttaatttcatcactctctttcttctcacacgtatccacctcaatgaagtaccggtatccgtagttccctttacggaaggcaagaataatatccggttcaatgttataagttttcggcttgtgtgtataacaaggcatgatgtagtcaatactgatagtgccgtctagttcgagcaacctcgctgacgtgtacacatcaagtatgtttgaatgatgacggtattcgatagacaattgcttgggaggtcttcctcccttatctaacaatctgtacccggctcggtcaagccaaatgtattgcggtgctgtaccctcccctaacggaagtctaggggagtatttatttacaaaatggtaatcatataacttacgaatgcggtcacgacatatttgtcgtcccttcaataatgtataaaatggtgcaagttctctcccgctcctgagagacgtcggtggtgtaagccaaaatagatgctctgtttttacaacaggaaactgcgccatgattcgaagcatctccacatcacggggtgtcaggataaaccgatgctggtctacccagtcgtctgttatcactgacacttcttctcgttctgagcgacctctacgtatcttccccaagtctcacaccctcctcgtcatctatatcgtcatatgatgctagtagttctttgtattgtttgatattcatcttgagattccgtgcgttattgtgaagccttacttcatcatagcttcgtggagcaaaatgcattcgccacttctcctctaattccttgtcacgaccaaactcttccggaacatctagaccatgaaagataaatggaggcagtgcgctcttaccaacatatgttttaaagtaccagtggtattccagttgttctaattgctttagttcttcgacgctgatgctaagttcttgcgccataatctttaggttctttaagtcgagtccaccaccaactaatatatgaggattcatcccgatgatttgttgccataacgcttcgttctttgatgacacttgcgatggattctgtatgaggaagttgtcgctcacgccgtatttccgtgacttcgatatagatagttcaaagataggtgtcgcatccatataggtctgcggttcgtcgtacatcttatgatgaatcggcggtagctttccttgttgcagtaatccttctcgcataagtgtcgctgtccagtaaccatagtttaggaagctcatcatcatgcgctgctcattctcaaggaagacaccgttcgataaattgtacagtacccacttgccttgattcatccagttgaagatgtcgagcttacctcctgttccgcagaagatcgggccgagtcgtttgttatattgcaaattatctaatctgtataaaataggttgcaggatttcagttacctgactatcactctttacaatcttcggaaactctgttttccaccaaaggaataattccggattactctttcgtatcgtaggcaagaacttacgacggtactcttcatctcgcataagaagaatcagttctaagaaagtcgcatccggattactaaagacagtcatcgctgctttccgtaagaaactattcatccggtgattgtgtgctgttttaaaatacacctcaaagaactctgcaaacatcgcccctattgtatcgctatctctacccaacgaggagaagttaaagatatggggatatttttcgtctgcgaaattaacaagtacaagttttccacgctcctcttccggtgtagattcgatagcttcgagataaagtctgccgtcagcaacatcgaatattgaccctccgtgaccagcttccattgctccgtttttgaagattttaaccgctgtagatttaccacttccgggaggtgcaatccagagtcgggctttgctcgcagtgtcacggttagcgacactaaatcccaccaatgatccctcggtagcatactcaatgctatgtccaattgggataatatctcgtgtaagtctttcatcgacagtcgtttcatccggcttcatcctctctaactttcgtgttgtaatctctttagttggaaggcgaagtaatacttgtagttcatcatcacataataaacttgctgtcgttagcaccggcatcttacgtgtacgcattgcctctagtgtagacttctttctaataacaggagacatttgccagctattataatagcccgtacttttaaaacaagatgcgatggattcaatgatgtcatttcggcgctcatacgtattcgcatagacagctactctcacaacaacatgatatccagtctgacgtaccttctgattgaactctttaatttcacgatgacgttttagtttcaaggaaggttcttcttttccgaaggacgatacaagatgtgtgatttgtttatcgagtttgcgtccgccacttttgagaaaggaacctccgctatcggcatcacctccattaatatagagttcgtaggcacgttctatctcgtcttgccatccatcctctaccggatcaatcagcagttgtacaaaaactttttcatccttttgtaattcttcggtaacgtacaagatgtctttgcataacatagaagtgtccatcctgttagggaagaaaggtgctttggttgtgtgaaggtaagagatgagcgtgttttgacagtctgatacgaacggccaagatgagtggatgtattccttcaccccgcttcctttatagacagattgaatcttctctttcagcatggtacggttccctactgggacggtatacatgtagaaactttcgttgctatctgcgtacagttcttgtgcaatgatgggttgaccgtcccttagggatttaaacatttgattgttacgtatacttgagaaggacttcatgagtgcatgtaagtcctcatttgtgttgtctgtagcaggtgcaaccttatacgatacatagtcatgtgttttaaatagaccaggagtgaatttacttttgattgagtttttcatttgttatcaccttcagtgtgccgtaaccaatcataagtagtaccatcgtatttacgccaagcagaatcgtctgcataaagataagtgtctgtaacattgtcatcctcctatcgtttcagtatggcgaatcgtgcaagccttccgattgttttgagtggtgctaaactgtttgcgcctgagtccatcatgccgcgaacccatcccggaggattcagcatcatgtgtagcccgccaatcgcaagcataatcttgataataccgtactccataccttgccatgcgctatgagtgggaagcagattcggactataaagtatgatggcaatcagtccaagcataaagctatgagcgacctgcacaaagaacttatgaatgttatctttcgtccacatagataagatactttcaaatggtttgaagatatagctgaccatcacaaacggtgtgaccagcatattaaacatgagtccgaaccaacgatacgcatgatagagaagtattttaaagaggtagaatccgagtgcgagtaagaagaagaatgtaagtacagacatgaatatactattgctaagacctgttagatgattcacgaatacatcgttaccttccattgctccgcctgtaatcagacccatatacgatacgagttcattcacgccaatcgccgtgtaataaaagataaagggtacaatagcagatacaacaatcgcaatcggaaagcgtacagcgatttgactcattggtgtatagctgagagataacgtgcgtttaagtccttctatcatcgtaagtaccgtaacgactgccatagagactccgctcatcttgaggagtataccaatcatagcagggttcttaaataactcgaagggagtaaagagtacgtactttaacatatcggtaaatatccagttcacgaactcgactacgaaccctctttctcttacgataagatccggtgcgctcatacaacccgataaaaacatcggtgcaatgagtaccgcacgtttctttagtttgtgtagttggggaataaggctagaaaacctcttacaatagtagctataacccctaacactgctggtgcagtaagaaccatcgtcatgcccgcaacggcatccttgtaacgtttcttcgcttcttctcttaaaccaatcatacggaagaatcctgctaccattgccatgatgattgcgacgctgacacatacgcctaaacagattatctgtaccttgagcagcacttcgtctacttccggtggtagctttgcttgatctactgtctctgctgatgttggcattgctatcatgacattcgtccaagtggcaagaacggttgtgtagatgtatacgataaccttgtttcgttttttcgtacgtaccgccttgccctttagaaaatttacaacatccttcgtgtcagaattgagattttgttttgcatgggtgagaatatcttccttgcatacagaaaccatgtcatgctctacagttccttttcgaccacgtatattgaatgcaggtgtcactttcttcacgtccttcctgtggttatctacatcatactgtatgacaatattccctcgtcttatgactgatagtagtttaaaataaatataaggaggtggaaaatgatggttcaaagtaatcgtgatgtaagtaatcaaggtgtatacaataacaatggattattaattggtgcaggtcttgcaggtgcaggaggtttagcgtgggcgttacttcgccgtggtaaaggtgcaggaaagatgatgggtcacgctgattcacaaacagcatcgtccgttgtagctccggtagagcagggatttcaaccacaaaactttctaaagaatgctgttgaatctgaaaagctaaaagaaacacgaattggtttaagtacctacttatctaaaaatcctgctaatgtagatggtgaagtatcacatgcactaaaatacagtcaggataaattcggtgctaaactatgggatgaatatgatggtagaccattagaacaagacaagtcgaaatggacaaaggattacttaggatacttaaaaagtgacctacgcaataacttcgccgaagaacgatttaatcacattctagatgtgggaagacaagtgagagggaagccaacacaattatagccggactcaatcgaacccggctaagatgttatctatgactacgccttcggatgcacttgtaacacccggcattcgtattttaatttgctctttaggttttggtttaagagggggagtcggtatcgtcttttggtcaagtcgcatcatcgcttcgtactttaacccatacctgaccaattgacgtagcctctttgatctatcctctatattttcaaacgcatctataatatccttatctcgtttcggggagagtcttgcttgtaaatcagccatgtgcaatcagccttccatacttcgcaaagcctcgtgcattaccgaactccggattatcatgcttcaatataatatctccgaactgactttggaagaatggtgttaatccagtagacccgccaccagtgagtaaaatctcatgaacatcatccggattcacccacactggaatctctccgctaatacttccaacatgcatatcaattgcagaagtaattaattcactgatgtctgttgcaatacctttaatgtataccttgcgctcacggtagatttcatctatctgtgtcaggttcttacgaataccatactctttagctaataaacgacgtaactctttatggaagttgtttgtagcggtatacagtgttgtagattcactacgactaaaccgaccattgttcatagttacaatctctgtcgtgcggtaaccaatgtttacaattaccgttttcggtttaacacgttccgtaatgacctgcccactttcatcgagaatgtaatcgtagtagtctccggctccttgcggaacgacaaccactttctgaatcgtgctacggcacttgcgtcctcggaacgaatagtcatagtccctgacaaattcagtttcaatcttatctttaacaaggaagtattcttccaccggtacgcctgtaaccataaggtcaatcttgttctgtcctgtctcatacaatagccccagtcctgtaagtaattttactgtctcgtctattgtatccactttgttagctaggaatgtataccggtgattggtagcaaattcggaagccttatctcctacgtaatacgtttcgtcatcatacgtaagccacaagtctttcattgtctctccatgatctgtagcgttctgtatctcaagtgaggtagggaagccaataatattcgggaacttgaactcaatgtcatcctctaagttaatgcctttaaactgtttgaaaccaatgtcgcaaccaaatatatacatggtaacaccctctcatgattgttgtactacatatctatggcataggaattgtccattataaccattacgcaaaaataacaggaatattatgtagtacaagtggatagactagcagtaagtgacattctcaaacatgacaggaggaaataaaagtgacagctaaagcaaagaaacctagatatgcaggtgcatataatcggttattcccggttaaagtatctacgagtacattcctgacaatctatattttacatttgttaacacgaaaggaacgtttatatggtaaggaaatcatcaacgaaatcgagaaccgcttcgccggtgagtggaaaccttcgcacggtttggtatatccgatactccgagaacttgaagcggagggtctagtaaaaggtcattggataggtgaaagttctaagaagacgattcgtacgtatagcatcacgaaaaagggtaaagcagcgtacatagaggagaaggagaagcacaaggatgcattctcgaaatcattctttatgatggaaacgttgttgggggatttgtatacagactttgagatgatagatatagaggatttaaaatagaggagcgaatgctcctcttatttgttgtagtacggttgcagacacatacgccagtgtttatcgtttaggttcttgttacctgcttcaatcataagttgtattacatctgtgttcggagcgccaccaataaaggtaaaatcctgtataccacgttcttgaatgtaatcacctaatgaccacggcaacttcgctgcttcgaactgcttatgataattcagcatcggctgattgcgaagaccagcaacaaccgaaccgcaataacaatctgaccagtgtgcatctgttgaaacataaataccattactgtaatttgtttgtactgctttatggaactccacggctgaacggtttctgtcaggtgcagatagtcgctccggattcggtaagttccattctacattctgaccgacatacctttcaataccaacgatgatagatgctttgaatctaccagccatagcctttgtatagtcggttaatgtaccttgacgggttaatatgattggtgtacgtgtttgagcagcgtacgaggcaatgccgtacgtcataagatgttcctcgccatttacaacgagtaccttcttctcacctgtaccaaaccatgtggatacattccctgatagttcataccgatcattcccgttaatgcggtctacctgtagtcccattgcacgaagctctgcttctacgttaccgccgattgaccactttgttcctacgacaatggcacgatttgcgccaagtctacgtatctcattcttgattggcgttggtacgccgtcatatggtacagggaatagtggcccattaacggtagcggcaagtggtacggcacataacatttcaggagaaccttgctcgccttccggtctaacacgacgcttctcaagtacatcgtactctagagaaagcatagttggtaaatcgactacgccgtcctcacgctgaatgttaaatggattactaccacgacgttgcttcgcttggtataaccatacggctaccttcgtcgcaatatcgaaacgaccatttaccattagaccagcttcaaggttacgctttgtaatgtcaccaacccagcgaccttgcatgtcgtcaccttcaccgcacatctcattaaacatcttcacgccaagcagtggccccgaaccatacttaccatcgtactgaatactaccgtcaggtgaacccatccatagaccaaacaatccgcccggatatttatctaagcgtttctgtaagtcgatttcccattgactgcggtaatcataacgaccaccaccgattgtttgatggatacgataaccatatgcagattcaggagcataacgtaaacagatataccattctcccgaccaacgtccaatggcacgattcatccacggattcgtgaatacagcgtccggcataattctatcccaaacacctgcgtcaccttgatagtatttgttccagttaaacttgattggatcccaacccattcccacttcacgaagacgttgttgtaattgtcgtatcgctgttgtttgcggtgcttcaatacaacgtgggtctaggttctgaaattcagggccatcccaatccgtttgtccaatgtgtaagttaagcggccagtgaaattctttatgcacatacacatctgttacttgctcttgtggttggggtaattgaagcggtgcaagtacgactgtacctgttccgccaccccagtgatgccgtgatacagcagcagccgtttcatatacatcggctccactcacaacttgaaatcccatgtcatcaccttgtttcttatgtagtaaggcgggtttgcctcactctattataacaagaaaagaggcatccgaagatacccctttgtttaagcgtaatacactgatggtggttctttcatcggctgtacattcatcttctctacttcatccactgtaaagaactttttgttagcaaccggaaaccactgatttgaaaatttccagtttggtgattgtgaacgaacatcatatctgttcgagtttaaattaataatgcgaacctgtaacccgtatagtcttgcgtttaatagcgctatacgatacgctagtaagtccggctctgctccgtaataaatcagtgccttcggatcgttgaccttcttcgcaaccgctaagattaagcgtccagttccgcagtatggatccagtagcgtttctgtattgtcgtaaagagttacggcataatgtaggtcaacatactgtgttaccttatctctagaaatgagagtgtcagatttgccaacacgttgctcatatacttcgccgaaccaatcccacatatcttcacgtagtagatcaaggtctaactctgcatctaagcgagtcatcacatcttgtcgtaaccacggtgtatcaatcatcgtttcgtagctaagtttataggctacgtattgaagcatggcatcgaacgcttgcgtagcgtcctctaccttatcttgctccatgatgatgtccatgattttattgagcatctttaactgctttgtcatctttcttctcctccttcttccatgtccaaatcttgatgtctaataacttgcggaagaagataagaacgaatgcaacgcctactgctggaatttgaataatcattaataatacatcgtacgcagttacatagtcacggttttgtgcaagctgactttgaattgctttcttgtaactcagtgtccacttcggcccattccatctatcttcgttgtatagtcttgcagattccataatgtttaataaacggaaccagcagaagattaaataaattgcaatgataactgctaaccaccaatacatgccataccaagttgctgtgaataagtctacgatattcattatttctccctcacaatcttatcttcatatttatggataatagcactcaatgaattgactaatgagtttgctaaatccacattgatctcgtctgtttcccagtaacggtcaatcttttctttcagttcttgcatatctacttcacgcatagtggtagacctcctcataattgattctgtcaattctgtcttgagcaagctgtttagctgctgaccaagaaccgaagtgacgataatatgtagaagagtgtggcagtcctgattctctatcacactctttcttactaggtagatgcccatatatatcagaaaaacgaatcagcaagtcaatcatctcatcgttatcgaatgcatgttgctcgccgtgtatcttctgattggcgcatagttctaagttactaatatcattgttatgtttattctcatctttgtgatgtacttgctcctcagaagttaagtacctgccaatgtgctgttccatcaccagtctatgtagataaacccttccatccgaatcagcatgtggatggtcgggttgtttgactctaatgtattgactcatgctaatcactcctataacttatgaacaatcatattggtagttgacggtacaatatcgaagtaatgttcttgatccattataacaatatttgcctctataatggtatccggaagcattggtgttccttcttgatatgaccggacagctatgtactgacgatactgatgcttacccattaagaccgttgccggctttagattttgcttcgccatctcttccatcttgtcttggattaattccagtatctcttggcactcttcggtgtatacaatctcaagcatatcttgaccggaagcatgacgagggaacataaacttagttggtcttggcgtcgtaggtttcactttcgctgtacctttcgtgctgataaaccctgcaccataacgttctaccgtcttatactggtcaagcacatcgtctgttgttttagctggttctttgttaacttcaaacaccggttgcggtatcggtaatccattctcgtcatataggttttgaggttttagcgtaccactatgcttcgaaaaatcatatattaccatgctgaatctctccttcatattgctcccaatccatatcgccaatgaagtattcattgtaatcattcggatacggcttcacgtatacaatacgcttaatgccagcttgattcaactgacgagtacagtacagacatggttcatgtgttacatacgctgttgcgccttctgatgatataccaaggcgtgcgcattgtagtaacgcattctcttctgcatgattggtacgaacgcagtgaccgtccttcattaagtgaccgatgtcgtcgcagtgcggcataccctttggactaccgttgtatccagttgcaatgacacggttctctttaacaagaacacaaccgactcttgcacggctacaggttccacgttctgctacatgaagtgcttgctcgatgaagtattgatcccacggcttacgaactcgctttggattcggtttcttttcttccgggaataattcatgataacgttcctcggtataatcttcaaagcagtacatacgaccataaaagaattttaagtgtctcatctcttcttctgtgcaacgtattaatatacttattgctaaggcattaggcgcaggattcggtacatttgttgcgatataaggctcgacatgtagctttaagatatttacatctagacctacttgcggattagacatcgttttccattttcgaactgcgttcatttcatcttcctgcggaatagttagcatataataatgaatgttactaacaaggtatctaggcatcctgcttcactcctttagtggtatagatgtggttctccacgactgtagtctttgttctcgttgtatatttcaagacgaaatacatctttcatttcgtttacaagggtatcgtattgctcctcggttggttcaagaacaaccgtaaattgaaagtattgcttcgtttgcatacgatactcaggatatactttgaatacttttacatctcccgaactaagtacaggtagggattcgattttctctatctctcggatcccttcaagacgctctgaatatggcacatcttttcgaagtggaattgtaacatacataactactttactggtcatattgttttatctccactgtgaacttatccgatggtttcgacaatgcttctagccatgcgttatgctgctcatctgttaattgaagtttcatttttacaacatcatacggcacatcataaaccgtatctattacttccgcctcgatgtcttcaaactgcaaactcttttgatttagtgttgcatctatccatgcttgacgctctgctgacgataacatatatggtactgggatattaactgtaactgtatgcatatcttttcctccttagacacaaagaaaggtgagccacaaaggactcacttcttcttgtatcgttctaattcttcgggggataatgtatacttcttcatctccccgcttaattccttccactcgtcaggtttttcatgaggtaatgttacgcccggtgatttatattcatagcagacaaaaccagtagagtgcatcgtttgtctacgaataccttttgactttacttcactcatttctgtttatcccacatgctaggtggtaacttaccaactgtactcatcaatgcttcataagctggtatgctctccatatcgaaacgatccggattgtccggatctgattcaccgtatccttctttgatagccattgttagtaggtcagcgaaagaaagttctttatcacgtaatgtgcgcattgctttcttccatgattgaatgtgtgggccatgatactcatataacgcttcccatttctcagggtcgatctgcgttacgatattccacatatcttttacaacatcgacagttgcgccctgcgctcctcgttcgaatatacgttgcgtgaagacgtttttgataagagcctcttcactgaactccattaagaatggtggcatgatataaccaactggaagtaaagaacgagccgcttgcatttgttgttttgcaatctcttcatcttgtcctttcttcactcgtgctaccatctcaaccgctttcaatcctgtctccatgaagtccccttttgcttctgatggtgactcaaatccccacggcatgttggcacgttgaccaccggcaatacgagaagtacgacctacgctatatgttactaagtggtcatacactggtttacctacgccaacgaataagaatagaatactttcgccacggattggttcatagctatgtccagccttcaccatgccgtttaagaagcggtctacgttatccatgttgttgtacttacctaagtaaatagcagccttgaatgcagagctaagacggtcagcgtccgttgcacgtaaagcaattacacgaccggattgaattgcactgaatccgtctttcgtatagcaaaggttaatgaacttttctaggtcatctagtgccttgtttgtatcgacaatatcatcgctgacaaattgtactctccctgtactgcgtaatgcttctctgtttaatacgaagtcttttgtcatctacttatcccctattctagtattctaattcgtgccacaatgccttcttctctgacaccagcgtcctctaaacataatcgcttaccagcgaatggtttgagttcttcattgccatactgcaataacttgtctacatcaaatgtatcgtctgcggttggcataaactcatgcaagtcttcgtcagtgaaactatatcctccaacttcacgagggaacagaataacatcgctaacacgtagttcacaagcacggtcatattggtaacggatttctaaatcaatatcagaagaacccactccgccgtacaatataatgccattcttttctggtacgatttcataatctgtaccagacccattcttataaacaacaatcgtgctttgcataatgtcgttaatgtcatggtcgaaaacaaatagtcttcttccataagacgggtctttctcgaagtcatacttctgttcatgtctacctagcttcttctcgtgtacaaacggcatacccggaagtggcttagataaaaggctacttggatacatcctttgattcttatttgaacctaacgtcataatcggtacgcttccaaacgtagtttcttttgttatcggcgcttgcagttcaccaacctctttaccattaataagtgctttcacactttcggttggagtagaccagccaatcttttcttttaacgttcgtttagacatcatgtatgactcactcgcattcgttgagttggattcacaatcttacgaaggtctgtaatccctccatcattgatgtgttcctgttgctctttgtattcggcaaaggcttgctcataaaataagcgtgccggatcatcctctgtaaagtcgagaactttcctacacgttaattcagtcatacccctattcatgcgcaaatcttcaatagtaaacattgccaaacatttaccattataaatcaatggaagcggtgtgtccattggataaacacggtaaccttctttttctactcgaagcattctgtggttcgcaatatcctcttcgtagttctgatattctctcttctgccattcggtctttgcgtccttttcagacgtcactatataccaattaaattctgtctgcatacccatttattcacctcctgaaaaacggactagagcattcaccctagtccattattattagtctgctaaatgatactcaatgtcgttgtagctattaccgaagtagtagtcaacaacatgaagtggagcgtgcttgtgatgtaattcatcagtatacgctgcttcgatgtaacctttcttcgcttcgaagttatcaatcgggttgattaatacttcacactttggcgctcccgggattgttacctttagtgccatgtactttgtatttgcaatgctatgattctttagtgcgtctaatagtgcttccattgataatttaggtatcatttttctagctcctttagtatatgagatacaaacaccgggaatttatccatgtctaattgctgtggtgcttgctcgacttttaatgtgactgcaaagtttgtatatgcttctaacgtcactgcacattcatctacagctccgcagaagattgggtaacatgtttcccctgtaccgaatgcgccggtaacaagatcacgaatgtcatcgttttggaatttctcaagtaccggcttaaatttcgtcggtaactctccatcgccccatgtatatgttccgagtattacagcgtcgtattcatgcaagatagattcatagaactcattatgcccgtaaacatctaccttacatccggcacgctggaaggctgcggataaaccgagcgcaacctcctttgtattaccggtgttggaacaatatacgatggctaccttatagtgacgcaaagttgtttttcttcgttgcgccagagtaatcacgggacttgttctcgaattggtcagttttcgtatgttccatcatctcatccgagaagacttgaatccacggcatcgggttgccacgttcttcatagatattatccatgcctaattgacgtacacgtttgtttgcaaggtactcgatgtagtggtcgaactcttctaagtctaaaccttcgatgtcagctaagatttcgcccgcccactctttttcagttgcgactgcttcttttaaagagtccacaatgtactgtgcattctcttgtgtatttagttccggattctctgttagcaatctacggatcaataagccgatgaagtatgcatgttgcatttcatcacgctggatatatgaaatcattgtactcgacttcatcatcttctgttgactcgcaagccagtagaagaaggcgaatcctgaatagaagtagataccttctaaattaatagactgcaccagcatacggaacaagttttgtaaggaaggattctctgcgaagtcctcatatacatctaatacaagtttgttacgtttcgctaatacagggtgtttcattgtttcattgaagtattgattctgaacaggtaagcgtacaagtgaacttgtaatgtaagaatacgaagttgtatgtgttgcttcctgctgtgcgataaatgcagagatgtttgcaagtgcagagttcgttgtgaatcgaatcacttcaagaattgcttttacttgtaaactatcaaggcaagcaagtgcagtaccgacacgcaagaatgtatcttgctcaattggagtcagtgaatcccacatcttctcgtcttctttcatcttgatctctacaggacgccagaagtttgcttctagtgtttgactaatgtcgtaccaaatctcatcttcatcgttccagtttagtaatccggaacattctccgccaacgattttcgttccacggttcggatgacttggttctagtagtctaggtcttttcagtgttgcaatagggttcatcggtattacctccatataaagtcccgccctgaaaatgggcgggttcagttagttttatgcgtgacacgcttcgcaatcagcttttaatttctgtgattcaccttttacgtagtaagttgttttgattcccaacttatgtgccgtcttatggatgtttaacagttcagcaccgtcaatgtcgcttgcaacatataagttaaagctaatagattggtcaatgtgacgttgcttcttcgccgtttgtttaacgctccataactggtcaatgtgatgacgagtcttacgatacacattgtacgtgttatggtctaagcccggagccgtaactttaaacttgaagttacctttctcctctgcaccttcaaccgctgtgattggatcgttaccgtcagtagagccaccaactttagcagtcgttgcattcggagcaagtgccatcatccagccattacgtacgccgaacttcgcaaccttctcttttaacgctttccacttagcagatacatagtcacgttggtcgaagtattcaccagtatgccatgcagaaccttcgaagtatttgtaagtacctttctcttttgcaagctctacgcttgagtcgattacatggtacgcaaactcttcatataatgtatccgcataatttactgctgcttctgattcccaatctaatcctaagatagcaagtagatgatgccatccatatgcacccattccaattggacggtagcgtttgtttgttataccagcttgcagtacgctgattgtattgatgtcgataatgttatcaagcatacgagtaacgatgcgaataacacgagctaatacaccagccggtacagctttcgctaagttcactgatgctaagttacatacaacgtattcgcccggagaacgtaccatatggatattctcattttcgtcaatgtactcgccaataatcatagatgaaacctgattctgtgcgatttctgtacaaaggttagaacaataaatcgctgtacgaccaataccatgaacgtgtttgttcgggttcatacggtttacttggtcacgatagaacatataaggcattcccgtttcaagctgaatttccataatgcgaaccatgatgtccattgcacgatgtgtgtcacgaggtagtaacgggttgtttactgcttccatatacttctcttcgtaatacttcgtatccttttcatcatagaagtcatctagcgttaataggttaccattctcatctttccattgcatgtgttgtttgatttgatgaggacagaatgtatgccattgaccgattgaacgaccattttcatcaacctcatctaacttcttcatgaatagatccggtacagctacacctgtgaagatttcccttgcacggtcagcttcgtcaccattgttcagtcctagttcaaggaacttcataatgtccttgtggtctagacacatatagatagcgaatgcacctttacgttgtcctaactggtcaactgtgattgccgtttggttaaacatcttaatccacggcgttgtacctgatgatttaccttcgaatcctttgattggtgaacccaatgcacgaaccttactaaagtataaggcaacgcctccgccatcttttgatagacgagcagcatccatgtttgatagatagataccatctaagctatcatccactttatctatgaagcaactagataattgaccgtagctcttacctgcgttagcgaacgttggagttgccgccgttgcatataagttacttagcgcccagtataattctttgatgtgcgctaaacgagttgcttttggttcattaatcattgttgttacagcaatcatcatcagacgttcctgtggtaactcgtataggctaccttgataatctgtagcagcatagcggtcatttaataagaacaacccaatgaagttaaataaactatccttgctagggtcaatcattctaccaatctcattaatctcttcttcgctgtacgacgctaagatagctggtgaatagattcccatctctgtcaacgtcttaattaacttatagaagctgccgtattttaaggaacggctgtatcctcggttttctgatgccgttttgtacatttgttgtagtttcatgttcgctgcaacgaatgtccaatcaggcgcacccatgtcgatgttatttaaggcatagaagatagagttatgaagtgctttatcttccgtatgagataaacgttttaccttctcaatgatcttcgttgcgtcaaggttgtactctttcgacgctcgttctaattctacgactacgcttgcaatgacttcttcactatgttcaactgtagggcttactgggttcatctttctgtctctctccttatatgtccaatttcttgttacatttagtgcatatgtaattcacatgtgtctcgactgacgggtgaacgatatgttgttcatcctgtcttcgctgacgcaaccctttgggaagagtcacaattttctcttcccgaaagtctgcgccgccacatttacatcgaataaactcttcgtaaattgctgccatactattcccctgctttcttcttcaagcgtaatacttctttatgaagctcctcggctgtcgccttaaatacatctcgttcgttctctaggtggcgattctctgctcgtagcatcttaatctcatgcaccatatcacatatcacatcgaagtcttgagccacatatgtcttacctgattggtcattcttataacggaatggtaatgcaggatatgtaccatatgtcttcgcttcctcttcgatcttctctagcatcgtctttggaatcgtcatggtcttctcacctttggagttcaccgtaccacgttccttacactctgctagaataaccgtatcagctacgtcacccggcatgaaccatatgttaccggaaccaagttgtctacgtgcttgccattcctttacggaaggtaaagcattcaacttcgcggctacatattcctcaagtgtctcacctgagttgtccgttatagaagtgaccatgttacggttaatcttcgaatgtgctttcttacgttctttatctccgggtaattttaagaatcgttggtcagggccacactgcaaacatttactgttattcttacagcgattataatactcgcaatcccaaatatcagtcacgtttacgcccctttccacgacctacgtcgcctcgtccttgtgcatccagtttcaatgcaccgttgccattgaccatcttatcactcttcttgagcaagttgtcgataacagaatccataaactctgctctctcctcccatcgctctacaagcgggataagtggttcactatcacccttaaatgaagacgtttctaggtactgtaagattaatgcatctcgttcatctaccgtcatacctgctttctttttgaaagtcagcttgagttgtttcttggcatttgataaacgagtagaataacgcttgagcatcttcttcgcatatccataatcaaaccttgcacgagtatatacttcgtcaatctcggagttgatgattaaaatatcagtcgcatcgagatcggcgggaacttcaattgcttgaacagcgtcaatgtagtaatcttcgaactcatcccactggtcatcggttactttccgtatgtccagctttccttcgtcgtcacttacacgctgttgtttcttcttatccattgcacaacctccttgttattacgagttagaagggatggataatctctacgacttcatcaatcgtcttatcagacgtgtcgatatggaacatagatagttttggttgacccgcatgttccttctcaaacgcaaacaggacttcatcatacttcgcttgttgcttcatgatctcctgcacgttttcgggaatcgcatcaaaaaggtcaaccttctcacgattgattctatggattaattcctgttcatctgagtgatgtaggtaatatatctctacttcgtcaaacacttcgaataactccttgcagaacatttcgtaatgcgtatcgaagtcatactgtttatacagttgagaatataccatctcactaaagaagaagcggtcaaagatgaacaccatatcttttgtaagcctttggttttctttcatcttctggaacatatcaaagaatcctctgtaatatgttgctatcttgagtaagccatctagaccatcgtcgctgaaaccagttggattaatccatgtgcttccccatatttcttctcgtagtcgtcttgaaactgtggacttcccggttgccctcggcccttctaacataattgccttcaacgacatctcctcctaacgaatatactgtcacaattgagagtccataaaagaagtttacgataaaacgcatcgtaaactctaatgtaatggattataggaatcatcatgcagttgcacattttgacgtaaatgaatcaaaactgtctgcacttcttgtgatgctcgctctaagtttgctatcgcaagttcagctttctcaatcaggttcggtaactcttcctcgtcgttacccgtgtatagaatcacgtttggctgggactgtgaagatgtttcttgaatctgtagtttaccctgttccactagcgttttaatcgcacgatgaacagttgcattggagtacccagtcttttcaccgatactcataacagattcttgcaacttgccgccaagatgattccatttgatatatctcaaaatgtcatgtaatgttcgcttattattagtcatacgcataatacgtcaccactcctactatatgttgtggctcggtcatgtaaaatcagtatactatgctagaaaaaattcagcaatcttaaaacaccttatttttcttagccttacgaagttcagtcgtcagacgttgcttcggttcttttgcggttttgtccgttaggatagagatgctttcttgcatatcaatcacttgtttatctgttgctaattctaccttatcgaagaagataactaagttaggggataacatagcaagcatcttctcgacgatagcttttctttcttcttgcttacgttttgtttttacgaagcgaagatgcagataatacacgtcatcttctgtgagattcatgcgcttccacgctgctctcagtttatccgcacactcttttgaaattggatcttttgtcacaacgacaatcttcggattatatgaacccgtaccaaactcaatgattgcattcgggaatttctttcttgccatcatatgcaggtcatcaatgcgcacatgcgtgcgaagataatccagtgcatacttacggtcttctttctcttgcccttttgttacagctagtaaactactttgaatacttttcataaactctttgtctgttgaacgatacatactatctctcctcaccaataactcattattgtcatcatcataatatagtaaacgaagacgatgaacaactcgccccatcctcctgttttaaacttcgctaatgaatacattttcttagacggccatagcaaaggtacgcctttcttattgaataaatctagcaataaatgtgtcatgtatcctacgaaaaacatgaagataaagctaggtacgatgaagaggagactgaatatgactgcacctagaagagaatgcgtaaaattcttcctgtgtggtttaaagaataaccataatggaatcaccctacctaaatacgattgttttaaatccgtatcggcaaataatcctccggctgtacatatcagtatataaaacgggtgtagctccaatttagatgccaatgcaccgcctgtaatagcagcaacggcaatatgtgctttacctgtcataccatcccctctctcggcttattacacctagtatagacaaatgtaatactctttattcaaagaatgtaacagtatatttacccggtgtatatttcggctcaatgtctgttacggtatgtgaatgcgatacaagtttctgtgtgatctgtaccgtcattggtaactttaattcatgtgctaattcttttgcacgtttcactgccgcaccttttgtttcgaatccaccttcttgtagttccatgtttgtgtattcaaactcattacgactaagcgggatacgtgtaccataagaataaatctcatagtaagtcttccactttcttgtgccttctgttggataatcccacgatgtacatagcttcgtttcctctgccggcatcgctgtattaatcgcacaaatgcgacccatatccacatcttctaagtctgccgctacacgagcagcgtgattacgacttaacgcttctacctgtatcttattatcctttctagagccaattaagatacttggtaaaggtttgttattcggttcttctttcttagcacctagtgttctcttcccaaccggtactccgctttttgtttttgctactggcttcttcttcgtttttggtaatggtttctttgttggtgtagccaattgactcgctcctttcaggttatcgtaaatatcttggtcatctagtcctaacttagactgtgtaaatgctcttgttggattgtaatgatcccattcatgaaacagtaattgcaactgacgacggctctcttgttcttttcgtttttccgcctgtggacgcttccactgtttcggtttcttattatcatatgttaactgtgctgacgtcatttttatcggttcttgacctgcacgaaccttatctaagcgtgctaaaaatatctgcgtatgcacaagcgggccagctttcaataatttaatctgatgtgccagtgccgcatgaagttttttaaatccttttccgtccgatgtcataaacgaatgaccgcttacccaatgtctaatatgtgtaattgcagtacgctcccacgcaagagtttcttcttggcggtttatcttcacttgatgattgatggcaaattgatgctgattatatctcttaccatctgttgtaataaatacttgtcttcctttcgcatctatttcagattcgagacggtcaaataagattgccatttttaattcgctccctcacattcataatcatgatgccgaggcgattcttacctttgccggttacactttgaccccagtatctatctctatcgctatgatgaatcagttccttatcaccagtgcttaataataatgttcgtagccattcatgttgcttgaacttatgtattagtccgacaaacatgacatgctctttcatgacatcccaatcctcatgaataggataatccgtacgctgacctaatcgtcttgcttcaaaaggattgtttgtcgctagaatctcctctaccggttccaaatcaataaacttctgcgcatgataataatgttctaacgtcggatattctgtgccagcaatgacaacagggtgtttactcatacaactaaactccgagtagttatctgacccggagtaaaatcgaatggcgttactttttatatttgctttcaacgtgttcaacctccttcgctatcttcaagaaattacgacgagacacgagaacacgatactctgtctctacaagctgtttatctaacgtcacatataccatgaccatatcatcttgtaagtgactattgttgtgataccaaaacgatgccgtataaagagacggatcgctgtcgataaacttatagcctaattcgattcgcttaccaatcattttattaaaggtttcttttcgtttcctcttctctgtattctgagcatctaatatatcctttatagacatatctcttacttgcttgttcatagactcactcctaaccgtttctcaaccatacttttaaatactactttctccgatggattctgtatactctcaagtattggtaacagttcatctaaggcttccatctgtgcgtcctgcatctttctgtcgaagtcagataatgtttgttctgctacgacttgcaacgctggttttgcatttcctttgatgaagtttagaatcgacgcttcgtcttgctttaatttattcatgacctctgccggatccataccaggagatacaacaattttgacacggaatccaaactcaagtaaatcttcaatagattccatcattgcatcaaagccagcagaatctccatcataccaaaagacaagattgcttgtgattcgttttagcttttccatttgttcacgagtaaatgcagtaccgcatgttgcaatactattttctaagcccgcttggtgggcgataatcacatctgtatatccttcgaataccattgcataccctcttgaacgaatcgccttacgagcgtgtgtaagaccatagagataatgtctcttctcataagcctctgacgtataatcgtttaggtactttggctgttcatcctttaatgtacgataagccattgcaattgttttagaagtcttcagattgaatccctcctcgactaaaccaaataccaatcgaccggcaaccttctgaccaaataacggattactattatcgtcctcgcttatgtatcccatgcggaacttcttaatagactgcttcgtaattcctcggctatacacatattccatgactgctttattcgctaataagtttctgcaatactcatagttcagcttatgtgttttcttcttcgcttgctctaacttatagtttacattcgctttcggcggtgtaattcccgcacgctccattaaaatcatacatgcttccgggaaagatacacgttccatattcataacgaacccaatgacattcgggccgccatcctcaatcttatgtccatccggaagcgttaggatgttgggctttgttttcttctcatccgcccacttacgtccggcactacacttcctgctaaaacagaaataagacgggtcttcgtccttcttctcaaagatgtgcaagttaccatgctcaccatcgttatgaaacggacacgttgccttattgctgctgttaaatactactccatattcctttgccacttcacgtaagtcattttcctctaatatttgacgaatatgatcgtcggttatcttaccgattttcatgttgctacctccttttacataactgtgcgtgcgaacgcacataaaaaagacaccctacaatgagagtgtcttttgcttttagacgaccttaacgaatttctcgcttgccgtgatatagaatacagcaccttttgagttctgtactttatattgcggtgagccgtctacatctacttgcccgatgattgtgaagccttgacctttcaagacaacaccggacacttttgatttgtcccaagttggttgagagtagaagtttaattggtctacgatagactcaacacgctttcctacataccatgcttgagttcctactacctcaacgaacgtatcgctcgctgtgatgtagaatgttgcacctgcggagttttgtaccttatactggaacgcatctccgacttgcagtttctctacgatcttagggaagacttgatctttttcaatcgtacccgccacatctgcatcagcccaagaaggtctgttgtagaatcgtaaagagtttgctttacatttcaattgtttccctgagtagtcgccacctaaagaagtaacattgtcattattgacaggtggaggagcgatttgattcgttttacctccgctaatgtaccactctaaagttttgtcacccattaatttattacggtcaactttccctacaccattcatccatccgcctgtttcaccatcagcatattgccagatgtcacatttgtacttcggttcagggccaccgtaacgtggaatccataagaagtcagccttcactttgtttaatccatacttctcatacatgtgatgagatacgtagaatccaaccatccagcctttcgacttacagcggtcaatgaatgcttgagatgcagcagcgatattatcagggccacatgatttcagcgtatcatcttccgtatccagtacaaggaactttgcattcgggcttacacgtttcatgaagtcgtcagcctctacaatcgcatctgcaacagagacaaagcatccgtaagcatatgcggcatgtggtacgccacgagcttctaattcacgtacaaagctatcatacaaacgatcctttaagttagaaccatattgcacacggcaaattgctagtcctaattgtggtgctagaacgttccaattaatattgtcgttccatttcgaaatgtcgattacttctctacccatactatcagctcccttgtgggagatagaggttggttacctcttatagttcgtcaactgacttcaagtcaccatacgatagaaatatgcgaaactcaaatgatagaggcgcataatcttttccgacaactcgtaaaacatagtcagtaacttcattatacatgttctcttcgtgtcctgcattaataaagaatgtatttgctccgccacgctgatgattaactcgaatcgctttatgtccgaactcaccctcatacgctttgattaacttacgattgatttcctctactaacgcctctttgtcgtatcctgcgtgtacgacaatattgaaagcgccgatttgcacttcatttgctcctatcggttgtggttttgccattgtgtttggctcccttctctttaatgtctttttgtatttctgataccaacactgcacaatcatcacaataccctgtgttgtgtagagactcacttgctggaatctccgttttacaacgtgtgcaatttgtcactacgttttcgaaataacgtttatctcttttcatcattatccaccatttactactttcttcggttttacccagctacacttgtcacatacgaatccatactttggtgttcccggaatacgatccatccaaagttctcctccgcaatgacataactggtcatcattcatttcatggtactttaacgtatcgaccttcgcttcgatgtagttcgctaaagtatttacttctttacattccggacattcaatcaattgctccatgtccgtttcttctttaatacgtttctctgttgattcatgtaatgcgtggtctttattaatttcgataatgccgttgtggaagattacttccgtcatctctttattttgtctgactgcatacggctcaccgttctttaatacaacatgcatctcatgtgcttgcgcacacgctggacatctaaacataatctaacctcctaaatatttgagtgccaccctagtttaccatcgacgataccaaatatcttttcatgtgtatcttcatactgctttgaaaagaactgcgggtgggttggtatcttctctcccggatgattcgggtcatcttgcattgaaccctctatgtacggtacgggttttaattctttacttggcggttcggattcctgcacttcgtaacccggcccataccaatagcgtaagcaaagcggtgtaccatcttgcgttacatgtaagtaatcacaaggcatgtggtggggatcaggatgttcgcatattgctgtcctgtcatttctagctccccaacctatgaagcgttcctcaaaatggtcacatttgctatctgctggcgtaggtgttcgatacccacgctgtcggtttgttgtgcgaccgattttaactttcgccatcggaactctcctttcttgataagactcacatagtaatcttatcaagaaaagaagccgccgactagggcgactgtcctttttatactgcttcttgttcttcctcttcttcgtcctcgtcccaatcaaagtcatcgtcgtcaccgacatgctctctgataaccgttttacctgcaacaatctgactgattaatgttttgtcagccgtcatcgctaggtgcggttggtgagtaaccatgataatctgacggttaaatgttttcgagatgaacgctaagaactcacctagcttcaccacatattcttctgatacgtgtttacccggttcatctaagatgatagggccttgtatcttcggttcgttgtatacaaccatcaccgcaacttgcaatgcaatcgctacgatgtcattgataccaccaccacgagaatcttgtggttcattctcaatcggctcaccattttcaccaactgaacatacgaggaatcgtgcggcaggtttcccgcctgattccgtcatctcaattttgaatgtgaaatcatgaccgaatatatgttgtaaggcacgagtaacaaccttctccatacgcttacgaccagcttcacgggctgagtctgccgtcttcacaagtagtgcagagatttgttccagcttcatacgttcctgtactacgccttcaatctttgcttctaggtctttcttttggtcagcaaggatattgcgctctgctacacgtttgttaaagtcatcttgcattcctactactttatgtgtaagtacgcttaaacgcatcagtacacctccttattgtgataaatcacttcaagttcctttaatccttttggcttgtattccttaatagtatcttgaatacgacttggtacagattctaattcatcatccgttaatgagcgatgatccacacgaacaataatagatgcttcgtccggataatactcaaagataacctgtacacttacgcccggaatctgttttaaatgatactcaagcatggagaggttatcatcacttaacccctccggctttgtttctttatagtacggcatacttaggcgagacatatattcaatcacacgtttcgcatctttatcataagagaattgacgacctaacttatgtgccaacatcttaatgatgatttgattgtccgtctgattaattaatcgtttgagttcatctacattgttcttctttagaaactgttctgtatcttgcccgattgcattagataagacactgcctttacccatcattgttcccggtacaatatagtcctgcgggatacccacattagcgtttttcatgagctgattaacagcatcctcaaaatcagttttggtgtaagatgcggcctcaggagaaggtttctcaaactgcttgaatatttctgcctcaagagctttcgcagtatcacgacgaatttgctgatctaactcggctgatatatctctatatggattggatgtagataattcattcttgcccatgatgactcctactcttcaaattccatatcgtcatcatcgtctgctacttcttcacccggtaaatattgacgagcgtcttccattacctctgttaagttcttttctttttcagcgataacagtagctaagtccttcggatcgtagcccatgtctttaattttcttggcagcttccttacgacgtttagatgcttcctctttacgagtttctaagcgcaccttatcactcttcgcttgctcaaattcgttctttaacatttttaattggcggtcaaagtttgtagtcatttgtatttcctccctatgcaatatgttctaacatttcatttactgcgtgttcagatacttcttgattacatgttggacacttgctacccatgtctttaaagatggattgaatatcttcttctagcttcgcaatatcttgcgttacaatttcgatactgccatcaatctctttacacacatagtcttcgccttcatattcttctagaagtttcttgagcgttgcaatttccgcaacttctttctcgattgcgtcgatgtcgatattatctaatgaagcatactccgctaagtcctcggtatgcagttcttctttcgcttctgatttaatgtactctctatttaattccttgagttcattaacctctttatagatgtactcggctgcacctaataaagtaagaccttgctcaagcattccgtactctttgtactccgaattaacagcagataaagtacgtgctgttttagcgaagtcatcttgcaactgttctagctctgcaatttcgcgagcgatttgctctgcttcattcacaagtaacataccacgttcaacataggcataatcatttaactctgcattgatttccttgatggcacgagacttctcttttaagatacgtctaaagtctttcaagtcttcaatctcatcgtcaaggttctgcgccgtcgttaataatccttttaggatatttaagcgcttctcaatttcaggaaggtcatcataattctttagcttctcatccagttctgcaatttgcttctcacttgcattactatcgacagtaagctttcgtatttccttccccttctctttgatcgcagcatcgacgatatggacgcccgtgagtcttccgattgtgttagcacgaacagcaggtgactctgagagtaagaagtgactgtcaagctgattggagaagttaatagctcgttcaacacctgttgacagttccactttaggcatttgatgagtattggcgatgtcgataggaatgttattcccaaaccccttgaactcagtctccttgccgtttgtatcacgtacgatgtactcaccggatgatgatttagttcgtgagcgagtaattgaagacccgtctgtaaaaccgactgtgaccgtagtccgactagccccgtggcgaataaagtcggctcctttaggctcgttatagaatacccatcgtaacgcacgtatgatggctgacttcccggagtccgaagcaccggtgattgcattaagtcccttgtcactaaaatcaaccctcgtctttttatgtgattggaagttggttacctccacccatgcgagtccaattgctttcgccttctcgatgtatccatctagcttactatcttggaagttcttctcttgatctacgatagcatccagtgcagttgataataatttcttcgagataccttttgtcttcgctagtatttctttcgggtcaaacgattcaaatgttaaagcatcctcaatcgtttgctcgaatgctgctaagtatttcacctgttgtttcttctgcacgatttgtgtacggtcaaatacgtctacacctttttgtgcaacttgatattctacatactctacatcgaagccatttgcgtctacgtcaatcatggcatactgcggaatacgcttcatactatcggcacttgcttcatcacgaccagtagaacccggattcgtaaatagtgtaccgttgatttcatgcatatcataacccggatgaaaatgaccattaaagactgcatcagcttccgttacaacatgttttgttaatgtatgttttacgtccggatggaacggtttttcaagtaacataccgtgagagaataggaagtcatagtcacctttcttcttcacgtagtaatctaacattgggttatcatcaatctctgaatgatattgtttaccatgcaggaatacggtgaatgcacccgtgtcaatcttatgcgcacgcttgtctgttaacagcgtaacaagacccgcattcgctaatgaaccgattgccgtttgattgattgtatccattgagtaaccatataaatcatgattaccgggtacaagatatactcgaatacctttcttgcgaatacgtcgcaagatgcgttgatgctcattatataatgaataagcgacacgaggcgtatggaaccagtctccgccatgaacgatgaaatctacatcatttgcatacgcaacttgttcgatttcaaggaacttattcaagatagcattgtgataatcatcaatacgagagccgggattacgacccatgtcgtgactatctgtgaaatatatacctttcaagagcgtcatctatacttcccccttacccttctttttgatatattcagcaatatcttttaccttcgacttcgcaagttgtcttttgaaatcttggttatgatactgacagataccattttcaacttgatcttgtttgcatcgagctacgccacattctctttctttaagctgaatgtattgctccatgctatctatattccctcttgagaaatgcagattaaagttcgcataatgtttcatgcacattccttttgcttctgctcgtaaaccgcaatgagggataaaacattgcacaccatgataggaaatgcggtttgttttacgtgggtcacctattttcaatcgtctttgattatgcattgcacatattccttttgcataagcgggacgctcacaatcctcaatactacaggtcttgcctttgtttatcagaacagggtcatcagttgtacccgtaagtcttagtctctgataatgttttggacaataccctctagcgccggtggcatcctctccacaaccttccactatacatatacgggtagcaactgtacacatttaaatctctcctctgttcttcttttctatatattcgtttgcatcttttgcattgacacctctatggtatctaaagtttagatgatgtttattacataagtggtgtgcaacagtctcttcattgcatccatcaattctgcatataggatccatttgccctctcagattttctacgtgatggacgtcaccatatttattaaaacgttgcagatgcacttggcacaaacgtttaccgtgcgcttctttttcgcaaccttctacgctacatatcttacccgaattaacatagtcagggtcgtctgtatttccgtgacgtaactcacgacggtaatgtttccaacataaacccctagccgccttcgcttcattttcacactcgtctattgtacaggtgttaccttgcttactcaggcgtggacgctttcccgctttttctttcttcctctcttgttctgctctccaagctgctgctttctctcggtcagcttttaactttgcttgccattgttcaacttccgcttgtcgagttgttaagattggtgctaggtttacgcaaaaatctaaatcagtctttgcttcccatacaccatctgtaagaagattcccgaagcggtcaatgtaccccttgcgtccttcatcaatcatcagaccgacttcccagtcactatgcgattcacgtttcaacatacacatcatgagaagacccattaccttctgttctttcttctcactcatcgtcatcgtcctcaggttcgtgtcctatgatttgaacaccaatcagataatcttctgctgtatctaaatcatgctcttcgtaatatggcccggtgtagaagcgaatgtattctacttcttcgtatgaatcctgctcatactcttcctcttcgaacgtacaatacttgtcgttctctccgtcgcacggtcttacatttagaccggagaataaatctgtatagcaagatagaatatctccgccacgagaattaccgcccattcttgctactacttttgccttcgcaacacccatttggaatgttagttccacaatatgtgtactatctccgccgggaatatactgctccatctgcatcataaaacctccttgtttctagtatgttacattttgtaatgcttatacctaccatgcctcatcatcaatagaaaccttcttctttttcttgagaaaggcaaggtcttttaaatctgtgcggtacatacgatggtcgtcttcatagggtttatctaattcgacctgcaccggtgacatgtgatgctgatagagagttggctggtcaacatatgtgactttgccttttatatcaatcgacttcacacgtacacggtcaccttcctttggaatgcgaaccttaccagtcatcccagtcaacctcctcttcaccatctaataacgcaacaagtgtcggaatccaatgctccgacactgtgccattgtcatctcgcttaacagttacatgcgattgacgattataatcaaacggaaagtcgattacgactcctgtccacggccatgcgatatgtttgactctgcatcctaatcgacatacatccattagaatttcttcctttgtacttggttgtttaattcttcttctttgcggtctaggtagttacggataccttcttctgcacttttcgctgttgatttattcataatcgacataacgatagcagaaccgattgcaccgccaaccatactagagaacgcaatgatgaagataaatgtccatataatatacattagcagaatctctccctccttctaaaacgatactcgattatctctcgtgactctaggtcataagcataagcgataccgtcccataggtctaccattgtgagtcgccagccaaaacctgcgcccgtgtcaatcgcaatcttcttggctttgttactcgtccatactctcgcatcttcttctgtaattgctttatggatacgataagtaggtgtgtgtccgaatacgatagtctggtcaaaagtgtgtgcattacgtgggttcgaccattctttacatcccataagtaatacttctctcttttgtaagtccatataagggactcgtggatcaagaccagcgtgaacgaagataaagttatcgtcttcatgataatcaaccatcttgtttaatacgccggacataatatctcctacggcttctgaaccgagtttcttttgtgctatatcaatagatttaagagtggtaagtcctccaatcgcttcatgtgagaattgttgttgcgtcatcttacctgtcagacattcacgaagccatgcatcatgattgccctctacgataactgcgccttcttgtagtaactggttacattcctcatatacttcaaaggattggagtcctctatctcctaaatcgccacatataactaattcctccgggtcaggatcaacataatccagtaaccgaaggaatggtgtggcgtttccgtgtatgtcacttattgcgtaacgtttgttcaaaatattcccctccattggcgaaaccccgccaacgagacagggtttagtttgcttctacatcaatgacatcgacacgcttgatgcttgcttctgtactgaatccttccggataacgttcttttagtttagcgatattagcaatagcaacttcttctaacgtaaatccatactcggctgctatacgagcaagataccacaagtcatcaccaagctcatctcgaacctttatcgggtcagcagggtgtttatgtcctacttctttcttaatgacgtcagctacctcacctgcttcaccagctatacctaaaccatatgtagcaatgcgaagttctctgctcatttcatggtctttcggcgcagtacgctccgtcaacttttgatactcattaaagttcactactattcccccttgatctctagcttgtgcgcccacaacgtgctactgtacttctgtatcagtaagtcgatgcttcgaccatcatacatagcttgcgcagctttttgaataagttcagctacttgtctacgttttggtgcagtttctacatcgtctgtgtgcgtatactgtaatgtatattcagcgaacaacttaccagcgttcttcccgcttttgtatcgcttaatactaagagtgttgatgttatctgctagtaattcatttgcaacatcgtttaagactggaattgttgcttctacttttggcatattctcacctcatttcatcagcccgcaccctctattataaggcatgagggctgatgcgactaggatttttagaagtcttcgtcatcaacggaagcggatttcttcttcgctggacgtttctttctagcaggtttttcctctacttcttcatccggctcttgatcggcaagttcagcattcattgcgtcaatgatttcttgcgcttccggttccgttaattcattcatcttgtctgtaccaatattgtcatacaagaagtcttctaactgtgtacggctaaagcctagctttttacctaaacgtttaatcttaccaatgctatcaacagatgctaaatcaggttcatatgcctgtagttcatcggctgttaccatacctacgtttaagaagtcacggaacgcacgacctttggcacgagttaatgccatacgtgggaatgatgctgctgtcattttaccgcagttattcacacttgcatcggcttcttccataccaatgcgtttaccttctttgtcgaagatttcggcacgagcataacatgtaaacttattctccgatgacggatactgcgtgacatatgcctcgatatgtccaaagcgtgggtcttcatgtgcaagggcaagcaaaccattatatagaattgcttctttcccttttggcccaccaatcttcatgataaattctttcttaaattcgggtgttaattctctacttttcacactgcaacctcctaattagccattaatccgtttgtcatagcaatagccacaaacatctaatcccggattcctcggctctgacttacaatcaacacacttcggttctagctctttataaatcgcttccttgatctctctccattcacctagttgcttctgatgatttgcaacttcacgtttgcgaccatcaattacaacttgtggtgtccgcatactttcaggaagagatttaatataagcactcgcttgttcacgacgagtaatctccgtattcacgagcttccgaagtttctttaattgtgcattagaaaacgggcgaccgtttaccatttctcgattctactttctacctgtaatgttgcagttaagtcgtcgtttgtacgaattactaattcaggagcattgtatccataagaaccttcatagtatttttggaatgtaccaccacgccataatggtgtcacacgaccttttgcaaacgtaccattctttttcgtaggaattttattgaaccattcttcaaacttattaccctcttgaatctcaataataagtgctttgatttcttcctcttccgcagcacgctctgcttctgttggacgtgtaaagtctgtgtccatatcacaagtattaatgccgtaatcctcatcttctggatcagcaccaatcatgctttgcagttcacgaatgatttcatccttgtttccttctaggttgaataccttatgacgctcatctgtctttgtgaataatactaccatttcactccactcccatcttcgccgcaattactacagctatttcttcttaaccagctatacagatgctcttttaggttatcttctagcggtttcccgtagtccttcataaagtcagtgacaacatcgtcaatacgcataatgacggcactacgttcctcccctgtatagaaaccagcatcagtatcacatacgagtttgatgatttcatccttaatacctacatcaattgccggctctaccggtgctaactcatatgcacgaatcatgcttgaatcaataccgtattgcttcgatgcaatttcaattgcaactgcggttgattcaacggaatacgtacctaaaataataccagcatgaatgtcacgccattcttgtacctcatagaaattctcttcgtcttgtataggctccgttaacacttgtatgtcacgagtgtattcttcgtctacaacgcatagataatcatttttgaccatgtggggtcaccaccttcaattgttttagttgttcaaatgcttcttcctctgttaattctcccgcatctaacttcgcaagcacttcatcaatgctattcattgcttccggatttcgaccaatttctgtttctaccgtatagctatccaatggttctacatattcacttgaattttccgccattagtttctctaatgttttacgtaattgatcgtcaggcaaggataaggcattctttatatcatcaactttaatgtattctgcatcctctgatgtttttaccgtatggtctggtttacccggacggacgtacagtgtatcagcggcgaaaccatcgtctgctgaacatgatctgtcgtatgctgcattatgtttctttaacaactgttccagttcttttatttcaccatcttgattatcttcttcttcaatcgagatgagcgttttctcttcatctacataacagttttctaatagtgatactttatctttgtaatctgccttcccgctatcgtaatattctttatcctttagatgctgtgctatctccggatgcaacatacgtgccgggaaagtaacttctacatgaaaatatcccatcttgttcacctcattacttttattgttaccatctgtaacgcttttatgcagagaaacaagaggcttttacacctcttgaattaactcatccggcaactcttccataaagcgtgaaggtgtttgccagttatacgtacggttataatccatacgacgctctgcatgagtgaaatacaatatctcctgcgcacgagtgatacctacatacgctaggcgacgttcttcttctaattgaactgtatcagatagactacgtgaatgcgggaataatccctcttccataccaatcataaatacgactggatactcaagtcctttcgaagtatgaagcgtcattaattgtactttgtcttgattgactactttatcttgctgagattgcagtgcgatttggtcaaggaagtcttgtagatgtgggtctttattctcttgttcataacccttcgcaatcttcgttaactccttgatattatcaatacggcggaagtcttcttccttaccagtttcctgcaacttctcaatgtactgcgtatatttaaagataagttctaacatgtctgtaatcggttggtcagatacgctcttacctgcttcatacaatgcatttaaacgagcgactgtatctgttaatcctacgaatgcagctttcgctttcgctgtcttcggattgaaatactccaccacttcatagatagacttgttcttttcgtttgccttatctttgatttgtttaatcgttgttttaccaacgcccgcttgcatgttcaagatacggtcacatgcaatatcgttgtcaggattaacagctaactgcatccatgacatgatgtccttgatttccttacgttcatagaaggagaatccggaaaccatattgtacggaatgttatggcggataaactgatcctcaaggagacgagattgaacgttcgtgcgatacagaagtgcaatgtcttcatacttgtatccatcgaatgtacataggttctgaatctcctgtgcaatgaagcgtgcttcttgttcatcgtccggcgcttcatacttacgtatcttctcacccattcctttatccgtgaagcaagttttattcatctgcttcgtattatgtttcatgatggcgttacctgcggttacaatcgtattggtagaacgataattctgctctagcttcacgaccttcgtacccggataatctttttggaagttgagtatcttctcaatgttcgctccacgccacccgtatatgccttgataatcatcgccaacgacaaatatgttattcttatcgccgacaatgtttttaatcatacggtactgacattcattcgtatcttgatactcatcacacataatatacttgtatcgtcgctggaacttcctgcgtacttccgtattgctttccagtaagataaccatcttcatgattaagtcatcaaagtctacggcattgttacgtgccattctgtcttgataacgctcatagattctcgcaaatgacttatcttgcggttccatttgcttacggaaatctttcggcagtatcatatcattcttcaagttactaatacgggactgcgttgcatatacgttctgctttgagtcatccatattgttactctttagtaactctttaatgactttgtttgtatcctctgaatcgaagatcgtccagttctttttcagacctgcttcactataatattgatgtaaccaacgtacagctagagagtggaatgtaccaatcataatcttcttggctctcgcttctcctacctcattgccgatacgttccttcatttctgttgctgctttatttgtaaacgtcacggttaatatacggcgtgggtcaataccattttctaaaatataccccacacgcttttgaagaacccttgtttttccacttcctgctccggcgataactaaactagggttctttactgttttaacaaccgcttccttctgagctggattcagaccatctaataacttacgtatccgtttccgctttctctttctttcttcatccattactaccacgttgcaacctcctatgctgactctttaatacttttccatgcaccttgcaacgcacggtcaccgtccataataggattatgttcccatacaatatcgccggtgtcgtttatacttcctaaataaataacgttctccattatgtcacctcctaaaatggaacatcaatcatgttttctggtgttacctctactgcgatgaatccatgttcatgtagtgcaggaagtatatcaagttctgtgtaattttcaccttcacatactttacggataactcctttaaattcctcttctgttgcatttgtttcgattagtttcatccacgtagaacgttttgaatcgctatcaaataaacggaatgtgttgaacatattatgcctcctgttcttcgtatttatcttgatacaagaagaatgcggtactgtctgcatgatgactatgaatgaagtccgataactcatcatacttgtgatgaaacccttcatttgagtattcatcctcaaggtcacttaataatcgtttagcaaactcattggctttcttttcatccttgaagctgtattcaccattattcacgaataatcgtttgactaattctaagttctctttatggtcatccatgatgaacctcctataatatactgtcgttacatgcccatattgcttttgatacatagcgtttatcaaacttttctacggtaggttcgattgctagtatcttttctccacggattctatttactgttgaatcatcagactcaattagctttcgaataccattttctacacgacgaatcgcatctgcttcatcttgtgcaaagataacaacattcaccgccatccaactttgtgcggaatattgaacaacataaggttctccatccgggtcaccaattaaaaacgattcatcttgcttgcgactccatctcattgaattaagaaacgggtctgtatatagttcaggcataacaaacctccttactagtttggcacgggaatccccgtatgataaaaagagagggagcattgttacatgcccctctactgttatacaatttcagttgtttctttcttctcggctggcttcttcgctggtgttttacgtgcaggtttcgtcgctggcttcttatctgctaccggctttttcgttgtaatcttctttgccgacgctttcttcaaagcagcttcttcctctgcggcaatttgcttctcgattttctcaagttcttcgatgtctttcatttcatcagcatcttgatacttcggaagtagttcaccttgttttactttcccgcgaatctcctgacgtaactgttcagcgaaccacggattatccgtcatgaactcacggaacttcgttttacccggaaacttcaatggtacattcttcgccatctgcggtactggatcatccttcttacgaacaagtgcctcggctgcttcaatctgctcaccgtcttctttctcgtaatagaaccatgagcctgacttacgcatgatgccagcaggttcaacgttctccataatctcagtaagcttatcgacaccctcaccataaataacgtaatattcacattttttgtaagggttgtcgtatacacatcggttcttcgcaacacgagcgctgattttcagtccatcgtcgtccgtaattgggtcactatcttgtagtttcagtttattcataccaaaacgtaatgacgcatagaacgctaacgcacgaccaccagttgtcacgtttggcttacacatttaagtgtaatggactatatcttcatcctctatcgaggagccttgcgcttcgaatggtgctaatctccatcctactctactcgcttccatcgtatgatgtgctttcgatagtctctacaccttccctattgggcttggcacggtattgggagccttccgtccaccgttagccgtcttacgacgacaccgcttttgcttgcgttcacaaggttattttacataccattactgatatgggtgacccaaaacttttgtatttgttgtttcaccttatctatatcatttaactcatcgtcttcgattgttaatacacggtatcctttactgtgtagttcagacaacttacgtttatccttatttacattcttttgtttactgtgccaatatacaccgtttacttctatcgctagtttcttacctaaatagaagtctacattccatccttcaacaacgtgatgtgtaaaatacacgatgtctaattcatcaagtatttcttttacacgacgttcaggtgttgttagatgtgtcgatgtatcaatttgtggcttaaacttaccgtgtcttttctttctccatcgttggatagcaacatcacttacgccatacttttcagcaagctcaattgatgaatactttggattatcaatatccttaaagaatgcttcatctcttggtgtcttccccggacgatagtgtgggttcttctcgatatattgcttcgtgtcgatacccaacttcttcattcgtttctgtacacctgcaagactaataccaatgttattggctaattcatttaaggatatttctcccttccaatctttaatcgctttacgttgttctgcgtttagtctatccttcggtacttttgtaaggtcatgctcatcagcgtatttcataagagcatcctttccgatacctaactctttcgcaagttcacgtatgctaatatcaggagaagcgtaaccgtcgataacgatttgtttttgttcaatagatggtgaccatgctcgtgacatattattacctccgactttaagtagttacattcgtttacttaaagtctaacataggtaaatactattgtcacgcacaaatacgagtccaatcaccgaagcgtgagtttacatctgtacgtaattggttgatgaagattgcatacaagtccttcttaccgataagagcagtccacttacgcattagcttactcatcatacgagcttgcagggcgatatcagactgtgctaagtccgtttcaacctcacgtttcggtactaaaccagctacggagtttacaacgaatcctgataagataccagaacgcattaacacttctaaacggtcaatagattcctctgcacctttaagcgagatatcgacatagattagacggtctaagtcaatgccgtgaacatctcgtgcatagtctgggtcgaaactttcttccgactcaagccaagcccaaagtgcatccggatcttgctccatatcttcaccaattgtttctaaacatagtgacgtttttcctgatgagttattaccaaagatttcagtaatacgaccacgagggataccgccaccagtcatgacattgatttcttcggaaggtgtaggcaagaacgttaatttctttgctacatcttttgcgaaaccaaccgctgttgaacccgccgttttattaataccatccattaacttcatcgcaattttgcgtttttcttccttcgttaacttcttatcgacttgagtttcttcggctgtttctatttgtttcttctttgccaatccgataacctccctataataccttctttagccgttgtagtttcggctttagtatacggttgccgaaactggcataggctttctcttttttcgaatatgcatcaacgtttgcgataatatcatcaacaattgcaacaggattagcgatgcttgttgctacaccattaaacgtaccattttctacatattctccatgaacttctgctatgtcttttgctacttgtgcttttgcatccttcgcactcatgccctcttcacgataactattaaagagatagacaatcgactgacgaaacatatgctttgcacctagttgttttggattattcatagattttcgaataacatctccttccgttatgatgcggaacgtccgttacaccccaagacggggcgaaagggttcttcgctttaggctttaactcgcccactgcaaagacccctttcgtttctcaatcttcgtcttcttcgtcgaagtagttgtcatcttcatcatcatcgaagtcgtcatcatcatagtcctcttcgtaatcctcatcctcgtcttcttcctcacggcgacgtttacgagacttactagcaggtttctttttcttcgcaccatcagatgctttaccgcttgcacgcttcttacggcgtggagcttcctcttcttcatcttcatcgtggtcatcataatcttcttcctcatcacgacgacggcgacgctcactacgtggtttacgttcgctacgctcgctcttgccacgagaagcacctgcacgtttagaaccactagacttcttacgacgacgtggtttctcttcttcctcttcatcgcctacgacttcacttacagcctcggacaatgcttttggaagtaagctaatcttacctgctttgaacgagatacgatcctcttctgcgtcatctggacggtacacatccatttcacaattgacgaatgtaacccaatgacctacacctgcatactcacgtaaatacgctgcatcgtgaccaaagatagtgactgctggatatacgtttggcatatccccttctcgcacgaattgtttacggcactcaacaactacctttaagatagcatccttaccgtccttgtcattgtcatgaatgattcttttgatttgccctgcaatatttgcactaaacatattgtgaacctcctaattgttcattaagtatagtatttccatttgtaataatcatatacacgaaagaaacgccctcgtacctttatcgcacgagggcatctgcctttattattttactgctgttttcaactctttaccagctttgaatgccggtacttttgttgcagcaatttcaatttcttcaccagtttgtgggttacgtcccttacgagccgcacggtcacgagtttcgaatgtaccaaagccgattaactgtactttgttaccttctgctaaagcgtttgtgataccgccaaatactgcgtctacagcttttgtagcatcctttttagagatttctgctgtttcagctacgtggttgattaattcagttttgttcatgataagtgtcctcactttttctctgatttgttttgtgtttctagtgtccttagtagttgtcatttctgactatcctcattttatcatccataaaagtaatgtcaagccttgacactaagccattttagaaaataataaacatgacagctccaataatgacaacccacttaaagataccttttgctacttccttgaatccttcccaaaatgattctgcaccttgcttcattcggtctagacctcgaattgctgatgagcgatgcattggtgtgctttcctttcgctcaatgacttccacttggtcaatcgtaatagtttccatcatataacctccttatacataacagggcgtgcgaacgccccgtcatttaaatgatgtagctttctacatctacctttccgcttttacgtttcttcttcctcttcggcgttggttcctcgtcgaatagatcggcaactgggtcagctttcttcactggtacagattgctcgactactgcaagttcctccttcggagcttctacctgtgccaaaggcatagtcgtcttgatacccatctgctttttcagcttcttcgcatcacgaacgagaactttgcttgcgatgaactggtctttctcctcaccattgtagtagctcttttctctataaccacggacaacgacaatattatccttatagagatgctgactacaagatttaaacacgtcagggaagaccgtaaattctagcggttccagttgtgtttcgatttccacgaacgccatatcctctttgcgcttcgtcttgatggtacgtacttttgtaatacgaccacctacatcatactccttcttgtcacggcgaccacgggatacatccatatcaagcagtggcgtaaatggtaagtcttcataaggagaaccggaaacatagataccgattaattcgtgttccatttgaagcattcgtttttcgttgaactgagctggatcgtaagcgaatgagtggttcgctttctctttcgaagtcaacaggtcttcttccagcccattccacactttatctttacgtaatgtaaagttgtagtggttcagtaagttatagcgatttcgttcaaacgtatcaaaacagccggcctcaatgagtgtacgtataacacctctattaatcggattactcttctgttcttcggtcttcacaaatttgacaacgcatctcgtatagaaatcatcaaagtctttatagtcgccattattatctctttcacgtacgatggcacgaacggctttctcaccaacgccagcgatagagaagattccaaaccggattgcccccgcatccggagagaaatcggcgtaagacttgttaatatcaggtgggagaatgcggatacccatacgttttgcttcttttagattctccattgtcttctctttgtcaccgccacgaacggacagtaacgcagtcatgaactcaacgggataatgtactttcagccacgcagtttggtatgtttcgtcagcgtagcaagccgagtgacttttattaaaagcgtacccggcaaaggctgccatctgtgagaacaggttctttgcgaatgactctttataattgtttgtcacggctcctacgatttcgtgggaatccattgctttcttattcttcttgattgtgttctcgatgtcttgcttacgtaagaacaaatcatttccgtcatagtcagactttatgatgacgccctcattgccatctttatctgtatgtttcggcatagaatcgtacttcgcaagtaagttctcgaacttttcaattacatacttatgaccatacataaactcgtcgtatagcttcggcatttcatcaatcttcttcttaccgatgacacgacggaagttgtcggcatgacctaaattgtatcctgcaattacttgaaccagtcccatgatttgttcttgataaaccatctgaccacgagattgcttgagtaaggcgcgcatatctggatgaacatcagaaaggtactcttttggctttcgactaccattgagtacatattgttgagcgattgtattaccgttttccagagttgcatctagtgggccgggacggaataatgctagaatatcaattacttcttcttgttttgcaggtgctacttgacgggtataggctgtaatccctcgtcctgcaacttggaacagaccatgtgtctctccctttgccataacttcgaatgcttttggatcgtcacgaccgattgaataaatatcaatacgtttgcctgtagattgttcaataaggtcaattgtcgttgagatgatacgtaacgtggatgtacgcaacaagtccatctttagtagccctacagactcacaatcgggataatcccacatcgtgattggtaacactgcttttgatttctctgttggtttaatcgttgggcaatccaagtctagcggacgttttgaaatgatgacaccgccggcatgaataccactcgtatctattgcaccctctatcttacctagatagtcattcagttcaggatactttgtcatcagcttacggaactgtttacttgcctcgacgaacttcgataacgaaccgccgtaacgagcatagttctccggatgaacagagatttccatcatgtctttgtaggtacagtctttttggtctggcatctttccttgatcgcctgtttcctttaatgtcgcttttacttcatcagcaaccttgtagccatattccaatacctttttcggattatccgggtcacgtttctcaagcgctttacaaattttatcaatcgaagacttcaatttgaacttcgtgaatgttaagacctgcgctgttttgtcacgtccataacgctgtgctacgaaatcgagcaacatgtcatgattgtcaccagggaaatcgacgtcaatcgcttacaccgtccctttcaggatactttaacactgcttccgcagtcgggctagactataccattgtctctagtgagacaccctttggtagtcgttgagggctactaatgtatccctgctgatcgtccattgttacgatgacgctgagttgtatcttcgtcatcatccttctacttgtttctgctctcgctccatataggcaagaaggctttaggatgttccagcatattcagggtacgcacggcacatcactgaaccgtgggactatacttttaagtagcgttgatactttcgaaataaatatatattagcgccattgtagatatagcgaccaaactttcttacatcatcttttgccataaactcaagtgagtaactgttcttcgtgtcagtatgtcgcttgtgcaatgtaggcttgtggtctatgtgttcagcaagagtcgttaagaactctcgtgtacccattatctttactgcgtatgtatcatctctcttaccggagtatgagaatgaaccatcgccatcaaagtaaccacgaatgaaatgatgtactaaatgtttcggtacaatatcttcgctaggaaacttgagaatcagtgacttgttttcgaatacacccttctcaactaagtgagtgtacatcgtcttactagtgaatctaaatcggcaatactttgtacctacgccataagagttaccttcgtattctcctattggatgcgtagcccgcaaagcctctttaaatcgtgataggtgacctttatcttttatgcctaatgcaattcctacattgtaatcgttacgtgtattggacacataaccatcggcatacataaagcctaaccagtaagccttctcttgagtgtcgattgtttcgaaaaatgattcatcaagtgtgtactttctgctattatctcttggagatcgcattagtccacgttctttcagggtgcgttgtagtttatttctaccgcatcctgttgccttccaagcgtctagcaagggcataccattcttataaaattcgatagctttttctaacttatccatgttataacctcctaatagggaaatcaacgtctataaatagactaacatggaattacgctacgttcaagtaataatcgggcggactatcacggtatggtgataagaaacgctcaaacattaaaccatattgtatcgggtcaagacgagtaatgtccaacaagaacgccatcatacttccggctgcggaacctcgaccggggcctactaggaactccggataaaaggcatacttcgcattcggtttcgatacgacttttcgtttgactttcttcttcttgcctgtctgcttatcctttactactttcacaacaacttcatgtacttctgcttcaatccattctttattctgcggataggataaccacttattacggtcggaagcccaaagtaaaatatcccatagcagtaagaagtatgaagggtactttttcattgttacgatacgtagttcatgttctacacgttcgatatattcttttacgttaatcttgtagccatttacttctacaaactccacgtagccgattaagttatcgaatagtagtttcttgagataagatgcatcagtatgaccatcaggacaatacgggaactcaggcatcaagtccttattgagttcaatatctacattacaattgtcggcaatccaacaagtattctcgatggcgctgtgaggtacattattttcatcagcccattgatacacttcatctgcacttgctagatgatacgtgtttgttccggagtacggtgcgcaatcatatacgctttgcttacgactaatcgcaaccagtgcatggtgagcgtctttatcgtccggctttacatagtgtacatcttttgcatacactaatggaacccctgtgtcattggaaattcttatcaatgcttcgtttacaatttcttgctcacgggatccgttatcttgtatctctaagaaatagcgatagaaaagattattacgacgcagtatctcttgttttgcctcctcttccttaccttctaagataagctgggacgtttcactcgcaagacatcctgttgtcgcaatgatgttcttacctagcccgttactttcgatatagttcatatcggtacgaggcaatggttttggtgcataatacacatactggttcgcatctgaaataaggtgctgtaaatcagcccatccttgattgtttactggaatcattgtaatgtgacgagatgcgttccagtttcctcttgcgcaatctgctttactaataacagttctatcttttgcttcgtacatttcacatgcaggaataaacttaatcccctcttcctcacatgctttcttgaactgaataacgccactgatattaccgtggtcagttaagataatcgtatcaatgccaagttgtttcgcacgcttcgctgtctctttcggtttacacattgcatcggcaatcgagaagatactatgcatatgaatgtgaacgatacgacgtggcttcttgttagatttgactgcatctaagtcgtccgagaaatcgaatatttcatcgtcgcttactacgtttagttttttctttctctttgtttttgttaccatgattcgtccaacctccctctgctttgctccaccataataccatataagtgagtcctgcatagaggctcaagaaataatgctcatatgttcggtgacctttctgttcattacatgtacggcaacaacatgctaaattctttgttaaaatcgagccacctaatcgtttgggtctaacatgatcgacagttaactctggatgatccttatgcatttgtgtaccgcataagtaacacgtatagttatcacgttcaaatgcttcctgcttaaatacagcttcatcttcatagtgatacaatacttgaatagtagtagggtctaaccaaatggatttatccgactgaacagaatgagccgccctgctaggcgttgtgtacccagcaggattgccattcttatttaaaacacggatacgtttagaactcgtcgtcgtccgagattggttctttttcttcttcatcttcgtcctcatcgtcccaagctacccattcatcttcctcttcctcggctgctgacgcaacttcttcctctgctaattcaatcgcaattggaggattatgaagactcgtcggattctctctaaacttcttgtcttccatttcaatttggtcaggagatggtaactcaccttttagttcactaatcttccatgaaccatacgtaagagttggcgtaaccgcacaatccactacgctcacaagatgagatgctttctttaattgtcctactacatattcgtattcttccttcgtaactcgtgcaactacgtgtcttgtatcaggcatcgtaatccagtatatcgtttgcatgataactgggtttgcactatggatattatcaacatacattgtttttgatgtcgctgtaatatccgcaatatctaccttgataacttctgtttcggatacttctttgcagacaatcttacgatttcgtgcattttcaggtagatcagcagtcgtatccttaattggtaatttctttttcacgtcggcagtaggcttcggctttgctttcttgcgtttagcctcttcttcctcgataatttctactacgtccaccgacttgtcttttgcggccttgaccttcttcttcgttcccatctcgattcctccctacataatgcttcctaacttcttgtctcgcaatttctcctgcaaccacccgcagaactgtttgtactaatgcttttaacaatttgtcatcaccctgtcgccaaaatacaagtgtgagccacactgctttccggtcgaagtgtaagtggttgtttatcacgaacaccaactgtaatcatatcagaatcaatatgttttacaacatcaattattcgtttcacgctgacactaacctcagttgcttcaccttgtagcttggtcattatgaactcggagaaattaccctcctgtgaatttcctgttagcgttacttttcccttttcaaactgtaatataccactaccatcaatcatgatagaggcacggtcaagtaagcccaacagttcgtctctgtccgctaggaactgtaaatcaaactctttaccgggtaagacgttatctgtgttcgggaagttacccgcaagtgttggaagatgatacgtcgcaagtccagtcttcacaataaaatgattgttttcacatgcttgcatcgttaccttctcatcatctgctactaccttcgcacaattgacgagcgacttcttacccagcaccacattaaatgggtctgacttaatctttcgcttgtaattcgcaatcgtaaagccgtctgtactagtcaatttcagaagattaccatctgatacaaactgtatgccctgtaagataggacgacttgcttcacggctagaatctgccgcaaatgctacgttattaatcagcttcttaaacactagacctgacatatcaaatgagaacttcgattgtagcttcggtgttttcgggaagctctcttcttgatacgaaataaacgtctgctgtcctttcgctggcttcgtcttgagttgcaggccaccgcctttgctaatggcaaacatgacagtctgtacgtctagcttcgatacgtatgcaatgaacttcttcgctttcactacaaatttaccagtttcttttacaccagcaggaacccatacttcggttgtgttactgccgtctgtcgttgttagcttaatacggttggttttcttactggcataaatcttaataccttcatgcccttgtgtctctgtaccaatggtatctttgacaacctgcaatcctttctttagttcgtcgagtgccacgtcaactttcatcgtaaaacctccttaaacatgcaaaaaagcccgtgcgagcgggcttcatcttatttattcaacattgcttttaattcagggtcagcatacggtttaccccatgcagctaacatgatctttgcttcttctgcgtattcaacaggaaaacgaccgcttactttcgtaatgtcacgctgtaatcgagagtcattatgtttcttcttctcgaatcccggtggtggcgtaatcgtattataatcgacatgtaatttctcatggtcgatacgtagcgtaagaagtcttgtttctcccgatggagctttatttaaacgctttgcgacagcttcgcatactgtatggaatctacttaattcatgtacaattgcatctttgtcttttgcgttatcatacttcccttttatctgtacgcctttcacattcacaatgacttcaaggttatcagataaagggaacgacttaatagcttcatcgtatacttcttcttttacacggaacccataggctccacgaatactattttgtgatgactgcgataatgtctcgttcattgattaccaagaactcctctccatctaagctaactggtacaccagccatgcgagagtaaataacacgttgtcccggttctacatccattgggatacgtccgccgtcaggtttacgttgaccgtcaccaatggcaacaacaacaccttcgaattttggatctttatcacctgttggtaagataataccacttgctgttgtatcagattctttttctagcttgattactgctctagctcctaatacttgaatgccttcatatccatctaaaaactttgccatttcctattcctcctacttgactgcatgtgctttaattgttgattttgctgcgataagagacttttcccagttctctcctcgcaacatctcatctaagtatacataaagagccgcattctttccagcttcgtctaagtcatacttgtcatcgtcaatcgtgcggaacacatggcgtacttcttttaacttgttttgtcctgccgaaatgtttccgattttcatgccgaaactattcaagaacttctcgaacgtagctggctccatttcttggctgaatgcttgttggtaagagccgttttctcccaagtattcaaccgtagcaatgatttcatacggctcttcttttgtgcgcgcgcctgttacaaaagcaatacgttcatctaataaatacgctgctcctgcaaaatataatgtatgttttgttccgattaattcactacgttgtgatgcaaataacatatcacatgccctccctatattacgctaacatatctgcgattggtttgatgcttccccataagtcattcgttcttgccttgaactcttctgtatctaagaagttacgtggatgataaacaggaatgacaggaatgcttttattccctaaatcaaaatcaaactcttcaccgattgctgtatctattttaattggttgttgtgctaatgctgatatagctgtcgtacctaatgcaatgataaccgttggcttcgttaccactatttcattatgcagatacttataccagtctttaatcaaactcatattaaccgcttctactggttctttaaatacttgtgtccagtatacatcgctaatcttaatcccgacacgatctaacaggttcattaagtagaaaccatacatgccaaatccactaaagccgaaaatctcttctgactgttccggtttgtcgctcacaatcattacttccggtgtcattgttccacttgagattactgtgccttgacgtggtaaatactgatgcattaaccctattgactcgtaatgaagaaacatctcccgtgagacacgggagacatctgattccttcattccttgttcaacaagagactgtatgactctatgctgcgtcatagacattacttaccgaataaagtctttttaatgtgagcttttacttcgacaactttaactttaccgtttttcgctttcggtggtttgataccgtgtttcttcatgatagcttggatttccgcttttgtttcaagagcgtcgatgtcctcaggagttaactcatcttcttcgtcttcctcatcatcatcgtagtcctcttcatcatcttcttcttcgtcatcctcttcctcatcgtcagactcgtcttcgtcctcatcttcttcctcttcctcgtcgtcctcttcttcttcatcgtcatcttctgcatcttcttcgtagccttcttcttcatcctctacgtcttctacagaatcgttttcgatttcagcatcgtcttcttcctcttcgtcgcccggtaatgcgaatccaagatattcagctaatgcttctgctaattcatccagtttagaacgagagttgataccttcaagagctagaggttttgcgaatgcccaaagtttctttgtacctaatttacgtaactcttcatagtttgttacgccttctggtgcttccactgtatcttcctcttcttcatcatcgtcgccgtctacatcttcgaactcatcttcgtcttcctcgtcctcttcaacttcctcgacaggctcttgttttgctttctttttctttttagctggcttttcttctacttcttcctcgtcgtcatcagcagctaaatcttcgttctcttctgctggtgctactttgcctgctgcaagttcttgtaagtaagttgttaatgccactaattcttcttgcgttaatttcgttggatctttacctactaatgctttcgcatcaataccgccttgtaatgattctaacttcactgttttaccggcacttgcatgtttaattttgcttgatacttttactgtttcgccaacgatttcaccgatttcttcaccagcttttgttgttgcgatgaacttgtcaccctttttaataatatggaatgcatcctttgcaattgctacgatacctgcgaatactgttgcacctgatttactttttaattgagtcattttgacttcctcctcgattttggtttaatactttttgatattcacttgccatctcgtcactgccatagaaaggagtttggcactcctctcccgtgacgacttcctctttactgccacgtatcaatcttattaaatcttttactatgtctatcgtccactcgaatacgagtatcaagacgacagataagacgaacgctacaattattgcaatcacgtctgctatcaagaggaaaacctcctatgattcaagcgacgactccgtgttcttcattaacttcattgcatcagctaattgattcgatagaaccaccagcttcttatcaagtatatcttcatactcggatgctctgtcttgtaaatccaccacacgttttgcatatcgctgaacagttttgacactggctgtgcctttgccaagtaagtctgcaaagtcggcaagcaattgatttgcttcctctgacatttgtgactcaacctgacgttgtaccatatcacgttgatcgctcgtatcaatcattggaataacttccataatcgacttcgacgttcccgtatggtaatcctctaagtcatcaatcacgccacgtaggttctctaacgtgttacgttgattacgaggaatgaatgtagccctaccattcggcatgatgcttacatggttcatgtcatttacgacacgacgtacaagattgtttacagtatccttagtatggaactgcatccattcattatacaaagcttgtgtatcagacagcaatacatcatacgggtattctgttaagaaacttgaatcccaactaatgtccattgtgcctgttttcttttgaagaatgaatttccccactgtagcatagcccagttgctcattcttcttatccactagctctcgtaccaagtgacgaatcaccatattgccatcggacttcacttcacgaaccatcagtcgtgcttcgctcggttttcctccgaagtctattgtcaccgttgacgttgcttttgcagtcgcccggcggtaagcatcatgagccttaatttcctgaggcatgaacttcgggtctaagctatttctagcaaagatttgccctaagtcatccttatggatacgaaggtctgaaatggtatggaactctaagaaccctaataattcggaagtttgcaagtctgcattacttcttactgctgctacgtttgccataactgtttcccctctcctagtagttacttaatttcaagagacaacttcatgtaggtatcaaagcccatctcgatgtcaaagaatgcaccgtattcagttgtcgtgtagctaaccgtttctgtattgaatagcaacggtaatacatggtttaagtttgtagatgggtctgtatctaacccttcacgcttcataatctcttctgcctttggcgttacatatccataatgtaattccacattatctacttcagcagaatagaccaaatccacttttacaatacctttcgtgcgaacgaaagtagaagctagagcttcaatcatcccttgtaaatcattatgtgttaagctgatttgtaccttgtcagctttaatcagagtgccaagtctgtttgtatatgccatgctataacctcctaccatagtacgtaatcgtcttttaaatgaacgagtttcttcgataataaatgaccgattacatttgttccgcctacaaagaatctcactactttccattcatcattctcaacaacaatggagtagatataagcgtcttgtctatccaagatataatttgtttcatctcttgttaagatgtaattgttactatttaatcccttgaatgcttttctcggaattaaatctgcttcgtgcataaagtcctgtatattatccactgtcggtgcagatgatttcaccggtggttcaggcttcttgtattcaattatttccggtacaacttcgtacgcttttaaatatgatattggaaggttctcactatgtgcgacagacttcatggcttcatgtactgacctcgcctgtatcttctttagcaagatttccgtatcaaacggttcatttgcttctgttgtattacctttgtcatcgacatcgtaaatacgagcgtctgcatacttttcatccaccatgactagatatgtcttcacaatcatccctccttgttacttatagtgttaccaaatgtaatgctaattatgcagcaaactctacataaaattcattcatgtctaccttcgctgcacctacaagatacccaatcacaccattcgtattaatcgtgtcatcagatacaatccagtgaccgtgtatattttctttcacggcatagaatctcaatgtacgacctgcttctacagataacacctgtacatggtcgagtgtgtgaagatactgtgcattaccaggaacacggtcagtcttacgaagaagattcgcggctataatgaaatcttcaaatgacatgtcggtacgatcatcgttaccctcttccatcattgcgttgatagtgtttaacatcctatctttgacagcatcatctatacccatttcaccatcgaaatactcgtcctcataatcaaaatccatatcctcattaacttgcggattcatcttcatcaccttcatcgtctccacaaattactaaatggaagtattcttctgtaaccagtttatccattgttgtttcatgcgtacatgtgtaacattcaacgtctgtcggttgtagttcttctgttgggctgacattaattgtcttaccgcttccatcacatacaacaacggctgtcatatttttgtggtacttaaatattccaccgccacattccgggcatacttttgtcatacaaatctaacctcctctggttcttaccactggttatctatatgaactagggcattaccctagagaatctacgtaatagtgcgtgcgaacgcacaaaagatgtttaaaacatgtgtcttcctttacgttttgcttccttcatcgccttacgtcgtgcttcacgattttgatatagttcaaccgggtacttcattgattcgtttatcttttctataaactgttcatctaaactttgtgcaactcttggagcagcgataatttcaatttgctcctgtatatcctgcaatacgatatggttacttatcttcgctaattccgccattgcctcaggacttaatttaccactttgagaaccaacaacgatgtatttcttttctttttcatttatttctgatattccttcataaaacgcctctaatgcatcaggatccattcggccactcgcatcatcaacgatgattaattgctctctttccacggttctccctcgcttctttacggcgttgcgcacggttcttaaaattcatcgcatcaatttgacgttgctctgctaagaccatcgcttcttgccatccatgtaaatgtacatcactcggatcaaccgcaagcattttgcctcgtcctcctgttgcaccaaacatttcaacaacaacaccttttggtatgtttagataagaataggcagcggagtgtgccggattaaacccgacaacacccgtgcctatagaacgcctcatcacttcgccaccagatgcgccatatcttttgcatctgtcgctttgatttcgatttcttctgtgcggtcaaactcttcatccgttaatgccatgaactgaatcatagcaggtttgaattgtcgccacgcaccacgttcatgacaccatgcgacaacttgaccattcttccaattgtgaggttcaattaaacgttttgatataccgccttttgcatctttgtattcaatcgctaacttcttacgttgcgttgcactcattacaatataagcgataaacatcgacgggtcatctgtttgtaggtcggaagtatttgccacttttgtagactctcccggcttctcgtatgacttctcgatgttggtgttgatgaacgctttatacatgttcgccatcacaccgtctggatcgttttccttttctactttgtatgcttccaccataagtttctgaatacgactaagctgtctatattcaactccgttatctgccactaatctcatcaaacttcgcctcctgtaatgtatcttggaactccattaaataatacgatgcaatttgtgctggcaagaagccctcgtccgggcgttctgtcacatacttgctatatgattttagtaagaaacgtaactgcatttcattacagtccttccaatttctcggccagttttgttttggatgaacattcatatccattaggtactcatctaggtaaccatgacttaatgcttctttaattacatcttcatgatatgctagtgtatctttacccggcttcttgccgtagtttggtaaatcacgcacatcgaatgtctccatttcaggctcatctacaacctgatagttgtatttcatcttctcttctaattcatcatgcatataagaatgaaagatggtaatgcctccatagacggttaacacttctgctggtacacgtttagatatatacggcattattcaacactcatttccggatatagtttcgttgctgactcaaagatggcattattacgtggatcagaatcattcatgagtttatcagcagcatgtttcgcaatcgcaaccatctgctcttttgtatactcaccgttttggaatacgttctctacttccgataaaatgtccatgtagtcataacgtacatgtccatcgaacgggaaaacttctacactttgcacttctgcattaggcccccatgcaccttcgatacctgcttgcacgcctttacaaagactagaatccgaaatcatatctactttatctaatgtcattgccacaagaaatgtcttcttcattaccacttcatcccctcttttggcgtatattcttttatattatgcatcgccgtttttgccttctccatccctccgcttacacgttcatgaagatattcattatgttgattattgatatgacttgtaatggttgctatattttcgttcgccatactcacggcatcaatcaatccattaatcgctcgctccatatgcgcaagtgacatacgagtattccgtactcgttcaacttctatttcatcattgagtgcaacattcgcttcacgagctagttgcgaaagttcccactcacccattgcttgaatgactttattggatacgataggtcgatagttcttttgtatatctgtattttgaaaggctttctgcaactgatacaattcttcttctgttgcatttagcatcgtaataaataacgtaggtctgtccgataaacttacaggacttgtttgattaatttcttccactcttcgtcatcctccaataccttgactacttcaaagttcgttgcttccatgcgtgccttcatccagtttaatgttaagatgatattcttcattgcatcttctaaggattgcacttggttatctacagcttcgatacctaccctatcattaccacgaactgcttcattacgaagatttgttaacctatcgaaatgacgttgaaactggtcagatgaactcatctctagatttacatccacatacatatggtcgggtttatacacatcccaacgtattcctgttacccacggtatcattgttctaatactatgcgggttgccatacccgtttagtgtatacccataagtcacttgtgtgagttcatcaaatatcttccactgcgttgactcatccagtgccaatgcagatggtggtaatgcaaggtcatccttgattgttaactctaaatcaacacaatacgtttcactcttactagggtcgatttcgaagttataaaagcaatgcataaggtcttcctcttcgaatagactgcccgaacctgcatctacctgtaatggtcgtacatactgcatatctgtaaagtttacaattgcttcggggaatgcaatgagtaatgcgccattttgattattctcataaatgaatccccttgatccacactcgacaccaatattgttttcaccattgggccaaaagtcctgacttgcttctacccagttctttaagtgaatcatttagcacgccacccttcctcgctgtgctgtactgctgataattgcagttggttccttcgctaacaacttttggtctgcctcacctgttgttttagcgtatataaaatctgaatgtggataaccatccacgtaatacttaatgtaatactcaaacatgactactaacctccttttgtcgtacaaaaagagccgtgcgaacggctccctgattttacatgactaagcgttatccgattgctggataattatctcttacgaactctgaaatgaagtatggtgtttcttcttttgtatcgttagcaagatagaatcccatttcttcttcaccactatctggattatagatgtactgcacggttgtttgacagatgtcatatgccttttcatccgctacatctaagaactgatagaacagtgtctcatcgtccgctaattgaatctttaatgccaatgtatttgtaatgctagatgttgccgtgttatccccttgtagtacagccgccatacgaccactcctttgctatgttacttttagtattaccatttgaaatactcttatgcatgttgtggtaaataattctcatgctccgccatgacctgctgacggtacttgctatgtggcttaaaccaaacatcatagaccatctttctaagtctccggtcaccgaataaccgtgctggtggtactggtcgtttctcgtcgatatacattgttactaatgttctgtcacgattatccacgacaatgttccattcgtgataatatcgtacttcggtgtgttctgtatcgccgggtctaccgtagattgagaacaacagcatgtagttcgctcgttttgttaactctgcgctacctaccttctgaaatcttttacgtgcttgtttctttttcgtagcctctgccatacgttgacggaagcggtcttcaaagtgttcacttgcttcgtaatacgttgtttgatagtagtaccgtctttttaccatatctctcacctcctctatagtataaagcaaaagacacggcataagtcaccgtgtcttgctgtaagcgaggaataagttatcgaaccatcctcattataacatcgtgaaatactgtataacaggtacaatattttacaatatcatacgtttttatccatcaaccgaataaccgtatcctccgttctgttcgcgaccatactaggattcctcgaattattatgtatcagaaatgggtacacgataaaccaacccttgttacccattaggatgagccaatctaaaaagggcgaaaagcaaatgctcattcaatacacgatacgctcatatactcaatgagaatttgatatagtgcaacgggcaggattcgaacctgcgactttctgtatactccggtgacctctgtcatgagcagtagtgacccatctccgtatacagactgttttacccctaaactaccggaagcatggagaagagatactatgcagtgcatcccttcaacagtgtgtaaaatttacataacgatgaatcggttaaagtcatcgtgacattttagcgagcctatcggttaaatatggctcttgtggtgggctttgcccatgatataaaaaggagaggatatttcacctctccgctccgagtcatgtgatcctatacaaataactcataagactaaactaacaataatcgtagaacccaataccaataataacctatagatagagactaacactttgaaaccaattaatatttttagctaattagaccattgaaaattaatgagatacaaaaaacatagccaacatatcaaggcaggtgtgatttgctttggagatagaaatttaggcgggcattacgcccataggagggaacacacgcaggactccccagttctcgtgcattcgcctttaggtcatcatgtaaggtttcgaaccccattctctgacattgttaggtcagcgctttacctattaagctacgatgacatgaactccggttacccggagaacaatataaatcagtttttgtgtatgtaacggtaaccttattatataaggcttatgtcacaattgcaacacctttttgtaacatttatttattatgatgatactcataatatggtgtacgtgaaccagcaataaagaagttcaaatacggatcaggtgtaagttctgcatcctgcgtcgcacgtcgtactttcatactcgtaccacaatcatgacagtttacttctttgacaccttcaggtacgaaatgatttgcttctgttttacaacgattgtttggacacttgtagcgtactttatatcgcttcgtgccgtcactatctattttgaatcctgtcttcgatggatcgttctcttctacgacaggttcttccttcttattcatagcagccttcatgcgttcaccgattgttgtaccgaaaccatttgattcttcgtgtaccacttctaatgaatcatgtttcttgttttctgctatcggattaaacggctcatatgattctgctatttctctagcggctcttagtgcttgtggtggtatatgttgcacgggtaaatcaacatcaatatcaggcatcgtttgtcgcttactcacacgttgtgatggtgatacatattccacctgcatacccttctctacaatcgtagggtctgaatcacctaatagcttactgaaatccatgataggtcgttctgcttgtgcaaattggaatgcgtgcatcaatgctttctctactgcaaattgttttagttgatcgcttggattcggaatctcaagtttaatctccggctccgatttaatatgtagcttcattttctctactgtgtccattacggttcctcctcgacaacagaaaaggcactctaattgagtacctttaactgcacgtttcgtacaccccaagattcagatgcttgaaatgacggcattaataagtctacacgatttcctttaatagcgccaccggtatctagtgccatcgcctcaccgtatccttccacccatactttactaccgagcggaatgacactagggtcaacggcaatcacttttgctccgggattcgctgataggtcataccctgtcttcgttaatactcgtcctccatatgtaccaccattttcactcggatgcgctgtataagccgtagcctttacagtgagtgttcgcccgccgtctgatggtggtatatcaccggcaggttcagccttttttgcttgtaagttcttgacttccttacgagcgttgtctaagtcagcttgtgctttagacaattcattttggagactgtcaatacgttcttttgactgttgcagttctgtttctttattgttcaggctattgtcgagaatggttttctcttctgtcgcctttgcaacttcgttctccaatcctgcaatggtattattctgcgtagagactttcttctccataacagaaatgactttctccttttcgagaagcgccttgtcgtacttcttaaattcgccttctacttgaaatgcggaatagattacaaaacctattgtaagtattactgcgaataatgttgcgacttggttcctattcattcgaacagtcctttctgaaaataacaatagcccccagttatgccgggggcaaggttacattctagtataacatacattaaggctgtgcgtttggatccggttgagcatcgaatttagaatcatcttctactaagttaaagatttgcttcgcattcactgggtctaagtcagcaatagaggatagtgtaatgatttggtcactaaacttctctactgttgcacgagatgcgccaccgtctaagttgattaagacagttaacacattgaactgcaattgctcttttttcttcttaaattctttcaaccactcatctgtgataccgcaatcgccatcggtgataaatacaatgtcacctttcttatagcggtcactttctagacaacgaatagattcatctaataccggcatgaatgttgtaccaccgctactatctaactctgcaatgtccatgatgtcgtcaggatccaactcaccggctggtatgtttttaacatgacttgctacaatctgatgctgatacggaatgtatccgaagttacgcttctctttctgcgcgacttctaacatcgctaatgtaagtgcagttgaccagtcatccttgtttcctctcatcgaacccgatttatcatggtctatgataatggggccacgaccaactttctttataccatctttcttgtataactgtagttgtttctcggagaactctttcatgaattggttacgtgttgctggacttgctaacttcattagtgacgttggcgtaacacgagataaatcatttcctgtttcgattgtacgaacagagtgaccatctggtgtacgctgcgtcttcttctgcaaggcaatagctttcatgcgaccaactaggtctgttaaattattaaggcgtggactgcgacgaatacgttcaattgccttcttacgtcggtcaatcgagatacgcatcgtactagacgtaccatccaagccccatgattccattgtgtcacggacttcacgaacttcttcaaacgccttcttcgacgcttgttccattgcttgttccatgccttgcttcatttcttcctgacctgatttgtttaggtcaatttcatcttctagatcttgtatttgctgttgcaagcgtgctagttcctcggcttctctttgtgttaaacctttaccgccggggccacccttttcatttaattcatcgacacggttctgtgcatctcgtgccttgtttaattggtcaatcaactcaccagcgtcaccgccgtcttgcttttcaccagtttgttgctgttgtatatactgctgtttgaaatactggattttctccattgcctgattctgcataacttctgtaccaatggcactgttcatcacatcaaactttgtattcttacgaagctggtcatactgctctgactccatcagtccttccatcatttgatggtttaaccgagagaaggcatccatgtcctcttcgtcgtgcatacgtgctttgtagctgaacaaactttggtaaatatcttctgacaggttaccgaaaggagcatactcgtctaaaccactttgcatgttctcttgcataacctcgtactgatctaccgtgtcatgaaataactcacggtcaaattccgtcatcttgatactattcttatgcttctgtaacgggtcaatgtcctttttcaatcctaacgtttcttgtactaagttaccctcattacgttttctacgtccccagctcatttcgctacctctttcaagttcgtttctttttcaattgatattttaatgagttctttgtctttgttacgatactgaatgtatccttcatctgtatctgttaatgatgtagcttcgacatcagtctccgttttcagagcagctacgatgatgtttaagacatgttctgcattaaacccactgatatgtgtaaagaagaacttcttgtactttgtgactgttgaaattcgataaggatgcaattggttttcgtccattgtatagcctccctagttagaatcaggagaaggcgctaagttccataagtctcttagttgcttctccataaattcacttacatttaatttcttatccccactgccgagaagacttgcggttcgtttgacgtaagcatgtagtagagatttcatttacgatttcttctacttttactaagtacgcatctaacttatcaagttctgcgtcagatgcgccgttacgttctgcttctttgatttcatcagacatcttatctaacatttcggatagagaagaatttgtttgaacagcagttccggcacgctctgtgcggttctcaatttccattgtagacttcttcaattcttccgctttcttcacgaactctttcatcttcgattcatgcggattcatatacttgcctaactctttatcaagaatttctagctccttgcggtctttgttccaaagtacgttacgtagtgccatgaagtcactaccatttactgtgtcacgaccttctaatagagcattagccatcatgataagctgtgctttgtaataacgtcggtcagagataaccatgttattagtgcctaatgtacgtactaaacgctcgaagtctttcgcaatacgagcagggaaatcaaccatatgtaccgcttgttgcagtaagtcgatttcatctaacgtgattgttgttttcggtaagatacctttcttcttcgatgcacgacctgctgtaaccattttgatacggttctgtgggtcttggatatattcaatccagtgacggaacatgaatcggtcatagaacgcctctaatccttcgtcctccggatattcatttgacgcacccataacgatacgtaattcaactggtacagctttcccatcgttatagaagataccttcattcagcatagataacagatagttcagtgacggttcattcgatttgaaaatctcatcaaggaaaccgacatgtgcttctggcaacttgcctgttgtgatacgtaagaacttatcgttctccataccttttacggaatacgggccaactaaatccgctgggtcagttgtacggttcattaaccatttgaatgtgcgagccttttcaacgtgacttgagaataaatcagcaagcatcgacttacctgttcccggaggccctaagaatagaatgttttctcctgcaattaatgcacgaagaatatctttaattacatgctcacgttcgaatacgtttgcattcatttcatcgaagatgtcttctaacttcggaacaaccgctaatgcaagttgcagtggattcgatttctttttcacggatgatttcgttgtagatactttctttttcttacgaacaggttttacttcttcaacctcttcttcttcgtaatcttcatcctcgtcatcccaatcatcttcgtctaactcatcatctactacgttgtagtcctcttctagttcttcctcttgctctcttagtctacgtctacgctttcttgctactgcctctgccataataaaataacctccttagaatgtataaaaattaattaaattctttgtatacgtcgcccattgggtcatcgccgttatcatctgatttgccaagaccagactgacgctccctgcgacttcttttcttcggtttctcatcttcttcccagctatcattgtcattgatatgattgccacgacgaacgccacgagaatcctttgtaacatggtcttgtaagtccagtgtatccactaggtcatccggatcgttttctacttcttcctccggctctttcttatcccagttacgcttttgtttcttgttgccatatttgctcatagatgtcttctgtaaattgtctacaaagtcatctaagttacttgcttcaaagtcttgataagaatcttgcatgtcttgtttgacttggttcgcaacatcaggtgttcctgctacgacgattaacacaccagcgattgcttgatgtaattgtttcatcatatccatgaactcatctgttgtttttacctgcaacatttcttcaatcaattcatgaatgtgcatgtataacatttctaaatctgagaagtctgtacctgaatactttgtaagtgccttacattcatcgtacaaaccttttacaaatgcatctagttgctctaattgctttttacggacagccatgttatcgacattgccttgaccgttattcccttttgccactagaatctcctcctttaaataaatcagctaactcttgcacgagttcttcctccatacggatcaagccgaactcccaattgatacgtgtacgcttcaagataactttacgtagtaaatcaatatgtggcaaggtttcttcctttggcaaatcttgcatagccatttgtttctcggacttcatcttttgaatccattccgcccgcttatcacgaaacatctgttgtaacgaaacaatgcgtggcaattcagtctccacttcttctttcgttttatgaaacagaatgtcgtcgatacgaaatgttcgctccatccatgtagccatcggtttacccatacgtggaacctgtgctttcggtttccggtgtaatgtaaatccttcaatttcaacccatcttctattcataaaatcaggtacatgtggactatcaataaatatatagtcgcctacctttagttggtctgcgatgactgtcatgtcgcacgctccttccgttacaattagtatcaccaaatgtaatacgaatatacattttaccatacaaaaaatgccgtgcgaacggcattaatgtttaatcaacccactcttcattatctacttgattctttataggtaatgtaacagtttcaacgggttcttgtgctttttgtaatgctctgacgaacacattatggtctgcacttagttccatatgacgacgttctaaatcatcgtattcaacttgcagactatcatatctcttttgcagaagatcataatgctcttgaagcgtgatatgatttagtttaaatttctcctcttcctctgccttctgcttgagtacatgaatgattgcatcataagacgtatctccaagtgtaataggttccggagcgtcctttaacatatcatgtaattgcatggcacgagcatattcatgtcgtaattcaccggagaaatgagaggcaactgccgctgctgttctctgcggtaagtgtagttcggaaataatagcagatgcatcagtttggttatatccttgttgggtatactcatgcatatgcttcatcaaaattaaatcttcttcttctgaccacgggccacgtttcttttcaactgcttccatgtatagttcatctcctcttaccactcatcatcgtcacttacgactttctttttctttttacgtttcttcttcacaatagattggtggaacttcgttactgcttcactattttcaagtgttgcatcaaagctcgcctgtttatcatctagtcgttcacgcatttgttcatcgacaataccctgattaatcaggttgatgatatgcaccctatcgtgcgtggaatctagtcttcgaatacgatatttacgctgttcccatatagcaggattgtgaggtaaatcgtagttaatcaagtatcgtccggcttgcaggtttaaccctgtactacctgcaccagttgcgattaatactcggcatgaaggatcggtattgaatttcatcttacggcgttccctcacggagtctttcaatccaccaacgtagcggacggcacgagtaaacttcgatatttctctatgtagcataacagtcatgcgtttactctctacgaatataacgaccttatgaccattgtctacaatctcctgtacacgctggatacactcttgaaacttcggtatgtcataaatgttcttcacggcatacttcttgtttcgctcacgtatcttcttattcttagacatctggaataggttcggatgacaactaccttgaatacgggcatttaataccgccttcattctgtcaccgactgctttcgcttccactttacgtcctgctcggagcagatcactcttctcggatattaaatccttttggtactgcttgagttctttatccagtttcaattggtttggatgtggctgaatcggtacatcttcttctatcatcttcggtagtgataagccaatctcggcatccgtacgtctaatgaagaacttctgtatcttgcgtacaaattcatcgaggtgttgatacccgataatgtctttatactcattcagtgttgtgtaacgttctctgaacttacgccaactacctagtatcttttctgacaccatgctcacaagtccaaacatatcatccggatagttcataactggtgtaccggtaacaagtaatgcatacggaatacctttgtggtatcctttcttgttacgattggtacgtgctttgattaatcgttttgcggccttgttcgttttactatctttgttcttaatgttgtgcgcttcatcaaagataatcatgtcaaagcccgctttttctagcatcagagcattctgttgcatggtttgataactcatgaccatgaacatatcatcactcttcttaaacgcttcgatacattgtttcttcttatcatcaatggattcagctacatagcttttaccattcttcttgcggtaggtcgcacggaagtcagcaaacagtacagctttctcgtgactaaacttttctatctccttcatccattgcagacgagtcgatgcaagaacaacaactaacgttttattgacatgaccggattgcttcatttgaagtgccgtcatgattgcctgaaacgttttccctgttcccattaagtcaccgagcagtgcgatgttttgcttcttgagtgaataggataagaagtttgctccgaacaactgaaagtctttcggtggcatcttgaggtcatgtatctcaacctctttgattttccttaggattgcaggtggttttggcgggtcaatatcaagtagttcgtgtctcggtactttgtatattaactctccttcaaatagttcgtctaaatggtcagcagactcgatagggagtacccatacatcattgtcgccgtcccatctcgcaccatccacctgtcgcatcctcttattgtagtcttcgtgaaaaggtgtcttcacttctacaatgtcatcggttcttaggtctacgaatattcccggcataatacacctctataactgaaagacaattggtttacttgcgtaattctcattaatgcctttcaaaagatataagtctggatgtttatccacatactctctcttgccttcatacacataatccagcaagtgtgcgtgaaatgacgtatcgtacgggtcacgcttcgtaaacatgctactgaacgattctgaaagcattgctcgtcgtactttccacggttgcggcccgacaagtgtagctgttttcccaatgtaataaatatcatgtaggattaaacgactaccatatccaatatatgcaccgtatattgtattttgattttgataagcaacgctcatatcatccggatcttcctgctcaaataagatgaaattaccattgtcattaaacacataccatcctgttcctttatttgcattcacaaggaacataacaaaatatgtttgagaaccatattgtataccagcttcacctaacggctctagtatcatcgtgtctgaatcaatgagtctttgcacaacgtcatctgtgtaaggacgtgatacctgtattgtagtaggattaatactttgcgccatgtttaacatgaactgcacataactagtatcaagagagtcttttggtgatgaatgtgcctcgtatgatgtacctagtctaaagtatcccttcttcactttctcgtctgcacgtttctgcgctgctttcgttgcgtcttctttcgtaggaaattcacctatcgtagtagattgtaggtttgcaccgttcctgccatagaaaccagtgacaccataagttgtttccccactatgcgggttttctgcttttgaagcaatgacgatatataccttgtcacttgtacctttatggtatcgtaagtctgttcgttgtacgatagtcgcaccgcttggtaaaccatatctaaaatccttgtagcctgttagcaactacaaaacctccttaaaaacggcaagctcccgaaggaacctgcctacttgtaatgccctacttcgattagatcaagacgaagttcggataacctgtctttcaattcaaatactaatacatcatctttgtcttgcatagcttgatgtaattctaacagtgtgtagtccacttctgtcagtaatgctttcgtgctgagaggttgccttgttgtcatgttcaacacctcattacatatagtattacaatcagaaagacagattatacattacatctcgtgcgaacgagattgaagtaccgtatggttgacaggcaattcgccttcttcttgctgacgatataaactcttgttaattgcttcaaccataatagtagatagtacaaccatgtcctccggattggttaaggagcattcgtttaatgtctccattactgcatttactgtaccgttgatacgattacgtaatgtaatcttctcctcttctgtacgacacatactatctacatgattaaagaacgtagattgaataatgtctgtgaatcccattagtacagtgttgaagttgtgcttgtcaggattgattgaattaagtatgtcgttcatattttgttgtcgttgtttacgactaatgaacgagcgctctgtattctttgcgtccatatgtacacctctttctttttggctacctacattatatcagaacaagtagtgccataaagcttttagtgtctttttccaagtaccatcgttctgcaattcataaccacccatatacaccttcccgtttacctgtgaaacgttattacatggaatgtgtttgggaatcgtaaacgactgtccgttcatgaccacgaatctatctgctgtgtcgatgtgtattccatcttgtttataaatcattggcatgtatgtttcctcctttcggtcaacctcctaaaataataaaaagactgacatgacgatagggtactcaggaggtttagagtaggcatccctgtcgttgaagcaattggctgtcgcttcactgtcagtctcatgtttatagtgcagtcactaacattataaccacactatgatgaaagtatacaagataaatttaatcattgtcagtaatgacttgaataccttgttgtcttaattgacctagtaagtcattcactttaccgtcatacaaccctaagtattccggtgcaatgtcctctcgattctttactagaccgtcagttatgtgctgcacttgttctagatcaagccgtaacaagaccactttcggtgtcaacgtcactgtttccaattggtgtcactcctcccgctttcgctaacggacttgtcgcaaattgattatacatttcatcagactcttcaaagacgcctttgacagtttgtctattcacttgtaagtcctcgaacacgataataccttctgttttatcatcgctgaactctttcagtacgtccgtgttcttccagttgatttcttcacccttgaatgcgataagatgaactttaccttcatcctttaattgaccatagccatcttcattcacgattgtttcgccgttctcatccttttgcatatcaaagcgacgagcagctaagaagccatacgctgtgtttgggtcatcgaataacataggcggttgaccattggctgtatcacctacacgtttttgcaattcaattctcttcttgtctgatgcgaagaacgggaagtaccatatacgtttacgtcttttctttgccattatttattcgctcctttgaagtcttttgagaagtctactgctaatgcttcttttgcgtattcaggtacactgtctacagatgcttgtgcatccggaatgaatgttgcgtttgcaatgtaatcatctgcaagtggtacgtcggccgcatattcttcttgttgcattaactcaatgaataagtcacgagcgttgctaaagtttaatccatgttcaagaatcatttctcgtgtctttaacacaagtggattctccatgttaggattgattaagattgtgctaagtacactgctgtacgtaatcttccatagtcctgcacgaataccaactgcaactgcaatcattgttaattcaggattgaataataacgggaagttcgggttgtttacaccgcccatgaactcttctactgttttaccaattgctactttacctacggatggatcagcagtcgctaaccatgttgggtagaataacatactatatgagattaatagagcagcacttggaatctccggctttaatgcaacctgtgctgatacgaaatcaataacaactgcatcttctggattatctcccatgtctctgtcaatcattgcagaacgtaatgatgtataaatactttcataaaaactagccattgatataatgccccctatgtaatgttttcgcccctatattgtaacaggcgggataaggtcttgtacagcttcgaatgcatcttgaccgtcttggtaaccttgatcgtactgttttcgatagtcgtcatgataattctctgtatcgaacggcgtaccatcgaacccttgtgcaaatccagtacgataggcagatgaatactgtgcatctaaatctgcacaaatatgacgttcatcaatcgtaccgttcttccattctgaaatcgtagcttctttgttcttgaattgttcatttgcttcttccatacgttgttttgtatagactgcacctaaaccacacccgcatccatcattaaataaaaactcaccctgtttaggagtgagtcccgtaaatataaatgcttcacgaacttgttcaggagtaatacgcttcatcgccttttacctgcctttcgtaaatccttgatttttagttgcacatcaactttacctttgtactcatttctatccggaacgaatacaatgtcgatgttcttcggtttccctaatgcttcatacttctcatacaggaagaaaccaatcgtactaaacgttttctttccgtcggacataagcgcccacaagtggttcttctcatctcctaccggcatctcacgaatcacacgcatcttctccatcataaacgatggttgcgcatgacctctgccaaacggctccatcttattcatctcatcaacaaagtctaagtcgatgatgttacctttgacaatcgcatccacgtaaaccttaggctgtaagtcaatctcactaatcatcgtatccgcaaggaactccatacgtgtcttcaactcttggaatttatcatttgtaacagataaaccacaagccattgcgtgaccgccgaatccttccatgacatcggtctgtctcataagtgatgtgaataaatcaaatcccgtaatgctacgaccactgcccttgtatagtcccggttcatctgcatctgtcattataatagatggtttgtagaagtggttcattaagcgagatgcaatgagtcccacaatgccttcgggtataccaggataatagtgtacgatgatgttcgttggattatctatttggtctgcttcgacactcttgataatgttaccaacgtacgtcttcgtgagttccttacgttccttgttgatctccacgagtttctttgcgcaacgataggctgctaacttattcgtagagattaacatatccaccgcttcttctgctgatacaatacgaccagcagcattaatacacggtgcaattgtccatccaatgtcctgcgatgtaatctgttttaattcattcgcacggaacaattggtataggcctctgtttggtttgcgattcatcatatcaatacctagtttaccaatgacacgattctcatcaatgatgtccatcatgtcggcaaatgtgccaagagccacaatatccatgtattgatacgcaccggctctcccagcaggatgaatcttgtcgcaaacggcgcagagtattttaaatgatacgcccacacccgcaagtcctttaaatggatacttgcagtccggacgttttggattaacgacgatggtatccggaatgagatagtcgccaatgtgttgccctaccttctgtacgtcatctttatacgcacttgattcttgtggttcatggtggtcagtgatgataatatccatgcccttattctttgcgtatgctacttgttggattgctgtgatgccagtatcacaagtaataaccaaatcaactccattgtcagcaaatagatccagtcctctctgatttacaccatacccatctttgaatcggtcaggtacgaagtacataacatctaatcccagttcacgtaatatcagtacccatatggctgtagaacttgcaccatcggcgtcataatcaccgaagatacctatcgtttctttcttttgaacagcttcaataatacgagtaacagctttcttcatatccctcatcttataaggattgtgaagcttcttcttggtgatacgaaggaactcttctacgtcctctataccacgagcttccaatatgtcatagattaaatcatccttatgactgatacgtaagccacctgtttgccatactgcgtctgccattcgttcacctactttccttagaatcgttattctaagtataaacgataaaagctaagatatacacgtataccctagcttaccgccttcttctttttctttttaaccggtttatcgcctgttgctttcgccggtttagatgattttggtttctttctcacacgttttgccttttgttcttcggctagtttctcctcattacgaatcttatttacttcgatattcataaggcgtgtgatagactcatggtcatggttaaaatcatacagtgacgtaggtaatggtttatctacatcaccatcagcaagcgcaagtaatggtgctacggtatcctttgccttcttgcgtcgctcaagttcttgcttttgtaatatcgcaaggacacgaggttctgctaggctacgtaaccgcactaactcttgctcgccgattggtacagcttctatacgaggcttatgatctttaagggtaagtatctgtactgattcaaattccttcatcagttcgataagctcactcataaactcattgtccaccgtatctacatgctcatataaatgccatgcaattttactcatcgggtgttcgtcacccataaaccaatcagcaggaacaactatcgtcataactgcaacctccttataaaagcatcattacatacgtgaatatgcagaatgctaacattaaatatttcttcacgatgtcccctcctattttagagcaaacaaaaagttccgtgcgaacggaacctctgtcttagccttctgctggtggttgtggtgctgcgccagccggtgcttcttctactttcgtaagtaaacgatccatgttgattagttcgtttaatacgtggtagccaccgttagcttgtaagtttagtgagttgcggatttcttccttcaactgaccaatcttaccctttaccgcatcaaatgcttcttgttgctcttttgataagttactagccatatgtatcacctcaataataggatagaggacacgcctcgttaccaattatatcttatttggatacataaggatatattcttttacatccgcttccacgatagcatgaagttcttccttcaacttctcaccatcttgtgagtataaatcatgtgcttgttgatagatatactcgtacggcatcatatcttctaacaagcgttgcgctgtatgaacggcaaagtcacccggaatcacattgtgtctataatccagcatctttgctcggtacaggtcatccgcatagataaggttatgcaatgtttcaaatccttcgtctgcaaacttcattaagatagtagctgtgcgatgaatgaacaccatttccttaccgtcatatgaaccatcgccacgtttctcatagtcctttacgaatcgcttcacacgatgctgtatgctatgatactgtgacttgtagaacttaaacagattcatgcgtacgatcttatctcgcatggtcagtatcttctttagaccaccagtatgctgtgcgaatggactaacgtccgaatacgatgcatagagcaactgcaagatgtcgagactttgtttgtatagtaattcaggcaagtcacggaaatcatatgtttttaaatcaatcatattttccggtgcgcataagtcttttaagtaacgcttaccttcatataaatctttgaacgttggatatacgatttgaatcacatcaaaatcagattctccacttactgctaggttgtattcatgactacctgttcccgcaacccatacggtatctctttctgcgatttgtaattggtcttggattcccatcctatttacctcctttattgaatactgttttatgtatagcgtttactgctgatttgattacatctgcacggttaacggaactcacaccgtggtctacataataatccattagctctcccatttgtttgcatgtttcatttggaacagctaccaactgtgtatcctttgtttctaaatttaaagcatatgcaatttgtcgtgctgctgattcatttacacaacgtgctactagaatttgttttgctgtatcttgtactgaccagctagttccttgaattgctacatatcttgccatgtcatttctctcccattcactattttattatgtacagccaatgattgttcattcgagtgtacgaattgttttgctgatagtgacatattctctgcccatgcacggtattctttattgatactgtgatgcacacgatccatatgcacaaataggatataatcaatgcagaagtatgtcggtatatttaatacctcacatatgcgtagtcctatatccattgcaacggcgaactgtcccttctctactttacgtagatagtcacgtttcgctcgtgtctttagcttaatcgcaagcatctcatatgagtaaccctttgcctctcgtgcttccttcatcgccaccgtaaacggatgtatgcgttccggttgcacgggcatagccttacgaccaacaatcttcttatataatgtcgtcaatgtgtatctcccctttgacataggtctttctaatgaacagagaaggaaaagtgaaagtaaagaagttgagtatagtattgtgtgtgaaaaatacccttctctgctcattacaaagggcgtgaccggagccacgcctatgtgctttgattaatgtacgatcttttcttgtgtaactgttgcttcgtctacaggcgtgccttcttcttcgccatcttcgtcatcatgaccaagtggcttaccgcaagagcatttaccttcacgagcgttttcttgtgctgcttctaattgctctaacatttcgattacattagggtcagtcgtcatcagttcttcgattttcgctctcgcttctactttcttagcttccactgctgcatcaagttctgcttccgtgataagacctttctcaaccaataggtcgattaacactgtaccgtgaccaatttcgcttacgatttcttcttctaattgctcacgtacttggtcttttactggacggtcaagagtaagacgtgggcctccgaatggtgataagatagctcctagtaattcttttaacatgattgtttcctcctagtatgatagttagtttagtttttacaaaacagttcgtgcgaacgaactagtcctagctaagtgtatatacttcgcctgttactgggtctgctgtaatgtcccatgcataagggatgttgtacttcttgcagatattcatccagcaacgattcttttcttgcactgctttttcaatctgtgcgaatggtacattatcagcaagctcaatcgcttggatatgttcccatgtacgaatgttttctgcttgttcttccggcgttaatacagcaagcactgtacccttcttcggtttctccttgaatacattcttcgcttttggtgttacttctttatttttctttttcatgacttagttccccgctttcaattattgtttcgtaacaaatgccttgatgcgttgtaacttttcttcacgactatgaccatcatgctctaccatttctaaattagacatgaagtaagcaatttgaccttgtggtaatacgatataagcgataatgtcgccagcccaatcaggctttttacgagacacgacttcatgtactttaccagctacaatgtctcctactttgcggaaccctgattgaaatccgcaatgctgcgccgtagctacacactgtgtaaccaaacggtcacgagttttgaaatcaccaattgcttcaaaggactcaatgcctagtaacatatctcgtaattcattttgtgcgttcatttggtcaagtgtatatcttggttttggcacacttgtagttttaccttctgtcaatttcaacaactcctgccttaaagaaattaccatgcttatcttggttcgggaatccaatacttgttgcatctcgaacctccatcatgtctccgcacttatgacacttcttctctttccattccggaaggatatacgcattgtctctgtgcttacatgttggacagttgtatcgtaagcgatacgtttctttccattcaccgtcgaacttcttatacttgatgccagtctctagatagtgttcgttctccggatgatcttccgcctcttctaagtgttcagggaagaatcgttccaaagctccttctaattctggtggatatggtgcttgaactcgtagtgcttttgctagtgctgtcattgctacgtatgactcatctcttgttaattgttcgccagtgaaccttacctcattgtgtttatttactacactaaatgttatgcttgtcgccatcagttggttctaccgacggttgttggttttcgtatgctttaaagaaatggtgcatacgatgctgtattaaatgagatgaaagtgctgtggcgatagggatatataagtcatgcgctaaaactttaacatcaggatgctggtcaacaaatgtaccaatcgtgttgttgattgtttctgctaatttaatacggaagttcgtatgtgattcaggattatgattctgtagttcgtcctccggaatcatttcatccatgtcacgagcgaataacgtaaagacagttgataatgcaagcaatggtacagtaatgtccattttctcgccctgatctttaccgaacttctccatcgcatcagcaatgacaacctgcatttctcttgttaatcgctctgcttcctgtaacttctcctgcgtcagttctgacagacctaatgcctgtgtgaagaactcaattggatttgttggaacgccagcttcttgctgtggtgtctctgccttaggcttaaatgagacaatgttgtctttttctgtagacataacttttctcccctttgaaaacataataaaggagggaacacataacgtatcccctcttcaaccggttgtgtatatcgtaggctagttaggtatatggtttactctgccctccgcagtccaaagcacgttccgcaaatcaactttgcatcaaagctcacagcttataggtcacacatgtaacttaactgtgtaggtatccgttttataattaaggaacattctgcaatgctaactccccaaccacatgtgacctataagccggacgaagaggtatgaattatgcaacaaataagtactcactcttatcttgttaattccttcgtcggcttatcgtaagttgttttccgcacggttcctcctattggatccagcgtacacaaggaaagaatctcctaccatctacttgtatgtattctcgaaacttcgttgcaaaaggcaagaatgccattagcagaggatacgagagactcactgttgtacgttaccgatatactttactttatggagtaatctactccaaagacaagagggaaaccatttgacttttaaccactttatatttaagccctcttatctttgcaatagactaatagaatacaataacgccacgacaagtgaccaaaaagacaacaacatattatcaaattttagtgtagaacatttcatcaatatgggctgtatcacttgccgtgtgtattgcattgtcgtagttttggagatagttgttatgagacaaactatcatggaatggtagtctagggggactacgcatttggagttattcactccatgtaaggtgtgtataggctattgaatacttgactattgggagatagtaaattttccacaccctacatggaaggaacaagtcctccattgtaagtatcaaactattttatgtaatagaaaaggtgaagcagtgaattgtccaagttacactgctggaatgggtcgcccaaaacccaaatcaacatacaaggtggaaaaatattagggttggaggaccccggcatcagccttaaaaaccaccttgtatgctcatttgagtagaggatgtaaatcctctctctacaacatagttcgtgcgaacgaacaaagcctattcttgtctaattttttcaataacctcgtcacgacgacgggcataaaattcagataagatactaagcacatgtgctgcacttaacagttcagccgttggagtcggcttcatgtcactacgattctcatccagtaaccgaatgagtttgtcaccaatgacaagaccttcgtccattgatttcttagcgtgtgcttcatcaggtgataaacctttcagtgaccattcatatgccacctgtcctaatgatgctattaataggaagagatccatacgctcatatttatactcttctttggtaaatgtctcaattgccttcttaattgcggttgagacttcttggatgttttctttcgtaaattgttgctcatatgtattttcgtttacagccatgtcttgtaccactccctcgtcttgtccctttttaaaaaaaaagagaacgatctcggcatagcggataaccgctacactgagtatacgttctctgtaagggactgtcaacactttgagggatatttgtaacattaagcgaagaagtcagcgtccttctgttttgaagatgctacttctttcttcgcttggttctgctgtttacgtccagcagattgttgctttttcttttgttgctgagtagagcgtccaccacgttgcgtttgctttttaggtttttcctctacctcttcaggttcttccacttcctcttggtcttcctcttcgaagttatcgtcatcctcaacattatcatccgcttcatcttcacctacgaactcgatgtcatcatcttcgtcgatgtcgtcgaaatcttcatcgtcagtatcaacactacgtccagtgttgcgtccctctgatgatggtacaaatgacgccgtttggattacgaactcatacgtattatatggtacaccgctaatcgttttgattttcgttgcaccattctttttgattttaatatcaacatccatttgcttctctgatttacgtacataaccagtaagcacaacaggatgaccagttttatcatctttaccatcggcactaaaatgctcgtctaagaaaccaactagaccgcctttgttaccgaagtctctaaagcaaactgcgtccatgaactcgtattcaccagtttcaggattacgataaccgatacgtccacggaataaagtgcggtcgtcattatcgttctttttgtcagtccactcataagctttaccattatataatttacctactacagttgtgttcatcattttaacatctctccaattcaacattttattttaatagataatacaaaacagctcgtgcgaacgagccacacaatacttagattcgttgtgttacaattctacacattgaatacgcctgtatcgtaaagacattataaatattgcgaagctggtcgtatgctttctcgtcgtcgctaaactcatcaatgagtatcgctaattcttcgagcattaagaaagcctttaaatcatccagtgcggtaactcctaaacgaattaactgatggactaaacgaaaatcattcaagcgtcctttcatgcgtgtgataatcttcgttcccaacacttcgaactcccactgaggcataatgaatgtttcagcttcctcgttctttacgtgctgtccaaataatacactgggtacaacctgttcgtttttcagtgctaatgatgacataataatctcctactttcatatagttttagttttacaaaagaagccgtgcgaacggctcctgctttactgacgacgagctttacctagcgtagccaaatcaataacagcttcacacaagaatgcggcggtatccacaacaccgtgagcgacatcaccaatcgtacctaacgtgttatcaagtattccttcacgtttcggtttctcggcgtccttaccaccatccatcgcaacagattgaatcttacctgttgccatgccctcgatgacacgctcaagaccttcgatgcgtcctgcgtgattctcaaggatttcctgatgttcggataacttctctttcactttgttgatacgtttgtttgtgtatgcattcgctttttgaacggatgttgatacacatttctgcataattatgtcgagttgttgctcggctgtcaattctgacacaccctcattttcctcgatttcttcgccttcaatctcttcgaagtcttcatcgtcttgaacctcgacagttactctttcttccgccatttccaacacttcccttctttttcgcctaatatcatccaataatgtgacaactttgtgaacgtttaggtcaaaaagtaacaaaatgacacttttccggataattatatatatagtgaggagggttctacaggggtcagcgactcgaatcgctcacaaaaagaaaaacctccccaaaccacatggtatagtggtcgtgttcaccttgcataaaacatgcaagacaaaaaccatcactacacactacaatgaagtaggatgtgagtttgataacttgatacactcgaactgaaactcgcatagcactaccgcacaacgaatgactcaagtgattcaagtaatttaagatggggctatatctaaggggagaacatcgcttcgcatacgctacgctgagtcctcccctttagaatcccctctccaatcatccataggataggtttgttggagaatgtatactgcgttcatttgaagacagtacaatacacgtcgtgcgaacgacgattaactatgtgactccggtgattccatttcagattccgattcatagtgcagttcaggattgatgttattatcaacaatggatgtaatcttatcaccgtcgttatctgcacctgcatagcttttcgatgcacgttcctctgcatcacgttgaaccatatcactataggcttccgcttctatatcacgttgagcgtcattctcccacattggttcctcgatagccttacgtaagtcacgcacgattgtaggaagtgcttgtgcgactttaagagaatccataatgtttaagccttcgatagaatactctagtttaccaatttcaatagttgcctcttgcgttgggacgtttactttaataccttctacgttaaatttaatgttcattatgatttcctcctaaaatggtttagagccacgtcgaatgtgaccacgtttttgtttttaaaaaataaagagcaggatctccgctccggattaataaccaaatgcaatgcgtgcgtcacgaatgaactcacgtctgacagcaggagatgcgttttcccacgctggtaactctctgcccgataacataactctcgctcgataattaatatacagggtacgagcgtaacccttaatttgatgtttaacgtctacgatttctaatgctgatactgtcatgattgacactctccatttcatatgtaattttcaaagaacaataatacataacaagccgtgcgaacggctgattaaaccactgctactgcatagccaccacgaattaaatcgacagcgacacgcataggtacatgaactttatcacctactgtgaattttgcaccataacctaatgaatcaataattttaagacataacatgcgtgtaccctcctataataaaatacataaaagcccgtgcgaacgggcattacgaggcgaataatttctctcctcttctaaccttacgcacgagctttctatttggttcaagcacaagcataggtagtttcttacgctgtgcatattttaaacagtttgttgttccactagaaccattccatacaccaatgattgccgtagcattatcaaccatatattcattacgtgcttgcatacaacgcatggtaaagggttgattacttaccatcgttacaacatcagctttggatttaatccacttataatgcttgcgtgcttctggtcgccatttgttaccgtgaccttcatgcgggacggcaatctctagtgtgatgtcgggatataacttacgtgcttcaattacaatttcagcagcccacgtatcgacgcccaacgcaccacctgtgatgaagtgtgcattcggatatttcttacagtatgctagtatcttcttcgtaagccatatatacaccatcttgttgtcgggattatctttatcgaatccgaacaagttctgcgggcgtgggcctgtaaatgttaatacttttctaccttgttggcgtggtgttaatgtcatgattatcgtccccttatcatattaatagcgattgcaaatactataagtcctatccaacccggaggggtacagaatattaatgttaatgctgttgcgattgcttccatgattacaatacaccatcctttagtttccacgtataaataccatgaacatctacagcttcatcacccatcaaacgacgagtttgttctgtatcatcatagaaatcatctttcttaaactgtccagcttcacttaaccatttccatgcatatgtttcattaataggcataatagcttcattcgcttcgatacatatctgtcttgcgattacctctctacttggattatgcattaagaactcttcaattgtcatagccactatctcccttgttatgtaataaaagaacagtccatgtcgtgcgaacgacacgaaccgtcctatcttatactattaagcttgtttgcgtaaaccttttgcttctacatataattcatagtgagcaaattgtaatactaatgcacggaatgcaggattcatgttctcacggcatacatcgaatacacctttagcaacagctttcacaccaacagatgctttacgtccgtcgcctagaacaacagttacaactgcacactcatgaccaacagctaatttaccagtttgttcaacagatacaccagcaacatccattgcttctgtatcaaattctgctgtccagtagtcagcacttaaatccatagagtcaagaacatcgttgttgaagtttaatgtgaatacttctgcttcttcttggtactcttctgtttccatgtcgtcatccatttcgataactgctttttcttcttccatgtatccagtgtaagcaggttcttctacatagtatggttgctcgttaacagatgttgtaacaacttcttcttggcgatccgcaacttctgttacttcctctttcttagctttttgtttcttcactagtacacggtcgaatgtcatttctaagaagaatgcttgcacttccttctctttacctgcacgcttcgccatgtaagttggttctaatttatctaagtttgttactgttaattcacatgcaccagtttccgctacgtattgagcaacgtttgctttttggtcacgagcaacagcaccgatacgttgtttaccagtatatacgttcacacgcatttcacggtcaactttcttttgaacaagacgaatcttaccttcgttcttttgtagtaatgcaagaacatcatgtgaagataaatcttcgaacttccatcctttaatttcgaatgtagcgaactgttcgattggacgctcaacttgacgcttctctgcaacagacttcgtacccatgatgatgaacacttgaccattacgtttgaatgttttgtaagtaccgtttggtaatttagactcaacacggtagtcttcaccttctgatacattacggtagaactttaattcagaaggtacatcatcaatatgaccatcgaactgaactttacgaagttttgtttttgtgtctgtagatttagacacgtttaataataaaccatcatagcaagttacccacgggaatgagatagacttaccttttgatgtagatgagaaatatgtgtgttgataagcagcaccagcgatgtcacggatgtcaccgtcgatagattgtactgctcttgtgtacttatccatgatatgagaaatatgtgcttgttcctcttcatcagttaactccatgtcacgcatgttcattaactctttacggtaagcaccttctaagtcagagatgataggacgaatacgatttacttctgcatcgtcaacatcagctaagaactcaagtgtgaagtcaacagttgtttcttttgtttcttgcttgaacttcgtttccatatactctttaacagcgttgaataattgacctaactgagacttagatacgaaaacatttttctctttgttcactttcttcattacgttgaactgtttccagtgtggttgctccatgatttctacatattcaggaacttcagggaagaatccagttttagcgaagtcaattgaccaaccttgcatgaaacgtaagatacaaacaatctcatcaatacgagcaggtgttaaagactcaccattgattttacgctctttcatgattttaggattgttcgcaatatctttccatgcagtagacatgtttgtaatttcaccaatagataatacatcttcgttgatgattgttgctaagtcatacttgaagatttgtgcttctgagttcgggctttcaccagctttgaaaccagcatcatataacattggtaaaccagtttcaactgcatcacaaatctcgtcgttctcaaccattgctactttatcaccatctgtatccgctccacccatacgtggtaatgtatcatcgaacgggttcatgattaagatgtcttggttaaatacatggtaagcaggtgcatcaacagtgtttagtaccactgcttctgatttatggattaatggagaacggaaagcagcgaagcgtgactctactccattgtagtaatattcaccagctttaagtacagcttttctgccgaactgtaattcagggcatgtaacgatataagggttcgcacctttcactgggatacgtcctttcgccatgtcacccacatacttctcacgagttttacggattgtttcttgtacgaaacgctcaccaatcatgtcaggattctccgttaataactcgataactttttgaatcttaccacctgcggcattcatcttcttatcgtactcaccagcatcacgtccagtgtcatacatacctaagaatgcttttgcatcatcagcgttagagaagatagtttcttttaactcttttaatgcacgtcctgcgaatgtcttaggagaaatcttgtcattgcgaagtgcttgccaatattggtaacccatgaacactgtatcgtttaagcggttcttctgccataatacaatctcaagttttacacgtttctctgatggacactcttcactcacgtagtaaggattgaagttttctttccacattgaatcagtgaacatgatgtctttcgtgaagtcatacttgtgtgtcttctcaccgaagaactccatgcatttaccagtgttacgagtcttacgtccgttgcagtcttttgtctcaaggttatgtgggtatacaagtaataaacctttagtgaaaccgaaacgaatttggaatgcagatggtactttactccatactttaaggaactcttcgttaccttctacttctgcaatacgtacatcttggttatagatttctaaccagtctaataacatttcacgttcgttacgagagattaactcaagacgatgtgctgtacgaactgcggcactcggtaacattgtaccttgaccatctaatggctcgtaagtatcaacatgttgtttgtatgtgcttagtttacgagtcttccagtcatatactttgatgtcatgtttacgttcgatagcgtagtcaggcataatgtcgaatgagaagtcttcgttcgtgatctcaatcgttgatgactctgctaaaccgtaacgtgcttccatgtttgcaatcttcacttcttccgggaactctgcaccataagatacaccatcacgtacttctttcactggtaagctagtcattagacgtttacctacacgagtctgtgatgctgatgttaatacacgacgatatactacacgagtgccgtctaacttaacgatagttaaacctttctttagaatacgacgaatagcaagtttcgcactctctttatcttctttagataaaccaccatatgagaagttcactacaatgaactcttctgcaagacgtacagttacttcttcgttcagttcgttgccgttctcatcaacaatagtcttaacacgtacttctgtcttatgagagaacatacggaatgcacggttttctgatacgaatgttttcactgatttgaatccacgttgaccatagtttgcttcatagaatacatagtcacgtccctctgcttgtacatctgcgatattgataattcctacttcaaattgattacgtttcttagccatgataaattccaccttttcattttttatagttttatagtttgattcatactctgtcgtgtcttgggcggacacaacaatcatcacttacacgcccaaccgtgcgaacggtgggcataacgtgcgtgatactaactttcggatttcattacgctgacattatgccaacttcaatcgttctcttgtgcgtttgtcgtccatctctatagcgtaacgaacaatcgctggtcgatatttgatatataatgcatatatctccttatcgctcttatactcataccagtggtcggcataaggaagtataaccatcaacttctctatcatgccttgcacttctctgctgcgagcaagtcgctgaccgtaatacataacactcacctcctttcaatttcgcgggcgacataacgtacccgatactaagaatatctatataaacctcctatacatacataagacatataagagaggaaagacataacgtgtgcgatactgtgaatacataacacatgcgatactagaatgacataacgtacgtgatacgagagacataacgtgtgcgatactgactctatatataagagacacatatgagagtgtcttgaatgacttaggtaacaatttagatacacttaggatatataacaacctaacacttgaatgacttgtgtgtgacaacacacaagagaaatacctcccaaccatgttcagcgtatgctacgactgtgcttatccacgtttggtacgacgaaccataaatggtacaacgtcccagtttccttgagtgacttgtggagcagggagagaaagggacgaaaagaaaactatttcgcccatatacgttcgtcgattgcccgatgatgcgtgcgaacgcaatcatataaagcatcaagcgtgcgaatgcgtttcgaatgaaagcaatgcgacgcaaaatgaagatgccatatatacagggacgacgaaccataactggtagagcgtaccagttgtacaagaacgacgctgggtggtgaacggctcgctctgctcgccgtcgtaggtcactgcgtttccccacaacacccttcctgcgaatacgccggaggcggggtatcacacaagcagaacctctattgactgcgaacacggtctgaatgtacgaccgtactacgctccacatcttaaccatgcatctcttatttcataaccggttggctgacgttcattgttgctaacgaacaactcacgaaattcgaccaaccacgatgcatctactgttccgctgtatcttgtcactagagtattttccttacgctccgcaggactcacattctcctgcttcggtttattatgcgtctgtactcgcttacgctcctcttgttttactcgtacggatgctggcatcttacgtactcggactaacgcttttactatgcgtgcaatgtcaccatgcactccattactgaatggcttaaatgaatcaatcgctgactgtatcatcattggtttcataggtttcccatccacctttatatatggtcgtgcatcagatggattccatccaatcttaactgttactcccttttgtttgaagacaacgcccagctcgttgctacgcacatactcaaatactttcgtgtacgctggtgctgtcttttcttccggctcgctgtcgctcgccgaatcatatcctagttcctctgctacatagaagtcattcagacactctatgcaaatgtgtccatcatcctctgtgtaacataggtgacgagactcctcattgcagagttcacattgctcgctgacgctcgcaagttttacagtcatcactttataactacgaccaagtttctttgttttccaatccatccataatacatatctctcttcattcactactgttaaattcatcattttaaattcctccaatttatatgttttttgtttaaagccggggactcattagtggtagcctgttcagggcatctcagacttcaccaatcgtgtccatgaagttgctaggtcaactgacactgactgaacaactccgtagagattgttttcacatgtgcgtgcgttatgtcttctagtcatgacgtactctccctcgaccaagagttagagtcggcgtcctagcttcttgcacttccctcgaaagggtggctatccactaggggcgtcctccattcttgttagtacggagagcagcaatgacatacatgatgtcacgcaatgaggactcgtgttgtcttaacccgaagcaatctgtgtcctcactgcttcgggatgttagctggttgctaagtttcctagtaccatgtttgtccgctaccctatcgcgctatcactgattacggagtgaccatccgggatactgaataaatgctacatatatctcacgacatactgcattgcctgattcctcaacagatctcttgtttataccgacgttgattcagacgtcgaggcatacttgtgaaacggtggatgcttaccgtgcctgataggtcgctactcgtctactgttatgagtaacaacgatgttaggtctgatgattctacgagggcaaccaaccctcctctctggtgtagttatagttgacaccataagagacacgatatgctcatgtctcctagctaccaactatattactgacgacctaacaacatagcttctaactgtgtatttaacgcttcttcatctcctgtctcatggaatagtgccatcgcatcacacaatggtgttgatactaacttctctgctgtcattcttaatacgccgttgtcatcgaagtctacatcagctacttgttcatgattgttgtacggccataacactaagtcttttgatacgaagccactgttcatcatgttctcttttgtatatacggtgtatcctctcttacgtaatgtatcagcaacgatttccacactgcctttatactcaacacgacctgtatataactcttcaatgaataaggaatgatcttcaccaatgcggattaagaatctaccaccccatctgttttcaccttcatcgaaatagccaacgattgtcgcaccggatacattacccaacacaccgatgttgttgtcattcaccttacctagttcccacttcatacatgacgtccactccttatctgttgatacacgtagtagatcatccttcataccactgaacttcacatgacctgtcttcttcgtattacgtgtttctacaatgtgttttactttgcctaatggaatacgaatgtcatcacccaatatttccatcacttgcggaaactctttttcaattctgttgatatacttcacgacgctgatctttctgccatcatctagttgtaccttgttctccatgaaatcatcctcatctaatcgttgtaatcgtaatagtctcatttggaatattactgtgtgtttgtgtaatggattctcataatcgacacgcaatgcacttgatacatctcgcaatgctgaatggaacatctcatgtgagtgattgattaactcaatgtcgaatgaatctacaagcaatggatgtgctgctacttgtttgtgattcgctatcttcatcatgtgattgattaatactcttggattgttatctaatcgtgctaagtaatacatatcaaccatcatcacattaccgatgtcgtacttacctgtgaatgtcatctctctatctacgtatgcatacttcttagttacatagttttgtagaacaccttgttcatatagatactcacaatagtctctgcttaacttgtattcatgtccgagtttgaatccatacttcttcgcacataatgatgctctgatataacctgttggttcgaacttgagtggacgaatgatacgttgtttgaatccaatctctgctggttctgtgataacaaccttgccgtcgtagtaacatgcagtgatgttaatgtcgaagtcctcttgtacatactgacgaatatcatctgtgttcaccatcatgatctctacacgatacttacctatcgtaccttttaatactgatacaattttactgatctcaatgacacgtactgattcatcatcatcttctactgtgatacttggacgttctctgattacctcgctgaacataaagcccttgcttaatacgtgatataatacctctgacttacatgcgtcggatgtagatacgaacacatcaatgtcattaggttctttacccatcaatgcatctaatacacatccacctgcgataatacctttgaaaccttttaccactttgtatacttctttaatacctgtacttgctgtcatgtttattctctccttttaatttaaataaggagccgagatatacccgactccgatgattgttattatttatttaatacttctgctactttcagtttccatacacctcttgttggcttaccttgtttacctaagtctctcatgtatgcttgatacccactgttgaacttactacctaccttcttgaacttgttcgctgcatatacttctgcaccaatctctttatgtgcttcgatgataagattagagattctttcatccattgctgattcatatactttaacttccttcacttcctcttcttcttcctgtggagtgataacaggctcttcttgttgtggcacgatacttacttgtccttgcatcataccttccacgataccacgcatgataccttgtactgcatctgctaattcctcttgtacaacttctcttactgttgctctgatctcctctttaaacactggcattacatcctctctaactgttttgatgattgtattctgtacgttgtttgcccacatttgtgttagtgtcccaatactatcacgagcgataacagcgaatatatcctctgttgatactggcttcttcttaatagttgatacgatttcttttgctacttccatttcagttttctgttttaacttacctgcacgaatgatttcttcatcacgcttgtttgcaccataacgtttattccatctgtctaaccctgattgtgtattcttcatagtcattagattgtatcctcctgtgtagatgctgtttcagccatcatcattatttgttcaaacataatctcgaatcttacttcacttcctccattaccttgtgcattcagtcgaataccatgatgtgcatacatttgtttaataatctttgcttcatttgtgctttttaatcttacgtttgttttgattcttgttacagtcaatctgtcgataccaagagataacatgtacgatacgcaatcttttaaataagccttagcttcttgttgttggtctaggattagcttttgtgctacgttgtgtgataataccccttcaatcattgagattgtttcatttgacaacttcattattccttatcccctttactaattagttcacttgttgcacgtaatgttcctgatgctaaatccactcctgcatgacctacttctccgcctagcttgataactgttctaccggcacgttctgttacattcaatacttctcttgttaagccgagtgcagattctaatacactgtttaataattgtgttcctctgtctacacctgcatgaccgtacttatctactacagcagctcctttcaatagcccttgtgcggcacttaatgttaaacctttgtacttcgttctgattaattcctgtacatcacctcccttcaatgcatgtaatttctcttccactgaacggtactctaatactagttcgttgtacttttgaggcttagtgatattcttgttcatctcttgttgaatatgacctagttgtttatctagcccatcaatgaccttttgcttatccccttctgttaataccaattcctgtactgcttcttctactacctgtatgctatcctctgctactgcctgtgctgttgcatctgttacttgttgcatcttctctgctgccatagccaatacttcttgttctttcttagctagtgcttgtttcgttaactctctatgctgtgctgttagttcttcctgcattgctttaacctcttcctctgtgtataccttaccttctgttgttgctgctacttgttctttagccatgttcattacctccatttgttgttgtattgcagtgaattgattgatgatctctttcagtatctctgtacgcatacccatgttgacgttagcacctaagttgttagcctgttgtactagtgataatcgttgactatgcattactgttactcgttgatatagcttgatgatgttctctttctttgttctatcaccttgatactctactaattgtttttctgctactaccttgatgtttaagcccatatccttacatagcgcccctgtaatacggttaatatcctttactgtcataccttcgattgtttttaagtctgtcataataattagctccttttataattagttgtttttgtttttattgattaactcacctagtgttggtgctagtttaataccttgtttctttttgactatgctagttgataccttaccgatacctgctttctccactgtgttaatgaatgtaactactgctctcttcttcatacctaactcctcctatgcaaataagtttttaatcttacctaatagactactgaatacaccctgcttaccttctcctggtggtaattcattgttgtcctttagtctcttgatgtagatgatacgtttctctaactcaggtactagatgatggatacctagtctgataccctctgctctcatgatgttaagcattagtaatacactctcctttccacactgttgtactgcatactcatcacacttaaactcttgctctgtcacctcgtgctgttctgttagatgtccagtaggatacatgatatgacctagttcatgagatactagtgcttgtcttacatactggttactgttgaatgatttatagtcatatacaataatcatgtaatctaagtagtatcctgttagttcagcgatttgttcattgtcatacactgtcatcattacagttaagatacgagtgtcgatgtggtgcttctcgatgtcctcaatggatagttgatgatgacttgctaagtcttgtggttgttcgatgatagcaacatgttctgtcttgccgttgatgtttacagtgaatacttctttcaccgattgtcttacttccattacttctgcttgcgttagtttgtctgtcatttgtcattcctccattagtttaggtgtagtcaaaaggggtggggcatgaaacaaccctatacccccattgctatatataagacg

(19) United States
(12) Patent Application Publication
(10) Pub. No.: US 2012/0020994 A1
(43) Pub. Date: Jan. 26, 2012

(54) MULTIVALENT, DRYING RESISTANT, EVOLUTION-BASED VACCINES

(76) Inventors: Philip Serwer, San Antonio, TX (US); Gurneet Kohli, Sayre, PA (US)

(21) Appl. No.: 13/131,394

(22) PCT Filed: Nov. 20, 2009
(86) PCT No.: PCT/US09/65362

§ 371 Date: Oct. 5, 2011

Related U.S. Application Data
(60) Provisional application No. 61/118,190, filed on Nov. 26, 2008.

(51) Int. Cl. A61K 39/00; A61P 37/04; A61P 31/04; A61P 25/00; A61P 31/10; A61P 33/00; A61P 25/28; C12N 7/01; A61P 31/12

(52) U.S. Cl. 424/199.1; 435/235.1

(57) ABSTRACT

Disclosed is a recombinant nonpermutated bacteriophage that includes a nucleic acid sequence that is at least 150 kb in length wherein the bacteriophage is made to display one or more surface antigens such as heterologous polypeptides, and compositions and kits that include the recombinant nonpermutated bacteriophages of the present invention. Also disclosed are methods of inducing an immune response in a subject that involve administration of a pharmaceutically effective amount of a composition comprising the recombinant nonpermutated bacteriophages of the present invention.

This application claims the benefit of the filing date of U.S. Provisional Patent Application Ser. No. 61/118,190, filed Nov. 26, 2008, the entire contents of which is hereby incorporated by reference.

### BACKGROUND OF THE INVENTION

1. Field of the Invention

The present invention relates generally to the fields of bacteriophage, bacteriophage therapy, vaccines, and induction of an immune response in a subject. More particularly, the invention concerns recombinant nonpermutated bacteriophages that include a nucleic acid sequence that is at least 150 kb in length wherein the bacteriophages display one or more surface antigens, such as heterologous polypeptides, and methods employing these bacteriophages in the treatment and prevention of disease.

2. Description of Related Art

Filamentous phage-based display systems (as described, for example, in Smith, 1985) have found widespread use in molecular biology, including many immunologic applications such as antigen presentation and the immuno-isolation of desired recombinants by “biopanning” (Marks et al., 1992; Smith et al., 1993; Williamson et al., 1993). However, with filamentous phages, peptides that may be displayed from the major coat protein are limited in size to 6-10 amino acid residues (Kishchenko et al., 1994; Iannolo et al., 1995), although somewhat longer peptides can be displayed by co-assembly with the wild-type coat protein (Perhan et al., 1995). Full-length polypeptides can be displayed on minor phage proteins, but only at very low copy number (Parmley and Smith, 1988). Moreover, the requirement that the fusion protein should pass through the secretion system of Escherichia coli may pose problems of toxicity for the host, or for correct folding of the displayed protein (Skerra and Pluckthun, 1991).

The DNA sequence and genomic annotation of a recently identified long genome bacteriophage named Bacillus thuringiensis phage 0305φ8-36 has recently been reported (Serwer et al., 2007b; Thomas et al., 2007). Studies to examine the comparative genomics of this phage have suggested descent in a novel ancient phage lineage (Hardies et al., 2007). Phage 0305φ8-36 was isolated from soil while targeting the isolation of large, unusual phages of unsampled or undersampled types (Serwer et al., 2004; Serwer et al., 2007a, Serwer et al., 2007b). Examination of phage 0305φ8-36 by electron microscopy revealed an unusually long contractile tail, and three large corkscrew shaped fibers emanating from the upper aspect of the baseplate (Serwer et al., 2007a). The genes of 0305φ8-36 have only distant homologues and the gene for the large terminase subunit was reported to be anciently derived (Serwer et al., 2007a). Among the functionally annoted gene products (Thomas et al., 2007a; Thomas et al., 2007b) are a putative RNA polymerase, DNA polymerase III and associated replicative and metabolic enzymes, two DNA primases, and virion proteins. A thorough survey by mass spectrometry identified 55 virion protein-encoding genes, and noted that this was an excess over the prototypical myovirus, T4, and particularly so if tabulated in terms of the total length and hence complexity of virion protein sequence (Hardies et al., 2007).

Bacteriophage 0305φ8-36 is a lytic, double-stranded DNA bacteriophage and does not have a lysogenic state. That is to say, 0305φ8-3 empirically does not co-exist and co-grow with the host and does not have the genes that normally must be present to do so. Bacteriophage 0305φ8-36 is also a myovirus, which means that it has a contractile tail that is used to inject its genome into a host cell at the beginning of an infection. Before the discovery of 0305φ8-36, two classes of lytic myoviruses were recognized, the T4 class (Desplats and Krisch, 2003; Sullivan et al., 2005) and the φKZ class (Krylov et al., 2007; Thomas et al., 2008). Bacteriophage 0305φ8-36 is the founding member of a new class (genus, perhaps). Bacteriophage 0305φ8-36 is the only bacteriophage in any of these three classes that has a unique-ended (non-permuted) genome. It is the only bacteriophage of any type that has a non-permuted genome that is longer than 121 Kb. Bacteriophage T5 (genome length=121 Kb; Wang et al., 2005) is the next longest bacteriophage (lytic) with a nonpermuted genome, as far as is known. Most double-stranded DNA bacteriophage genomes have a sequence at one end that is a repeat of the sequence at the other end (terminal repeat).

The closest homologues of most of the virion protein encoding genes and a few replicative genes were found to reside in a single segment of the chromosome of Bacillus thuringiensis serovar israelensis. A smaller segment also appears in the chromosome of a closely related species, B. weihenstephanensis. These two phage-like regions are termed BtI1 and BwK1, respectively (Thomas et al., 2007a). Hardies et al., 2007 describes a detailed study of the genomic organization and vertical descent of phage 0305φ8-36 in comparison with BtI1/BwK1.

Among the long-genome phages with Gram-positive hosts, phage 0305φ8-36, infective for Bacillus thuringiensis, has several unusual characteristics. These include plaque formation only in ultra-dilute gels and aggregation, as visualized by fluorescence microscopy. This phage has a 221-kb genome, as assessed by pulse-field gel analysis (Serwer et al., 2007a). The tail of 0305φ8-36 is remarkably long, 486 nm in length, making it more than three times the length of the tail of T4 (Kostyuchenko et al., 2005). However, the most notable feature of the 0305φ8-36 tail is the presence of three “curly” fibers (approximately 187 nm long and 10 nm in diameter) that are joined to the contractile tail near the baseplate (Serwer et al., 2007b). The dimensions of 0305φ8-36 are almost identical to those of the B. cereus phage Bace-11, a classified myovirus (Ackermann et al., 1995; Fauquet et al., 2005). Aside from the curly fibers, there are other notable shared morphological features of 0305φ8-36 and Bace-11, including baseplates that appear to be elaborate. Hence, the structure and function of 0305φ8-36 and Bace-11 curly fibers are likely to be homologous (Thomas et al., 2007a; Thomas et al., 2007b).

Despite the current level of understanding of bacteriophages, there is the need for improved bacteriophage-based vaccines and methods to treat diseases, such as infectious diseases.

### SUMMARY OF THE INVENTION

The present invention provides for the display of antigens on recently identified bacteriophages, where the bacteriophage are exceptionally large (i.e., have a nucleic acid sequence that is at least 150 kb in length). Therefore, these bacteriophage will have a comparatively large amount of DNA that can be deleted to make room for DNA needed for encoding displayed protein. Fifty-five proteins have been found present in the phage particle. Any of these proteins might be an improved antigen display vehicle. Specifically, sheath fibers that should be ideal for display of antigens are thought to be present, based on informatic analysis of the gene sequences. Display in sheath fibers would facilitate multivalency and increased surface exposure. Also, Bacillus Thuringiensis phage 0305φ8-36 was isolated from soil that reached temperatures of 60° C. Thus, as a vaccine, this phage and others like it would be expected to have improved elevated temperature resistance. Sheath fibers of 0305φ8-36 are believed to be present because the genome has several open reading frames whose products were found by mass spectrometry to be part of the bacteriophage particle and were found by informatics to be fibrous in character (Thomas et al., 2007; Hardies et al., 2007). If they exist, sheath fibers are anticipated to be optimal for antigen display because fibers project away from the bacteriophage particle and because fibers, in general, are under comparatively low steric constraint and, therefore, are likely to have low stringency for what and how much is displayed.

Bacteriophage sheath fibers involved are known to exist on other bacteriophages (Eiserling, 1967; Belyaeva and Azizbekyan, 1968). But, no indication exists that they were to be used for protein display.

Certain embodiments of the present invention generally concern recombinant nonpermutated bacteriophages that include a nucleic acid sequence that is at least 150 kb in length wherein the bacteriophage displays on its surface one or more antigens. The antigens can be displayed on the surface of the bacteriophage using any method known to those of ordinary skill in the art. Examples of such methods for display are discussed in the specification below. In particular embodiments, the bacteriophage displays on its surface two or more antigens. The antigens can be identical or distinct. In specific embodiments, the recombinant nonpermutated bacteriophage is Bacillus Thuringiensis phage 0305φ8-36. Information regarding Bacillus Thuringiensis phage 0305φ8-36 can be found in U.S. Ser. No. 12/188,941, herein specifically incorporated by reference in its entirety. Bacteriophage 0305φ8-36 was isolated from soil at the King Ranch, Kingsville, Tex. The procedure of isolation is described in Serwer et al. (2004). The complete genomic sequence of bacteriophage 0305φ8-36 is found in Gen-Bank:EF583821 (SEQ ID NO:1).

A “nonpermuted” genome is a genome that has unique ends and a terminal repeat. Other bacteriophage genomes have a single sequence with terminal repeat that, however, has ends that vary in position because the genome was cut from end-to-end “concatemer” of mature genomes; genomes with variable ends are called permuted. The cutting to form a permuted genome is not at a unique place; the degree of randomness of the cutting varies among the bacteriophages. The cutting always includes more than one genome's quantity of DNA, thereby also generating a terminal repeat for permuted genomes. The length of DNA cut to form a permuted genome is determined by the volume of the container into which this genome will be packaged. The container is a protein shell, sometimes called the head of the bacteriophage. The length of the genome is one “headful”, so to speak. If one removes a gene from a permuted genome, the mature genome length is still one headful and, therefore does not change; the terminal repeat gets longer (Streisinger et al., 1967). However, if one removes a gene from a non-permuted genome, the genomic DNA molecule does become shorter because the cleavage from a concatemer is at a unique nucleotide sequence. Nonetheless, the head does not change in volume. Thus, the packing density of a nonpermuted genome decreases when a gene is deleted. A consequence of the decreased packing density is that the DNA pressure on the head is decreased. Thus, a mutant with less DNA (deletion mutant) is more stable to elevated temperature than the original (wild-type) bacteriophage, in the case of a non-permuted genome.

To isolate deletion mutants, all one does is to raise the temperature in conditions such that the wild-type bacteriophages are killed and some deletion mutants remain alive. This has been done this with 0305φ8-36 and a deletion mutant has been isolated with a genome that is 6.585 Kb shorter than the wild-type genome. Even more DNA can presumably be deleted. The more DNA deleted, the more room the bacteriophage has for DNA cloned in the bacteriophage for the purposes described below. Because the 0305φ8-36 genome is about 4× longer than the genomes of bacteriophages usually used as cloning vectors, eventually much more DNA will probably be deleted from 0305φ8-36 than has ever been deleted from any other bacteriophage. The open reading frames and many other features of the 0305φ8-36 genome are described in Thomas et al. (2007) and Hardies et al. (2007).

The term “antigen” as used herein refers to a molecule that can initiate a humoral and/or cellular immune response in a recipient of the recombinant nonpermutated bacteriophage. Non-limiting antigens are discussed in the specification below. In particular embodiments, the antigen is a heterologous polypeptide. A “heterologous polypeptide” in the context of the present invention is a polypeptide that is not normally found on the surface of the bacteriophage.

In some embodiments, the nucleic acid sequence is between 150 kb and 500 kb in length. In more particular embodiments, the nucleic acid sequence is between 150 kb and 300 kb in length. In even more particular embodiments, the nucleic acid sequence is between 150 kb and 250 kb in length.

Non-limiting examples of heterologous polypeptides contemplated as antigens in the context of the present invention include a bacterial protein, a viral protein, a fungal protein, a mammalian polypeptide, a protozoal polypeptide, or a polypeptide derived from a prion. Other antigens include those antigens associated with biological warfare, such as a toxin.

Non-limiting examples of bacterial polypeptides include polypeptides derived from pertussis toxin, filamentous hemagglutinin, pertactin, FIM2, FIM3, diptheria toxin, diptheria toxoid, tetanus toxin, tetanus toxoid, an M protein, heat shock protein 65 (HSP65), antigen 85A, and pneumolysin.

Non-limiting examples of viral polypeptides include polypeptides derived from picornavirus, coronavirus, togavirus, flavirvirus, rhabdovirus, paramyxovirus, orthomyxovirus, bunyavirus, arenavirus, reovirus, retrovirus, papilomavirus, parvovirus, herpesvirus, poxvirus, hepatitis A virus, hepatitis B virus, hepatitis C virus, spongiform virus, influenza, herpes simplex virus 1, herpes simplex virus 2, measles, dengue, smallpox, polio and HIV.

Non-limiting examples of polypeptides derived from parasites include a polypeptide derived from a trypanosome, a tapeworm, a roundworm, a helminth, or a malaria parasite. Non-limiting examples of polypeptides derived from fungi include a candida fungal polypeptide, a histoplasma fungal polypeptide, a cryptococcal fungal polypeptide, a coccidiodes fungal polypeptide, or a tinea fungal polypeptide. Non-limiting examples of mammalian polypeptides include polypeptides such as tumor markers and other markers of disease.

In particular embodiments, the recombinant nonpermutated bacteriophage includes a nucleic acid sequence that includes a region encoding the heterologous polypeptide. In further particular embodiments, the recombinant nonpermutated bacteriophage further comprises a deletion of its genome.

The present invention also generally concerns pharmaceutical compositions that include a recombinant nonpermutated bacteriophage of the present invention, including any of the aforementioned recombinant nonpermuated bacteriophages. The compositions can be dried and stored at room temperature, for subsequent reconstitution. The compositions include a pharmaceutically acceptable carrier. Any such carrier known to those of ordinary skill in the art is contemplated for inclusion in the compositions of the present invention.

Further aspects of the present invention concerns methods of inducing an immune response in a subject that involves administering to the subject a pharmaceutically effective amount of a composition that includes a recombinant nonpermutated bacteriophage of the present invention, including 0305φ8-36. Also included in the present invention are uses of the compositions of the present invention for inducing an immune response in a subject.

The immune response may be any type of immune response. For example, the immune response may be a cell-mediated immune response or a humoral immune response. In particular embodiments, the immune response is directed against a bacteria, a virus, a fungus, a tumor, a protozoan, or a prion.

In some embodiments, the composition that includes the bacteriophage further comprises a polymer. Information regarding polymers contemplated by the present invention can be found in U.S. Ser. No. 12/188,941, herein specifically incorporated by reference. Non-limiting examples of polymers include a polymer derived from agar, agarose, a dextran, a cyclodextran, a copolymer of poly-N-isopropylacrylamide, a methylcellulose, a chitosan, a collagen, a tri-block copolymer of poly(ethylene glycol)-poly(lactic-co-glycolic acid)-poly(ethylene glycol), a tri-block copolymer of poly(propylene glycol)-poly(ethylene glycol)-poly (propylene glycol), poly(N-isopropyl acrylamide, hyaluronic acid, alginate, carboxymethylcellulose, polyvinyl pyrrolidone, polyvinyl alcohol, a polyethylene glycol, a water-soluble polyacylamide, a substituted polyacrylamide, a polydimethylacrylamide, a polyvinyl pyrrolidone, gelatin, polyvinyl alcohol, polylysine, carageenan, and an analog thereof. In some embodiments, the concentration of polymer in the composition is about 0.001% to about 0.1%.

In particular embodiments, the subject is a mammal, but any subject is contemplated by the present invention, including birds and amphibians. Non-limiting examples of mammals include a mouse, a rat, a pig, a dog, a cat, a rabbit, a goat, a sheep, a horse, a cow, a primate, and a human. In specific embodiments, the mammal is a human.

The methods set forth herein may further be defined as a method of reducing the risk of development of a disease in a subject. Thus, for example, a vaccine comprising any of the recombinant nonpermutated bacteriophages of the present invention may be administered to a subject for the purpose of reducing the risk of development of a disease in the subject.

In further embodiments, the method is further defined as a method of treating a subject with a disease. The disease may be any disease for which vaccine therapy may be beneficial. For example, non-limiting examples of such diseases include a bacterial infection, a viral infection, a fungal infection, a protozoal infection, an autoimmune disease, a neurodegenerative disease, or a tumor. In specific embodiments, the subject is a cow and the disease to be treated or prevented is bovine mastitis.

Other aspects of the present invention concern kits that include a sealed container that includes a recombinant nonpermutated bacteriophage that includes a nucleic acid sequence that is at least 150 kb in length wherein the bacteriophage displays on its surface an antigen. The bacteriophage may be any of the aforementioned bacteriophages. In specific embodiments, the bacteriophage is Bacillus Thuringiensis page 0305φ8-36. The bacteriophage may be univalent or multivalent (i.e., displaying a single antigen or two or more distinct antigens).

The present invention also provides for methods for improving the potency of a vaccine. This may be done by immobilizing antigen-recognizing protein on a column and then using this column to selectively bind bacteriophage particles that had improved antigenic character. Such particles would preferentially adhere to the column and would be subsequently eluted and propagated to enrich for bacteriophages that encode for improved antigen.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

Further embodiments include methods of treating infectious disease of plants using a recombinant nonpermutated bacteriophage of the present invention. The recombinant bacteriphages set forth herein can be applied in the treatment of diseases of trees and vines. For example, one such disease is Pierce's disease of fruit trees and grape vines. Other bacterial infections of plants contemplated for treatment with the bacteriophage set forth herein include infections due to Erwinia, Xanthomonas and Pseudomonas. Examples of infectious diseases of plants include viral disease such as exocortis, xyloporosis, tresteza, psorosis, disease due to tobacco mosaic virus, and disease due to wheat yellow mosaic virus.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1. Electron microscopy of 0305φ8-36.

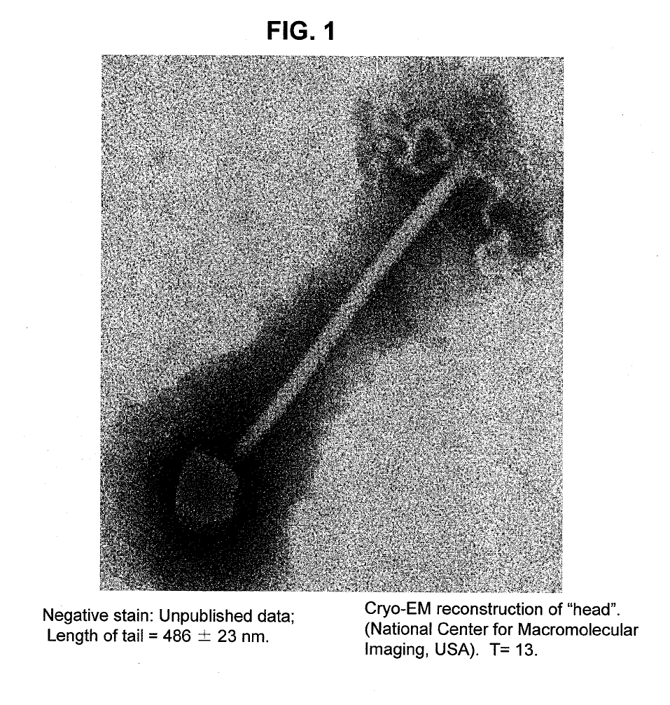

FIG. 2. Low Resolution Genome Map (218.948 Kb).

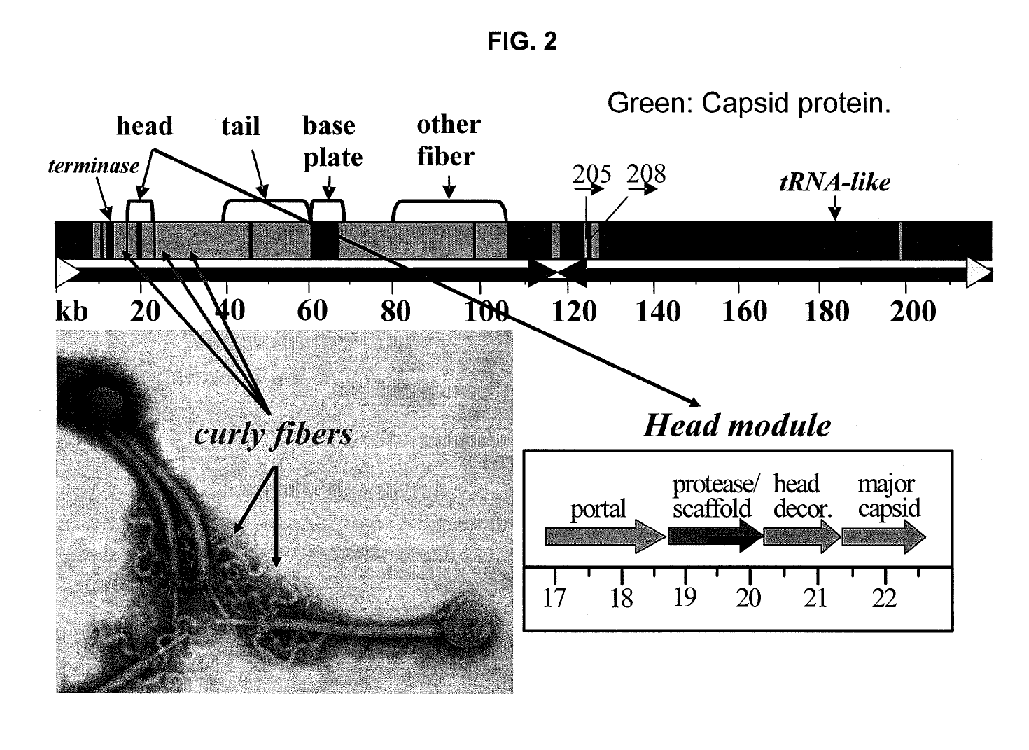

### DESCRIPTION OF ILLUSTRATIVE EMBODIMENTS

The present invention is based on the finding that certain nonpermutated bacteriophages that are exceptionally large (i.e., have long genomes on the order of at least 150 kb in length) are likely to be ideal candidates for the display of antigens, and that these bacteriophages will, therefore, be useful as vaccines in the treatment and prevention of disease. The bacteriophage can have comparatively large amounts of DNA removed to make room for DNA needed for encoding antigenic protein, thus allowing for multivalency. Therefore, they can be used in the treatment of disease and in methods of inducing a

### A. Definitions

The terms “protein,” “polypeptide,” or “peptide” as used herein refers to a biopolymer composed of amino acid or amino acid analog subunits, typically some or all of the 20 common L-amino acids found in biological proteins, linked by peptide intersubunit linkages, or other intersubunit linkages. The protein has a primary structure represented by its subunit sequence, and may have secondary helical or pleat structures, as well as overall three-dimensional structure. Although “protein” commonly refers to a relatively large polypeptide, e.g., containing 100 or more amino acids, and “peptide” to smaller polypeptides, the terms are used interchangeably herein. That is, the term protein may refer to a larger polypeptide, as well as to a smaller peptide, and vice versa.

The term “nucleic acid” and “nucleic acid sequence” includes RNA, DNA and cDNA molecules. It will be understood that, as a result of the degeneracy of the genetic code, a multitude of nucleotide sequences encoding given peptides such as antibody fragments may be produced. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiou-racil, 5-carboxymethylaminomethyl-2-thiouracil, 5-carboxymethylaminomethyl-uracil, dihydrouracil, inosine, N6-isopentenyladenine, 1-methyladenine, 1methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “heterologous” denotes sequences (such as polypeptides or nucleic acid sequences) that are not normally associated with a particular host. Thus, a “heterologous” region of a nucleic acid construct is an identifiable segment of nucleic acid within or attached to another nucleic acid molecule that is not found in association with the other molecule in nature. A “heterologous polypeptide” on the surface of a bacteriophage is a polypeptide that is not normally found on the surface of the bacteriophage. Similarly, a host cell transformed with a construct which is not normally present in the host cell would be considered heterologous for purposes of this invention.

The term “isolated”, when used in relation to a nucleic acid or protein, refers to a molecules that are identified and separated from at least one contaminant with which typically associated in the natural source. Isolated nucleic acid or protein is present in a form or setting that is different from that in which it is found in nature. In contrast, non-isolated nucleic acids and proteins are in the state in which they exist in nature.

The term “purified” or “purify” refers to the removal of contaminants from a sample.

As used herein, “coding sequence” or a sequence which “encodes” a particular polypeptide, is a nucleic acid sequence which is transcribed (in the case of DNA) and translated (in the case of mRNA) into a polypeptide in vitro or in vivo, when placed under the control of appropriate regulatory sequences. The boundaries of the coding sequence are determined by a start codon at the 5′ (amino) terminus and a translation stop codon at the 3′ (carboxy) terminus. A coding sequence may include, but is not limited to, cDNA from prokaryotic or eukaryotic mRNA, genomic DNA sequences from prokaryotic or eukaryotic DNA, and synthetic DNA sequences. A transcription termination sequence will typically be located 3′ to the coding sequence.

The term “vaccine” refers to a formulation that contains a recombinant nonpermutated bacteriophage of the present invention that is capable of inducing an immune response in a subject. The vaccine will typically be in a form that is capable of being administered to a subject and induces a protective or therapeutic immune response sufficient to induce immunity to prevent and/or ameliorate an infection and/or to reduce at least one symptom of an infection and/or to enhance the efficacy of another therapy or prophylactic. Typically, a vaccine comprises a conventional saline or buffered aqueous solution medium in which the composition of the present invention is suspended or dissolved, although administration of dry powder, for example by inhalation, and even formulation with an additional adjuvant, such as alum, is also contemplated. The composition of the present invention can be used conveniently to prevent, ameliorate, or otherwise treat a disease such as an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

As used herein, “prophylactic” and “preventive” vaccines are vaccines that are designed and administered to prevent infection, disease, and/or any related sequela(e) caused by or associated with a pathogenic organism. “Prevent” and “prevention” of disease refers to reduction of the likelihood of development of an infection, disease, and/or any related sequela(e) caused by or associated with a pathogenic organism or blockage of onset of an infection, disease, and/or any related sequena(e). As used herein, “therapeutic” vaccines are vaccines that are designed and administered to patients already infected with a pathogenic organism.

### B. Antigens

The term “antigen” as used herein refers to a molecule that can initiate a humoral and/or cellular immune response in a recipient of the antigen. The antigen may be an agent that causes a disease for which a vaccination would be advantageous treatment. The antigen may be a heterologous polypeptide as discussed above.

Antigens include any type of biologic molecule, including, for example, simple intermediary metabolites, sugars, lipids and hormones as well as macromolecules such as complex carbohydrates, phospholipids, nucleic acids and proteins. Common categories of antigens include, but are not limited to, viral antigens, bacterial antigens, fungal antigens, protozoal and other parasitic antigens, tumor antigens, antigens involved in autoimmune disease, allergy and graft rejection, and other miscellaneous antigens.

In some embodiments, the antigen is capable of inducing the development of specific antibodies and/or a specific T-cell response in animals or humans. Alternatively said compound is capable of inducing the development of a cytotoxic T-cell response in animals or humans, or the compound is capable of inducing the development of an allergic response. Furthermore the antigen may be capable of reacting with pre-existing antibodies or T-cells, or is a compound capable of binding to the IgE antibody on mast cells or mediating a type I allergic response in a previously sensitised mammal. The antigen may be capable of inducing the development of immunity against one or more infectious agent(s) or allergen(s) in an animal or a human. Alternatively, the antigen is capable of inducing the development of immunity against autoimmune diseases in animals or humans. In a further embodiment the antigen is one that operates as cancer antigens in animals or humans.

Examples of antigens that may be delivered using the bacteriophage set forth herein include viral antigens, bacterial antigens, fungal antigens and parasitic antigens. Viruses include picornavirus, coronavirus, togavirus, flavirvirus, rhabdovirus, paramyxovirus, orthomyxovirus, bunyavirus, arenavirus, reovirus, retrovirus, papilomavirus, parvovirus, herpesvirus, poxvirus, hepadnavirus, and spongiform virus. Other viral targets include influenza, herpes simplex virus 1 and 2, measles, dengue, smallpox, polio or HIV. Other examples include: HIV envelope proteins and hepatitis B surface antigen.

Pathogens include trypanosomes, tapeworms, roundworms, helminthes, malaria. Tumor markers, such as fetal antigen or prostate specific antigen, may be targeted in this manner.

Non-limiting examples of bacterial antigens include pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components, Helicobacter pylori bacterial antigen components, pneumococcal bacterial antigens such as pneumolysin, pneumococcal capsular polysaccharides and other pneumococcal bacterial antigen components, haemophilus influenza bacterial antigens such as capsular polysaccharides and other haemophilus influenza bacterial antigen components, anthrax bacterial antigens such as anthrax protective antigen and other anthrax bacterial antigen components, rickettsiae bacterial antigens such as rompA and other rickettsiae bacterial antigen component.

Also included with the bacterial antigens described herein are any other bacterial, mycobacterial, mycoplasmal, rickettsial, or chlamydial antigens. Partial or whole pathogens may also be: haemophilus influenza; Plasmodium falciparum; neisseria meningitidis; streptococcus pneumoniae; neisseria gonorrhoeae; salmonella serotype typhi; shigella; vibrio cholerae; Dengue Fever; Encephalitides; Japanese Encephalitis; lyme disease; Yersinia pestis; west nile virus; yellow fever; tularemia; hepatitis (viral; bacterial); RSV (respiratory syncytial virus); HPIV 1 and HPIV 3; adenovirus; and small pox.

Fungal antigens contemplated by the present invention include, but are not limited to, candida fungal antigen components, histoplasma fungal antigens such as heat shock protein 60 (HSP60) and other histoplasma fungal antigen components, cryptococcal fungal antigens such as capsular polysaccharides and other cryptococcal fungal antigen components, coccidiodes fungal antigens such as spherule antigens and other coccidiodes fungal antigen components, and tinea fungal antigens such as trichophytin and other coccidiodes fungal antigen components.

Examples of protozoal and other parasitic antigens include, but are not limited to, plasmodium falciparum antigens such as merozoite surface antigens, sporozoite surface antigens, circumsporozoite antigens, gametocyte/gamete surface antigens, blood-stage antigen pf 155/RESA and other plasmodial antigen components, toxoplasma antigens such as SAG-1, p30 and other toxoplasmal antigen components, schistosomae antigens such as glutathione-S-transferase, paramyosin, and other schistosomal antigen components, leishmania major and other leishmaniae antigens such as gp63, and its associated protein and other leishmanial antigen components, and trypanosoma cruzi antigens such as the 75-77 kDa antigen, the 56 kDa antigen and other trypanosomal antigen components.

Other examples of antigens that may be delivered include tumor proteins, such as mutated oncogenes, viral proteins associated with tumors, and tumor mucins and glycolipids. The antigens may be viral proteins associated with tumors would be those from the classes of viruses noted above. Certain antigens may be characteristic of tumors (one subset being proteins not usually expressed by a tumor precursor cell), or may be a protein which is normally expressed in a tumor precursor cell, but having a mutation characteristic of a tumor. Other antigens include mutant variant(s) of the normal protein having an altered activity or subcellular distribution, mutations of genes giving rise to tumor antigens.

Non-limiting examples of tumor antigens include: CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), Pmel 17(gp100), GnT-V intron V sequence (N-acetylglucoaminyltransferase V intron V sequence), Prostate Ca psm, PRAME (melanoma antigen), .beta.-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67.

In addition, the immunogenic molecule can be an autoantigen involved in the initiation and/or propagation of an autoimmune disease, the pathology of which is largely due to the activity of antibodies specific for a molecule expressed by the relevant target organ, tissue, or cells, such as SLE or MG. In such diseases, it can be desirable to direct an ongoing antibody-mediated immune response to the relevant autoantigen towards a cellular immune response. Autoantigens of interest include, without limitation: (a) with respect to SLE, the Smith protein, RNP ribonucleoprotein, and the SS-A and SS-B proteins; and (b) with respect to MG, the acetylcholine receptor. Examples of other miscellaneous antigens involved in one or more types of autoimmune response include endogenous hormones such as luteinizing hormone, follicular stimulating hormone, testosterone, growth hormone, prolactin, and other hormones.

Antigens involved in autoimmune diseases, allergy, and graft rejection can be used in the compositions and methods of the invention. For example, an antigen involved in any one or more of the following autoimmune diseases or disorders can be used in the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis.

Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease.

### C. Methods for Displaying a Polypeptide on the Surface of a Bacteriophage

Bacteriophage may be genetically engineered to express heterologous proteins. Smith first demonstrated in 1985 that filamentous phage tolerate foreign protein fragments inserted in their gene III protein (pIII), and could show that the protein fragments are presented on the phage surface (Smith, 1985). Ladner extended that concept to the screening of repertoires of (poly)peptides and/or proteins displayed on the surface, of phage (WO 88/06630; WO 90/02809) and, since then, phage display has experienced a dramatic progress and resulted in substantial achievements.

Various formats have been developed to construct and screen (polypeptide/protein phage-display libraries, and a large number of review articles and monographs cover and summarise these developments.

Most often, filamentous phage-based systems have been used. Initially proposed as display of single-chain Fv (scFv) fragments (WO 88/06630; see additionally WO 92/01047), the method has rapidly been expanded to the display of bovine pancreatic trypsin inhibitor (BPTI) (WO 90/02809), peptide libraries (WO 91/19818), human growth hormone (WO 92/09690), and of various other proteins including the display of multimeric proteins such as Fab fragments (WO 91/17271; WO 92/01047).

To anchor the peptide or protein to the filamentous bacteriophage surface, mostly genetic fusions to phage coat proteins are employed. Preferred are fusions to gene III protein (Parmley & Smith, 1988) or fragments thereof (Bass et al., 1990), and gene VIII protein (Greenwood et al., 1991). In one case, gene VI has been used (Jespers et al., 1995), and recently, a combination of gene VII and gene IX has been used for the display of Fv fragments (Gao et al., 1999).

Furthermore, phage display has also been achieved on phage lambda. In that case, gene V protein, gene J protein, and gene D protein have been used.

Besides using genetic fusions, foreign peptides or proteins have been attached to phage surfaces via association domains. In WO 91/17271, herein incorporated by reference, it was suggested to use a tag displayed on phage and a tag binding ligand fused to the peptide/protein to be displayed to achieve a non-covalent display.

When screening phage display libraries in biopanning, one issue is how best to recover phage which have bound to the desired target. Normally, this is achieved by elution with appropriate buffers, either by using a pH- or salt gradient, or by specific elution using soluble target. However, the most interesting binders which bind with high affinity to the target might be lost by that approach. Several alternative methods have been devised which try to overcome that problem, either by providing a cleavage signal between the (poly)peptide/protein being displayed and its fusion partner, or between the target of interest and its carrier which anchors the target to a solid surface.

Furthermore, all the approaches referred to hereinabove require to use fusion proteins comprising at least part of a phage coat protein and a foreign polypeptide. Transformation efficiency is a crucial factor for the production of very large libraries. Additionally, for the characterization of polypeptides obtained after selection from a phage display library, the polypeptides may be recloned into expression vectors in order to remove the phage coat protein fusion partner, or in order to create new fusion proteins such as by fusion to enzymes for detection or to multimerization domains.

The term “phage display” refers to a set of techniques for the display and selection of polypeptides on the surface of particles produced from a replicable genetic package (e.g., a bacteriophage). As first described by Smith in 1985 for the display oiEcoR1 endonuclease, phage display methods comprise expressing a polypeptide of interest as a fusion protein attached to a bacteriophage coat protein. Progeny bacteriophage are extruded from host bacteria (e.g., E. coli), and “panning” techniques that involve binding of the polypeptide of interest to a cognate binding partner are used to enrich those bacteriophage displaying the polypeptide of interest relative to other bacteriophage in the population. Smith initially reported that selection methods could be used to enrich phage displaying an EcoR1 endonuclease-pIH fusion over 1000-fold. This display-and-select methodology has been extended and advanced, so that today large libraries (>107 to as many as >1010) individual polypeptide variants may be rapidly and conveniently screened for a particular binding property of interest. See, e.g., WO 91/19818; WO 91/18989; WO 92/01047; WO 92/06204; WO 92/18619; Han et al, 1995; Donovan et al, 1987.

Phage display often employs E. coli filamentous phage such as M13, fd, fl, and engineered variants thereof (e.g., fd-tet, which has a 2775-bp BgLTL fragment of transposon TnIO inserted into the BanïHï site of wild-type phage fd; because of its TnIO insert, fd-tet confers tetracycline resistance on the host and can be propagated like a plasmid independently of phage function) as the displaying replicable genetic package. Non-filamentous phage (e.g., lambda), spores, eukaryotic viruses (e.g., Moloney murine leukemia virus, baculovirus), and phagemids offer important alternative genetic packages for use in phage display. Likewise, bacteria such as E. coli, S. typhimurium, B. subtilis, P. aeruginosa, V. cholerae, K. pneumonia, N. gonorrhoeae, N. meningitides, etc., offer alternatives to the use of bacteriophage for display of polypeptides.

Considering M13 as an exemplary filamentous phage, the phage virion consists of a stretched-out loop of single-stranded DNA (ssDNA) sheathed in a tube composed of several thousand copies of the major coat protein pVIH (product of gene VIII). Four minor coat proteins are found at the tips of the virion, each present in about 4-5 copies/virion: pin (product of gene EI), pIV (product of gene IV), pVII (product of gene VII), and pIX (product of gene IX). Of these, pill and pVIH (either full length or partial length) represent the most typical fusion protein partners for polypeptides of interest. A wide range of polypeptides, including random combinatorial amino acid libraries, randomly fragmented chromosomal DNA, cDNA pools, antibody binding domains, receptor ligands, etc., may be expressed as fusion proteins e.g., with pIH or pVIH, for selection in phage display methods. In addition, methods for the display of multichain proteins (where one of the chains is expressed as a fusion protein) are also well known in the art.

Ward et al. (1996), reported the introduction of a proteolytic cleavage site into a phage display vector encoding a pIII/sFv fusion protein for use in enzymatically eluting bacteriophage bound to a solid substrate during the selection (panning) phase. Cleavage at the protease site introduced between the pill and sFv sequences was reported to not alter infectivity of the bacteriophage as compared to treatment of the same bacteriophage with either 100 mM glycine, pH 3.0 or a pH 8.0 buffer.

Kristensen and Winter (1998) reported the introduction of a proteolytic cleavage site into a phage display vector encoding a pill/enzyme fusion protein for use in identifying proteolytically stable enzyme sequences.

A variety of resources are available that describe the many protocols, reagents and variant phage genomes (and variant phage genes) that find use in phage-display technology. See, e.g., Smith and Petrenko (1997); Sidhu (2001); Rodi and Makowski (1999); and Willats (2002).

### D. Bacteriophage-Based Vaccines and Methods for Production

Bacteriophage-based vaccines are of two basic types, (1) DNA vaccines introduce the gene for an antigen and rely on the recipient to manufacture the antigen from the DNA introduced. In this case, the gene is attached to an expression-promoting sequence that mimics one of the recipient's expression-promoting sequences. One way to introduce antigen-inducing DNA is to clone the DNA in a packaged bacteriophage genome and introduce the bacteriophage particles. Protein (and other non-DNA) vaccines introduce the antigen directly. This can be done by inserting either part or all of the gene for the antigenic protein in one of the bacteriophage genes that encodes a component of the mature bacteriophage that projects outward from the bacteriophage (protein display). The altered, protein-displaying bacteriophage is called a display vector. In either case, the first requirement is to delete DNA from the wild-type bacteriophage, as already done with 0305φ8-36. Deletion of DNA makes room for the DNA to be spliced into the genome to make either a DNA or other vaccine.

In the case of bacteriophage-based protein vaccines, the antigen is displayed on the surface of the bacteriophage particle as part of a bacteriophage protein. Bacteriophage 0305φ8-36 particles have 55 proteins, over twice the number in any of the bacteriophages even considered as possible protein display vectors. Thus, one has a comparatively large range of choices for which protein to use for display in the case of 0305φ8-36.

Cloning the appropriate DNA in 0305φ8-36 cannot currently be done by conventional procedures because these procedures, including plasmid cloning, followed by introduction to the host and then bacteriophage genomes, have not yet been developed for 0305φ8-36. The following is an alternative option (described in Khan et al., 1997): Introduce chemically and PCR constructed DNA fragments by in vitro recombination, followed by in vitro packaging of the recombinant DNA into bacteriophage particles.

### E. Kits

Any of the bacteriophage and compositions described herein may be comprised in a kit. The kits will thus comprise, in suitable container means, a bacteriophage of the present invention or a composition that comprises a bacteriophage of the present invention and a pharmaceutically acceptable carrier. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the containers in close confinement for commercial sale. Such means may include injection or blow-molded plastic containers into which the desired vials are retained.

The kits may include members of a phage display library, e.g., phage particles, vectors, and/or cells containing phage. The assay kits may additionally include any of the other components described herein for the practice of methods or assays of the invention. Such materials include, but are not limited to, helper phage, one or more bacterial or eukaryotic cell lines, buffers, antibiotics, labels, and the like.

In addition, the kits may optionally include instructional materials containing directions or protocols disclosing the methods described herein. While the instructional materials typically comprise written or printed materials, they are not limited to such. Any medium capable of storing such instructions and communicating them to an end user is contemplated by this invention. Such media include, but are not limited to electronic storage media, e.g., magnetic discs, tapes, cartridges, chips, and/or optical media such as CD ROMS, and the like. Such media may include addresses to internet sites that provide such instructional materials.

The kits may further comprise agents to increase stability, shelf-life, inhibit or prevent product contamination and/or increase detection rates. Useful stabilizing agents include water, saline, alcohol, glycols including polyethylene glycol, oil, polysaccharides, salts, glycerol, stabilizers, emulsifiers and combinations thereof. Useful antibacterial agents include antibiotics, bacterial-static and bacterial-toxic chemicals. Agents to optimize speed of detection may increase reaction speed such as salts and buffers.

### F. Treatment of Disease

1. Definitions

“Treatment” and “treating” as used herein refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

The term “pharmaceutically acceptable carrier” refers to a carrier that does not cause an allergic reaction or other untoward effect in subjects to whom it is administered. Suitable pharmaceutically acceptable carriers include, for example, one or more of water, saline, phosphate buffered saline, dextrose, glycerol, ethanol, or the like and combinations thereof. In addition, if desired, the vaccine can contain minor amounts of auxiliary substances such as wetting or emulsifying agents, pH buffering agents, and/or adjuvants which enhance the effectiveness of the vaccine. Examples of adjuvants that may be effective include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE and RIBI, which contains three components extracted from bacteria, monophosphoryl lipid A, trehalose dimycolate and cell wall skeleton (MPL+TDM+CWS) in a 2% squalene/Tween 80 emulsion. Other examples of adjuvants include DDA (dimethyldioctadecylammonium bromide), Freund's complete and incomplete adjuvants and QuilA. In addition, immune modulating substances such as lymphokines (e.g., IFN-.gamma., IL-2 and IL-12) or synthetic IFN-.gamma. inducers such as poly I:C can be used in combination with adjuvants described herein.

2. Non-Limiting Examples of Diseases to be Treated or Prevented

In some embodiments, the methods set forth herein pertain to methods of reducing the risk of development or progression of an infection in a subject. For example, the subject may be a subject in need of a medical device. The infection to be prevented may be, for example bacteremia, pneumonia, meningitis, osteomyelitis, endocarditis, sinusitis, arthritis, urinary tract infections, tetanus, gangrene, colitis, acute gastroenteritis, bronchitis, an abscess, an opportunistic infection, or a nosocomial infection. Examples of bacterial pathogens include Gram-positive cocci such as Staphylococcus aureus, coagulase negative staphylocci such as Staphylococcus epidermis, Streptococcus pyogenes (group A), Streptococcus spp. (viridans group), Streptococcus agalactiae (group B), S. bovis, Streptococcus (anaerobic species), Streptococcus pneumoniae, and Enterococcus spp.; Gram-negative cocci such as Neisseria gonorrhoeae, Neisseria meningitidis, and Branhamella catarrhalis; Gram-positive bacilli such as Bacillus anthracis, Corynebacterium diphtheriae and Corynebacterium species which are diptheroids (aerobic and anerobic), Listeria monocytogenes, Clostridium tetani, Clostridium difficile, Escherichia coli, Enterobacter species, Proteus mirablis and other spp., Pseudomonas aeruginosa, Klebsiella pneumoniae, Salmonella, Shigella, Serratia, and Campylobacter jejuni. The antibiotic resistant bacteria that can be killed by the antiseptic coated devices of the present invention include Staphylococci (methicillin-resistant strains), vancomycin-resistant enterococci (Enterococcus faecium), and resistant Pseudomonas aeruginosa.

Fungal infections may have cutaneous, subcutaneous, or systemic manifestations. Superficial mycoses include tinea capitis, tinea corporis, tinea pedis, onychomycosis, perionychomycosis, pityriasis versicolor, oral thrush, and other candidoses such as vaginal, respiratory tract, biliary, eosophageal, and urinary tract candidoses. Systemic mycoses include systemic and mucocutaneous candidosis, cryptococcosis, aspergillosis, mucormycosis (phycomycosis), paracoccidioidomycosis, North American blastomycosis, histoplasmosis, coccidioidomycosis, and sporotrichosis. Fungal infections include opportunistic fungal infections, particularly in immunocompromised patients such as those with AIDS. Fungal infections contribute to meningitis and pulmonary or respiratory tract diseases.

Other pathogenic organisms include dermatophytes (Microsporum canis and other M. spp.; and Trichophyton spp. such as T. rubrum, and T. mentagrophytes), yeasts (e.g., Candida albicans, C. Parapsilosis, C. glabrata, C. Tropicalis, or other Candida species including drug resistant Candida species), Torulopsis glabrata, Epidermophytonfloccosum, Malassezia fuurfur (Pityropsporon orbiculare, or P. ovale), Cryptococcus neoformans, Aspergillus fumigatus, and other Aspergillus spp., Zygomycetes (Rhizopus, Mucor), hyalohyphomycosis (Fusarium Spp.), Paracoccidioides brasiliensis, Blastomyces dermatitides, Histoplasma capsulatum, Coccidioides immitis, and Sporothrix schenckii. Other examples include Cladosporium cucumerinum, Epidermophyton floccosum, and Microspermum ypseum.

As discussed in this specification, the disease may be a disease of animals or plants. The disease may be any infection known to those of ordinary skill in the art. Non-limiting examples of animal pathogens include various pathogens, including swine influenza, avian influenza and swine hepatitis E viruses; Brucella; Coxiella burnetii; avian and feline Chlamydia psittaci; methicillin-resistant Staphlococcus aureus; and Bartonella bacteria.

3. Pharmaceutical Compositions

The present invention also concerns pharmaceutical compositions comprising a bacteriophage of the present invention. Pharmaceutical compositions according to the present invention can be prepared by admixing a quantity of a purified bacteriophage stock composition with a pharmaceutically acceptable carrier. For example, the compositions of the present invention are administered in the form of injectable compositions. A typical composition for such purpose comprises a pharmaceutically acceptable carrier. For example, the composition may contain about 10 mg of human serum albumin and from about 20 to 200 micrograms of the bacteriophage stock composition per milliliter of phosphate buffer containing NaCl. When the bacteriophage stock composition comprises sugars according to the present invention, the sugar concentration should be adapted to reach a non-toxic concentration as known to one skilled in the art. Other pharmaceutically acceptable carriers include aqueous solutions, non-toxic excipients, including salts, preservatives, buffers and the like, as described in Remington's Pharmaceutical Sciences, 15^{th Ed}. (1975) and The National Formulary XIV (1975), the contents of which are hereby incorporated by reference.

Examples of non-aqueous solvents include propylene glycol, polyethylene glycol, vegetable oil and injectable organic esters such as ethyloleate. Aqueous carriers can include water, alcoholic/aqueous solutions, saline solutions, parenteral vehicles such as sodium chloride, Ringer's dextrose, and the like. Intravenous vehicles include fluid and nutrient replenishers. Preservatives include antimicrobials, anti-oxidants, chelating agents and inert gases. The pH and exact concentration of the various components of the bacteriophage pharmaceutical compositions of the invention can be adjusted according to routine known in the art. See Goodman and Gilman's The Pharmacological Basis For Therapeutics (7^{th} Ed.).

Alternatively, the bacteriophage pharmaceutical compositions of the present invention can be in the form of liposomes, lipophilic microcapsules, dendrimers or the like for oral administration to treat systemic infections. Those skilled in the art are capable of preparing the bacteriophage compositions of the present invention in the form of a lipophilic microcapsule, a dendrimer or a liposome using conventional techniques known in the art. The skilled artisan also is capable of providing a bacteriophage composition that can be administered intranasally, rectally, transdermally, topically, or other known routes of administration of medicaments.

The compositions of the present invention can be used to treat mammals having bacterial infections, such as a cow with bovine mastitis. Suitable bacteriophage-containing compositions can be prepared that will be effective in killing, obliterating or reducing the quantity of any of the bacterial microorganisms using the guidelines set forth herein.

The compositions of the present invention preferably are administered intravenously, intranasally, orally, topically, or in any manner known to those of ordinary skill in the art in an amount and for a period of time effective to treat the disease.

The expression “treating bacterial infections,” as it is used throughout this description, denotes either (i) killing or obliterating sufficient bacterial microorganisms to render the microorganisms ineffective in infecting the host, or (ii) reducing a sufficient quantity of bacterial microorganisms so as the render the microorganisms more susceptible to treatment using conventional antibiotics. Determining an effective amount of host-specific, non-toxic purified bacteriophage composition to be administered in accordance with the present invention entails standard evaluations. An assessment in this regard would generate data concerning bioavailability, absorption, metabolism, serum and tissue levels and excretion, as well as microorganism levels, markers, and cultures. The appropriate dosage and duration of treatment can be ascertained by those skilled in the art using known techniques.

According to one embodiment, bacteriophage compositions prepared according to the present invention can be used to reduce but not entirely obliterate a population of microorganisms, thereby rendering the infectious focus more susceptible to other chemotherapeutic antibiotics and thus reducing in combination therapy duration, side effects, and risks of the latter. Thus, the bacteriophage pharmaceutical compositions of the present invention can be used in combination with known antibiotics such as aminoglycosides, cephalosporins, macrolides, erythromycin, monobactams, penicillins, quinolones, sulfonamides, tetracycline, and various anti-infective agents. Those skilled in the art can refer to the Physician's Desk Reference, (1996), or similar reference manuals for a more complete listing of known antibiotics which could be used in combination with the bacteriophage compositions.

### G. Dose and Administration

The dosage to be administered depends to a great extent on the body weight and physical condition of the subject being treated as well as the route of administration and frequency of treatment.

Administration of the therapeutic bacteriophage to a patient will follow general protocols for the administration of chemotherapeutics, taking into account the toxicity, if any, of the vector. It is anticipated that the treatment cycles would be repeated as necessary. It also is contemplated that various standard therapies, as well as surgical intervention, may be applied in combination with the described gene therapy (?).

Depending on the particular disease to be treated, administration of therapeutic compositions according to the present invention will be via any common route so long as the target tissue is available via that route in order to maximize the delivery of antigen to a site for maximum (or in some cases minimum) immune response. Administration will generally be by orthotopic, intradermal, subcutaneous, intramuscular, intraperitoneal or intravenous injection. Other areas for delivery include: oral, nasal, buccal, rectal, vaginal or topical. Topical administration would be particularly advantageous for treatment of skin disease or disease of a body surface such as mucosal surface. Such compositions would normally be administered as pharmaceutically acceptable compositions that include physiologically acceptable carriers, buffers or other excipients.

Vaccine or treatment compositions of the invention may be administered parenterally, by injection, for example, either subcutaneously or intramuscularly. Additional formulations which are suitable for other modes of administration include suppositories, and in some cases, oral formulations or formulations suitable for distribution as aerosols. In the case of the oral formulations, the manipulation of T-cell subsets employing adjuvants, antigen packaging, or the addition of individual cytokines to various formulation that result in improved oral vaccines with optimized immune responses. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25-70%.

The bacteriophage of the invention may be formulated into the vaccine or treatment compositions as neutral or salt forms. Pharmaceutically acceptable salts include the acid addition salts (formed with free amino groups of the peptide) and which are formed with inorganic acids such as, for example, hydrochloric or phosphoric acids, or with organic acids such as acetic, oxalic, tartaric, maleic, and the like. Salts formed with the free carboxyl groups can also be derived from inorganic bases such as, for example, sodium, potassium, ammonium, calcium, or ferric hydroides, and such organic bases as isopropylamine, trimethylamine, 2-ethylamino ethanol, histidine, procaine, and the like.

Vaccine or treatment compositions are administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a range from about 0.1 mg to 1000 mg, such as in the range from about 1 mg to 300 mg, and preferably in the range from about 10 mg to 50 mg. Suitable regiments for initial administration and booster shots are also variable but are typified by an initial administration followed by subsequent inoculations or other administrations. Precise amounts of active ingredient required to be administered depend on the judgment of the practitioner and may be peculiar to each subject.

The compositions can be given in a single dose schedule or in a multiple dose schedule. A multiple dose schedule is one in which a primary course of vaccination may include, e.g., 1-10 separate doses, followed by other doses given at subsequent time intervals required to maintain and or reinforce the immune response, for example, at 1-4 months for a second dose, and if needed, a subsequent dose(s) after several months. Periodic boosters at intervals of 1-5 years, usually 3 years, are desirable to maintain the desired levels of protective immunity.

Information regarding bacteriophage and their application as therapies can be found in U.S. Patent App. Pub. Nos. 20070190033, 20080026008, 20080038322, 20080057038, 20080118468, 20080124355, 20030026785, 20030180319, 20030235560, 20070248573, 20070054357, 20070154459, each of which is herein specifically incorporated by reference in its entirety. Additional information can be found in U.S. Pat. No. 7,141,241, U.S. Pat. No. 7,374,874, and U.S. Pat. No. 5,736,388, each of which is herein specifically incorporated by reference in its entirety.

### H. Secondary Treatment

Certain embodiments of the present invention provide for the administration or application of one or more secondary forms of therapies for the treatment or prevention of a disease. The secondary form of therapy may be administration of one or more secondary pharmacological agents that can be applied in the treatment or prevention of a disease. If the secondary therapy is a pharmacological agent, it may be administered prior to, concurrently, or following administration of the bacteriophage of the present invention.

The interval between the bacteriophage administration and the secondary therapy may be any interval as determined by those of ordinary skill in the art. For example, the interval may be minutes to weeks. In embodiments where the agents are separately administered, one would generally ensure that a significant period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations. In some embodiments, the timing of administration of a secondary therapeutic agent is determined based on the response of the subject to the bacteriophage of the present invention.

In particular embodiments, the secondary therapy is an antimicrobial agent. In some embodiments of the invention, the antimicrobial agent is an antibacterial agent. While any antibacterial agent may be used in the preparation of the instant antimicrobial solutions, some non-limiting exemplary antibacterial agent(s) include those classified as aminoglycosides, beta lactams, quinolones or fluoroquinolones, macrolides, sulfonamides, sulfamethaxozoles, tetracyclines, streptogramins, oxazolidinones (such as linezolid), clindamycins, lincomycins, rifamycins, glycopeptides, polymxins, lipo-peptide antibiotics, as well as pharmacologically acceptable sodium salts, pharmacologically acceptable calcium salts, pharmacologically acceptable potassium salts, lipid formulations, derivatives and/or analogs of the above.

Each of these classes of antibacterial agents have different mechanisms of action and are represented by several antibiotics a discussion of which is presented below. However, the skilled artisan will recognize that the invention is in no way limited to the agents set forth here and that these agents are described merely as examples.

The aminoglycosides are bactericidal antibiotics that bind to the 30S ribosome and inhibit bacterial protein synthesis. They are typically active against aerobic gram-negative bacilli and staphylococci. Exemplary aminoglycosides that may be used in some specific aspects of the invention include amikacin, kanamycin, gentamicin, tobramycin, or netilmicin.

Beta lactams are a class of antibacterials that inhibit bacterial cell wall synthesis. A majority of the clinically useful beta-lactams belong to either the penicillin group (penam) or cephalosporin (cephem) groups. The beta-lactams also include the carbapenems (e.g., imipenem), and monobactams (e.g., aztreonam). Inhibitors of beta-lactamase such as clavulanic acid and its derivatives are also included in this category.

Non-limiting examples of the penicillin group of antibiotics that may be used in the solutions of the present invention include amoxicillin, ampicillin, benzathine penicillin G, carbenicillin, cloxacillin, dicloxacillin, piperacillin, or ticarcillin, etc. Examples of cephalosporins include ceftiofur, ceftiofur sodium, cefazolin, cefaclor, ceftibuten, ceftizoxime, cefoperazone, cefuroxime, cefprozil, ceftazidime, cefotaxime, cefadroxil, cephalexin, cefamandole, cefepime, cefdinir, cefriaxone, cefixime, cefpodoximeproxetil, cephapirin, cefoxitin, cefotetan etc. Other examples of beta lactams include mipenem or meropenem which are extremely active parenteral antibiotics with a spectrum against almost all gram-positive and gram-negative organisms, both aerobic and anaerobic and to which Enterococci, B. fragilis, and P. aeruginosa are particularly susceptible.

Examples of beta lactamase inhibitors include clavulanate, sulbactam, or tazobactam. In some aspects of the present invention, the antibacterial solutions may comprise a combination of at least one beta lactam and at least one beta lactamase inhibitor.

Macrolide antibiotics are another class of bacteriostatic agents that bind to the 50S subunit of ribosomes and inhibit bacterial protein synthesis. These drugs are active against aerobic and anaerobic gram-positive cocci, with the exception of enterococci, and against gram-negative anaerobes. Exemplary macrolides include erythromycin, azithromycin, clarithromycin.

Quinolones and fluoroquinolones typically function by their ability to inhibit the activity of DNA gyrase. Examples include nalidixic acid, cinoxacin, trovafloxacin, ofloxacin, levofloxacin, grepafloxacin, trovafloxacin, sparfloxacin, norfloxacin, ciprofloxacin, moxifloxacin and gatifloxacin.

Sulphonamides are synthetic bacteriostatic antibiotics with a wide spectrum against most gram-positive and many gram-negative organisms. These drugs inhibit multiplication of bacteria by acting as competitive inhibitors of p-aminobenzoic acid in the folic acid metabolism cycle. Examples include mafenide, sulfisoxazole, sulfamethoxazole, and sulfadiazine.

The tetracycline group of antibiotics include tetracycline derivatives such as tigecycline which is an investigational new drug (IND), minocycline, doxycycline or demeclocycline and analogs such as anhydrotetracycline, chlorotetracycline, or epioxytetracycline. The present inventors have previously shown that minocycline has a higher penetration of the microbial biofilm layer than vancomycin and that EDTA is unique in effectively preventing and dissolving polysaccharide-rich microbial glycocalyx (U.S. Pat. No. 5,362,754).

The streptogramin class of antibacterial agents is exemplified by quinupristin, dalfopristin or the combination of two streptogramins.

Drugs of the rifamycin class typically inhibit DNA-dependent RNA polymerase, leading to suppression of RNA synthesis and have a very broad spectrum of activity against most gram-positive and gram-negative bacteria including Pseudomonas aeruginosa and Mycobacterium species. An exemplary rifamycin is rifampicin.

Other antibacterial drugs are glycopeptides such as vancomycin, teicoplanin and derivatives thereof. Yet other antibacterial drugs are the polymyxins which are exemplified by colistin.

In addition to these several other antibacterial agents such as prestinomycin, chloramphenicol, trimethoprim, fusidic acid, metronidazole, bacitracin, spectinomycin, nitrofurantion, daptomycin or other leptopeptides, oritavancin, dalbavancin, ramoplamin, ketolide etc. may be used in preparing the compositions described herein. Of these, metronidazole is active only against protozoa, such as Giardia lamblia, Entamoeba histolytica and Trichomonas vaginalis, and strictly anaerobic bacteria. Spectinomycin, is a bacteriostatic antibiotic that binds to the 30S subunit of the ribosome, thus inhibiting bacterial protein synthesis and nitrofurantoin is used orally for the treatment or prophylaxis of UTI as it is active against Escherichia coli, Klebsiella-Enterobacter species, staphylococci, and enterococci.

In other embodiments, the antimicrobial agent is an antifungal agent. Some exemplary classes of antifungal agents include imidazoles or triazoles such as clotrimazole, miconazole, ketoconazole, econazole, butoconazole, omoconazole, oxiconazole, terconazole, itraconazole, fluconazole, voriconazole (UK 109,496), posaconazole, ravuconazole or flutrimazole; the polyene antifungals such as amphotericin B, liposomal amphoterecin B, natamycin, nystatin and nystatin lipid formualtions; the cell wall active cyclic lipopeptide antifungals, including the echinocandins such as caspofungin, micafungin, anidulfungin, cilofungin; LY121019; LY303366; the allylamine group of antifungals such as terbinafine. Yet other non-limiting examples of antifungal agents include naftifine, tolnaftate, mediocidin, candicidin, trichomycin, hamycin, aurefungin, ascosin, ayfattin, azacolutin, trichomycin, levorin, heptamycin, candimycin, griseofulvin, BF-796, MTCH 24, BTG-137586, pradimicins (MNS 18184), benanomicin; ambisome; nikkomycin Z; flucytosine, or perimycin.

In still other embodiments of the invention, the antimicrobial agent is an antiviral agent. Non-limiting examples of antiviral agents include cidofovir, amantadine, rimantadine, acyclovir, gancyclovir, pencyclovir, famciclovir, foscarnet, ribavirin, or valcyclovir. In some embodiments the antimicrobial agent is an innate immune peptide or proteins. Some exemplary classes of innate peptides or proteins are transferrins, lactoferrins, defensins, phospholipases, lysozyme, cathelicidins, serprocidins, bacteriocidal permeability increasing proteins, amphipathic alpha helical peptides, and other synthetic antimicrobial proteins.

In other embodiments of the invention, the antimicrobial agent is an antiseptic agent. Several antiseptic agents are known in the art and these include a taurinamide derivative, a phenol, a quaternary ammonium surfactant, a chlorine-containing agent, a quinaldinium, a lactone, a dye, a thiosemicarbazone, a quinone, a carbamate, urea, salicylamide, carbanilide, a guanide, an amidine, an imidazoline biocide, acetic acid, benzoic acid, sorbic acid, propionic acid, boric acid, dehydroacetic acid, sulfurous acid, vanillic acid, esters of p-hydroxybenzoic acid, isopropanol, propylene glycol, benzyl alcohol, chlorobutanol, phenylethyl alcohol, 2-bromo-2-nitropropan-1,3-diol, formaldehyde, glutaraldehyde, calcium hypochlorite, potassium hypochlorite, sodium hypochlorite, iodine (in various solvents), povidone-iodine, hexamethylenetetramine, noxythiolin, 1-(3-choroallyl)-3,5,7-triazo 1-azoniaadamantane chloride, taurolidine, taurultam, N(5-nitro-2-furfurylidene)-1-amino-hydantoin, 5-nitro-2-furaldehyde semicarbazone, 3,4,4′-trichlorocarbanilide, 3,4′,5-tribromosalicylanilide, 3-trifluoromethyl-4,4′-dichlorocarbanilide, 8-hydroxyquinoline, 1-cyclopropyl-6-fluoro-1,4-dihydro-4-oxo-7-(1-piperazinyl)-3-quinolinecarboxylic acid, 1,4-dihydro-1-ethyl-6-fluoro-4-oxo-7-(1-piperazinyl)-3-quinolinecarboxylic acid, hydrogen peroxide, peracetic acid, phenol, sodium oxychlorosene, parachlorometaxylenol, 2,4,4′-trichloro-2′-hydroxydiphenol, thymol, chlorhexidine, benzalkonium chloride, cetylpyridinium chloride, silver sulfadiazine, or silver nitrate.

### H. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,736,388
U.S. Pat. No. 7,141,241
U.S. Pat. No. 7,374,874
U.S. patent Ser. No. 12/188,941
U.S. Patent Publn. 20030026785
U.S. Patent Publn. 20030180319
U.S. Patent Publn. 20030235560
U.S. Patent Publn. 20070054357
U.S. Patent Publn. 20070154459
U.S. Patent Publn. 20070190033
U.S. Patent Publn. 20070248573
U.S. Patent Publn. 20080026008
U.S. Patent Publn. 20080038322
U.S. Patent Publn. 20080057038
U.S. Patent Publn. 20080118468
U.S. Patent Publn. 20080124355
Ackermann et al., Can. J. Microbiol., 41:294-297, 1995.
Bass et al., Proteins, 8(4):309-314, 1990.
Donovan et al, J. Mol. Biol., 196:1-10, 1987.
Fauquet et al., In: Virus Taxonomy, VII^{th} Report of the International Committee on Taxonomy of Viruses. Elsevier/Academic Press, Oxford, 2005.
Gao et al, Proc. Natl. Acad. Sci. USA, 96(11):6025-6030, 1999.
Goodman And Gilman's The Pharmacological Basis For Therapeutics, 7^{th} Ed.
Greenwood et al., J. Mol. Biol., 217(2):223-227, 1991.
Han et al, Proc. Natl. Acad. Sci. USA, 92:9747-9751, 1995.
Hardies et al., Virol. J., 4:97, 2007.
Iannolo et al., J. Mol. Biol., 248:835-844, 1995.
Jespers et al., Biotechnology, 13(4):378-382, 1995.
Kishchenko et al., J. Mol. Biol., 241:208-213, 1994.
Kostyuchenko et al., Nat. Struct., Mol. Biol., 12:810-813, 2005.
Marks et al., J. Biol. Chem., 267:16007-16010, 1992.
Parmley and Smith, Gene, 73(2):305-318, 1988.
PCT Appln. WO 01/40306
PCT Appln. WO 88/06630
PCT Appln. WO 90/02809
PCT Appln. WO 91/17271
PCT Appln. WO 91/18989
PCT Appln. WO 91/19818
PCT Appln. WO 92/01047
PCT Appln. WO 92/06204
PCT Appln. WO 92/09690
PCT Appln. WO 92/18619
Perhan et al., FEMS Microbiol. Rev., 17:25-31, 1995.
Physician's Desk Reference, 50^{th} Ed., Medical Economics, 1996.
Remington's Pharmaceutical Sciences, 15^{th} Ed. Easton, Mack Publishing Co. 1405-1412 and 1461-1487, 1975.
Rodi and Makowski, Curr. Opin. Biotech., 10:87-93, 1999.
Serwer et al., Electrophoresis, 28:1896-1902, 2007a.
Serwer et al., Virology J., 4:21, 2007b.
Serwer et al., Virology, 329:412-424, 2004.
Sidhu, Bimolecular Engineering, 18:57-63, 2001.
Skerra and Pluckthun, Protein Eng., 4:971-979, 1991.
Smith and Petrenko, Chem. Rev., 97:391-410, 1997.
Smith et al., Gene, 128:37-42, 1993.
Smith, Science, 228:1315-1317, 1985.
The National Formulary XIV., 14th Ed. Washington: American Pharmaceutical Association, 1975.
Thomas et al., complete genomic sequence of Bacillus thuringiensis phage 0305φ8-36 [Gen-Bank:EF583821], 2007b.
Thomas et al., Virology, 368:405-421, 2007a.
Ward et al, J. Immunol. Meth., 189:73-82, 1996.
Willats, Plant Molec. Biol., 50:837-854, 2002.
Williamson et al., Proc. Natl. Acad. Sci. USA, 90:4141-4145, 1993.
Belyaeva, N. N. and Azizbekyan, R. R. (1968) Fine structure of new Bacillus subtilis phage AR9 with complex morphology. Virology 34, 176-179.
Desplats, C. and Krisch, H. M. (2003) The diversity and evolution of the T4-type bacteriophages. Res. Microbiol. 154, 259-267.
Eiserling, F. A. (1967) The structure of Bacillus subtilis bacteriophage PBS1. J. Ultrastruct. Res. 17, 342-347.
Hardies, S, C, Thomas, J. A. and Serwer, P. (2007) Comparative genomics of Bacillus thuringiensis phage 0305f8-36: defining patterns of descent in a novel ancient phage lineage. Virol. J. 4, 97.
Khan, S. A., Watson, R. H., Hayes, S. J. and Serwer, P. (1997) PCR-directed formation of viral hybrids in vitro. Virology 227, 409-419.
Krylov, V. N., Dela, Cruz, D. M., Hertveldt, K. and Ackermann, H. W. (2007) “fKZ-like viruses”, a proposed new genus of myovirus bacteriophages. Arch. Virol. 152, 1955-1959.
Streisinger, G., Emrich, J. and Stahl, M. (1967) Chromosome structure in phage T4. III. Terminal redundancy and length determination. Proc. Natl. Acad. Sci., USA 57, 292-295.
Sullivan, M. B., Coleman, M. L., Weigele, P., Rohwer, F. and Chisholm, S. W. (2005) Three Prochlorococcus cyanophage genomes: signature features and ecological interpretations. Plos Biology. 3, e144.
Thomas, J. A., Hardies, S. C., Rolando, M., Hayes, S. J., Lieman, K., Carroll, C. A., Weintraub, S. T. and Serwer, P. (2007) Complete genomic sequence and mass spectrometric analysis of highly diverse, atypical Bacillus thuringiensis phage 0305φ8-36. Virology 368, 405-421.
Thomas, J. A., Rolando, M. R., Carroll, C. A., Shen, P. S., Belnap, D. M., Weintraub, S. T., Serwer, P. and Hardies, S. C. (2008) Characterization of Pseudomonas chlororaphis myovirus 201f2-1 via genomic sequencing, mass specrometry and electron microscopy. Virology 376, 330-338.
Wang, J., Jiang, Y., Vincent, M., Sun, Y., Yu, H., Wang, J., Bao, Q., Kong, H. and Hu, S. (2005) Complete genome sequence of bacteriophage T5. Virology. 332, 45-65.

1. A recombinant nonpermutated bacteriophage comprising a nucleic acid sequence that is at least 150 kb in length wherein said bacteriophage displays on its surface one or more antigens.

2. The recombinant nonpermutated bacteriophage of claim 1, wherein at least one antigen is a heterologous polypeptide.

3. The recombinant nonpermutated bacteriophage of claim 1, wherein the nucleic acid sequence is between 150 kb and 500 kb in length.

4. The recombinant nonpermutated bacteriophage of claim 3, wherein the nucleic acid sequence is between 150 kb and 300 kb in length.

5. The recombinant nonpermutated bacteriophage of claim 4, wherein the nucleic acid sequence is between 150 kb and 250 kb in length.

6. The recombinant nonpermutated bacteriophage of claim 1, wherein the heterologous polypeptide is a bacterial protein, a viral protein, a fungal protein, a mammalian polypeptide, a protozoal polypeptide, or a polypeptide derived from a prion.

7. The recombinant nonpermutated bacteriophage of claim 6, wherein the heterologous polypeptide is a bacterial polypeptide.

8. The recombinant nonpermutated bacteriophage of claim 7, wherein the bacterial polypeptide is a pertussis toxin polypeptide, a filamentous hemagglutinin polypeptide, a pertactin polypeptide, a FIM2 polypeptide, a FIM3 polypeptide, a diptheria toxin polypeptide, a diptheria toxoid polypeptide, a tetanus toxin polypeptide, a tetanus toxoid polypeptide, an M protein polypeptide, a heat shock protein 65 (HSP65) polypeptide, an antigen 85A polypeptide, or a pneumolysin polypeptide.

9. The recombinant nonpermutated bacteriophage of claim 6, wherein the heterologous polypeptide is a viral polypeptide.

10. The recombinant nonpermutated bacteriophage of claim 9, wherein the viral polypeptide is a polypeptide derived from picornavirus, coronavirus, togavirus, flavirvirus, rhabdovirus, paramyxovirus, orthomyxovirus, bunyavirus, arenavirus, reovirus, retrovirus, papilomavirus, parvovirus, herpesvirus, poxvirus, hepatitis A virus, hepatitis B virus, hepatitis C virus, spongiform virus, influenza, herpes simplex virus 1, herpes simplex virus 2, measles, dengue, smallpox, polio or HIV.

11. The recombinant nonpermutated bacteriophage of claim 6, wherein the heterologous polypeptide is a polypeptide from a parasite.

12. The recombinant nonpermutated bacteriophage of claim 11, wherein the parasite is a trypanosome, a tapeworm, a roundworm, a helminth, or a malaria parasite.

13. The recombinant nonpermutated bacteriophage of claim 6, wherein the heterologous polypeptide is a fungal polypeptide.

14. The recombinant nonpermutated bacteriophage of claim 13, wherein the fungal polypeptide is a candida fungal polypeptide, a histoplasma fungal polypeptide, a cryptococcal fungal polypeptide, a coccidiodes fungal polypeptide, or a tinea fungal polypeptide.

15. The recombinant nonpermutated bacteriophage of claim 2, wherein the heterologous protein is a mammalian polypeptide.

16. The recombinant nonpermutated bacteriophage of claim 15, wherein the mammalian polypeptide is a tumor marker.

17. The recombinant nonpermutated bacteriophage of claim 1, wherein the bacteriophage comprises a nucleic acid sequence comprising a region encoding the heterologous polypeptide.

18. (canceled)

19. The recombinant nonpermutated bacteriophage of claim 1, wherein the bacteriophage is Bacillus Thuringiensis page 0305φ8-36.

20. (canceled)

21. A pharmaceutical composition comprising a recombinant nonpermutated bacteriophage of claim 1.

22. A method for inducing an immune response in a subject, comprising administering to the subject a pharmaceutically effect amount of a composition comprising a recombinant nonpermutated bacteriophage comprising a nucleic acid sequence that is at least 150 kb in length wherein said bacteriophage displays on its surface one or more antigens and a pharmaceutically acceptable carrier.

23.-49. (canceled)
